(19) United States
(12) Patent Application Publication
(10) Pub. No.: US 2025/0205157 A1
(43) Pub. Date: Jun. 26, 2025

(54) TOPICAL RUXOLITINIB FOAM

(71) Applicant: Incyte Corporation, Wilmington, DE (US)

(72) Inventors: Indushekhar PERSAUD, Wilmington, DE (US); Reinilda CATUBIG, Narre Warren South (AU); Biljana ROUGHAN, Rowville Victoria (AU); Richard BUCHTA, Wantirna South Victoria (AU); Rose YE, Ormond Victoria (AU); Steven CHAN, Clayton Victoria (AU); James LEE, Devon, PA (US)

(73) Assignee: Incyte Corporation, Wilmington, DE (US)

(21) Appl. No.: 18/956,677

(22) Filed: Nov. 22, 2024

Related U.S. Application Data
(63) Continuation-in-part of application No. 18/670,394, filed on May 21, 2024.
(60) Provisional application No. 63/503,490, filed on May 21, 2023.

(51) Int. Cl. A61K 9/12; A61K 9/00; A61K 9/107; A61K 47/22; A61P 17/00

(52) U.S. Cl. CPC A61K 9/122; A61K 9/0014; A61K 9/107; A61K 47/22; A61P 17/00

(57) ABSTRACT

The present disclosure is directed to foamable composition suitable for application as a foam to a body surface area affected by an inflammatory or autoimmune skin or hair disease in a human patient, comprising a foamable carrier component and a propellant component; wherein the foamable carrier component comprises a compound, which is ruxolitinib or deuruxolitinib, or a pharmaceutically acceptable salt of any of the aforementioned. The present disclosure is further directed to a foamable composition suitable for application as a foam to a body surface area affected by an inflammatory or autoimmune skin or hair disease in a human patient, comprising a foamable carrier component and a propellant component; wherein the foamable carrier component comprises a compound, which is ruxolitinib or deuterated ruxolitinib, or a pharmaceutically acceptable salt of any of the aforementioned, a hydroethanolic mixture, an emollient component, one or more C₁₆₋₁₈ fatty alcohols, and an emulsifier component. The present disclosure is also directed to methods of using the same.

This application is a continuation-in-part of U.S. application Ser. No. 18/670,394, filed May 21, 2024, which claims priority to U.S. Provisional Application No. 63/503,490, filed May 21, 2023, the content of each application is incorporated herein by reference in its entirety.

The present disclosure is directed to a foamable composition suitable for application as a foam to a body surface area of a human patient, comprising a foamable carrier component and a propellant component, wherein the foamable carrier component comprises a compound (i.e., an active pharmaceutical ingredient) which is ruxolitinib or deuterated ruxolitinib, or a pharmaceutically acceptable salt of any of the aforementioned, a hydroethanolic mixture, an emollient component, one or more C₁₆₋₁₈ fatty alcohols, an emulsifier component; and methods of using the same.

### BACKGROUND

In the normal cycle of hair growth, human hair follicles are continuously transformed in a cycle of organ construction and deconstruction. During anagen, which for scalp hair lasts 1 to 8 years, a pigmented hair shaft is generated. This phase of active growth consists of six stages (I through VI). Anagen is followed by catagen, a rapid, apoptosis-driven organ-involution phase that lasts several weeks, during which melanogenesis is switched off and the hair shaft is transformed into a “club hair.” The hair follicle then enters telogen, a phase of relative quiescence that varies in duration (e.g., lasting several months on the scalp), and then returns to anagen. In the disordered, shortened hair cycle in patients with alopecia areata (AA), a characteristic inflammatory-cell infiltrate attacks only (or at least primarily) pigment-producing hair follicles (predominantly those in stages III through VI of anagen). The mixed inflammatory-cell infiltrate contains T cells, mast cells, natural killer (NK) cells, and dendritic cells, among which CD8+ T cells are typically the first inflammatory cells seen to be entering the anagen hair-bulb epithelium.

Loss of immune privilege during the anagen phase allows hair follicles to be targeted by CD8+ T cells and NKG2D+ cells, followed by a marked IFN-γ response and upregulation of γ-chain cytokines (IL-15, IL-2, IL-7, and IL-21). Secretion of IL-15 by follicular epithelial cells leads to the recruitment and activation of cytotoxic T cells, which secrete IFN-γ. This causes a positive feedback loop, as IFN-γ binds to follicular epithelial cell receptors to activate JAK-STAT signaling for further secretion of IL-15. These elevated levels of cytokines at hair follicles feed the cycle of an overactive JAK-STAT signaling cascade causing inflammation and hair loss.

Alopecia areata (AA) is a common condition and generally involves the scalp but can also affect any body area. For example, hair loss may be patches across the scalp (most common). The US Food and Drug Administration recently approved baricitinib for treatment for AA. There, however, are several non-FDA approved therapies still in use and still a need for useful treatments for AA. For example, intralesional steroid injections demonstrate strong efficacy and durability for patients with limited patches but are infeasible for treating patients with extensive hair loss (i.e., >50% hair loss). There are other non-FDA approved therapies that are used intermittently to treat patients such as topical immunomodulators, with varying levels of success. As such, there continues to be an unmet need for treating alopecia areata with a topical composition.

Ruxolitinib (INCB018424) is a potent JAK1/JAK2 inhibitor, which has previously been described in U.S. Pat. No. 7,598,257, which is incorporated herein by reference in its entirety. Ruxolitinib phosphate was previously described in U.S. Pat. No. 8,722,693, which is incorporated herein by reference in its entirety. Ruxolitinib is approved in the US as an oral dosage form (JAKAFI®) for treatment of myelofibrosis, polycythemia vera, and acute and chronic GVHD, as well as a topical skin cream (OPZELURAR) for treatment of atopic dermatitis and vitiligo.

Ruxolitinib was previously shown to be effective in a mouse model in treating alopecia areata (Xing, et al., “Alopecia areata is driven by cytotoxic T lymphocytes and is reversed by JAK inhibition”, Nat Med, 1043-1049 (2014)). A well-established graft model of AA was used in which skin grafts from mice with spontaneous AA are transferred onto the backs of unaffected 10-week-old recipient C₃H/HeJ mice. In this model, AA develops reliably in 95-100% of grafted recipients within 6-10 weeks. When administered systemically at the time of grafting, ruxolitinib was shown to prevent the development of AA. Further, complete hair regrowth was observed in grafted mice with long-standing AA (more than 8 weeks) after being treated once daily for 12 weeks to affected skin on the dorsal back with ruxolitinib in 1:10 DMSO:Aquaphor mixture (0.5% ruxolitinib).

A later Phase 2a study of ruxolitinib cream was not shown to have a significant effect on AA. The study was conducted in AA patients with 25%-99% hair loss using ruxolitinib cream at a 1.5% strength dosed BID and evaluated for 24 weeks (https://clinicaltrials.gov/ct2/show/NCT02553330; Elise A. Olsen, et al., “Ruxolitinib cream for the treatment of patients with alopecia areata: A 2-part, double blinded, randomized vehicle-controlled phase 2 study”, J. Am. Acad. Dematol., 52:412-9 (2020)). The authors, however, concluded that the topical 1.5% ruxolitinib cream in AA did not show “a significant effect.” In contrast to topical application of a ruxolitinib cream, deuterated ruxolitinib administered orally has been shown to be efficacious for treatment of alopecia. In Phase 3 studies, oral dosage forms of deuruxolitinib (CTP-543; (3R)-3-(2,2,3,3,4,4,5,5-D8) cyclopentyl-3-[4-(7H-pyrrolo[2,3-d]pyrimidin-4-yl)-1H-pyrazol-1-yl]propanenitrile) met the clinical endpoint when administered at a dose of 8 mg and 12 mg BID orally (https://ir.concertpharma.com/news-releases/news-release-details/concert-pharmaceuticals-announces-presentation-ctp-543-thrive #; https://ir.concertpharma.com/news-releases/news-release-details/late-breaking-phase-3-data-aad-2023-show-oral-investigational).

The present application addresses the need for a formulation that can topically deliver ruxolitinib or deuterated ruxolitinib to the hair follicles to treat an inflammatory or autoimmune skin or hair disease, for example, alopecia. The inflammatory or autoimmune skin or hair disease is alopecia, a scalp condition, or a skin disease. The scalp condition is frontal fibrosing alopecia, lichen planopilaris, chronic cutaneous lupus erythematosus, or folliculitis decalvans. The skin disease is lichen planus (LP), hidradenitis suppurativa (HS), lichen sclerosus (LS), prurigo nodularis (PN), atopic dermatitis (AD), vitiligo, or psoriasis.

### BRIEF DESCRIPTION OF DRAWING(S)

FIG. 1 graphically illustrates a manufacturing process embodiment directed to the topical foam composition of the present disclosure.

FIG. 2 graphically illustrated a Phase 2, randomized, double-blind, placebo-controlled, dose-ranging study of the efficacy and safety of rux foam or deuterated rux foam in participants with mild to moderate alopecia areata (“rux foam” means ruxolitinib phosphate containing foam; “deuterated rux foam” means deuruxolitinib phosphate containing foam).

FIG. 3 graphically illustrates a Phase 2, randomized, double-blind, placebo-controlled, study of the efficacy and safety of adjunctive rux foam with deuterated oral rux in participants with severe alopecia areata (“deuterated oral rux” means deuruxolitinib phosphate on a free base basis; “rux foam” means ruxolitinib phosphate containing foam).

FIG. 4A are pictures of the foam appearances of formulations, 268-9-05, 268-9-09, and 268-9-10 at time 0 to 2 minutes; FIG. 4B are pictures of the foam appearances of formulations, 268-9-05, 268-9-09, and 268-9-10 at time 32 minutes.

FIGS. 5A and 5B are pictures of the foam appearances of the 268-10-01 and the 268-10-03 formulation, respectively.

FIGS. 6A and 6B are pictures of F268-16-06 Lot 268-19-01 with 4.18% Gas P75 in front and rear view, respectively.

FIGS. 7A and 7B are pictures of F268-16-12 Lot 268-19-03, 1% emollient & Transcutol-P foams, 3.5% and 4.0% P75 propellant, respectively.

FIGS. 8A and 8B shows the appearance of the foamable compositions of F/L 268-20-02 and F/L 268-20-01 from both the front and back views.

FIG. 9 is a bar graph of results from several formulations with 1.5% ruxolitinib illustrating the mean cumulative amount (ng) of ruxolitinib recovered from the epidermis.

FIG. 10 is a bar graph of results from several formulations with 2.5% ruxolitinib illustrating the mean cumulative amount (ng) of ruxolitinib recovered from the epidermis.

FIG. 11 is a bar graph of results from several formulation with 1.5% ruxolitinib illustrating the mean cumulative amount (ng) of ruxolitinib recovered from the dermis.

FIG. 12 is a bar graph of results from several formulation with 2.5% ruxolitinib illustrating the mean cumulative amount (ng) of ruxolitinib recovered from the dermis.

FIG. 13 is a bar graph of results from several formulations with 1.5% deuruxolitinib illustrating the mean cumulative amount (ng) of ruxolitinib recovered from the epidermis.

FIG. 14 is a bar graph of results from several formulations with 2.5% deuruxolitinib illustrating the mean cumulative amount (ng) of ruxolitinib recovered from the epidermis.

FIG. 15 is a bar graph of results from several formulation with 1.5% deuruxolitinib illustrating the mean cumulative amount (ng) of ruxolitinib recovered from the dermis.

FIG. 16 is a bar graph of results from several formulation with 2.5% deuruxolitinib illustrating the mean cumulative amount (ng) of ruxolitinib recovered from the dermis.

FIG. 17 is a representative image of Ki-67 expression in hair follicles.

FIGS. 18A and 18B show the expression in hair matrix, fold change to normal non-lesional skin.

FIG. 19 is a representative image of MHC-1 expression.

FIGS. 20A and 20B show the MHC-1 expression in the proximal outer root sheath.

FIGS. 21A and 21B show the MHC-1 expression in the germinative hair matrix.

FIGS. 22A (Donor 1-1) and 22B (Donor 4-2) graphically illustrate the melanin clumping, as the number of melanin clumps per hair follicle for the vehicle non-lesional and lesional and AP-1 and AP-2 non-lesional and lesional.

FIGS. 23-A-23C (Donor 1-1) and 23D-23F (Donor 4-2) graphically illustrate the hair matrix keratinocyte proliferation (anagen hair follicles (HFs)), the hair matrix keratinocyte proliferation (anagen and catagen HFs), and the hair matrix keratinocyte apoptosis (anagen and catagen HFs). Note: ** indicates p<0.01

FIGS. 24A (Donor 1-1) and 24A (Donor 4-2) graphically illustrate the macroscopic hair cycle staging as the % of hair follicles in each stage.

FIGS. 25A-D (Donor 1-1) and 25E-H (Donor 4-2) graphically illustrate the MHC-I expression shown in the proximal outer root sheath, the germinative hair matrix, the dermal cup, and the dermal papilla stalk, respectively for each donor and only for anagen hair follicles (HFs). Note: * indicates p<0.05 and ** indicates p<0.01

FIGS. 26A-D (Donor 1-1) and 26E-H (Donor 4-2) graphically illustrate the MHC-II expression in the proximal outer root sheath, the germinative hair matrix, the dermal cup, and the dermal papilla stalk, respectively for each donor and only for anagen hair follicles (HFs).

FIGS. 27A and 27 B (Donor 1-1) and 27C and 27D (Donor 4-2) graphically illustrate CD8+ cells in the epithelium and in the CTS and perifollicular tissue. Note: * indicates p<0.01

FIGS. 28A-C (Donor 1-1) and 28D-F (Donor 4-2) graphically illustrate CD8/CD69 double positive cells in the CTS and perifollicular tissue, CD69 positive cells and perifollicular tissue, and the percentage of CD8/CD69 double positive cells amongst CD8+ cells in the CTS and perifollicular tissue. Note: * indicates p<0.05

FIGS. 29A-C (Donor 1-1) and 29D-F (Donor 4-2) graphically illustrate the CD8/NKG2D double positive in the CTS and perifollicular tissue, NKG2D positive cells in the CTS and perifollicular tissue, and the percentage of CD8/NKG2D double positive cells amongst CD8+ cells in the CTS and perifollicular tissues. Note: * indicates p<0.05

FIGS. 30A and 30 B (API-1, chronic donor) and 30C and 30D (API-2, chronic donor) graphically illustrate CD3+ cells in the epithelium and CD8+ cells in the epithelium. Note: * indicates p<0.05

FIGS. 31A and 31B (API-1, chronic donor) and 31C and 31D (API-2, chronic donor) graphically illustrate CD3+ cells in the CTS and perifollicular tissue and CD8+ cells in the CTS and perifollicular tissue. Note: * indicates p<0.05

FIGS. 32A (API-1, chronic donor) and 32B and 32C (API-2, chronic donor) graphically illustrate CD3/Ki-67 double positive cells in the epithelium.

FIGS. 33A and 33B (API-1, chronic donor) and 33C and 33D (API-2, chronic donor) graphically illustrate CD3/Ki-67 double positive cells in the CTS and perifollicular tissues, and CD3/CD8/Ki-67 triple positive cells in the CTS and perifollicular tissue.

FIGS. 34A-34C (API-1, chronic donor) and 34D-34F (API-2, chronic donor) graphically illustrate CD3/CD69 double positive cells in the epithelium, CD69 positive cells in the epithelium, and the percentage of CD3/CD69 double positive cells amongst CD3+ cells in the epithelium.

FIGS. 35A-35C (API-1, chronic donor) and 35D-35F (API-2, chronic donor) graphically illustrate CD3/CD69 double positive cells in the CTS and perifollicular tissue, CD69 positive cells in the CTS and perifollicular tissue, and the percentage of CD3/CD69 double positive cells amongst CD3+ cells in the CTS and perifollicular tissue.

FIGS. 36A-36C (API-1, chronic donor) and 36D-36F (API-2, chronic donor) graphically illustrate CD8/NKG2D double positive cells in the CTS and perifollicular tissue, NKG2D positive cells in the CTS and perifollicular tissue, and the percentage of CD8/NKG2D double positive cells amongst CD8+ cells in the CTS and perifollicular tissue. Note: * indicates p<0.05

FIGS. 37A (API-1, chronic donor) and 37B (API-2, chronic donor) graphically illustrate CD3/FoxP3 double positive cells in the dermis. Note: * indicates p<0.05

FIG. 38 is a graphical heatmap showing the average fold change of each cytokine secretion into the supernatant after 48 h and 96 h treatment with API-1 or API-2, compared to the vehicle-treated control in lesional skin from acute (Donor 1-1; Donor 4-2) and chronic (Donor 2-1, 6-1, 3-2, 5-2) AA donors.

FIG. 39A graphically shows IFN-γ production, FIG. 39B graphically shows MDC (CCL22) production, FIG. 39C graphically shows IP-10 (CXCL10) production, and FIG. 39D graphically shows IL-17A production were examined at 48 hours and 96 hours.

FIG. 40 is a graphical heatmap showing the average fold change of each cytokine secretion into the supernatant after 48 h and 96 h treatment with API-1 or API-2, compared to the vehicle-treated control in lesional skin from acute (Donor 1-1; Donor 4-2) and chronic (Donor 2-1, 6-1, 3-2, 5-2) AA donors.

FIGS. 41A-41C graphically illustrate Rantes (CCL-5), MP-1a, and MIP-1B (CCL4) productions, respectively.

FIG. 42 is a graphical heatmap showing the average fold change of each cytokine secretion into the supernatant after 48 h and 96 h treatment with API-1 or API-2, compared to the vehicle-treated control in lesional skin from acute (Donor 1-1; Donor 4-2) and chronic (Donor 2-1, 6-1, 3-2, 5-2) AA donors.

FIGS. 43A, 43B, and 43C graphically illustrate IL-10, MCP-1 (CCL2), and MCP-3 (CC7) productions, respectively, with API-1 and API-2.

### SUMMARY

The present disclosure is directed to a foamable composition suitable for application as a foam to a body surface area affected by an inflammatory or autoimmune skin or hair disease in a human patient, comprising a foamable carrier component and a propellant component, wherein the foamable carrier component comprises: a compound, which is ruxolitinib or deuterated ruxolitinib (e.g., deuruxolitinib), or a pharmaceutically acceptable salt thereof, a hydroethanolic mixture, an emollient component, one or more C₁₆₋₁₈ fatty alcohols, and an emulsifier component, wherein the hydroethanolic mixture is a mixture of ethanol and water. In some embodiments, the foamable composition and the foamable carrier composition do not comprise an organic amine pH adjusting agent.

In some embodiments, the hydroethanolic mixture comprises about 65% to about 99% by weight of the foamable carrier component; the ethanol comprises about 40% to about 90% by weight of the hydroethanolic mixture; the emulsifier component is present in an amount ranging from about 0.25% to about 5% by weight of the foamable carrier component; the emollient component is present in an amount of from about 0.1% to about 4% by weight of the foamable carrier component; the one or more C₁₆₋₁₈ fatty alcohols is present in an amount ranging from about 0.5% to about 10% by weight of the foamable carrier component; and the compound is present in an amount from about 0.5% to about 5% by weight of the foamable carrier component on a free base basis.

The present disclosure is also directed to a foam produced by expelling the foamable composition as described herein for application as a foam to a body surface area affected by an inflammatory or autoimmune skin or hair disease in a human patient.

The present disclosure is also directed to methods for treating an inflammatory or autoimmune skin or hair disease in a human patient in need thereof comprising administering to a body surface area affected by the disease of the patient the foam as described herein.

In some embodiments, the inflammatory or autoimmune skin or hair disease is alopecia.

In some embodiments, the inflammatory or autoimmune skin or hair disease is a scalp condition, and the scalp condition is frontal fibrosing alopecia, lichen planopilaris, chronic cutaneous lupus erythematosus, or folliculitis decalvans.

In some embodiments, the inflammatory or autoimmune skin or hair disease is a skin disease and the skin disease is lichen planus (LP), hidradenitis suppurativa (HS), lichen sclerosus (LS), prurigo nodularis (PN), atopic dermatitis (AD), vitiligo, or psoriasis.

Additionally, the present disclosure is directed to methods of inducing hair growth in a human patient suffering from alopecia, comprising administering to a body surface area affected by the alopecia of the patient the foam as described herein.

The present disclosure is directed to a foamable composition suitable for application as a foam to a body surface area affected by alopecia in a human patient, comprising a foamable carrier component and a propellant component, wherein the foamable carrier component comprises a compound (i.e., an active pharmaceutical ingredient) which is ruxolitinib or deuterated ruxolitinib, or a pharmaceutically acceptable salt of any of the aforementioned

The present disclosure is also directed to a foam suitable for application to a body surface area affected by alopecia in a human patient, comprising a compound, which is ruxolitinib or deuterated ruxolitinib, or a pharmaceutically acceptable salt of any of the aforementioned.

The present disclosure is further directed to a foam produced by expelling any of the foamable compositions described herein from a pressurized container. In some embodiments, the foamable composition is aerosolized.

The present disclosure is further directed to a foamable carrier component, comprising a compound, which is ruxolitinib or deuterated ruxolitinib, or a pharmaceutically acceptable salt of any of the aforementioned.

The present disclosure is also directed to methods of treating alopecia in a patient in need thereof, comprising administering a foam as described herein to the patient.

The present disclosure is further directed to use of a foam as described herein for preparation of a medicament for use in treatment of alopecia.

The present disclosure is further directed to use of a foamable composition as described herein for preparation of a medicament for use in treatment of alopecia.

The present disclosure is also directed to a foam as described herein for use in treatment of alopecia.

The present disclosure is also directed to a foamable composition as described herein for use in treatment of alopecia.

A foamable composition suitable for application as a foam to a body surface area affected by alopecia areata in a human patient, comprising a foamable carrier component and a propellant component;
wherein the foamable carrier component comprises a compound, which is ruxolitinib or deuterated ruxolitinib, or a pharmaceutically acceptable salt of any of the aforementioned;
wherein the body surface area is the patient's scalp; and
wherein the foamable composition does not comprise an organic amine pH adjusting agent.

A foamable composition suitable for application as a foam to a body surface area affected by alopecia in a human patient, comprising a foamable carrier component and a propellant component;
wherein the foamable carrier component comprises a compound, which is ruxolitinib or deuterated ruxolitinib, or a pharmaceutically acceptable salt of any of the aforementioned; and
wherein the foamable carrier component further comprises:
from about 40% to about 65% of ethanol by weight of the foamable carrier component;
from about 30% to about 60% of water by weight of the foamable carrier component;
from about 0.5% to about 5% of stearyl alcohol by weight of the foamable carrier component;
from about 0.5% to about 5% of cetyl alcohol by weight of the foamable carrier component; and
from about 2% to about 20% of propylene glycol.

A foamable composition suitable for application as a foam to a body surface area affected by alopecia in a human patient, comprising a foamable carrier component and a propellant component;
wherein the foamable carrier component comprises a compound, which is ruxolitinib or deuterated ruxolitinib, or a pharmaceutically acceptable salt of any of the aforementioned; and
wherein the foamable carrier component further comprises water, a solvent component, and an oil phase;
wherein the oil phase comprises about 0.5% to about 20% by weight of the foamable carrier composition;
wherein the oil phase comprises at least one fatty alcohol, wherein the at least one fatty alcohol is cetyl alcohol or stearyl alcohol, or a mixture thereof;
wherein the solvent component comprises from about 30% to about 95% by weight of the foamable carrier component,
wherein the solvent component comprises ethanol;
wherein the solvent component comprises polyethylene glycol;
wherein the solvent component comprises PEG200 or PEG300;
the water comprises from about 20% to about 60% by weight of the foamable carrier component; and
wherein the propellant component comprises from about 2% to about 20% of the foamable composition.

In some of the preceding embodiments, the alopecia is alopecia areata (e.g., more preferably patchy alopecia areata). In a further embodiment, the alopecia areata is acute. In a further embodiment, the alopecia areata is chronic. In a further embodiment, the disclosure pertains to a foam produced by expelling the foamable composition of the preceding embodiments from a pressurized container. In a still further embodiment, the disclosure pertains to a method for treating alopecia (e.g., preferably alopecia areata, or more preferably patchy alopecia areata) in a human patient in need thereof comprising administering to a body surface area affected by alopecia of the patient a foam produced from the foamable compositions of the preceding embodiments.

In some further embodiments, the present disclosure is directed to a foamable composition suitable for application as a foam to a body surface area affected by an inflammatory or autoimmune skin or hair disease in a human patient, comprising a foamable carrier component and a propellant component, wherein the foamable carrier component comprises: from about 0.5% to about 3%, of a compound, which is ruxolitinib or deuruxolitinib, or a pharmaceutically acceptable salt thereof, by weight of the foamable carrier composition, from about 80% to about 90% of a hydroethanolic mixture, by weight of the foamable carrier composition, from about 1% to about 3% of an emollient component, by weight of the foamable carrier composition, wherein the emollient component comprises at least one emollient and at least one co-solvent, from about 1% to about 5% of one or more C₁₆₋₁₈ fatty alcohols, by weight of the foamable carrier composition, from about 0.5% to about 3% of an emulsifier component, by weight of the foamable carrier composition, and from about 4% to about 6% of a solvent, by weight of the foamable carrier composition; wherein the hydroethanolic mixture is a mixture of ethanol and water, the ethanol is present in amount ranging from about 50% to about 70% of the hydroethanolic mixture, and the water is present in an amount ranging from about 30% to about 50% of the hydroethanolic mixture.

In some embodiments of the foamable composition, the emulsifier component is polysorbate 60 (Tween 60); the emollient is glycerin and the co-solvent is polyethylene glycol 300; the one or more C₁₆₋₁₈ fatty alcohols are cetyl alcohol and stearyl alcohol; the solvent is propylene glycol; and the compound is ruxolitinib phosphate in an amount of about 2.5% on a free base basis, of the foamable carrier component.

In some embodiments of the foamable composition, the emulsifier component is polysorbate 60 (Tween 60); the emollient is glycerin and the co-solvent is polyethylene glycol 300; the one or more C₁₆₋₁₈ fatty alcohols are cetyl alcohol and stearyl alcohol; the solvent is propylene glycol; and the compound is ruxolitinib phosphate in an amount of about 1.5% on a free base basis, of the foamable carrier component.

In some embodiments of the foamable composition, the emulsifier component is polysorbate 60 (Tween 60); the emollient is glycerin and the co-solvent is polyethylene glycol 300; the one or more C₁₆₋₁₈ fatty alcohols are cetyl alcohol and stearyl alcohol; the solvent is propylene glycol; and the compound is deuruxolitinib phosphate in an amount of about 2.5% on a free base basis, of the foamable carrier component.

In some embodiments of the foamable composition: the emulsifier component is polysorbate 60 (Tween 60); the emollient is glycerin and the co-solvent is polyethylene glycol 300; the one or more C₁₆₋₁₈ fatty alcohols are cetyl alcohol and stearyl alcohol; the solvent is propylene glycol; and the compound is deuruxolitinib phosphate in an amount of about 1.5% on a free base basis, of the foamable carrier component.

In some embodiments of the foamable composition, the emulsifier component is polysorbate 60 (Tween 60); the emollient is myristyl lactate and the co-solvent is transcutol-P; the one or more C₁₆₋₁₈ fatty alcohols are cetyl alcohol and stearyl alcohol; the solvent is propylene glycol; and the compound is ruxolitinib phosphate in an amount of about 2.5% on a free base basis, of the foamable carrier component.

In some embodiments of the foamable composition, the emulsifier component is polysorbate 60 (Tween 60); the emollient is myristyl lactate and the co-solvent is transcutol-P; the one or more C₁₆₋₁₈ fatty alcohols are cetyl alcohol and stearyl alcohol; the solvent is propylene glycol; and the compound is ruxolitinib phosphate in an amount of about 1.5% on a free base basis, of the foamable carrier component.

In some embodiments of the foamable composition, the emulsifier component is polysorbate 60 (Tween 60); the emollient is myristyl lactate and the co-solvent is transcutol-P; the one or more C₁₆₋₁₈ fatty alcohols are cetyl alcohol and stearyl alcohol; the solvent is propylene glycol; and the compound is deuruxolitinib phosphate in an amount of about 2.5% on a free base basis, of the foamable carrier component.

In some embodiments of the foamable composition, the emulsifier component is polysorbate 60 (Tween 60); the emollient is myristyl lactate and the co-solvent is transcutol-P; the one or more C₁₆₋₁₈ fatty alcohols are cetyl alcohol and stearyl alcohol; the solvent is propylene glycol; and the compound is deuruxolitinib phosphate in an amount of about 1.5% on a free base basis, of the foamable carrier component.

In some embodiments of the foamable composition, the compound or the salt is present in an amount of 1.5% or 2.5%, by weight of the foamable carrier composition, the hydroethanolic mixture is present in an amount from about 80% to about 90%, by weight of the foamable carrier composition, the emollient component is present in an amount from about 2% to about 3%, by weight of the foamable carrier composition, wherein the emollient component comprises about 0.3% to about 0.6% of an emollient and about 1.8% to about 2.2% of a co-solvent, by weight of the foamable carrier composition, the one or more C₁₆₋₁₈ fatty alcohols are cetyl alcohol and stearyl alcohol, the cetyl alcohol is present in an amount from about 2% to about 2.5%, by weight of the foamable carrier composition, the stearyl alcohol is present in an amount from about 0.25% to about 0.5%, by weight of the foamable carrier composition, and the emulsifier component is present in an amount from about 1% to about 2%, by weight of the foamable carrier composition, the solvent is present in an amount from about 4% to about 6%, by weight of the foamable carrier composition, wherein the hydroethanolic mixture is a mixture of ethanol and water, the ethanol is present in amount ranging from about 55% to about 65% of the hydroethanolic mixture, and the water is present in an amount ranging from about 35% to about 45% of the hydroethanolic mixture.

In some embodiments of the foamable composition, the emulsifier component is polysorbate 60 (Tween 60); the emollient is glycerin and the co-solvent is polyethylene glycol 300, the solvent is propylene glycol; and the compound is ruxolitinib phosphate in an amount of about 2.5% on a free base basis, of the foamable carrier component.

In some embodiments of the foamable composition, the emulsifier component is polysorbate 60 (Tween 60); the emollient is glycerin and the co-solvent is polyethylene glycol 300; the solvent is propylene glycol; and the compound is ruxolitinib phosphate in an amount of about 1.5% on a free base basis, of the foamable carrier component.

In some embodiments of the foamable composition, the emulsifier component is polysorbate 60 (Tween 60); the emollient is glycerin and the co-solvent is polyethylene glycol 300; the solvent is propylene glycol; and he compound is deuruxolitinib phosphate in an amount of about 2.5% on a free base basis, of the foamable carrier component.

In some embodiments of the foamable composition, the emulsifier component is polysorbate 60 (Tween 60); the emollient is glycerin and the co-solvent is polyethylene glycol 300; the solvent is propylene glycol; and the compound is deuruxolitinib phosphate in an amount of about 1.5% on a free base basis, of the foamable carrier component.

In some embodiments of the foamable composition, the compound or the salt is present in an amount of 1.5% or 2.5%, by weight of the foamable carrier composition, the hydroethanolic mixture is present in an amount from about 80% to about 90%, by weight of the foamable carrier composition, the emollient component is present in an amount from about 1% to about 2%, by weight of the foamable carrier composition, wherein the emollient component comprises about 0.8% to about 1.2% of an emollient and about 0.3% to about 0.6% of a co-solvent, by weight of the foamable carrier composition, the one or more C₁₆₋₁₈ fatty alcohols are cetyl alcohol and stearyl alcohol; the cetyl alcohol is present in an amount from about 2% to about 2.5%, by weight of the foamable carrier composition, the stearyl alcohol is present in an amount of from about 0.6% to about 0.9%, by weight of the foamable carrier composition, the emulsifier component is present in an amount from about 1% to about 2%, by weight of the foamable carrier composition, and the solvent is present in an amount from about 4% to about 6%, by weight of the foamable carrier composition, wherein the hydroethanolic mixture is a mixture of ethanol and water, the ethanol is present in amount ranging from about 55% to about 65% of the hydroethanolic mixture, and the water is present in an amount ranging from about 35% to about 45% of the hydroethanolic mixture.

In some embodiments of the foamable composition, the emulsifier component is polysorbate 60 (Tween 60); the emollient is myristyl lactate and the co-solvent is transcutol-P, the solvent is propylene glycol; and the compound is ruxolitinib phosphate in an amount of about 2.5% on a free base basis, of the foamable carrier component.

In some embodiments of the foamable composition, the emulsifier component is polysorbate 60 (Tween 60); the emollient is myristyl lactate and the co-solvent is transcutol-P; the solvent is propylene glycol; and the compound is ruxolitinib phosphate in an amount of about 1.5% on a free base basis, of the foamable carrier component.

In some embodiments of the foamable composition, the emulsifier component is polysorbate 60 (Tween 60); the emollient is myristyl lactate and the co-solvent is transcutol-P; the solvent is propylene glycol; and the compound is deuruxolitinib phosphate in an amount of about 2.5% on a free base basis, of the foamable carrier component.

In some embodiments of the foamable composition, the emulsifier component is polysorbate 60 (Tween 60); the emollient is myristyl lactate and the co-solvent is transcutol-P, the solvent is propylene glycol; and the compound is deuruxolitinib phosphate in an amount of about 1.5% on a free base basis, of the foamable carrier component.

In some embodiments of the foamable composition, the emollient component comprises PEG 300 in an amount of about 2% and glycerin in an amount of about 0.5% by weight of the foamable carrier component.

In some embodiments of the foamable composition, the emollient component comprises myristyl lactate in an amount of about 1%, by weight of the foamable carrier component and transcutol-P in an amount of about 0.5% by weight of the foamable carrier component.

In some embodiments of the foamable composition, the pH of the foamable composition ranges from about 5.0 to about 8.0. In some embodiments of the foamable composition, the pH of the foamable composition ranges from about 5.0 to about 6.0. In some embodiments, the pH is adjusted by addition of trolamine to the foamable carrier composition.

In some embodiments, the present disclosure is directed to a foam produced by expelling the foamable composition from a pressurized container.

In some embodiments of the foam, the foamable composition is aerosolized.

In some embodiment, the present disclosure is directed to a method for treating an inflammatory or autoimmune skin or hair disease in a human patient in need thereof comprising administering to a body surface area affected by the disease of the patient a foam. In some embodiments, the inflammatory or autoimmune skin or hair disease is alopecia. In some embodiments, the alopecia is alopecia areata. In some embodiments, the alopecia areata is mild to moderate. In some embodiments, the alopecia areata is severe. In some embodiments, the alopecia areata is acute. In some embodiments, the alopecia areata is chronic. In some embodiments, the inflammatory or autoimmune skin or hair disease is seborrheic dermatitis.

### DESCRIPTION

### Definitions

As used herein, “a body surface area affected by an inflammatory or autoimmune skin or hair disease” or “a body surface area affected by alopecia” refers to an area of the patient's skin or scalp having hair loss from the inflammatory or autoimmune skin or hair disease, such as alopecia (e.g., alopecia areata) as described herein.

As used herein, “ruxolitinib phosphate” means the phosphoric acid salt of ruxolitinib, wherein the ruxolitinib and phosphoric acid are in a 1:1 ratio.

As used herein, “deuterated ruxolitinib” means ruxolitinib, wherein one or more hydrogen atoms of the ruxolitinib are replaced by deuterium atoms. Deuruxolitinib is a compound which is a deuterated ruxolitinib. Deuruxolitinib is also CTP-543 or (3R)-3-(2,2,3,3,4,4,5,5-D8) cyclopentyl-3-[4-(7H-pyrrolo[2,3-d]pyrimidin-4-yl)-1H-pyrazol-1-yl]propanenitrile. In each embodiment or claim reciting “deuterated ruxolitinib”, the embodiment also provides support for an embodiment that recites “deuruxolitinib” specifically.

As used herein, “deuruxolitinb phosphate” means the phosphoric acid salt of deuruxolitinib, wherein the deuruxolitinib and phosphoric acid are in a 1:1 ratio.

As used herein, “foamable composition” means a composition that forms a foam when expelled from a pressurized container containing a propellant component.

As used herein, “foamable carrier component” means a composition that is combined with a propellant component in a pressurized container to form a foamable composition.

As used herein, an “alkanol amine” is an HO—(C₂₋₆ alkyl) n amine, wherein n is 1, 2, or 3 and the C₂₋₆ alkyl groups are independently selected and can be branched or straight chain alkyl groups.

As used herein, “topical formulation,” “pharmaceutical composition,” or “pharmaceutical formulation” are used interchangeably and refer to compositions, and/or dosage forms, which are, within the scope of sound medical judgment, suitable for use in contact with tissues of humans and animals. As used herein, “statistically significant” means a p-value of <0.05 (such as <0.001, and further for example, <0.0001).

As used herein, the phrase “pharmaceutically acceptable” means those compounds, materials, compositions, and/or dosage forms, which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

The presently claimed subject matter also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the presently claimed subject matter include the conventional non-toxic salts of the parent compound formed, for example, from non-toxic inorganic or organic acids. The pharmaceutically acceptable salts of the presently claimed subject matter can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. In some embodiments, the pharmaceutically acceptable salt is a phosphoric acid salt, a sulfuric acid salt, or a maleic acid salt.

As used herein, the term “emulsifier component” refers, in one aspect, to a substance, or mixtures of substances that maintains an element or particle in suspension within a fluid medium. In some embodiments, the emulsifier component allows an oil phase to form an emulsion when combined with water. In some embodiments, the emulsifier component refers to one or more non-ionic surfactants.

As used herein, the term “occlusive agent component” refers to a hydrophobic agent or mixtures of hydrophobic agents that form an occlusive film on skin that reduces transepidermal water loss (TEWL) by preventing evaporation of water from the stratum corneum.

As used herein, the term “stiffening agent component” refers to a substance or mixture of substances that increases the viscosity and/or consistency of the cream or improves the rheology of the cream.

As used herein, the term “emollient component” refers to an agent that softens or soothes the skin or soothes an irritated internal surface.

As used herein, the term “stabilizing agent component” refers to a substance or mixture of substances that improves the stability of the cream and/or the compatibility of the components in the cram.

As used herein, the term “solvent component” is a liquid substance or mixture of liquid substances capable of dissolving ruxolitinib, deuterated ruxolitinib, or a pharmaceutically acceptable salt thereof, or other substances in the cream. In some embodiments, the solvent component is a liquid substance or mixture of liquid substances in which, ruxolitinib, deuterated ruxolitinib, or a pharmaceutically acceptable salt thereof, has reasonable solubility. For example, a solvent is a substance or mixture thereof, in which ruxolitinib, deuterated ruxolitinib, or a pharmaceutically acceptable salt thereof (whichever is used), has a solubility of at least about. 5% or greater, 1% or greater, 10 mg/mL or greater, at least about 15 mg/mL or greater, or at least about 20 mg/mL or greater.

As used herein, the term “co-solvent component” is one or more substances that is capable of stabilizing or dissolving one or more excipients (e.g., emollient component) or active pharmaceutical substances (e.g., ruxolitinib, deuruxolitinib, or a pharmaceutically acceptable salt thereof) in the foamable carrier component and/or foamable composition.

As used herein, the phrase “antimicrobial preservative component” is a substance or mixtures of substances, which inhibits microbial growth in the cream.

As used herein, the phrase “chelating agent component” refers to a compound or mixtures of compounds that has the ability to bind strongly with metal ions.

As used herein, “% by weight of the formulation” means the percent concentration of the component in the formulation is on weight/weight basis. For example, 1% w/w of component A=[(mass of component A)/(total mass of the formulation)]×100.

As used herein, “% by weight of the emulsion on a free base basis” of a compound described herein (e.g., such as ruxolitinib, deuruxolitinib, or a pharmaceutically acceptable salt of any of the aforementioned, means that the % w/w is calculated based on the weight of the free base of the compound in the foamable composition or foamable carrier component. For example, “1.5% w/w on a free base basis” of ruxolitinib phosphate means that for 100 grams of total formulation, there are 1.98 grams of ruxolitinib phosphate in the foamable composition or foamable carrier component (which equates to 1.5 grams of the free base, ruxolitinib). If not already indicated in the Examples, the percentages of ruxolitinib phosphate can be converted to a free base basis by multiplying by the conversion factor of 0.7575 (FB refers to free base basis).

As used herein, the term “component” can mean one substance or a mixture of substances.

As used herein, the term “fatty acid” refers to an aliphatic acid that is saturated or unsaturated. In some embodiments, the fatty acid is in a mixture of different fatty acids. In some embodiments, the fatty acid has between about eight to about thirty carbons on average. In some embodiments, the fatty acid has about 12 to 20, 14-20, or 16-18 carbons on average. Suitable fatty acids include, but are not limited to, cetyl acid, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, behenic acid, isobehenic acid, and arachidonic acid, or mixtures thereof.

As used herein, the term “fatty alcohol” refers to an aliphatic alcohol that is saturated or unsaturated. In some embodiments, the fatty alcohol is in a mixture of different fatty alcohols. In some embodiments, the fatty alcohol has between about 12 to about 20, about 14 to about 20, or about 16 to about 18 carbons on average. Suitable fatty alcohols include, but are not limited to, stearyl alcohol, lauryl alcohol, palmityl alcohol, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “polyalkylene glycol”, employed alone or in combination with other terms, refers to a polymer containing oxyalkylene monomer units, or copolymer of different oxyalkylene monomer units, wherein the alkylene group has 2 to 6, 2 to 4, or 2 to 3 carbon atoms. As used herein, the term “oxyalkylene”, employed alone or in combination with other terms, refers to a group of formula —O-alkylene-. In some embodiments, the polyalkylene glycol is polyethylene glycol.

As used herein, the term, “sorbitan fatty ester” includes products derived from sorbitan or sorbitol and fatty acids and, optionally, poly (ethylene glycol) units, including sorbitan esters and polyethoxylated sorbitan esters. In some embodiments, the sorbitan fatty ester is a polyethoxylated sorbitan ester.

As used herein, the term “sorbitan ester” refers to a compound, or mixture of compounds, derived from the esterification of sorbitol and at least one fatty acid. Fatty acids useful for deriving the sorbitan esters include, but are not limited to, those described herein. Suitable sorbitan esters include, but are not limited to, the Span™ series (available from Uniqema), which includes Span 20 (sorbitan monolaurate), 40 (sorbitan monopalmitate), 60 (sorbitan monostearate), 65 (sorbitan tristearate), 80 (sorbitan monooleate), and 85 (sorbitan trioleate). Other suitable sorbitan esters include those listed in R. C. Rowe and P. J. Shesky, Handbook of pharmaceutical excipients, (2006), 5th ed., which is incorporated herein by reference in its entirety.

As used herein, the term “polyethoxylated sorbitan ester” refers to a compound, or mixture thereof, derived from the ethoxylation of a sorbitan ester. The polyoxethylene portion of the compound can be between the fatty ester and the sorbitan moiety. As used herein, the term “sorbitan ester” refers to a compound, or mixture of compounds, derived from the esterification of sorbitol and at least one fatty acid. Fatty acids useful for deriving the polyethoyxlated sorbitan esters include, but are not limited to, those described herein. In some embodiments, the polyoxyethylene portion of the compound or mixture has about 2 to about 200 oxyethylene units. In some embodiments, the polyoxyethylene portion of the compound or mixture has about 2 to about 100 oxyethylene units. In some embodiments, the polyoxyethylene portion of the compound or mixture has about 4 to about 80 oxyethylene units. In some embodiments, the polyoxyethylene portion of the compound or mixture has about 4 to about 40 oxyethylene units. In some embodiments, the polyoxyethylene portion of the compound or mixture has about 4 to about 20 oxyethylene units. Suitable polyethoxylated sorbitan esters include, but are not limited to the Tween™ series (available from Uniqema), which includes Tween 20 (POE (20) sorbitan monolaurate), 21 (POE (4) sorbitan monolaurate), 40 (POE (20) sorbitan monopalmitate), 60 (POE (20) sorbitan monostearate), 60K (POE (20) sorbitan monostearate), 61 (POE (4) sorbitan monostearate), 65 (POE (20) sorbitan tristearate), 80 (POE (20) sorbitan monooleate), 80K (POE (20) sorbitan monooleate), 81 (POE (5) sorbitan monooleate), and 85 (POE (20) sorbitan trioleate). As used herein, the abbreviation “POE” refers to polyoxyethylene. The number following the POE abbreviation refers to the number of oxyethylene repeat units in the compound. Other suitable polyethoxylated sorbitan esters include the polyoxyethylene sorbitan fatty acid esters listed in R. C. Rowe and P. J. Shesky, Handbook of pharmaceutical excipients, (2006), 5th ed., which is incorporated herein by reference in its entirety. In some embodiments, the polyethoxylated sorbitan ester is a polysorbate. In some embodiments, the polyethoxylated sorbitan ester is polysorbate 20.

As used herein, the term “glyceryl fatty esters” refers to mono-, di- or triglycerides of fatty acids. The glyceryl fatty esters may be optionally substituted with sulfonic acid groups, or pharmaceutically acceptable salts thereof. Suitable fatty acids for deriving glycerides of fatty acids include, but are not limited to, those described herein. In some embodiments, the glyceryl fatty ester is a mono-glyceride of a fatty acid having 12 to 18 carbon atoms. In some embodiments, the glyceryl fatty ester is glyceryl stearate.

As used herein, the term “triglycerides” refers to a triglyceride of a fatty acid. In some embodiments, the triglyceride is medium chain triglycerides.

As used herein, the term “alkylene glycol” refers to a group of formula —O-alkylene-, wherein the alkylene group has 2 to 6, 2 to 4, or 2 to 3 carbon atoms. In some embodiments, the alkylene glycol is propylene glycol (1,2-propanediol).

As used herein, the term “polyethylene glycol” refers to a polymer containing ethylene glycol monomer units of formula —O—CH₂—CH₂—. Suitable polyethylene glycols may have a free hydroxyl group at each end of the polymer molecule or may have one or more hydroxyl groups etherified with a lower alkyl, e.g., a methyl group. Also suitable are derivatives of polyethylene glycols having esterifiable carboxy groups. Polyethylene glycols useful in the present disclosure can be polymers of any chain length or molecular weight and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer.

As used herein, “contains” is equivalent to “comprises”.

As used herein, the term “subject,” “individual,” or “patient,” used interchangeably, refers to humans. In some embodiments, the “subject,” “individual,” or “patient” is in need of said treatment.

In some embodiments, the compounds, or pharmaceutically acceptable salts thereof, or pharmaceutical formulations thereof, topical formulations thereof, as described herein are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease; or (3) preventing the disease, condition or disorder in an individual who may be predisposed to the disease, condition or disorder but does not yet experience or display the pathology or symptomatology of the disease. In some embodiments, treating refers to inhibiting or ameliorating the disease. In some embodiments, treating is preventing the disease.

In some embodiments, the components are present in exactly the ranges specified (e.g., the term “about” is not present). In some embodiments, “about” means plus or minus 10% of the value.

It is further appreciated that certain features of the present application, which are, for clarity, described in the context of separate embodiments, can also be provided in combination in a single embodiment (while the embodiments are intended to be combined as if written in multiply dependent form). Conversely, various features of the present application which are, for brevity, described in the context of a single embodiment, can also be provided separately or in any suitable subcombination. Thus, it is contemplated as features described as embodiments of the foamable compositions, foamable carrier components, and foams, along with any methods of use or processes of producing thereof, can be combined in any suitable combination.

### Hydroethanolic Foamable Compositions and Foams

The present disclosure is directed to, inter alia, a foamable composition suitable for application as a foam to a body surface area of a human patient, comprising a foamable carrier component and a propellant, wherein the foamable carrier component comprises:
a compound, which is ruxolitinib or deuterated ruxolitinib, or a pharmaceutically acceptable salt thereof,
a hydroethanolic mixture,
an emollient component,
one or more C₁₆₋₁₈ fatty alcohols, and
an emulsifier component,
wherein the hydroethanolic mixture is a mixture of ethanol and water.

The present disclosure is directed to, inter alia, a foamable composition suitable for application as a foam to a body surface area of a human patient, comprising a foamable carrier component and a propellant, wherein the foamable carrier component comprises:
a compound, which is ruxolitinib or deuruxolitinib, or a pharmaceutically acceptable salt thereof,
a hydroethanolic mixture,
an emollient component,
one or more C₁₆₋₁₈ fatty alcohols, and
an emulsifier component,
wherein the hydroethanolic mixture is a mixture of ethanol and water.

The present disclosure is further directed to, inter alia, a foamable composition suitable for application as a foam to a body surface area affected by an inflammatory or autoimmune skin or hair disease in a human patient, comprising a foamable carrier component and a propellant, wherein the foamable carrier component comprises:
a compound, which is ruxolitinib or deuterated ruxolitinib, or a pharmaceutically acceptable salt thereof,
a hydroethanolic mixture,
an emollient component,
one or more C₁₆₋₁₈ fatty alcohols, and
an emulsifier component,
wherein the hydroethanolic mixture is a mixture of ethanol and water.

The present disclosure is further directed to, inter alia, a foamable composition suitable for application as a foam to a body surface area affected by an inflammatory or autoimmune skin or hair disease in a human patient, comprising a foamable carrier component and a propellant, wherein the foamable carrier component comprises:
a compound, which is ruxolitinib or deuruxolitinib, or a pharmaceutically acceptable salt thereof,
a hydroethanolic mixture,
an emollient component,
one or more C₁₆₋₁₈ fatty alcohols, and
an emulsifier component,
wherein the hydroethanolic mixture is a mixture of ethanol and water.

In some embodiments:
the hydroethanolic mixture comprises about 65% to about 99% by weight of the foamable carrier component;
the ethanol comprises about 40% to about 90% by weight of the hydroethanolic mixture;
the emulsifier component is present in an amount ranging from about 0.25% to about 5% by weight of the foamable carrier component;
the emollient component is present in an amount of from about 0.1% to about 4% by weight of the foamable carrier component;
the one or more C₁₆₋₁₈ fatty alcohols is present in an amount ranging from about 0.5% to about 10% by weight of the foamable carrier component; and
the compound is present in an amount from about 0.5% to about 5% by weight of the foamable carrier component on a free base basis.

In some embodiments:
the hydroethanolic mixture comprises about 70% to about 95% by weight of the foamable carrier component;
the ethanol comprises about 50% to about 90% by weight of the hydroethanolic mixture;
the emulsifier component is present in an amount ranging from about 0.5% to about 5% by weight of the foamable carrier component;
the emollient component is present in an amount of from about 0.1% to about 2% by weight of the foamable carrier component;
the one or more C₁₆₋₁₈ fatty alcohol is present in an amount ranging from about 0.5% to about 5% by weight of the foamable carrier component; and
the compound is present in an amount from about 0.5% to about 3% by weight of the foamable carrier component on a free base basis.

In some embodiments:
the hydroethanolic mixture comprises about 75% to about 95% by weight of the foamable carrier component;
the ethanol comprises about 50% to about 90% by weight of the hydroethanolic mixture;
the emulsifier component is present in an amount ranging from about 0.5% to about 5% by weight of the foamable carrier component;
the emollient component is present in an amount of from about 0.1% to about 2% by weight of the foamable carrier component;
the one or more C₁₆₋₁₈ fatty alcohol is present in an amount ranging from about 0.5% to about 5% by weight of the foamable carrier component; and
the compound is present in an amount from about 0.5% to about 3% by weight of the foamable carrier component on a free base basis.

In some embodiments:
the hydroethanolic mixture comprises about 80% to about 90% by weight of the foamable carrier component;
the ethanol comprises about 55% to about 65% by weight of the hydroethanolic mixture;
the emulsifier component is present in an amount ranging from about 0.5% to about 2% by weight of the foamable carrier component;
the emollient component is present in an amount of from about 0.2% to about 1% by weight of the foamable carrier component;
the one or more C₁₆₋₁₈ fatty alcohol is present in an amount ranging from about 1% to about 5% by weight of the foamable carrier component; and
the compound is present in an amount from about 1.5% to about 2.5% by weight of the foamable carrier component on a free base basis.

In some embodiments:
the emulsifier component comprises sorbitan monolaurate (Span 20) and polyethylene glycol sorbitan monostearate (Tween 60);
the emollient component is glycerin or myristyl lactate; and
at least one of the one or more C₁₆₋₁₈ fatty alcohols is stearyl alcohol.

In some embodiments:
the emulsifier component comprises sorbitan monolaurate (Span 20) and polyethylene glycol sorbitan monostearate (Tween 60);
the emollient component is glycerin or myristyl lactate; and
the one or more C₁₆₋₁₈ fatty alcohols comprises cetyl alcohol and stearyl alcohol.

In some embodiments, the foamable composition and/or the foamable carrier component does not comprise an organic amine pH adjusting agent. In some embodiments, an organic amine pH adjusting agent is an aromatic amine, a tertiary amine, a secondary amine, a primary amine, ammonia, or an alkanol amine, such as a mono-di- or tri-alkanolamine, e.g., a trialkanolamine or trolamine. In some embodiments, the organic amine pH adjusting agent is trolamine, tris, ethanolamine, diethanolamine, ammonia, diisopropanolamine, 1-amino-2-propanol, 2-amino-2-ethyl-1,3-propanediol, 2-amino-2-methyl-1-propanol, diisopropylamine, imidazole, and pyridine.

The foamable composition and foamable carrier component can include additional excipients as noted below, including a co-solvent component and/or a penetration enhancer.

In some embodiments, the body surface area affected comprises a bodily surface with hair follicles, i.e., epidermis or dermis. Hair follicles originate in the first and second layers of the skin-epidermis and dermis. For example, the bodily surface with hair follicles includes, but is not limited to, scalp, arms, legs, eyebrows, eyelashes, back of neck, face, armpits, face, or a combination thereof. In some embodiments, the body surface area is the scalp.

### Hydroethanolic Mixture

In some embodiments, the foamable carrier component comprises a hydroethanolic mixture. In some embodiments, the hydroethanolic mixture is a mixture of ethanol and water. In some embodiments, the hydroethanolic mixture comprises about 65% to about 99% by weight of the foamable carrier component. In some embodiments, the hydroethanolic mixture comprises about 70% to about 95% by weight of the foamable carrier component. In some embodiments, the hydroethanolic mixture comprises about 70% to about 99% by weight of the foamable carrier component. In some embodiments, the hydroethanolic mixture comprises about 75% to about 95% by weight of the foamable carrier component. In some embodiments, the hydroethanolic mixture comprises about 80% to about 90% by weight of the foamable carrier component.

In some embodiments, the ethanol comprises about 30% to about 80% by weight of the hydroethanolic mixture. In some embodiments, the ethanol comprises about 40% to about 90% by weight of the hydroethanolic mixture. In some embodiments, the ethanol comprises about 40% to about 80% by weight of the hydroethanolic mixture. In some embodiments, the ethanol comprises about 40% to about 60% by weight of the hydroethanolic mixture. In some embodiments, the ethanol comprises about 50% to about 90% by weight of the hydroethanolic mixture. In some embodiments, the ethanol comprises about 50% to about 80% by weight of the hydroethanolic mixture. In some embodiments, the ethanol comprises about 50% to about 75% by weight of the hydroethanolic mixture. In some embodiments, the ethanol comprises about 50% to about 70% by weight of the hydroethanolic mixture. In some embodiments, the ethanol comprises about 55% to about 70% by weight of the hydroethanolic mixture. In some embodiments, the ethanol comprises about 55% to about 65% by weight of the hydroethanolic mixture. In some embodiments, the ethanol comprises about 58% to about 64% by weight of the hydroethanolic mixture. Each of the embodiments described in this paragraph can be combined with any of the embodiments in the preceding paragraph as if written in independent form.

In some embodiments, the foamable carrier component comprises ≤50% water by weight of the foamable carrier component (e.g., below 50%). In some embodiment, the water is present in an amount from about 1% to about 50%, from about 5% to about 50%, from about 5% to about 50%, from about 10% to about 50%, from about 20% to about 50%, from about 25% to about 40%, from about 25% to about 35%, and from about 30% to 35%, by weight of the foamable carrier component.

In some embodiments, the water comprises about 10% to about 50% by weight of the foamable carrier component. In some embodiments, the water comprises about 20% to about 40% by weight of the foamable carrier component. In some embodiments, the water comprises about 25% to about 35% by weight of the foamable carrier component.

In some embodiments, the foamable carrier component comprises ≤50% water by weight of the foamable carrier component (e.g., below 50%). In some embodiment, the water is present in an amount from about 1% to about 50%, from about 5% to about 50%, from about 5% to about 50%, from about 10% to about 50%, from about 20% to about 50%, from about 25% to about 40%, from about 25% to about 35%, and from about 30% to 35%, by weight of the foamable carrier component.

In some embodiments, the foamable carrier component comprises an amount of water less than an amount of one or more C₁₋₄ aliphatic alcohol. In some embodiments, the foamable carrier component comprises an amount of water less than an amount of ethanol, when present in the foamable carrier component. In some embodiments, the foamable carrier component comprises ethanol. In some embodiments, the ethanol is present in an amount ranging from about 30% to about 80% of the foamable carrier component. In some embodiments, the ethanol is present in an amount ranging from about 40% to about 60% of the foamable carrier component. In some embodiments, the ethanol is present in an amount ranging from about 45% to about 55% of the foamable carrier component.

In some embodiment, ethanol is present in an amount greater than water. In some embodiments, a ratio of ethanol to water ranges from 55:45 to 95:5, such as from 60:40 to 80:20. The ethanol to water ratio is calculated by dividing the amount of ethanol by the total sum of water plus ethanol and is not the % w/w of ethanol and water based on the total weight of the foamable carrier component.

In some embodiments, where the foamable composition has an ethanol to water ratio with a higher amount of ethanol to water, the ratio of ethanol to watyer provides for a foamable carrier component allowing for the solubility of the active, e.g., ruxolitinib phosphate. In some embodiments, the foamable carrier component has a ratio of ethanol to water of 60:40. In some embodiments, the foamable carrier component has a ratio of ethanol to water of 80:20.

### Emulsifier Component

In some embodiments, the foamable carrier component comprises an emulsifier component. In some embodiments, the emulsifier component comprises one or more anionic surfactants. In some embodiments, the emulsifier component is laureth-4.

In some embodiments, the emulsifier component comprises one or more nonionic emulsifiers. Various emulsifiers have different HLB numbers. For example, a lower HLB value indicates better oil (i.e., non-polar) solubility and a higher HLB value indicates better water (i.e., polar) solubility. The hydrophilic-lipophilic turning point is HLB 10. HLB less than 10 is oleophilic and greater than 10 is hydrophilic. Emulsifiers with low HLB values are more suitable for water-in-oil emulsions (W/O), for example, 3 to 7; emulsifiers with high HLB values are more suitable for oil-in-water emulsions (O/W), for example, 7 to 19. In some embodiments, the one or more nonionic emulsifiers have a HLB value ranging from 1 to 16. In some embodiments, the one or more nonionic emulsifiers have a HLB value ranging from 5 to 16. In some embodiments, the one or more nonionic emulsifier comprises sorbitan monolaurate (Span 20) (HLB 8.6), polyethylene glycol sorbitan monostearate (Tween 60) (HLB 14.9), or lauryl alcohol (HLB 5-18).

Examples of common emulsifiers with their HLB values include, but are not limited to:

| Trade Name | Type | HLB Value |
|---|---|---|
| Span 85 sorbitan trioleate | Nonionic | 1.8 |
| Span 65 soibitan tristearate | Nonionic | 2.1 |
| Propylene glycol fatty acid ester | | 3.4 |
| Hydroxylatedanolin | Nonionic | 3.8 |
| Span 80 sorbitan monooleate | Nonionic | 4.3 |
| Span 60 sorbitan monostearate | Nonionic | 4.7 |
| Glaurin diethylene glycol monolaurate | Nonionic | 6.5 |
| Span 20 Sorbitan monolaurate | Nonionic | 8.6 |
| Brij 30 poloxyethylene lauryl ether | Nonionic | 9.5 |
| Tween 81 polyoxyethylene | Nonionic | 10.0 |
| sorbitan monooleate | | |
| Myrj 45 polyoxyethylene monostearate | Nonionic | 11.1 |
| PEG 400 monooleate | Nonionic | 11.4 |
| polyoxyethylene monooleate | | |
| S-541 polyoxyethylene monostearate | Nonionic | 11.6 |
| Myrsityl laurate | Nonionic | 12 |
| PEG 400 monolurate | Nonionic | 13.1 |
| polyoxyethylene monolaurate | | |
| Tween 21 polyoxyethylene | Nonionic | 13.3 |
| sorbitan monolaurate | | |
| Tween 60 polyoxyethylene | Nonionic | 14.9 |
| sorbitan monostearate | | |
| Tween 80 polyoxyethylene | Nonionic | 15.0 |
| sorbtain monostearate | | |
| Myrj 51 polyoxyethylene | Nonionic | 16.0 |
| monostearate | | |
| Tween 20 polyoxyethylene | Nonionic | 16.7 |
| sorbitan monolaurate | | |

In some embodiments, the emulsifier component comprises one or more emulsifiers in the preceding table. In some embodiments, the emulsifier component comprises two of the emulsifiers in the preceding table. In some embodiments, the emulsifier component is a mixture of two non-ionic emulsifiers. In some embodiments, the emulsifier component is a mixture of two non-ionic emulsifiers. In some embodiments, the emulsifier component is a mixture of a non-ionic emulsifier having a HLB of 4-10 and a non-ionic emulsifier having an HLB of 10-16. In some embodiments, the emulsifier component comprises sorbitan monolaurate (Span 20) and polyethylene glycol sorbitan monostearate (Tween 60).

In some embodiments, the emulsifier component is present in an amount ranging from about 0.05% to about 8% by weight of the foamable carrier component. In some embodiments, the emulsifier component is present in an amount ranging from about 0.25% to about 5% by weight of the foamable carrier component. In some embodiments, the emulsifier component is present in an amount ranging from about 0.5% to about 5% by weight of the foamable carrier component. In some embodiments, the emulsifier component is present in an amount ranging from about 0.5% to about 4% by weight of the foamable carrier component. In some embodiments, the emulsifier component is present in an amount ranging from about 0.5% to about 3% by weight of the foamable carrier component. In some embodiments, the emulsifier component is present in an amount ranging from about 0.5% to about 2% by weight of the foamable carrier component.

In some embodiments, the emulsifier component comprises sorbitan monolaurate (Span 20) in an amount of about 0.25% to about 2% by weight of the foamable carrier component and polyethylene glycol sorbitan monostearate (Tween 60) in an amount of about 0.25% to about 2% by weight of the foamable carrier component.

### Emollient Component

In some embodiments, the foamable carrier component comprises an emollient component. In some embodiments, the emollient component includes, but is not limited to, petrolatum, glycerin, isopropyl palmitate, isopropyl myristate, mineral oil, and combinations thereof. In some embodiments, the emollient component comprises one or more substances chosen from, but not limited to, PEG-6 caprylic capric glycerides (Glycerox 767), glyceryl caprylate, glyceryl caprate, isostearic acid, glycerol monolaurate, glycerin, PPG stearyl ether, diisopropyl adipate (DIPA), Arlamol PS11E pharma (propoxylate), oleic acid, and myristyl lactate, and combinations thereof. In some embodiments, the emollient component comprises glycerin. In some embodiments, the emollient component comprises myristyl lactate. In some embodiments, the emollient component is selected from glycerin and myristyl lactate.

In some embodiments, the emollient component is present in an amount of from about 0.1% to about 4% by weight of the foamable carrier component. In some embodiments, the emollient component is present in an amount of from about 0.1% to about 3% by weight of the foamable carrier component. In some embodiments, the emollient component is present in an amount of from about 0.1% to about 2% by weight of the foamable carrier component. In some embodiments, the emollient component is present in an amount of from about 0.1% to about 1% by weight of the foamable carrier component. In some embodiments, the emollient component is present in an amount of from about 0.2% to about 1% by weight of the foamable carrier component.

In some embodiments, when the emollient component is glycerin, it is present in an amount ranging from about 0.1% to about 2% by weight of the foamable carrier component. In some embodiments, when the emollient component is glycerin, it is present in an amount ranging from about 0.2% to about 1.5% by weight of the foamable carrier component. In some embodiments, when the emollient component is glycerin, it is present in an amount ranging from about 0.2% to about 1% by weight of the foamable carrier component. In some embodiments, when the emollient component is glycerin, it is present in an amount ranging from about 0.5% to about 1% by weight of the foamable carrier component.

In some embodiments, when the emollient component is myristyl lactate, it is present in an amount ranging from about 0.1% to about 2% by weight of the foamable carrier component. In some embodiments, when the emollient component is myristyl lactate, it is present in an amount ranging from about 0.1% to about 1.5% by weight of the foamable carrier component. In some embodiments, when the emollient component is myristyl lactate, it is present in an amount ranging from about 0.1% to about 1% by weight of the foamable carrier component. In some embodiments, when the emollient component is myristyl lactate, it is present in an amount ranging from about 0.25% to about 1% by weight of the foamable carrier component.

### Co-Solvent Component

In some embodiments, the foamable carrier component further comprises a co-solvent component. For example, the co-solvent component may help solubilize or stabilize the emollient component in the foamable carrier component or foamable composition. In some embodiments, the co-solvent component comprises polyethylene glycol or transcutol-P. In some embodiments, the co-solvent component comprises polyethylene glycol. In some embodiments, the co-solvent component comprises PEG300 (polyethylene glycol 300). In some embodiments, the co-solvent component comprises transcutol-P. In some embodiments, when the emollient component comprises glycerin, the co-solvent component comprises polyethylene glycol. In some embodiments, when the emollient component comprises glycerin, the co-solvent component comprises PEG300. In some embodiments, when the emollient component comprises myristyl lactate, the co-solvent component comprises transcutol-P. In some embodiments, the co-solvent component also functions as a penetration enhancer.

In some embodiments, the co-solvent component is present in an amount of about 0.1% to about 10% by weight of the foamable carrier component. In some embodiments, the co-solvent component is present in an amount of about 0.1% to about 8% by weight of the foamable carrier component. In some embodiments, the co-solvent component is present in an amount of about 0.1% to about 7% by weight of the foamable carrier component. In some embodiments, the co-solvent component is present in an amount of about 0.1% to about 6% by weight of the foamable carrier component. In some embodiments, the co-solvent component is present in an amount of about 0.1% to about 5% by weight of the foamable carrier component.

In some embodiments, the co-solvent component is present in an amount of about 0.1% to about 10% by weight of the foamable carrier component. In some embodiments, the co-solvent component is present in an amount of about 0.25% to about 8% by weight of the foamable carrier component. In some embodiments, the co-solvent component is present in an amount of about 0.25% to about 7% by weight of the foamable carrier component. In some embodiments, the co-solvent component is present in an amount of about 0.25% to about 6% by weight of the foamable carrier component. In some embodiments, the co-solvent component is present in an amount of about 0.25% to about 5% by weight of the foamable carrier component. In some embodiments, the co-solvent component is present in an amount of about 0.5% to about 10% by weight of the foamable carrier component. In some embodiments, the co-solvent component is present in an amount of about 0.5% to about 8% by weight of the foamable carrier component. In some embodiments, the co-solvent component is present in an amount of about 0.5% to about 7% by weight of the foamable carrier component. In some embodiments, the co-solvent component is present in an amount of about 0.5% to about 6% by weight of the foamable carrier component. In some embodiments, the co-solvent component is present in an amount of about 0.5% to about 5% by weight of the foamable carrier component.

In some embodiments, the co-solvent component is PEG300 and is present in an amount of about 0.5% to about 8% by weight of the foamable carrier component. In some embodiments, the co-solvent component is PEG300 and is present in an amount of about 0.5% to about 7% by weight of the foamable carrier component. In some embodiments, the co-solvent component is PEG300 and is present in an amount of about 1% to about 6% by weight of the foamable carrier component. In some embodiments, the co-solvent component is PEG300 and is present in an amount of about 1% to about 5% by weight of the foamable carrier component. In some embodiments, the co-solvent component is PEG300 and is present in an amount of about 1% to about 4% by weight of the foamable carrier component.

In some embodiments, the co-solvent component is transcutol-P and is present in an amount of about 0.1% to about 5% by weight of the foamable carrier component. In some embodiments, the co-solvent component is transcutol-P and is present in an amount of about 0.1% to about 4% by weight of the foamable carrier component. In some embodiments, the co-solvent component is transcutol-P and is present in an amount of about 0.1% to about 3% by weight of the foamable carrier component. In some embodiments, the co-solvent component is transcutol-P and is present in an amount of about 0.25% to about 2% by weight of the foamable carrier component.

### C₁₆₋₁₈ Fatty Alcohol

In some embodiments, the foamable carrier component comprises one or more C₁₆₋₁₈ fatty alcohols. In some embodiments, the one or more C₁₆₋₁₈ fatty alcohols is a mixture of fatty alcohols with 16 carbon atoms (cetyl alcohol) and 18 carbon atoms (stearyl alcohol). In some embodiments, the one or more C₁₆₋₁₈ fatty alcohols comprises cetyl alcohol or stearyl alcohol. In some embodiments, the one or more C₁₆₋₁₈ fatty alcohols comprises cetyl alcohol or stearyl alcohol, or a mixture thereof. In some embodiments, the one or more C₁₆₋₁₈ fatty alcohols comprises cetyl alcohol. In some embodiments, the one or more C₁₆₋₁₈ fatty alcohols comprises stearyl alcohol. In some embodiments, the one or more C₁₆₋₁₈ fatty alcohols comprises comprises cetyl alcohol and stearyl alcohol.

In some embodiments, the one or more C₁₆₋₁₈ fatty alcohols is present in an amount ranging from about 0.5% to about 10% by weight of the foamable carrier component. In some embodiments, the one or more C₁₆₋₁₈ fatty alcohols is present in an amount ranging from about 0.75% to about 8% by weight of the foamable carrier component. In some embodiments, the one or more C₁₆₋₁₈ fatty alcohols is present in an amount ranging from about 0.25% to about 10% by weight of the foamable carrier component. In some embodiments, the one or more C₁₆₋₁₈ fatty alcohol is present in an amount ranging from about 0.25% to about 8% by weight of the foamable carrier component. In some embodiments, the one or more C₁₆₋₁₈ fatty alcohol is present in an amount ranging from about 0.25% to about 5% by weight of the foamable carrier component. In some embodiments, the one or more C₁₆₋₁₈ fatty alcohol is present in an amount ranging from about 0.5% to about 5% by weight of the foamable carrier component. In some embodiments, the one or more C₁₆₋₁₈ fatty alcohol is present in an amount ranging from about 1% to about 4% by weight of the foamable carrier component. In some embodiments, the one or more C₁₆₋₁₈ fatty alcohol is present in an amount ranging from about 1% to about 5% by weight of the foamable carrier component. In some embodiments, the one or more C₁₆₋₁₈ fatty alcohol is present in an amount ranging from about 1.5% to about 3.5% by weight of the foamable carrier component.

In some embodiments, when one of the one or more C₁₆₋₁₈ fatty alcohol is cetyl alcohol, it is present in an amount ranging from about 0.5% to about 5% by weight of the foamable carrier component. In some embodiments, when one of the one or more C₁₆₋₁₈ fatty alcohol is cetyl alcohol, it is present in an amount ranging from about 0.5% to about 3% by weight of the foamable carrier component. In some embodiments, when one of the one or more C₁₆₋₁₈ fatty alcohol is cetyl alcohol, it is present in an amount ranging from about 0.5% to about 3.5% by weight of the foamable carrier component. In some embodiments, when one of the one or more C₁₆₋₁₈ fatty alcohol is cetyl alcohol, it is present in an amount ranging from about 1% to about 2.5% by weight of the foamable carrier component.

In some embodiments, when one of the one or more C₁₆₋₁₈ fatty alcohol is stearyl alcohol, it is present in an amount ranging from about 0.1% to about 2% by weight of the foamable carrier component. In some embodiments, when one of the one or more C₁₆₋₁₈ fatty alcohol is stearyl alcohol, it is present in an amount ranging from about 0.1% to about 1.5% by weight of the foamable carrier component. In some embodiments, when one of the one or more C₁₆₋₁₈ fatty alcohol is stearyl alcohol, it is present in an amount ranging from about 0.2% to about 1% by weight of the foamable carrier component. In some embodiments, when one of the one or more C₁₆₋₁₈ fatty alcohol is stearyl alcohol, it is present in an amount ranging from about 0.25% to about 1% by weight of the foamable carrier component.

### Active Pharmaceutical Ingredient

In some embodiments, the compound (i.e., the active pharmaceutical ingredient) in the foamable carrier component is ruxolitinib, deuterated ruxolitinib, or a pharmaceutically acceptable salt of any of those aforementioned. In some embodiments, the compound is ruxolitinib, or a pharmaceutically acceptable salt thereof. In some embodiments, the compound is ruxolitinib phosphate. In some embodiments, the compound is ruxolitinib sulfate. In some embodiments, the compound is ruxolitinib maleate.

In some embodiments, the compound is deuterated ruxolitinib, or a pharmaceutically acceptable salt thereof. In some embodiments, the compound is deuruxolitinib, or a pharmaceutically acceptable salt thereof. In some embodiments, the compound is deuruxolitinib phosphate.

In some embodiments, the compound is present in an amount from about 0.5% to about 5% by weight of the foamable carrier component on a free base basis. In some embodiments, the compound is present in an amount from about 0.5% to about 4% by weight of the foamable carrier component on a free base basis. In some embodiments, the compound is present in an amount from about 0.5% to about 3% by weight of the foamable carrier component on a free base basis. In some embodiments, the compound is present in an amount from about 1% to about 5% by weight of the foamable carrier component on a free base basis. In some embodiments, the compound is present in an amount from about 1.5% to about 3.5% by weight of the foamable carrier component on a free base basis. In some embodiments, the compound is present in an amount from about 1.5% to about 3% by weight of the foamable carrier component on a free base basis. In some embodiments, the compound is present in an amount from about 1.5% to about 2.5% by weight of the foamable carrier component on a free base basis.

In some embodiments, the compound is present in an amount from about 0.5% to about 5% by weight of the foamable carrier component on a free base basis of the compound, which is ruxolitinib or deuterated ruxolitinib, or a pharmaceutically acceptable salt of any of the aforementioned. In some embodiments, the foamable carrier component comprises about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, about 2.0%, about 2.5%, about 3.0%, about 3.5%, about 4.0%, about 4.5%, or about 5.0% by weight of the foamable carrier component on a free base basis of the compound, or a pharmaceutically acceptable salt thereof. In some embodiments, the compound is present in an amount from about 0.05% to about 5% by weight of the foamable carrier component on a free base basis of the compound, which is ruxolitinib or deuterated ruxolitinib, or a pharmaceutically acceptable salt of any of the aforementioned. In some embodiments, the foamable carrier component comprises from about 1% to about 5% by weight of the foamable carrier component on a free base basis of the compound, or a pharmaceutically acceptable salt thereof. In some embodiments, the foamable carrier component comprises from about 1.5% to about 3.5% by weight of the foamable carrier component on a free base basis of the compound, or a pharmaceutically acceptable salt thereof.

In some embodiments, the foamable carrier component comprises 1.5% by weight of the foamable carrier component on a free base basis of the compound, or a pharmaceutically acceptable salt thereof. In some embodiments, the foamable carrier component comprises 2.5% by weight of the foamable carrier component on a free base basis of the compound, or a pharmaceutically acceptable salt thereof. In some embodiments, the foamable carrier component comprises 3.0% by weight of the foamable carrier component on a free base basis of the compound, or a pharmaceutically acceptable salt thereof. In some embodiments, the foamable carrier component comprises 3.5% by weight of the foamable carrier component on a free base basis of the compound, or a pharmaceutically acceptable salt thereof. In some embodiments, the foamable carrier component comprises 4.0% by weight of the foamable carrier component on a free base basis of the compound, or a pharmaceutically acceptable salt thereof.

In some embodiments, the compound in each of the embodiments of this section is ruxolitinib, or a pharmaceutically acceptable salt thereof. In some embodiments, the compound in each of the embodiments of this section is ruxolitinib phosphate. In some embodiments, the compound in each of the embodiments of this section is ruxolitinib chloride. In some embodiments, the compound in each of the embodiments of this section is deuruxolitinib, or a pharmaceutically acceptable salt thereof. In some embodiments, the compound in each of the embodiments of this section is deuruxolitinib phosphate.

### Penetration Enhancers

In some embodiments, the foamable carrier component comprises one or more penetration enhancers. In some embodiments, the one or more penetration enhancers are present in an amount ranging from about 0.05% to about 10% by weight of the foamable carrier component. In some embodiments, the one or more penetration enhancers are present in an amount ranging from about 0.5% to about 8% by weight of the foamable carrier component. In some embodiments, the one or more penetration enhancers are present in an amount ranging from about 1% to about 8% by weight of the foamable carrier component. In some embodiments, the one or more penetration enhancers are present in an amount ranging from about 2% to about 8% by weight of the foamable carrier component. In some embodiments, the one or more penetration enhancers are present in an amount ranging from about 3% to about 8% by weight of the foamable carrier component. In some embodiments, the one or more penetration enhancers are present in an amount ranging from about 4% to about 7% by weight of the foamable carrier component.

In some embodiments, the one or more penetration enhancers is propylene glycol. In some embodiments, propylene glycol is present in an amount from about 2.5% to about 8% by weight of the foamable carrier component. In some embodiments, propylene glycol is present in an amount from about 3% to about 8% by weight of the foamable carrier component. In some embodiments, propylene glycol is present in an amount from about 4% to about 6% by weight of the foamable carrier component.

In some embodiments, the one or more penetration enhancers is diethylene glycol monoethyl ether (transcutol-P). In some embodiments, diethylene glycol monoethyl ether (transcutol-P) is present in an amount from about 0.05% to about 2.5% by weight of the foamable carrier component. In some embodiments, diethylene glycol monoethyl ether (transcutol-P) is present in an amount from about 0.2% to about 2% by weight of the foamable carrier component. In some embodiments, diethylene glycol monoethyl ether (transcutol-P) is present in an amount from about 0.5% to about 1.5% by weight of the foamable carrier component.

In some embodiments, the one or more penetration enhancers comprise sulfoxides (e.g., dimethylsulfoxide (DMSO)), azones (e.g., laurocapram), pyrrolidines (e.g., 2-pyrrolidone, 2P), alcohols and alkanols (ethanol or decanol), glycols (e.g., propylene glycol, PG), surfactants, fatty acids, terpenes, and combinations thereof. In some embodiments, the one or more penetration enhancers comprise a polyol such as polyethylene glycol (PEG, e.g., polyethylene glycol 300 (PEG300), polyethylene glycol 400 (PEG 400)), glycerol (glycerin), maltitol, sorbitol etc.; diethylene glycol monoethyl ether (DEGEE or Transcutol), diethylene glycol ethyl ether, azone, benzalkonium chloride (ADBAC), cetylperidium chloride, cetylmethylammonium bromide, dextran sulfate, lauric acid, menthol, methoxysalicylate, oleic acid, phosphatidylcholine, polyoxyethylene, polysorbate 80, sodium glycholate, sodium lauryl sulfate, sodium salicylate, sodium taurocholate, sodium taurodeoxycholate, sulfoxides, sodium deoxycholate, sodium glycodeoxycholate, sodium taurocholate and surfactants such as sodium lauryl sulfate, laureth-9, cetylpyridinium chloride and polyoxyethylene monoalkyl ethers, benzoic acids, such as sodium salicylate and methoxy salicylate, fatty acids, such as lauric acid, oleic acid, undecanoic acid and methyl oleate, fatty alcohols, such as octanol and nonanol, laurocapram, cyclodextrins, thymol, limonene, urea, chitosan and other natural and synthetic polymers.

### pH Adjustment

The present disclosure also provides for the foamable carrier component having a pH of from about 4.0 to about 8.0, from about 4.0 to about 7.0, from about 4.0 to about 6.0, about 5.0 to about 8.0, from about 5.5 to about 7.5, from about 5.5 to about 7.0, from about 5.5 to about 6.5, from about 5.0 to about 6.0, and at about 5.5. In some embodiments, the for the foamable carrier component has a pH of from about 4.0 to about 8.0. In some embodiments, the foamable carrier component has a pH of from about 4.0 to about 7.0. In some embodiments, the foamable carrier component has a pH of from about 4.0 to about 6.0. In some embodiments, the foamable carrier component has a pH of about 5.0 to about 8.0. In some embodiments, the foamable carrier component has a pH of from about 5.5 to about 7.5. In some embodiments, the foamable carrier component has a pH of from about 5.5 to about 7.0. In some embodiments, the foamable carrier component has a pH from about 5.0 to about 7.0. In some embodiments, the foamable carrier component has a pH of from about 5.5 to about 6.5. In some embodiments, the foamable carrier component has a pH of from about 5.0 to about 6.0. In some embodiments, the foamable carrier component has a pH of about 5.5.

In some embodiments, the foamable carrier component is pH adjusted by one or more buffering agents. In some embodiments, the one or more buffering agents comprises one or more independently selected from sodium hydroxide, phosphoric acid, hydrochloric acid, boric acid, tetra boric acid, acetic acid, tartaric acid, citric acid, carbonic acid, and their alkali metal salts or ammonium salts (including acid salts in the case of polybasic acids), organic amines (e.g., ethanolamine and triethanolamine) and their quaternary salts, glycine, etc. In some embodiments, the buffering agent is ethanolamine. In some embodiments, the buffering agent is triethanolamine.

### Propellant Component

In some embodiments, the foamable composition comprises a propellant component. In some embodiments, the propellant component comprises about 2% to about 10% of the foamable composition. In some embodiments, the propellant component comprises about 2% to about 8% of the foamable composition. In some embodiments, the propellant component comprises about 3% to about 5% of the foamable composition.

In some embodiments, the propellant component comprises one or more hydrofluorocarbons (HFCs) or hydrofluoroolefins (HFOs). In some embodiments, the propellant component comprises one or more hydrofluorocarbons (HFCs). In some embodiments, the propellant component comprises one or more hydrofluoroolefins (HFOs). In some embodiments, the propellant component comprises HFA-134. In some embodiments, the propellant comprises HFO-1234ze. In some embodiments, the propellant component comprises R152a (CAS #: 75-37-6) or R134a (CAS #: 811-97-2).

In some embodiments, the foamable composition comprises a non-volatile propellant. In some embodiments, the non-volatile propellant comprises nitrogen, nitrous oxide, carbon dioxide, dimethyl ether, 1,3,3,3-tetrafluoroprop-1-ene, 1,1,1,2-tetrafluoroethane, 1,1-difluoroethane or any combination thereof. In some embodiments, the non-volatile propellant comprises nitrogen, nitrous oxide, carbon dioxide or mixture of these propellants. In some embodiments, the non-volatile propellant comprises from about 2% to about 10% of the foamable composition.

In some embodiments, the foamable composition comprises of a volatile propellant. In some embodiments, the volatile propellant comprises propane, iso-butane, n-butane, or any combination thereof. In some embodiments, the propellant is AP22, AP30, AP104, AP40, AP46, AP58, AP70, P70, or P75. In some embodiments, the propellant is P70, P75, or a mixture thereof. In some embodiments, the propellant is P75, wherein the propellant is P75 and is present in an amount of about 4% of the foamable composition.

In some embodiments, the propellant component comprises a mixture of two to three compressed or liquefied gases selected from:

| propane (% by weight | iso-butane (% by weight | n-butane (% by weight |
|---|---|---|
| of the propellant | of the propellant | of the propellant |
| component) | component) | component) |
| 0 | 30 | 67 |
| 11 | 29 | 60 |
| 95 | 3 | 2 |
| 22 | 24 | 54 |
| 28 | 23 | 49 |
| 31 | 23 | 46 |
| 55 | 15 | 30 |

In some embodiments, a mixture of volatile propellant comprises a percentage of propane 0%-99%, iso-butane 0%-99%, and/or n butane 0%-99% by weight of the propellant component, wherein the mixture of the volatile propellants is greater than 0%. In some embodiments, the volatile propellant comprises P75 propellant; P75 propellant is a mixture of propane (52.24%), iso-butane (21.12%) and butane (26.64%). P45 propellant is a mixture of propane (20.4%), iso-butane (35.2%) and butane (44.4%). In some embodiments, the propellant is P45 or P75. For example, hydrocarbon propellant P45 and/or P75 can be sourced from Ensign Laboratories (490-500 Wellington Road, Mulgrave, Victoria 3170, Australia).

In some embodiments, the propellant is selected from AP 105, AP22, AP30, AP40, AP46, AP48, or AP70 (compositions shown in the table below). the propellant component is selected from HFA-134, HFO-1234ze, R152a, AP22, AP30, AP40, AP46, AP48, AP58, AP70, AP104, AP105, AP22, AP30, P45, or P75, or a mixture thereof.

| Name | Propane % | Iso-butane | n-butane |
|---|---|---|---|
| AP105 | 95 | 3 | 2 |
| AP22 | 0 | 30 | 67 |
| AP30 | 11 | 29 | 60 |
| AP40 | 22 | 24 | 54 |
| AP46 | 28 | 23 | 49 |
| AP48 | 31 | 23 | 46 |
| AP70 | 55 | 15 | 30 |

In some embodiments, the propellant is P75 or P45, or a mixture thereof. In some embodiments, the propellant is P75. In some embodiments, the propellant is P75 and is present in an amount of about 4% of the foamable composition.

In some embodiments, the propellant component comprises about 2% to about 10% of the foamable composition. In some embodiments, the propellant component comprises about 2% to about 8% of the foamable composition. In some embodiments, the propellant component comprises about 3% to about 7% of the foamable composition. In some embodiments, the propellant component comprises about 3% to about 5% of the foamable composition. In some embodiments, the propellant component comprises about 2% of the foamable composition. In some embodiments, the propellant component comprises about 3% of the foamable composition. In some embodiments, the propellant component comprises about 3.5% of the foamable composition. In some embodiments, the propellant component comprises about 4% of the foamable composition. In some embodiments, the propellant component comprises about 4.5% of the foamable composition. In some embodiments, the propellant component comprises about 5% of the foamable composition.

In some embodiments, the present disclosure provides a foamable composition comprising any of the foamable carrier components described herein and a propellant component.

In some embodiments, the present disclosure provides a foam produced by any of the foamable compositions described herein. In some embodiments, the foam of the present disclosure has a foam collapse property to ensure, e.g., retention on the subject's affected skin area. In some embodiments, the components of the topical foam composition are utilized to generate the needed foam collapse to be treat the subject's affected skin area. Foam collapse indicates how quickly and how long the foam will come in contact with the subject's affected skin area. When the foam breaks, the active pharmaceutical ingredient comes directly in contact with the subject's affected skin area but may also limit the overall duration of contact between the skin are and the foam.

In some embodiments, the foam collapse is measured using a water bath as a temperature-controlled environment. A sample contained with a screw capped is used with clamps to hold it in the water bath at, e.g., 32° C.-37° C., halfway submerged. Samples to be tested are attached with a metering valve. The samples are evaluated based on, e.g., visual appearance and time to collapse. In some embodiments, a further foam collapse measurement can be taken when the foam of the present disclosure is placed in a drying over set to a temperature of about 36° C.-37° C. and time lapse is measured until bubbles of the foam have broken or liquified. In some embodiments, the foam collapse occurs within 1 minute or less of application to the affected area.

In some embodiments, the foamable composition has a foam collapse rate of about 2.5 minutes, such as ≥3 minutes, and further for example, ≥5 minutes. In some embodiments, the foamable composition has a foam collapse rate ranging from about 2.5 minutes to about 6.5 minutes. In some embodiments, the foamable composition has a foam collapse rate of 2.5 minutes, 3.0 minutes, 3.5 minutes, 4.0 minutes, 4.5 minutes, 5.0 minutes, 5.5 minutes, 6.0 minutes, and/or 6.5 minutes.

### Additional Components

In some embodiments, the foamable carrier component or the foamable composition comprise one or more additional components. In some embodiments, the one or more additional components comprise stabilizing agents, occlusive agents, stiffing agents, preservatives, thickening agents, gelling agents, viscosity building agents, co-solvent, antioxidants, and combinations thereof.

### Additional Foams and Foamable Compositions

The present disclosure is directed to, inter alia, a foamable composition suitable for application as a foam to a body surface area affected by alopecia in a human patient, comprising a foamable carrier component and a propellant component, wherein the foamable carrier component comprises a compound, which is ruxolitinib or deuterated ruxolitinib, or a pharmaceutically acceptable salt of any of the aforementioned.

The present disclosure is also directed to a foam suitable for application to a body surface area affected by alopecia in a human patient, comprising a compound, which is ruxolitinib or deuterated ruxolitinib, or a pharmaceutically acceptable salt of any of the aforementioned.

The present disclosure is further directed to foamable carrier component, comprising a compound, which is ruxolitinib or deuterated ruxolitinib, or a pharmaceutically acceptable salt of any of the aforementioned.

In some embodiments, the foamable carrier component further comprises water, a solvent component, and an oil phase.

In some embodiments, the foamable carrier component further comprises water, a solvent component, a foaming agent, and an oil phase.

In some embodiments, the foamable carrier component is an emulsion. In some embodiments, the foamable composition is an emulsion. In some embodiments, the emulsion is an oil-in-water emulsion.

In some embodiments, the foamable carrier component is a homogeneous emulsion. In some embodiments, the foamable composition is a homogeneous emulsion.

In some embodiments, the foamable carrier component comprises an oil phase. In some embodiments, the oil phase comprises at least one fatty alcohol. In some embodiments, the at least one fatty alcohol comprises from about 1% to about 10% by weight of the foamable carrier component. In some embodiments, the at least one fatty alcohol comprises from about 0.5% to about 5% by weight of the foamable carrier component. In some embodiments, the oil phase comprises at least one C₁₆₋₁₈ fatty alcohol. In some embodiments, the oil phase comprises cetyl alcohol or stearyl alcohol. In some embodiments, the oil phase comprises cetyl alcohol. In some embodiments, the foamable carrier component comprises stearyl alcohol. In some embodiments, the oil phase comprises cetyl alcohol and stearyl alcohol. In some embodiments, the fatty alcohol functions as an emollient. In some embodiments, the fatty alcohol functions as a foaming agent. In some embodiments, the fatty alcohol functions as an emollient and a foaming agent.

In some embodiments, the oil phase comprises an emollient component. In some embodiments, the emollient component comprises from about 1% to about 10% by weight of the foamable carrier component. In some embodiments, the emollient component comprises from about 0.5% to about 5% by weight of the foamable carrier component. In some embodiments, the emollient component comprises at least one fatty alcohol. In some embodiments, the emollient component comprises at least one C₁₆₋₁₈ fatty alcohol. In some embodiments, the emollient component comprises cetyl alcohol or stearyl alcohol. In some embodiments, the emollient component comprises cetyl alcohol and stearyl alcohol. In some embodiments, the emollient component comprises cetyl alcohol. In some embodiments, the emollient component comprises stearyl alcohol.

In some embodiments, the oil phase comprises a foaming agent component. In some embodiments, the oil phase comprises from about 1% to about 10% by weight of the foamable carrier component. In some embodiments, the oil phase comprises from about 0.5% to about 5% by weight of the foamable carrier component. In some embodiments, the foamable carrier component comprises a foaming agent component. In some embodiments, the foaming agent component comprises at least one fatty alcohol. In some embodiments, the foaming agent component comprises at least one C₁₆₋₁₈ fatty alcohol. In some embodiments, the foaming agent component comprises cetyl alcohol or stearyl alcohol. In some embodiments, the foaming agent component comprises cetyl alcohol.

In some embodiments, the foamable carrier component comprises a solvent component. In some embodiments, the solvent component comprises about 30% to about 95% by weight of the foamable carrier component. In some embodiments, the solvent component comprises about 40% to about 90% by weight of the foamable carrier component. In some embodiments, the solvent component comprises about 40% to about 80% by weight of the foamable carrier component. In some embodiments, the solvent component comprises a C₁₋₄ aliphatic alcohol. In some embodiments, the solvent component comprises ethanol. In some embodiments, the solvent component comprises polyethylene glycol. In some embodiments, the solvent component comprises a polyalkylene glycol. In some embodiments, the solvent component comprises PEG200 or PEG300. In some embodiments, the solvent component comprises an alkylene glycol. In some embodiments, the solvent component comprises propylene glycol. In some embodiments, the solvent component comprises a C₁₋₄ aliphatic alcohol, a polyalkylene glycol, or an alkylene glycol, or a mixture of any of the foregoing. In some embodiments, the solvent component comprises ethanol, polyethylene glycol, or propylene glycol, or a mixture of any of the foregoing. In some embodiments, the solvent component comprises ethanol, PEG200, PEG300, or propylene glycol, or a mixture of any of the foregoing.

In some embodiments, the solvent component comprises about 0.05% to about 20% of a permeation enhancer. In some embodiments, the solvent component comprises about 0.5% to about 10% of a permeation enhancer. In some embodiments, the solvent component comprises about 2% to about 20% of a permeation enhancer. In some embodiments, the permeation enhancer is propylene glycol. In some embodiments, the permeation enhancer is diethylene glycol monoethyl ether (Transcutol P).

In some embodiments, the water comprises about 20% to about 70% by weight of the foamable carrier component. In some embodiments, the water comprises about 30% to about 60% by weight of the foamable carrier component.

In some embodiments, the foamable carrier component comprises ≥50% water by weight of the foamable carrier component (e.g., 70% to 80%). In some embodiments, the foamable carrier component comprises ≤50% water by weight of the foamable carrier component (e.g., below 50%).

In some embodiments, the foamable carrier component is present in an amount of about 70% to about 99.99% of the foamable composition. In some embodiments, the foamable carrier component is present in an amount of about 80% to about 99% of the foamable composition. In some embodiments, the foamable carrier component is present in an amount of about 50% to about 98% of the foamable composition. In some embodiments, the foamable carrier component is present in an amount of about 50% to about 95% of the foamable composition. In some embodiments, the foamable carrier component is present in an amount of about 60% to about 95% of the foamable composition. In some embodiments, the foamable carrier component is present in an amount of about 70% to about 95% of the foamable composition. In some embodiments, the foamable carrier component is present in an amount of about 75% to about 98% of the foamable composition. In some embodiments, the foamable carrier component is present in an amount of about 75% to about 95% of the foamable composition. In some embodiments, the foamable carrier component is present in an amount of about 80% to about 90% of the foamable composition.

In some embodiments, the foamable composition comprises a propellant component. In some embodiments, the propellant component comprises about 2% to about 20% of the foamable composition. In some embodiments, the propellant component comprises about 5% to about 15% of the foamable composition. In some embodiments, the propellant component comprises about 2% to about 15% of the foamable composition. In some embodiments, the propellant component comprises about 2% to about 10% of the foamable composition. In some embodiments, the propellant component comprises about 5% to about 10% of the foamable composition.

In some embodiments, the foamable composition and/or foamable carrier component does not comprise an organic amine pH adjusting agent. In some embodiments, an organic amine pH adjusting agent is an aromatic amine, a tertiary amine, a secondary amine, a primary amine, ammonia, or an alkanol amine, such as a mono-di- or tri-alkanolamine, e.g., a trialkanolamine or trolamine. In some embodiments, the organic amine pH adjusting agent is trolamine, tris, ethanolamine, diethanolamine, ammonia, diisopropanolamine, 1-amino-2-propanol, 2-amino-2-ethyl-1,3-propanediol, 2-amino-2-methyl-1-propanol, diisopropylamine, imidazole, and pyridine.

In some embodiments, the foamable carrier component comprises:
from about 40% to about 90% of solvent component by weight of the foamable carrier component;
from about 30% to about 60% of water by weight of the foamable carrier component; and
from about 0.5% to about 10% of an oil phase by weight of the foamable carrier component.

In some embodiments, the foamable carrier component comprises:
from about 40% to about 65% of ethanol by weight of the foamable carrier component;
from about 30% to about 60% of water by weight of the foamable carrier component;
from about 0.5% to about 5% of stearyl alcohol by weight of the foamable carrier component;
from about 0.5% to about 5% of cetyl alcohol by weight of the foamable carrier component; and
from about 2% to about 20% of propylene glycol.

In some embodiments, the foamable carrier component comprises:
from about 40% to about 95% of solvent component by weight of the foamable carrier component;
from about 30% to about 60% water by weight of the foamable carrier component; and
from about 0.5% to about 10% of at least one fatty alcohol. by weight of the foamable carrier component.

In some embodiments, the foamable carrier component comprises:
from about 40% to about 65% of ethanol by weight of the foamable carrier component;
from about 30% to about 60% of water by weight of the foamable carrier component;
from about 0.25% to about 5% of stearyl alcohol by weight of the foamable carrier component;
from about 0.5% to about 5% of cetyl alcohol by weight of the foamable carrier component; and
from about 2% to about 10% of propylene glycol.

In some embodiments, the foamable carrier component comprises:
from about 40% to about 60% of solvent component by weight of the foamable carrier component;
from about 30% to about 50% water by weight of the foamable carrier component; and
from about 0.5% to about 10% of an oil phase by weight of the foamable carrier component.

In some embodiments, the foamable carrier component comprises:
≥50% solvent component by weight of the foamable carrier component;
≤50% water by weight of the foamable carrier component; and
from about 0.5% to about 10% of an oil phase by weight of the foamable carrier component.

### Active Pharmaceutical Ingredient

In some embodiments, the compound (i.e., the active pharmaceutical ingredient) in the foamable carrier component is ruxolitinib, deuterated ruxolitinib, or a pharmaceutically acceptable salt of any of those aforementioned. In some embodiments, the compound is ruxolitinib, or a pharmaceutically acceptable salt thereof. In some embodiments, the compound is ruxolitinib phosphate. In some embodiments, the compound is ruxolitinib sulfate. In some embodiments, the compound is ruxolitinib maleate.

In some embodiments, the compound is deuterated ruxolitinib, or a pharmaceutically acceptable salt thereof. In some embodiments, the compound is deuruxolitinib, or a pharmaceutically acceptable salt thereof. In some embodiments, the compound is deuruxolitinib phosphate.

In some embodiments, the compound is present in an amount from about 0.05% to about 5.5% by weight of the foamable carrier component on a free base basis of the compound, which is ruxolitinib or deuterated ruxolitinib, or a pharmaceutically acceptable salt of any of the aforementioned. In some embodiments, the compound is present in an amount from about 1% to about 4% by weight of the foamable carrier component on a free base basis of the compound, which is ruxolitinib or deuterated ruxolitinib, or a pharmaceutically acceptable salt of any of the aforementioned. In some embodiments, the compound is present in an amount from about 0.05% to about 1.5% by weight of the foamable carrier component on a free base basis of the compound, which is ruxolitinib or deuterated ruxolitinib, or a pharmaceutically acceptable salt of any of the aforementioned. In some embodiments, the foamable carrier component comprises about 0.05%, about 0.06%, about 0.07%, about 0.08%, about 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, about 2.0%, about 2.5%, about 3.0%, about 3.5%, about 4.0%, about 4.5%, about 5.0%, or about 5.5% by weight of the foamable carrier component on a free base basis of the compound, or a pharmaceutically acceptable salt thereof. In some embodiments, the compound is present in an amount from about 1% to about 4% by weight of the foamable carrier component on a free base basis of the compound, which is ruxolitinib or deuterated ruxolitinib, or a pharmaceutically acceptable salt of any of the aforementioned. In some embodiments, the compound is present in an amount from about 1% to about 2% by weight of the foamable carrier component on a free base basis of the compound, which is ruxolitinib or deuterated ruxolitinib, or a pharmaceutically acceptable salt of any of the aforementioned. In some embodiments, the compound is present in an amount from about 2% to about 3% by weight of the foamable carrier component on a free base basis of the compound, which is ruxolitinib or deuterated ruxolitinib, or a pharmaceutically acceptable salt of any of the aforementioned. In some embodiments, the foamable carrier component comprises from about 1.0% to about 3.0% by weight of the foamable carrier component on a free base basis of the compound, or a pharmaceutically acceptable salt thereof. In some embodiments, the foamable carrier component comprises from about 0.5% to about 1.5% by weight of the foamable carrier component on a free base basis of the compound, or a pharmaceutically acceptable salt thereof. In some embodiments, the foamable carrier component comprises from about 0.3% to about 1% by weight of the foamable carrier component on a free base basis of the compound, or a pharmaceutically acceptable salt thereof. In some embodiments, the foamable carrier component comprises from about 0.3% to about 0.8% by weight of the foamable carrier component on a free base basis of the compound, or a pharmaceutically acceptable salt thereof. In some embodiments, the compound in each of the embodiments of this paragraph is ruxolitinib, or a pharmaceutically acceptable salt thereof. In some embodiments, the compound in each of the embodiments of this paragraph is ruxolitinib phosphate. In some embodiments, the compound in each of the embodiments of this paragraph is ruxolitinib chloride. In some embodiments, the compound in each of the embodiments of this paragraph is deuruxolitinib, or a pharmaceutically acceptable salt thereof. In some embodiments, the compound in each of the embodiments of this paragraph is deuruxolitinib phosphate.

In some embodiments, the foamable carrier component comprises 1.5% by weight of the foamable carrier component on a free base basis of the compound, or a pharmaceutically acceptable salt thereof. In some embodiments, the foamable carrier component comprises 2.5% by weight of the foamable carrier component on a free base basis of the compound, or a pharmaceutically acceptable salt thereof. In some embodiments, the foamable carrier component comprises 3.0% by weight of the foamable carrier component on a free base basis of the compound, or a pharmaceutically acceptable salt thereof. In some embodiments, the foamable carrier component comprises 3.5% by weight of the foamable carrier component on a free base basis of the compound, or a pharmaceutically acceptable salt thereof. In some embodiments, the foamable carrier component comprises 4.0% by weight of the foamable carrier component on a free base basis of the compound, or a pharmaceutically acceptable salt thereof. In some embodiments, the compound in each of the embodiments of this paragraph is ruxolitinib, or a pharmaceutically acceptable salt thereof. In some embodiments, the compound in each of the embodiments of this paragraph is ruxolitinib phosphate. In some embodiments, the compound in each of the embodiments of this paragraph is ruxolitinib chloride. In some embodiments, the compound in each of the embodiments of this paragraph is deuruxolitinib, or a pharmaceutically acceptable salt thereof. In some embodiments, the compound in each of the embodiments of this paragraph is deuruxolitinib phosphate.

### Water

In some embodiments, the foamable carrier component comprises water or a water phase. In some embodiments, the foamable carrier component comprises water. In some embodiments, the water comprises about 20% to about 70% by weight of the foamable carrier component. In some embodiments, the water comprises about 30% to about 60% by weight of the foamable carrier component.

In some embodiments, the foamable carrier component comprises ≥50% water by weight of the foamable carrier component (e.g., 70% to 80%). In some embodiments, the foamable carrier component comprises ≤50% water by weight of the foamable carrier component (e.g., below 50%).

In some embodiments, the foamable carrier component comprises ≥50% water by weight of the foamable carrier component (e.g., 70% to 80%). In some embodiments, the water is present in an amount from about 50% to about 99.9%, from about 50% to about 90%, from about 50% to about 80%, from about 50% to about 70%, and from about 50% to about 60%, by weight of the foamable carrier component.

In some embodiments, the foamable carrier component comprises ≤50% water by weight of the foamable carrier component (e.g., below 50%). In some embodiment, the water is present in an amount from about 1% to about 50%, from about 5% to about 50%, from about 5% to about 50%, from about 10% to about 50%, from about 20% to about 50%, from about 30% to about 50%, from about 40% to about 50%, and from about 45% to 50%, by weight of the foamable carrier component.

In some embodiments, the foamable carrier component further comprises one or more additives. In some embodiments, the one or more additives are miscible in water. In some embodiments, the one or more additives are chosen from, but not limited to, polysorbate 60 (HLB 14.9), propylene glycol, glycerin, Transcutol-P, SP Tween 60 MBAL (ethoxylated (2) sorbitan ester), and combinations thereof. In some embodiments, the one or more additives are present in an amount ranging from 1 w/w % to 20 w/w %, of the water phase. In some embodiments, the one more additives are present in an amount ranging from 10 w/w % to 18 w/w %, of the water phase.

### Oil Phase

In some embodiments, the foamable carrier component comprises an oil phase. In some embodiments, the oil phase is present in an amount of about 3% to about 70% by weight of the foamable carrier component. In some embodiments, the oil component is present in an amount of about 5% to about 60% by weight of the foamable carrier component. In some embodiments, the oil phase is present in an amount of about 5% to about 50% by weight of the foamable carrier component. In some embodiments, the oil phase is present in an amount of about 5% to about 40% by weight of the foamable carrier component. In some embodiments, the oil phase is present in an amount of about 5% to about 30% by weight of the foamable carrier component. In some embodiments, the oil phase is present in an amount of about 10% to about 60% by weight of the foamable carrier component. In some embodiments, the oil phase is present in an amount of about 10% to about 50% by weight of the foamable carrier component. In some embodiments, the oil phase is present in an amount of about 10% to about 40% by weight of the foamable carrier component. In some embodiments, the oil phase is present in an amount of about 10% to about 30% by weight of the foamable carrier component.

In some embodiments, the oil phase is present in an amount of about 0.5% to about 30% by weight of the foamable carrier component. In some embodiments, the oil phase is present in an amount of about 0.5% to about 20% by weight of the foamable carrier component. In some embodiments, the oil phase is present in an amount of about 0.5% to about 10% by weight of the foamable carrier component.

In some embodiments, the oil phase comprises at least one or one or more fatty alcohols. In some embodiments, the fatty alcohol comprises from about 1% to about 10% by weight of the foamable carrier component. In some embodiments, the fatty alcohol comprises from about 0.5% to about 5% by weight of the foamable carrier component. In some embodiments, the oil phase comprises at least one C₁₆₋₁₈ fatty alcohol. In some embodiments, the oil phase comprises cetyl alcohol or stearyl alcohol. In some embodiments, the oil phase comprises cetyl alcohol or stearyl alcohol, or a mixture thereof. In some embodiments, the oil phase comprises cetyl alcohol. In some embodiments, the oil phase comprises stearyl alcohol. In some embodiments, the oil phase comprises cetyl alcohol and stearyl alcohol. In some embodiments, the fatty alcohol functions as an emollient. In some embodiments, the fatty alcohol functions as a foaming agent. In some embodiments, the fatty alcohol functions as an emollient and a foaming agent.

In some embodiments, the oil phase comprises an emollient component. In some embodiments, the emollient component comprises from about 1% to about 10% by weight of the foamable carrier component. In some embodiments, the emollient component comprises from about 0.5% to about 5% by weight of the foamable carrier component. In some embodiments, the emollient component comprises at least one fatty alcohol. In some embodiments, the emollient component comprises at least one C₁₆₋₁₈ fatty alcohol. In some embodiments, the emollient component comprises cetyl alcohol or stearyl alcohol. In some embodiments, the emollient component comprises cetyl alcohol and stearyl alcohol. In some embodiments, the emollient component comprises cetyl alcohol. In some embodiments, the emollient component comprises stearyl alcohol.

In some embodiments, the oil phase comprises a foaming agent component. In some embodiments, the oil phase comprises from about 1% to about 10% by weight of the foamable carrier component. In some embodiments, the oil phase comprises from about 0.5% to about 5% by weight of the foamable carrier component. In some embodiments, the foamable carrier component comprises a foaming agent component. In some embodiments, the foaming agent component comprises at least one fatty alcohol. In some embodiments, the foaming agent component comprises at least one C₁₆₋₁₈ fatty alcohol. In some embodiments, the foaming agent component comprises cetyl alcohol or stearyl alcohol. In some embodiments, the foaming agent component comprises cetyl alcohol. In some embodiments, the emollient component comprises cetyl alcohol and stearyl alcohol.

In some embodiments, the oil phase further comprises an emulsifier or stabilizer component, or an emulsifier or wetting agent component.

In some embodiments, the emulsifying or wetting agent component is present in amount of about 1% to about 40% by weight of the foamable carrier component. In some embodiments, the emulsifying or wetting agent component is present in amount of about 1% to about 30% by weight of the foamable carrier component. In some embodiments, the emulsifying or wetting agent component is present in amount of about 1% to about 20% by weight of the foamable carrier component. In some embodiments, the emulsifying or wetting agent component is present in amount of about 2% to about 20% by weight of the foamable carrier component. In some embodiments, the emulsifying or wetting agent component is present in amount of about 5% to about 20% by weight of the foamable carrier component. In some embodiments, the emulsifying or wetting agent component is present in amount of about 10% to about 20% by weight of the foamable carrier component.

In some embodiments, the oil phase further comprises one or more substances selected from fatty alcohols (e.g., cetyl alcohol, stearyl alcohol, cetostearyl alcohol (such as Kolliphor CSA50), and octodecanol (Kolliphor OD)), fatty acids, fatty esters (isopropyl myristate, sorbitan laurate), glyceryl fatty esters (e.g., glyceryl monostearate (Kolliwax GMS II)), sorbitan fatty esters (e.g., polysorbate 20, polysorbate 80 (Span 80)), polyethylene glycol fatty ethers (e.g., PEG 100 stearate (component of Arlacel 165), polyethylene glycol 300 (PEG300), polyethylene glycol 400 (PEG 400)), polyethylene glycol hexadecyl ether (Cetomacrogol 1000), polyethylene glycol octadecyl ether (Brij S2), polyoxyethylene stearyl ether (Brij S721), ethoxylated stearic and cetyl alcohols (Kolliphor CS20)), waxes (e.g., paraffin (soft white paraffin), emulsifying waxes (Polawax)), mineral, natural, hydrogenated, and silicone oils (e.g., light mineral oil, castor oil, silicone oils (e.g., cyclomethicone, dimethicone), hydrogenated castor oils (Kolliphor HCO), fatty ester (cocoyl caprylocaprate (Kollicream 3C)), and triglycerides (caprylic/capric triglyceride (Crodamol GTCC), medium chain triglycerides), or combinations thereof. In some embodiments, the oil component comprises one or more substances selected from fatty acids (e.g., lanolin acid), fatty alcohols (e.g., lanolin alcohol), hydrocarbon oils & waxes (e.g., petrolatum), polyhydric alcohols (e.g., propylene glycol), silicones (e.g., dimethicone), sterols (e.g., cholesterol), xanthan gum, vegetable or animal fat (e.g., cocoa butter), vegetable wax (e.g., Carnauba wax), and wax ester (e.g., bees wax), or combinations thereof.

In some embodiments, the oil phase further comprises an emulsifier or stabilizer component, or an emulsifier or wetting agent component. In some embodiments, the emulsifying or wetting agent component comprises one or more substances selected from fatty alcohols (e.g., cetyl alcohol, stearyl alcohol, cetostearyl alcohol (such as Kolliphor CSA50), and octodecanol (Kolliphor OD)), fatty acids, fatty esters, glyceryl fatty esters (e.g., glyceryl monostearate (Kolliwax GMS II)), sorbitan fatty esters (e.g., polysorbate 20, polysorbate 80 (Span 80)), polyethylene glycol fatty ethers (e.g., PEG 100 stearate (component of Arlacel 165)), polyethylene glycol 300 (PEG300), polyethylene glycol 400 (PEG 400), polyethylene glycol hexadecyl ether (Cetomacrogol 1000), polyethylene glycol octadecyl ether (Brij S2), polyoxyethylene stearyl ether (Brij S721), ethoxylated stearic and cetyl alcohols (Kolliphor CS20)), and emulsifying waxes (Polawax)). In some embodiments, the emulsifying or wetting agent component comprises one or more substances selected from fatty alcohols (e.g., cetyl alcohol, stearyl alcohol, and cetostearyl alcohol (such as Kolliphor CSA50)), fatty esters, glyceryl fatty esters (e.g., glyceryl monostearate (Kolliwax GMS II)), sorbitan fatty esters (e.g., polysorbate 20, polysorbate 80 (Span 80)), polyethylene glycol fatty ethers (e.g., PEG 100 stearate (component of Arlacel 165)), polyethylene glycol 300 (PEG300), polyethylene glycol 400 (PEG 400), polyethylene glycol hexadecyl ether (Cetomacrogol 1000), polyethylene glycol octadecyl ether (Brij S2), and polyoxyethylene stearyl ether (Brij S721), ethoxylated stearic and cetyl alcohols (Kolliphor CS20)).

In some embodiments, the oil phase further comprises one or more stabilizing agents. In some embodiments, the one or more stabilizing agents comprises one or more substances independently selected from polysaccharides. In some embodiments, the one or more stabilizing agents is xanthan gum.

Additionally, in some embodiments, the oil phase further comprises one or more independently selected from occlusive agent, stiffening agent, and emollient. In some embodiments, the oil phase comprises one or more occlusive agent. In some embodiments, the oil phase comprises one or more stiffening agent. In some embodiments, the oil phase comprises one more emollient.

In some embodiments, the oil component comprises one or more emollient. In some embodiments, the emollient comprises one or more substances chosen from, but not limited to, PEG-6 caprylic capric glycerides (Glycerox 767), glyceryl caprylate, glyceryl caprate, isostearic acid, glycerol monolaurate, glycerin, PPG stearyl ether, diisopropyl adipate (DIPA), Arlamol PS11E pharma (prpoxylate), oleic acid, polyethylene glycol (PEG 300), myristyl lactate, diethylene glycol monoethyl ether (Transcutol-P), and combinations thereof. In some embodiments, the emollient comprises glycerin and PPG15 stearyl ether. In some embodiments, the emollient comprises glycerin and DIPA. In some embodiments, the emollient comprises glycerin and oleic acid. In some embodiments, the emollient comprises PEG300 and glycerin. In some embodiments, the emollient comprises DIPA and oleic acid. In some embodiments, the emollient comprises myristyl lactate and Transcutol-P. In some embodiments, the emollient comprises PPG 15 SE and Transcuol-P. In some embodiments, the emollient comprises PEG300, glycerin, and myristyl lactate. In some embodiments, the emollient comprises myristyl lactate. In some embodiments, the emollient comprises PEG 300, mysrityl lactate and Transcutol-P. In some embodiments, the emollient comprises mysrityl lactate and Transcutol-P.

In some embodiments, the one or more emollients are present in an amount ranging from about 0.5% to 10% by weight of the foamable carrier component. In some embodiments, the one or more emollients are present in an amount ranging from about 1% to 8% by weight of the foamable carrier component. In some embodiments, the one or more emollients are present in an amount ranging from about 1% to 5% by weight of the foamable carrier component.

In some embodiments, the oil phase further comprises an occlusive agent component. In some embodiments, the occlusive agent is present in an amount of about 0.1% to about 15% by weight of the formulation.

In some embodiments, the occlusive agent component comprises one or more substances selected from fatty acids (e.g., lanolin acid), fatty alcohols (e.g., lanolin alcohol), hydrocarbon oils & waxes (e.g., petrolatum), polyhydric alcohols (e.g., propylene glycol), silicones (e.g., dimethicone), sterols (e.g., cholesterol), vegetable or animal fat (e.g., cocoa butter), vegetable wax (e.g., Carnauba wax), and wax ester (e.g., bees wax).

In some embodiments, the occlusive agent component comprises one or more substances selected from lanolin acid fatty alcohols, lanolin alcohol, petrolatum, propylene glycol, dimethicone, cholesterol, cocoa butter, Carnauba wax, and bees wax. In some embodiments, the occlusive agent component comprises petrolatum. In some embodiments, the occlusive agent component comprises white petrolatum.

In some embodiments, the oil phase further comprises a stiffening agent. In some embodiments, the stiffening agent component comprises one or more substances independently selected from fatty alcohols. In some embodiments, the stiffening agent component comprises one or more substances independently selected from C₁₂₋₂₀ fatty alcohols. In some embodiments, the stiffening agent component comprises one or more substances independently selected from C₁₆₋₁₈ fatty alcohols. In some embodiments, the stiffening agent component comprises one or more substances independently selected from cetyl alcohol and stearyl alcohol.

### Solvent

In some embodiments, the foamable composition and/or the foamable carrier component further comprises a solvent component. In some embodiments, the solvent component comprises about 30% to about 95% by weight of the foamable carrier component. In some embodiments, the solvent component comprises about 40% to about 90% by weight of the foamable carrier component.

In some embodiments, the foamable carrier component further comprises a solvent component. In some embodiments, the solvent component is present in amount of about 5% to about 70% by weight of the foamable carrier component. In some embodiments, the solvent component is present in amount of about 5% to about 60% by weight of the foamable carrier component. In some embodiments, the solvent component is present in amount of about 5% to about 50% by weight of the foamable carrier component. In some embodiments, the solvent component is present in amount of about 10% to about 70% by weight of the foamable carrier component. In some embodiments, the solvent component is present in amount of about 10% to about 60% by weight of the foamable carrier component. In some embodiments, the solvent component is present in amount of about 10% to about 50% by weight of the foamable carrier component. In some embodiments, the solvent component is present in amount of about 10% to about 40% by weight of the foamable carrier component. In some embodiments, the solvent component is present in amount of about 20% to about 70% by weight of the foamable carrier component. In some embodiments, the solvent component is present in amount of about 20% to about 60% by weight of the foamable carrier component. In some embodiments, the solvent component is present in amount of about 20% to about 50% by weight of the foamable carrier component.

In some embodiments, the solvent component comprises about 40% to about 80% by weight of the foamable carrier component. In some embodiments, the solvent component comprises a C₁₋₄ aliphatic alcohol. In some embodiments, the solvent component comprises ethanol. In some embodiments, the solvent component comprises polyalkylene glycol. In some embodiments, the solvent component comprises a polyethylene glycol. In some embodiments, the solvent component comprises PEG200 or PEG300. In some embodiments, the solvent component comprises an alkylene glycol. In some embodiments, the solvent component comprises propylene glycol. In some embodiments, the solvent component comprises a C₁₋₄ aliphatic alcohol, a polyalkylene glycol, or an alkylene glycol, or a mixture of any of the foregoing. In some embodiments, the solvent component comprises ethanol, polyethylene glycol, or propylene glycol, or a mixture of any of the foregoing. In some embodiments, the solvent component comprises ethanol, PEG200, PEG300, or propylene glycol, or a mixture of any of the foregoing.

In some embodiments, the solvent component comprises one or more hydroxylated solvents. In some embodiments, the solvent component comprises one or more substances selected from dimethyl glycol, diethylene glycol diethers (e.g., diethylene glycol monoethyl ether (Transcutol P)), glycerol, alkylene glycols (e.g., propylene glycol), or polyethylene glycols (e.g., polyethylene glycol 300 (PEG300), polyethylene glycol 400 (PEG 400)).

In some embodiments, the solvent component comprises one or more independent selected from polyols, diethylene glycol monoethyl ethers, alkylene glycols, polyalklene glycols, propylene glycols, and polyethylene glycols.

In some embodiments, the solvent component comprises polyols. In some embodiments, the polyols are glycerol. In some embodiments, the glycerol is present in an amount ranging from about 10% to about 20%, by weight of the foamable carrier component.

In some embodiments, the solvent component comprises diethylene glycol monoethyl ethers. In some embodiments, the diethylene glycol monoethyl ethers (DEGEE) are Transcutol® P. In some embodiments the Transcutol® P is present in an amount ranging from about 13% to about 40% %, by weight of the foamable carrier component.

In some embodiments, the solvent component comprises ethanol. In some embodiments, when the solvent component comprises ethanol, a ratio of ethanol to water ranges from 55:45 to 95:5, such as from 60:40 to 80:20. The ethanol to water ratio is calculated by dividing the amount of ethanol by the total sum of water plus ethanol and is not the % w/w of ethanol and water based on the total weight of the foamable carrier component.

In some embodiments, where the foamable composition has an ethanol to water ratio with a higher amount of ethanol to water, the ratio of ethanol to water provides for a foamable carrier component allowing for the solubility of the active, e.g., ruxolitinib phosphate. In some embodiments, the foamable carrier component has a ratio of ethanol to water of 60:40. In some embodiments, the foamable carrier component has a ratio of ethanol to water of 80:20.

### Additional Components

In some embodiments, depending on the selected foamable composition and/or the selected foamable carrier component, one or more additional excipients as described herein may be necessary, e.g., pH adjusting agents, chelating agents, preservatives, co-solvents, penetration enhancers, humectants, thickening agents, gelling agents, viscosity building agents, surfactants, fragrances, colorants, carriers, antioxidants, or any combination or mixture thereof.

In some embodiments, the foamable carrier component further comprises one or more preservatives. In some embodiments, the one or more preservatives are benzyl alcohol, methyl paraben, propyl paraben, phenoxyethanol, and combinations thereof.

In some embodiments, the foamable carrier component further comprises a chelating agent component. In some embodiments, the chelating agent is present in an amount from about 0.01% to about 15% by weight of the foamable carrier component. In some embodiments, the chelating agent component comprises edetate disodium. In some embodiments, the edetate disodium is present in an amount of about 0.001% to about 5% by weight of the foamable carrier component.

In some embodiments, the foamable carrier component further comprises a humectant. In some embodiments, the humectant is present in an amount from about 0.01% to about 20% by weight of the formulation. In some embodiments, the humectant is glycerol. In some embodiments, the glycerol is present in an amount of about 0.01% to about 20% by weight of the formulation. In some embodiments, the glycerol is present in an amount of about 0.1% to about 20% by weight of the foamable carrier component.

In some embodiments, the foamable carrier component further comprises a surfactant. In some embodiments, the surfactant is present in an amount from about 0.01% to about 20% by weight of the foamable carrier component. A surfactant is a compound that lowers the surface tension between two liquids (e.g., between the polar solvent component and the oil component). Surfactant may be a mixture of two or more surfactants. Exemplary surfactants include, but are not limited to, ethoxylated fatty alcohol ether (e.g., steareth-2, steareth-10, steareth-20, ceteareth-2, ceteareth-10, and the like), PEG esters (e.g., PEG-4 dilaurate, PEG-20 stearate, and the like), Glyceryl esters or derivatives thereof (e.g., glyceryl dioleate, glyceryl stearate, and the like), polymeric ethers (e.g., poloxamer 124, poloxamer 181, poloxamer 182, poloxamer 407, and the like), sorbitan derivatives (e.g., polysorbate 80, sorbitan monostearate, and the like), fatty alcohols (e.g., cetyl alcohol, stearyl alcohol, cetearyl alcohol, and the like), and emulsifying wax (e.g., emulsifying wax NF, mixtures of mixture of cetearyl alcohol and polysorbate 60, and the like).

In some embodiments, the foamable carrier component comprises one or more non-ionic emulsifying agents and emulsifying waxes, or combinations thereof. In some embodiments, the foamable carrier component comprises one or more substances selected from fatty alcohols (e.g., cetyl alcohol, stearyl alcohol, cetostearyl alcohol (such as Kolliphor CSA50), and octodecanol (Kolliphor OD)), fatty acids, fatty esters, glyceryl fatty esters (e.g., glyceryl monostearate (Kolliwax GMS II)), sorbitan fatty esters (e.g., polysorbate 20, polysorbate 80 (Span 80)), polyethylene glycol fatty ethers (e.g., polyethylene glycol hexadecyl ether (Cetomacrogol 1000), polyethylene glycol octadecyl ether (Brij S2), polyoxyethylene stearyl ether (Brij S721)), and emulsifying waxes (Polawax)), or combinations thereof.

In some embodiments, the surfactant is polysorbate 80. In some embodiments, the surfactant is polysorbate 80 is present in an amount of about 0.01% to about 15% by weight of the formulation. In some embodiments, the surfactant is polysorbate 80 is present in an amount of about 0.1% to about 15% by weight of the foamable carrier component.

In some embodiments, the foamable composition and/or the foamable carrier component further comprises about 0.05% to about 20% of a permeation enhancer. In some embodiments, the foamable composition and/or the foamable carrier component further comprises about 0.5% to about 10% of a permeation enhancer. In some embodiments, the foamable composition and/or the foamable carrier component further comprises about 2% to about 20% of a permeation enhancer. In some embodiments, the foamable composition and/or the foamable carrier component further comprises about 0.5% to about 5% of a permeation enhancer. In some embodiments, the permeation enhancer is propylene glycol. In some embodiments, the foamable composition and/or the foamable carrier component further comprises about 0.5% to about 1% of a permeation enhancer. In some embodiments, the permeation enhance is diethylene glycol monoethyl ether (transcutol-P) or polypropylene glycol.

In some embodiments, a permeation enhancer comprises a polyol such as polyethylene glycol (PEG, e.g., polyethylene glycol 300 (PEG300), polyethylene glycol 400 (PEG 400)), glycerol (glycerin), maltitol, sorbitol etc.; diethylene glycol monoethyl ether, azone, benzalkonium chloride (ADBAC), cetylperidium chloride, cetylmethylammonium bromide, dextran sulfate, lauric acid, menthol, methoxysalicylate, oleic acid, phosphatidylcholine, polyoxyethylene, polysorbate 80, sodium glycholate, sodium lauryl sulfate, sodium salicylate, sodium taurocholate, sodium taurodeoxycholate, sulfoxides, sodium deoxycholate, sodium glycodeoxycholate, sodium taurocholate and surfactants such as sodium lauryl sulfate, laureth-9, cetylpyridinium chloride and polyoxyethylene monoalkyl ethers, benzoic acids, such as sodium salicylate and methoxy salicylate, fatty acids, such as lauric acid, oleic acid, undecanoic acid and methyl oleate, fatty alcohols, such as octanol and nonanol, laurocapram, cyclodextrins, thymol, limonene, urea, chitosan and other natural and synthetic polymers.

In some embodiments, the foamable carrier component comprises a thickening agent. In some embodiments, the thickening agent is present in an amount from about 0.01% to about 15% by weight of the foamable carrier component. In some embodiments, a thickening agent comprises beeswax, hard paraffin or cetyl alcohol, xanthan gum, carboxymethyl cellulose, hydroxypropyl cellulose, carbomer, or povidone (e.g., Kollidon 90F).

In some embodiments, the foamable carrier component comprises a gelling agent. In some embodiments, the gelling agent is present in an amount from about 0.01% to about 15% by weight of the foamable carrier component. In some embodiments, a gelling agent is a material that can swell or expand when in contact with water. In some embodiments, the gelling agent comprises swellable polymers such as osmopolymers or hydrogels. In some embodiments, the gelling agent is non-cross linked or lightly cross-linked. In some embodiments, the gelling agent is olyhydroxyalkylcellulose having a molecular weight greater than 50,000, such as hydroxyl propylmethylcellulose (METHOCEL K 100M available from Dow Chemical); poly (hydroxyalkylmethacrylate) having a molecular weight of from 5,000 to 5,000,000; poly (vinylpyrrolidone) having a molecular weight of from 100,000 to 3,000,000; anionic and cationic hydrogels; poly (electrolyte) complexes; poly (vinylalcohol) having a low acetate residual; a swellable mixture of agar and carboxymethyl cellulose; a swellable composition comprising methyl cellulose mixed with a sparingly cross-linked agar; a polyether having a molecular weight of from 10,000 to 6,000,000; a water-swellable copolymer produced by a dispersion of a finely divided copolymer of maleic anhydride with styrene, ethylene, propylene, or isobutylene; a water-swellable polymer of N-vinyl lactams, and the like.

In some embodiments, the foamable carrier component further comprises a viscosity building agent. In some embodiments, the viscosity building agent is present in an amount from about 0.01% to about 15% by weight of the foamable carrier component. In some embodiments, the viscosity building agent includes, but is not limited to, natural or synthetic waxes such as carnauba wax, cetyl ester wax, microcrystalline wax, white wax, yellow wax, bees wax, ozokerite, paraffin, ceresin, esparto wax, ouricury wax, and rezowax, hard fats (e.g., hydrogenated vegetable glycerides), hydrogenated vegetable oils, C₁₂-C₆₀ alcohols, C₁₂-C₆₀ acids, alpha-hydroxy fatty acids, polyhydroxy fatty acid esters, polyhydroxy fatty acid amides and combinations thereof.

In some embodiments, the foamable carrier component further comprises one or more co-solvents. In some embodiments, the one or more co-solvents comprise one or more additional hydroxylated solvents. In some embodiments, the solvent component comprises one or more substances selected from diethylene glycol diethers (e.g., diethylene glycol monoethyl ether (Transcutol P)), alkylene glycols (e.g., propylene glycol), or polyethylene glycols (e.g., PEG400). In some embodiments, the co-solvent is present in an amount from about 0.01% to about 15% by weight of the foamable carrier component.

In some embodiments, foamable carrier components can contain one or more conventional carriers as described herein. In some embodiments, the carrier is present in an amount from about 0.01% to about 15% by weight of the foamable carrier component. In some embodiments, the foamable carrier component can contain water and one or more hydrophobic carriers selected from, for example, liquid paraffin, polyoxyethylene alkyl ether, propylene glycol, white petrolatum, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like.

The present disclosure also provides for foamable carrier component having a pH of from about 4.0 to about 8.0, from about 4.0 to about 7.0, from about 4.0 to about 6.0, about 5.0 to about 8.0, from about 5.5 to about 7.5, from about 5.5 to about 7.0, from about 5.5 to about 6.5, from about 5.0 to about 6.0, and at about 5.5. In some embodiments, the foamable carrier component has a pH of from about 4.0 to about 8.0. In some embodiments, the foamable carrier component has a pH of from about 4.0 to about 7.0. In some embodiments, the foamable carrier component has a pH of from about 4.0 to about 6.0. In some embodiments, the foamable carrier component has a pH of about 5.0 to about 8.0. In some embodiments, the foamable carrier component has a pH of from about 5.5 to about 7.5. In some embodiments, the foamable carrier component has a pH of from about 5.5 to about 7.0. In some embodiments, the foamable carrier component has a pH from about 5.0 to about 7.0. In some embodiments, the foamable carrier component has a pH of from about 5.5 to about 6.5. In some embodiments, the foamable carrier component has a pH of from about 5.0 to about 6.0. In some embodiments, the foamable carrier component has a pH of about 5.5.

In some embodiments, the foamable carrier component is pH adjusted by one or more buffering agents. In some embodiments, the one or more buffering agents comprises one or more independent selected from sodium hydroxide, phosphoric acid, hydrochloric acid, boric acid, tetra boric acid, acetic acid, tartaric acid, citric acid, carbonic acid, and their alkali metal salts or ammonium salts (including acid salts in the case of polybasic acids), organic amines and their quaternary salts, glycine, etc. In some embodiments, the buffering agent is citric acid, anhydrous. In some embodiments, the buffering agent is sodium citrate dihydrate.

In some embodiments, the buffering agent is sodium hydroxide.

In some embodiments, the foamable carrier component further comprises an antioxidant component. In some embodiments, the antioxidant component comprises one or more independent selected from butylated hydroxytoluene (BHT), butylated hydroxyanisole (BHA), propyl gallate, tocopherol, ethylenediaminetetraacetic acid (EDTA), and tocofersolan (TPGS).

In some embodiments, the antioxidant component is present in an amount of from about 0.001% to about 3%, from about 0.05% to about 2.5%, from about 0.05% to about 2%, or about 0.05% to about 1% by weight of the foamable carrier component. In some embodiments, the antioxidant component is present in an amount of from about 0.001% to about 3%, by weight of the foamable carrier component. In some embodiments, the antioxidant component is present in an amount of from about 0.05% to about 2%, by weight of the foamable carrier component. In some embodiments, the antioxidant component is present in an amount of from about 0.05% to about 2%, by weight of the foamable carrier component.

In some embodiments, propyl gallate is present in amount of about 0.05%, by weight of the foamable carrier component. In some embodiments, butylated hydroxyanisole (BHA) is present in an amount ranging from about 0.1% to about 0.5%, by weight of the foamable carrier component. In some embodiments, ethylenediaminetetraacetic acid (EDTA) is present in an amount of about 0.5%, by weight of the foamable carrier component. In some embodiments, tocofersolan (TPGS) is present in an amount of about 2%, by weight of the foamable carrier component.

### Propellant Component

In some embodiments, the foamable carrier component comprises a propellant component. In some embodiments, the propellant component comprises about 2% to about 15% of the foamable composition. In some embodiments, the propellant component comprises about 5% to about 15% of the foamable composition. In some embodiments, the propellant component comprises about 5% to about 10% of the foamable composition. In some embodiments, the propellant component comprises about 2% to about 15% of the foamable composition.

In some embodiments, the propellant component comprises one or more hydrofluorocarbons (HFCs) or hydrofluoroolefins (HFOs). In some embodiments, the propellant component comprises one or more hydrofluorocarbons (HFCs). In some embodiments, the propellant component comprises one or more hydrofluoroolefins (HFOs). In some embodiments, the propellant component comprises HFA-134. In some embodiments, the propellant comprises HFO-1234ze.

In some embodiments, the foamable composition comprises a non-volatile propellant. In some embodiments, the non-volatile propellant comprises nitrogen, nitrous oxide, carbon dioxide, dimethyl ether, 1,3,3,3-tetrafluoroprop-1-ene, 1,1,1,2-tetrafluoroethane, 1,1-difluoroethane or any combination thereof. In some embodiments, the non-volatile propellant comprises nitrogen, nitrous oxide, carbon dioxide or mixture of these propellants. In some embodiments, the non-volatile propellant comprises from about 3% to about 20% of the foamable composition.

In some embodiments, the foamable composition comprises of a volatile propellant. In some embodiments, the volatile propellant comprises propane, iso-butane, n-butane, or any combination thereof. In some embodiments, the propellant is AP22, AP30, AP104, AP40, AP46, AP58, AP70, P70, or P75. In some embodiments, the propellant component comprises a mixture of two to three compressed or liquefied gases selected from:

| propane (% by weight | iso-butane (% by weight | n-butane (% by weight |
|---|---|---|
| of the propellant | of the propellant | of the propellant |
| component) | component) | component) |
| 0 | 30 | 67 |
| 11 | 29 | 60 |
| 95 | 3 | 2 |
| 22 | 24 | 54 |
| 28 | 23 | 49 |
| 31 | 23 | 46 |
| 55 | 15 | 30 |

In some embodiments, a mixture of volatile propellant comprises a percentage of propane 0-99%, iso-butane 0-99%, and/or n butane 0-99% by weight of the propellant component, wherein the mixture of the volatile propellants is greater than 0%. In some embodiments, the volatile propellant comprises P75 propellant; P75 propellant comprises P70 propellant which has a composition of propane (55%), iso-butane (15%), and n-butane (30%) by weight of the propellant component.

In some embodiments, the propellant component comprises about 2% to about 20% of the foamable composition. In some embodiments, the propellant component comprises about 5% to about 15% of the foamable composition. In some embodiments, the propellant component comprises about 5% to about 10% of the foamable composition. In some embodiments, the propellant component comprises about 2% to about 10% of the foamable composition. In some embodiments, the propellant component comprises about 2% to about 8% of the foamable composition. In some embodiments, the propellant component comprises about 2% to about 5% of the foamable composition. In some embodiments, the propellant component comprises about 2% of the foamable composition. In some embodiments, the propellant component comprises about 3% of the foamable composition. In some embodiments, the propellant component comprises about 3.5% of the foamable composition. In some embodiments, the propellant component comprises about 4% of the foamable composition. In some embodiments, the propellant component comprises about 4.5% of the foamable composition. In some embodiments, the propellant component comprises about 5% of the foamable composition.

In some embodiments, the present disclosure provides a foamable composition comprising any of the foamable carrier components described herein and a propellant component.

In some embodiments, the present disclosure provides a foam produced by any of the foamable compositions described herein. In some embodiments, the foam of the present disclosure has a foam collapse property to ensure, e.g., retention on the subject's affected skin area. In some embodiments, the components of the topical foam composition are utilized to generate the needed foam collapse to be treat the subject's affected skin area. Foam collapse indicates how quickly and how long the foam will come in contact with the subject's affected skin area. When the foam breaks, the active pharmaceutical ingredient comes directly in contact with the subject's affected skin area but may also limit the overall duration of contact between the skin are and the foam.

In some embodiments, the foam collapse is measured using a water bath as a temperature-controlled environment. A sample contained with a screw capped is used with clamps to hold it in the water bath at, e.g., 32° C.-37° C., halfway submerged. Samples to be tested are attached with a metering valve. The samples are evaluated based on, e.g., visual appearance and time to collapse. In some embodiments, a further foam collapse measurement can be taken when the foam of the present disclosure is placed in a drying over set to a temperature of about 36° C.-37° C. and time lapse is measured until bubbles of the foam have broken or liquified. In some embodiments, the foam collapse occurs within 1 minute or less of application to the affected area.

In some embodiments, the foamable composition has a foam collapse rate of about 2.5 minutes, such as ≥3 minutes, and further for example, ≥5 minutes. In some embodiments, the foamable composition has a foam collapse rate ranging from about 2.5 minutes to about 6.5 minutes. In some embodiments, the foamable composition has a foam collapse rate of 2.5 minutes, 3.0 minutes, 3.5 minutes, 4.0 minutes, 4.5 minutes, 5.0 minutes, 5.5 minutes, 6.0 minutes, and/or 6.5 minutes.

In some embodiments, the foamable composition suitable for application as a foam to a body surface area of a human patient, comprising a foamable carrier component and a propellant, wherein the foamable carrier component comprises: a compound, which is ruxolitinib or deuterated ruxolitinib, or a pharmaceutically acceptable salt thereof, a hydroethanolic mixture, an emollient component, one or more C₁₆₋₁₈ fatty alcohols, and an emulsifier component, wherein the hydroethanolic mixture is a mixture of ethanol and water. In some embodiments, the compound of the foamable composition is ruxolitinib or a pharmaceutically acceptable salt thereof. In some embodiments, the compound of the foamable composition is ruxolitinib phosphate. In some embodiments, the ruxolitinib foam formulations are stable from about 1 month to about 6 months at a temperature ranging from 5° C. to 40° C. In some embodiments, the ruxolitinib foam formulations are stable for about 1 month at a temperature ranging from 5° C. to 40° C. In some embodiments, the ruxolitinib foam formulations are stable for about 2 months at a temperature ranging from 5° C. to 40° C. In some embodiments, the ruxolitinib foam formulations are stable for about 3 months at a temperature ranging from 5° C. to 40° C. In some embodiments, the ruxolitinib foam formulations are stable for about 4 months at a temperature ranging from 5° C. to 40° C. In some embodiments, the ruxolitinib foam formulations are stable for about 5 months at a temperature ranging from 5° C. to 40° C. In some embodiments, the ruxolitinib foam formulations are stable for about 6 months at a temperature ranging from 5° C. to 40° C. In some embodiments, ruxolitinib foam formulations are stable for about 6 months at a temperature of about 40° C.

### Methods

The present disclosure is directed to methods of treating an inflammatory or autoimmune skin or hair disease in a human patient in need thereof comprising administering to a body surface area affected by the disease of the patient a foam as described herein. In some embodiments, the inflammatory or autoimmune skin or hair disease is alopecia. In some embodiments, the inflammatory or autoimmune skin or hair disease is alopecia areata. In some embodiments, the alopecia areata is acute. In some embodiments, the alopecia areata is chronic. In some embodiments, the inflammatory or autoimmune skin or hair disease is a scalp condition, and the scalp condition is frontal fibrosing alopecia, lichen planopilaris, chronic cutaneous lupus erythematosus, or folliculitis decalvans. In some embodiments, the inflammatory or autoimmune skin or hair disease is a skin disease, and the skin disease is lichen planus (LP), hidradenitis suppurativa (HS), lichen sclerosus (LS), prurigo nodularis (PN), atopic dermatitis (AD), vitiligo (i.e., non-segmental vitiligo), or psoriasis. In some embodiments, the inflammatory or autoimmune skin or hair disease is a skin disease, and the skin disease is seborrheic dermatitis.

The present disclosure is further directed to a method of treating alopecia in a human patient, comprising administering to a body surface area affected by the alopecia of said patient, a foam produced by any of the foam compositions described herein. The present disclosure is further directed to a method of treating acute alopecia areata in a human patient, comprising administering to a body surface area affected by the acute alopecia areata of said patient, a foam produced by any of the foam compositions described herein. The present disclosure is further directed to a method of treating chronic alopecia areata in a human patient, comprising administering to a body surface area affected by the chronic alopecia areata of said patient, a foam produced by any of the foam compositions described herein. In some embodiments, the method of treating acute alopecia areata in a human patient, comprising administering to a body surface area affected by the acute alopecia areata of said patient, a foam produced by any of the foam compositions described herein, wherein the foam composition comprises ruxolitinib or a pharmaceutically acceptable salt thereof. In some embodiments, the method of treating chronic alopecia areata in a human patient, comprising administering to a body surface area affected by the acute alopecia areata of said patient, a foam produced by any of the foam compositions described herein, wherein the foam composition comprises deuruxolitinib or a pharmaceutically acceptable salt thereof.

The present disclosure is directed to a method of inducing hair growth in a human patient suffering from alopecia, comprising administering to a body surface area affected by the alopecia of said patient, a foam produced by any of the foam compositions described herein. The present disclosure is directed to a method of inducing hair growth in a human patient suffering from acute alopecia areata, comprising administering to a body surface area affected by the acute alopecia areataof said patient, a foam produced by any of the foam compositions described herein. The present disclosure is directed to a method of inducing hair growth in a human patient suffering from chronic alopecia areata, comprising administering to a body surface area affected by the chronic alopecia areata of said patient, a foam produced by any of the foam compositions described herein.

In some embodiments, the body skin area affected by alopecia comprises the patient's scalp.

In some embodiments, the alopecia is alopecia areata. In some embodiments, the alopecia areata is patchy alopecia areata. In some embodiments, the alopecia is alopecia totalis (hair loss across the entire scalp), alopecia undersalis (hair loss across the entire body including eyebrows, and eyelashes), alopecia barbae (affects the beard, often leading to sudden hair loss in small circular patches), diffuse alopecia areata (thinning of the hair all over the scalp), or alopecia ophiasis (hair loss in a band along the sides and back of the head). In some embodiments, the alopecia aereata is patchy alopecia areata. In some embodiments, the alopecia is alopecia totalis. In some embodiments, the alopecia is alopecia undersalis. In some embodiments, the alopecia is alopecia barbae. In some embodiments, the alopecia is diffuse alopecia areata. In some embodiments, the alopecia is alopecia ophiasis. In some embodiments, the alopecia is alopecia universalis.

In some embodiments, the alopecia areata is acute alopecia areata. In some embodiments, the alopecia areata is chronic alopecia areata. Alopecia areata can progress through acute, subacute, and chronic stages. For example, the acute stage, hair loss appears suddenly and is characterized by a “bee-swarm pattern” of lymphocytic infiltrates around hair follicles. With subacute, there is an increase in velluslike miniaturized hairs. The chronic stage shows an increase in velluslike miniaturized hairs and biopsies reveal follicular miniaturization.

In some embodiments, the patient's alopecia areata is mild to moderate. In some embodiments, the alopecia areata is mild. In some embodiments, the alopecia areata is moderate. In some embodiments, the alopecia aereata is severe. In some embodiments, the patient's alopecia areata is acute. In some embodiments, the patient's alopecia areata is chronic. Acute diffuse and total alopecia is a type of alopecia areata that causes a sudden onset of widespread hair loss, while mild alopecia areata is characterized by small, round patches of hair loss.

The present disclosure is also directed to a method of treating a scalp condition in a human patient, comprising administering to a body surface area affected by the scalp condition of said patient, a foam produced by any of the foam compositions described herein. In some embodiments, the scalp condition is one or more of frontal fibrosing alopecia, lichen planopilaris, chronic cutaneous lupus erythematosus, and folliculitis decalvans. In some embodiments, the scalp condition is frontal fibrosing alopecia. In some embodiments, the scalp condition is lichen planopilaris. In some embodiments, the scalp condition is chronic cutaneous lupus erythematosus. In some embodiments, the scalp condition is folliculitis decalvans.

The present disclosure is also directed to a method of treating a condition in a human patient, comprising administering to a body surface area affected by the condition of said patient, a foam produced by any of the foam compositions described herein. In some embodiments, the condition is one or more of lichen planus (LP), hidradenitis suppurativa (HS), lichen sclerosus (LS), prurigo nodularis (PN), atopic dermatitis (AD), vitiligo (i.e., non-segmental vitiligo), and psoriasis. In some embodiments, the condition is lichen planus (LP). In some embodiments, the condition is hidradenitis suppurativa (HS). In some embodiments, the condition is mild hidradenitis suppurativa (HS). In some embodiments, the condition is lichen sclerosus (LS). In some embodiments, the condition is prurigo nodularis (PN). In some embodiments, the condition is atopic dermatitis (AD). In some embodiments, the condition is vitiligo. In some embodiments, the condition is psoriasis. In some embodiments, the condition is atopic dermatitis (AD). In some embodiments, the condition is non-segmental vitiligo.

The present disclosure is directed to methods of treating an inflammatory or autoimmune skin or hair disease in a human patient in need thereof comprising administering to a body surface area affected by the disease of the patient a foam as described herein. In some embodiments, the body surface area affected comprises a bodily surface with hair follicles, i.e., epidermis or dermis. Hair follicles originate in the first and second layers of the skin-epidermis and dermis. For example, the bodily surface with hair follicles includes, but is not limited to, scalp, arms, legs, eyebrows, eyelashes, back of neck, face, armpits, face, or a combination thereof.

The present disclosure is directed to methods of treating an inflammatory or autoimmune skin or hair disease in a human patient in need thereof comprising administering to a body surface area affected by the disease of the patient a foam as described herein. In some embodiments, the skin disease is seborrheic dermatitis. In some embodiments, the foam comprises ruxolitinib, deuruxolitinib, or a pharmaceutically acceptable salt thereof.

In some embodiments, the method further comprises administering oral ruxolitinib or oral deuterated ruxolitinib. In some embodiments, the oral ruxolitinib or oral deuterated ruxolitinib is an amount of 8 mg two times per day.

In some embodiments, the method further comprises administering oral deuruxolitinib in an amount of 8 mg BID. In some embodiments, the method further comprises administering oral deuruxolitinib in an amount of 16 mg per day.

In some embodiments, the method further comprises administering oral deuruxolitinib. In some embodiments, the oral deuruxolitinib is an amount of 12 mg two times per day. In some embodiments, the method further comprises administering oral deuruxolitinib in an amount of 12 mg BID. In some embodiments, the method further comprises administering oral deuruxolitinib in an amount of 24 mg per day.

In some embodiments, inducing hair growth is on the patient's scalp, the patient's body, or combinations thereof. In some embodiments, inducing hair growth is on the patient's scalp. In some embodiments, topically administering is to the patient's scalp, the patient's body, or combinations thereof.

In some embodiments, the patient has alopecia areata. In some embodiments, the alopecia areata is patchy alopecia areata. In some embodiments, the alopecia areata is acute alopecia areata. In some embodiments, the alopecia areata is chronic alopecia areata. In some embodiments, the alopecia isalopecia totalis, alopecia undersalis, alopecia barbae, diffuse alopecia areata, or alopecia ophiasis. In some embodiments, the alopecia is alopecia totalis. In some embodiments, the alopecia is alopecia undersalis. In some embodiments, the alopecia is alopecia barbae. In some embodiments, the alopecia is diffuse alopecia areata. In some embodiments, the alopecia is alopecia ophiasis. In some embodiments, the alopecia is alopecia universalis.

In some embodiments, the present disclosure is further directed to a method of making a foamable composition, as described herein. In some embodiments, the method comprises:
mixing in a tank (tank 3) ruxolitinib, deuterated ruxolitinib, or a
pharmaceutically acceptable salt thereof with an alcohol (e.g., propylene glycol) while stirring until the ruxolitinib, deuterated ruxolitinib, or a pharmaceutically acceptable salt of any of the aforementioned is dissolved to generate an active phase;
mixing in a further tank (tank 2) a water phase, wherein the water phase comprises water, an alcohol (e.g., ethanol), and a solubilizer (e.g., PEG300, PEG400) and heating the tank to a temperature ranging about 60° C. to about 65° C.;
mixing in a final tank (tank 1) an oil phase, wherein the oil phase comprises one or more fatty alcohols (e.g., cetyl alcohol, stearyl alcohol) and heating the final tank to a temperature ranging about 70° C. to about 75° C.;
transferring the water phase to the oil phase and equilibrating to a temperature ranging about 70° C. to about 75° C. with stirring to generate a 2-phase mixture;
cooling the 2-phase mixture to a temperature of about 60° C.;
transferring the active phase to the 2-phase mixture and mixing to generate a final phase emulsion;
adjusting the final phase emulsion with a pH buffer to a pH ranging from about 5 to about 6;
homogenizing the final phase emulsion to generate a bulk emulsion;
cooling the bulk emulsion to a temperature ranging from about 30° C. to about 35° C.;
filing the bulk emulsion into containers; and
aerosolizing the bulk emulsion with a propellant component in the containers.

In some embodiments, the method further comprises after mixing in the tank ruxolitinib, deuterated ruxolitinib, or a pharmaceutically acceptable salt thereof with the alcohol, heating the tank to a temperature ranging from 60° C. to about 65° C. to dissolve the ruxolitinib, deuterated ruxolitinib, or a pharmaceutically acceptable salt of any of the aforementioned.

In some embodiments, the method further comprises after mixing in the further tank the water phase, adding a quantity sufficient of water to compensate for any loss of water due to evaporation.

### Kits

The present disclosure also includes pharmaceutical kits useful, for example, in the treatment hair loss, which include one or more containers containing the topical foam composition, as described herein. Instructions, either as inserts or as labels, indicating quantities of the components to be administered, guidelines for administration, and/or guidelines for mixing the components, can also be included in the kit.

As will be appreciated, some components of the formulation described herein can possess multiple functions. For example, a given substance may act as both an emulsifying agent component and a stabilizing agent. In some such cases, the function of a given component can be considered singular, even though its properties may allow multiple functionalities. In some embodiments, each component of the formulation comprises a different substance or mixture of substances.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination.

Without limitation, the following are some embodiments of the present disclosure including:

Embodiment 1: A foamable composition suitable for application as a foam to a body surface area affected by alopecia in a human patient, comprising a foamable carrier component and a propellant component; wherein the foamable carrier component comprises a compound, which is ruxolitinib or deuterated ruxolitinib, or a pharmaceutically acceptable salt of any of the aforementioned.

Embodiment 2: The foamable composition according to embodiment 1, wherein the foamable carrier component is a homogeneous emulsion.

Embodiment 3: The foamable composition according to embodiment 1, wherein the foamable composition is a homogeneous emulsion.

Embodiment 4: The foamable composition according to embodiment 1, wherein the foamable carrier component further comprises water, a solvent component, and an oil phase.

Embodiment 5: The foamable composition according to embodiment 4, wherein the oil phase comprises about 0.5% to about 20% by weight of the foamable carrier composition.

Embodiment 6: The foamable composition according to embodiment 4, wherein the oil phase comprises about 0.5% to about 10% by weight of the foamable carrier composition.

Embodiment 7: The foamable composition according to embodiment 4, wherein the oil phase comprises about 1% to about 10% by weight of the foamable carrier composition.

Embodiment 8: The foamable composition according to any one of embodiments 4-7, wherein the oil phase comprises at least one fatty alcohol.

Embodiment 9: The foamable composition according to embodiment 8, wherein the at least one fatty alcohol comprises at least one C₁₆₋₁₈ fatty alcohol.

Embodiment 10: The foamable composition according to embodiment 8, wherein the at least one fatty alcohol is cetyl alcohol or stearyl alcohol, or a mixture thereof.

Embodiment 11: The foamable composition according to any one of embodiments s 8-10, wherein the fatty alcohol functions as an emollient

Embodiment 12: The foamable composition according to any one of embodiments 8-10, wherein the at least one fatty alcohol functions as a foaming agent.

Embodiment 13: The foamable composition according to any one of embodiments 4-12, wherein the solvent component comprises from about 30% to about 95% by weight of the foamable carrier component.

Embodiment 14: The foamable composition according to embodiment 13, wherein the solvent component comprises a C₁₋₄ aliphatic alcohol.

Embodiment 15: The foamable composition according to embodiment 13, wherein the solvent component comprises ethanol.

Embodiment 16: The foamable composition according to any one of embodiments 13-15, wherein the solvent component comprises polyethylene glycol.

Embodiment 17: The foamable composition according to any one of embodiments 13-15, wherein the solvent component comprises PEG200 or PEG300.

Embodiment 18: The foamable composition according to any one of embodiments 13-18, wherein the solvent component comprises an alkylene glycol.

Embodiment 19: The foamable composition according to any one of embodiments 13-18, wherein the solvent component comprises a C₁₋₄ aliphatic alcohol, a polyalkylene glycol, or an alkylene glycol, or mixtures of any of the foregoing.

Embodiment 20: The foamable composition according to any one of embodiments 4-12, wherein the solvent component comprises ethanol, PEG200, PEG300, or propylene glycol, or mixtures of any of the foregoing.

Embodiment 21: The foamable composition according to any one of embodiments 4-20, wherein the water comprises from about 20% to about 70%, by weight of the foamable carrier component.

Embodiment 22: The foamable composition according to any one of embodiments 4-20, wherein the foamable carrier component comprises ≥50% water, by weight of the foamable carrier component.

Embodiment 23: The foamable composition according to any one of embodiments 4-20, wherein the water comprises from about 20% to about 70% by weight of the foamable carrier component.

Embodiment 24: The foamable composition according to any one of embodiments 4-20, wherein the water comprises from about 20% to about 60% by weight of the foamable carrier component.

Embodiment 25: The foamable composition according to any one of embodiments 1-24, wherein the foamable carrier component is present in an amount from about 70% to about 99.9% of the foamable composition.

Embodiment 26: The foamable composition according to any one of embodiments 1-25, wherein the propellant component comprises from about 2% to about 20% of the foamable composition.

Embodiment 27: The foamable composition according to any one of embodiments 1-25, wherein the propellant component comprises from about 5% to about 10% of the foamable composition.

Embodiment 28: The foamable composition according to any one of embodiments 1-27, wherein the propellant component comprises a non-volatile propellant.

Embodiment 29: The foamable composition according to any one of embodiments 1-27, wherein the propellant component comprises a volatile propellant.

Embodiment 30: The foamable composition according to any one of embodiments 1-27, wherein the propellant component comprises propane, iso-butane, n-butane, or any combination thereof.

Embodiment 31: The foamable composition according to any one of embodiments 1-27, wherein the propellant component is AP22, AP30, AP104, AP40, AP46, AP58, AP70, P70, or P75.

Embodiment 32: The foamable composition according to any one of embodiments 1-3, wherein the foamable carrier component comprises: from about 40% to about 90% of solvent component by weight of the foamable carrier component; rom about 30% to about 60% of water by weight of the foamable carrier component; and from about 0.5% to about 10% of an oil phase by weight of the foamable carrier component.

Embodiment 33: The foamable composition according to any one of embodiments 1-3, wherein the foamable carrier component comprises: om about 40% to about 65% of ethanol by weight of the foamable carrier component; from about 30% to about 60% of water by weight of the foamable carrier component; from about 0.5% to about 5% of stearyl alcohol by weight of the foamable carrier component; from about 0.5% to about 5% of cetyl alcohol by weight of the foamable carrier component; and from about 2% to about 20% of propylene glycol.

Embodiment 34: The foamable composition according to any one of embodiments 1-3, wherein the foamable carrier component comprises: from about 40% to about 95% of solvent component by weight of the foamable carrier component; from about 30% to about 60% water by weight of the foamable carrier component; and from about 0.5% to about 10% of at least one fatty alcohol by weight of the foamable carrier component.

Embodiment 35: The foamable composition according to any one of embodiments 1-34, wherein the foamable composition is stable at a temperature ranging from 5° C. to 40° C. for about 6 months.

Embodiment 36: The foamable composition according to any one of embodiments 1-34, wherein the compound is ruxolitinib, or a pharmaceutically acceptable salt thereof.

Embodiment 37: The foamable composition according to any one of embodiments 1-34, wherein the compound is ruxolitinib phosphate.

Embodiment 38: The foamable composition according to any one of embodiments 1-34, wherein the compound is deuterated ruxolitinib or a pharmaceutically acceptable salt thereof.

Embodiment 39: The foamable composition according to any one of embodiments 1-34, wherein the compound is deuruxolitinib or a pharmaceutically acceptable salt thereof.

Embodiment 40: The foamable composition according to any one of embodiments 1-34, wherein the compound is deuruxolitinib phosphate.

Embodiment 41: The foamable composition according to any one of embodiments 35-40, wherein the compound is present in an amount ranging from about 0.05% to about 1.5% by weight of the foamable carrier component on a free base basis of the compound, which is ruxolitinib or deuterated ruxolitinib, or a pharmaceutically acceptable salt of any of the aforementioned.

Embodiment 42: The foamable composition according to any one of embodiments 35-40, wherein the compound is present in an amount ranging from about 0.3% to about 0.8% by weight of the foamable carrier component on a free base basis of the compound, which is ruxolitinib or deuterated ruxolitinib, or a pharmaceutically acceptable salt of any of the aforementioned.

Embodiment 43: The foamable composition according to any one of embodiments 1-42, wherein the foamable composition and foamable carrier component does not comprise an organic amine pH adjusting agent.

Embodiment 44: A foam produced by expelling the foamable composition of any one of the preceding embodiments from a pressurized container.

Embodiment 45: The foam according to embodiment 44, wherein the foamable composition is aerosolized.

Embodiment 46: A foam suitable for application as a foam to a body surface area affected by alopecia in a human patient, comprising a compound, which is ruxolitinib or deuterated ruxolitinib, or a pharmaceutically acceptable salt of any of the aforementioned.

Embodiment 47: The foam according to embodiment 46, further comprising a foamable composition according to any one of embodiments 1-43.

Embodiment 48: A foamable carrier component comprising a compound, which is ruxolitinib or deuterated ruxolitinib, or a pharmaceutically acceptable salt of any of the aforementioned.

Embodiment 49: The foamable carrier component according to embodiment 48, further comprising water, a solvent component, and an oil phase.

Embodiment 50: A foamable carrier component as described in any one of embodiments 4-43.

Embodiment 51: A method for treating alopecia in a human patient in need thereof comprising administering to a body surface area affected by the alopecia of the patient a foam produced according to any one of embodiments 44-47.

Embodiment 52: The method according to embodiment 51, wherein the alopecia is alopecia areata.

Embodiment 53: The method according to embodiment 52, wherein the alopecia areata is mild to moderate.

Embodiment 54: The method according to embodiment 52, wherein the alopecia areata is severe.

Embodiment 55: The method according to embodiment 52, wherein the alopecia areata is chosen from patchy alopecia areata.

Embodiment 56: The method according to any one of embodiments 51-55, wherein the body surface area affected is the patient's scalp.

Embodiment 57: The method according to any one of embodiments 51-56, further comprising administering deuruxolitinib orally to the patient.

Embodiment 58: The method according to embodiment 57, wherein the deuruxolitinib is administered in an amount of 8 mg two times per day to the patient.

Embodiment 59: The method according to any one of embodiments 51-56, further comprising administering deuruxolitinib orally to the patient.

Embodiment 60: The method according to embodiment 59, wherein the deuruxolitinib is administered in an amount of 12 mg two times per day to the patient.

Embodiment 61: A method of inducing hair growth in a human patient suffering from alopecia, comprising administering to a body surface area affected by the alopecia of the patient a foam produced according to any one of embodiments 44-47.

Embodiment 62: The method according to embodiment 61, wherein the alopecia is alopecia areata.

Embodiment 63: The method according to embodiment 62, wherein the alopecia areata is mild to moderate.

Embodiment 64: he method according to embodiment 62, wherein the alopecia areata is severe.

Embodiment 65: The method according to embodiment 62, wherein the alopecia areata is patchy alopecia areata.

Embodiment 66: The method according to any one of embodiments 61-66, wherein the body surface area affected is the patient's scalp.

Embodiment 67: The method according to any one of embodiments 51-56, further comprising administering deuruxolitinib orally to the patient.

Embodiment 68: The method according to embodiment 67, wherein the deuruxolitinib is administered in an amount of 8 mg two times per day to the patient.

Embodiment 69: The method according to any one of embodiments 61-66, further comprising administering deuruxolitinib orally to the patient.

Embodiment 70: The method according to embodiment 69, wherein the deuruxolitinib is administered in an amount of 12 mg two times per day to the patient.

Embodiment 71: Use of a foam according to embodiments 44-47 for a preparation of a medicament for use in treatment of alopecia.

Embodiment 72: Use of a foamable composition according to embodiments 1-43 for preparation of a medicament for use in treatment of alopecia.

Embodiment 73: A foamable composition suitable for application as a foam to a body surface area affected by alopecia areata in a human patient, comprising a foamable carrier component and a propellant component; wherein the foamable carrier component comprises a compound, which is ruxolitinib or deuterated ruxolitinib, or a pharmaceutically acceptable salt of any of the aforementioned; wherein the body surface area is the patient's scalp; and wherein the foamable composition does not comprise an organic amine pH adjusting agent.

Embodiment 74: A foamable composition of embodiment 73 comprising the foamable carrier component as described in any one of embodiments 1-43 and 48-50.

Embodiment 75: A foam comprising the foamable composition of embodiment 73 or 74.

Embodiment 76: A method for treating alopecia in a human patient in need thereof comprising administering to a body surface area affected by the alopecia of the patient a foam according to embodiment 75.

Embodiment 77: The method according to embodiment 51, wherein the alopecia areata is acute.

Embodiment 78: The method according to embodiment 51, wherein the alopecia areata is chronic.

Embodiment 79: The method according to embodiment 77, wherein the compound is ruxolitinib or a pharmaceutically acceptable salt thereof.

Embodiment 80: The method according to embodiment 78, wherein the compound is deuruxolitinib or a pharmaceutically acceptable salt thereof.

Embodiment 81: A foamable composition suitable for application as a foam to a body surface area affected by seborrheic dermatitis in a human patient, comprising a foamable carrier component and a propellant component; wherein the foamable carrier component comprises a compound, which is ruxolitinib or deuterated ruxolitinib, or a pharmaceutically acceptable salt of any of the aforementioned.

Embodiment 82: The foamable composition according to embodiment 81, wherein the foamable carrier component is a homogeneous emulsion.

Embodiment 83: The foamable composition according to embodiment 81, wherein the foamable composition is a homogeneous emulsion.

Embodiment 84: The foamable composition according to embodiment 81, wherein the foamable carrier component further comprises water, a solvent component, and an oil phase.

Embodiment 85: The foamable composition according to embodiment 84, wherein the oil phase comprises about 0.5% to about 20% by weight of the foamable carrier composition.

Embodiment 86: The foamable composition according to embodiment 84, wherein the oil phase comprises about 0.5% to about 10% by weight of the foamable carrier composition.

Embodiment 87: The foamable composition according to embodiment 84, wherein the oil phase comprises about 1% to about 10% by weight of the foamable carrier composition.

Embodiment 88: The foamable composition according to any one of embodiments 81-87, wherein the compound is ruxolitinib, or a pharmaceutically acceptable salt thereof.

Embodiment 89: The foamable composition according to any one of embodiments 81-87, wherein the compound is ruxolitinib phosphate.

Embodiment 90: The foamable composition according to any one of embodiments 81-87, wherein the compound is deuterated ruxolitinib or a pharmaceutically acceptable salt thereof.

Embodiment 91: The foamable composition according to any one of embodiments 81-90, wherein the compound is ruxolitinib or a pharmaceutically acceptable salt thereof.

Embodiment 92: The foamable composition according to any one of embodiments 81-90, wherein the compound is deuruxolitinib or a pharmaceutically acceptable salt thereof.

Embodiment 93: A method for treating seborrheic dermatitis in a human patient in need thereof comprising administering to a body surface area affected by the seborrheic dermatitis of the patient a foam produced according to any one of embodiments 81-90.

Embodiment 94: The method according to embodiment 93, wherein the compound is ruxolitinib or a pharmaceutically acceptable salt thereof.

Embodiment 95: The method according to embodiment 93, wherein the compound is deuruxolitinib or a pharmaceutically acceptable salt thereof.

Embodiment 96: A foamable composition suitable for application as a foam to a body surface area affected by an inflammatory or autoimmune skin or hair disease in a human patient, comprising a foamable carrier component and a propellant component, wherein the foamable carrier component comprises: from about 0.5% to about 3%, of a compound, which is ruxolitinib or deuruxolitinib, or a pharmaceutically acceptable salt thereof, by weight of the foamable carrier composition, from about 80% to about 90% of a hydroethanolic mixture, by weight of the foamable carrier composition, from about 1% to about 3% of an emollient component, by weight of the foamable carrier composition, wherein the emollient component comprises at least one emollient and at least one co-solvent, from about 1% to about 5% of one or more C₁₆₋₁₈ fatty alcohols, by weight of the foamable carrier composition, from about 0.5% to about 3% of an emulsifier component, by weight of the foamable carrier composition, and from about 4% to about 6% of a solvent, by weight of the foamable carrier composition; wherein the hydroethanolic mixture is a mixture of ethanol and water, the ethanol is present in amount ranging from about 50% to about 70% of the hydroethanolic mixture, and the water is present in an amount ranging from about 30% to about 50% of the hydroethanolic mixture.

Embodiment 97: The foamable composition according to embodiment 96, wherein: the emulsifier component is polysorbate 60 (Tween 60); the emollient is glycerin and the co-solvent is polyethylene glycol 300; the one or more C₁₆₋₁₈ fatty alcohols are cetyl alcohol and stearyl alcohol; the solvent is propylene glycol; and the compound is ruxolitinib phosphate in an amount of about 2.5% on a free base basis, of the foamable carrier component.

Embodiment 98: The foamable composition according to embodiment 96, wherein: the emulsifier component is polysorbate 60 (Tween 60); the emollient is glycerin and the co-solvent is polyethylene glycol 300; the one or more C₁₆₋₁₈ fatty alcohols are cetyl alcohol and stearyl alcohol; the solvent is propylene glycol; and the compound is ruxolitinib phosphate in an amount of about 1.5% on a free base basis, of the foamable carrier component.

Embodiment 99: The foamable composition according to embodiment 96, wherein: the emulsifier component is polysorbate 60 (Tween 60); the emollient is glycerin and the co-solvent is polyethylene glycol 300; the one or more C₁₆₋₁₈ fatty alcohols are cetyl alcohol and stearyl alcohol; the solvent is propylene glycol; and the compound is deuruxolitinib phosphate in an amount of about 2.5% on a free base basis, of the foamable carrier component.

Embodiment 100: The foamable composition according to embodiment 96, wherein: the emulsifier component is polysorbate 60 (Tween 60); the emollient is glycerin and the co-solvent is polyethylene glycol 300; the one or more C₁₆₋₁₈ fatty alcohols are cetyl alcohol and stearyl alcohol; the solvent is propylene glycol; and the compound is deuruxolitinib phosphate in an amount of about 1.5% on a free base basis, of the foamable carrier component.

Embodiment 101: The foamable composition according to embodiment 96, wherein: the emulsifier component is polysorbate 60 (Tween 60); the emollient is myristyl lactate and the co-solvent is transcutol-P; the one or more C₁₆₋₁₈ fatty alcohols are cetyl alcohol and stearyl alcohol; the solvent is propylene glycol; and the compound is ruxolitinib phosphate in an amount of about 2.5% on a free base basis, of the foamable carrier component.

Embodiment 102: The foamable composition according to embodiment 96, wherein: the emulsifier component is polysorbate 60 (Tween 60); the emollient is myristyl lactate and the co-solvent is transcutol-P; the one or more C₁₆₋₁₈ fatty alcohols are cetyl alcohol and stearyl alcohol; the solvent is propylene glycol; and the compound is ruxolitinib phosphate in an amount of about 1.5% on a free base basis, of the foamable carrier component.

Embodiment 103: The foamable composition according to embodiment 96, wherein: the emulsifier component is polysorbate 60 (Tween 60); the emollient is myristyl lactate and the co-solvent is transcutol-P; the one or more C₁₆₋₁₈ fatty alcohols are cetyl alcohol and stearyl alcohol; the solvent is propylene glycol; and the compound is deuruxolitinib phosphate in an amount of about 2.5% on a free base basis, of the foamable carrier component.

Embodiment 104: The foamable composition according to embodiment 96, wherein: the emulsifier component is polysorbate 60 (Tween 60); the emollient is myristyl lactate and the co-solvent is transcutol-P; the one or more C₁₆₋₁₈ fatty alcohols are cetyl alcohol and stearyl alcohol; the solvent is propylene glycol; and the compound is deuruxolitinib phosphate in an amount of about 1.5% on a free base basis, of the foamable carrier component.

Embodiment 105: The foamable composition according to embodiment 96, wherein: the compound or the salt is present in an amount of 1.5% or 2.5%, by weight of the foamable carrier composition, the hydroethanolic mixture is present in an amount from about 80% to about 90%, by weight of the foamable carrier composition, the emollient component is present in an amount from about 2% to about 3%, by weight of the foamable carrier composition, wherein the emollient component comprises about 0.3% to about 0.6% of an emollient and about 1.8% to about 2.2% of a co-solvent, by weight of the foamable carrier composition, the one or more C₁₆₋₁₈ fatty alcohols are cetyl alcohol and stearyl alcohol, the cetyl alcohol is present in an amount from about 2% to about 2.5%, by weight of the foamable carrier composition, the stearyl alcohol is present in an amount from about 0.25% to about 0.5%, by weight of the foamable carrier composition, and the emulsifier component is present in an amount from about 1% to about 2%, by weight of the foamable carrier composition, the solvent is present in an amount from about 4% to about 6%, by weight of the foamable carrier composition, wherein the hydroethanolic mixture is a mixture of ethanol and water, the ethanol is present in amount ranging from about 55% to about 65% of the hydroethanolic mixture, and the water is present in an amount ranging from about 35% to about 45% of the hydroethanolic mixture.

Embodiment 106: The foamable composition according to embodiment 105, wherein: the emulsifier component is polysorbate 60 (Tween 60); the emollient is glycerin and the co-solvent is polyethylene glycol 300, the solvent is propylene glycol; and the compound is ruxolitinib phosphate in an amount of about 2.5% on a free base basis, of the foamable carrier component.

Embodiment 107: The foamable composition according to embodiment 105, wherein: the emulsifier component is polysorbate 60 (Tween 60); the emollient is glycerin and the co-solvent is polyethylene glycol 300; the solvent is propylene glycol; and the compound is ruxolitinib phosphate in an amount of about 1.5% on a free base basis, of the foamable carrier component.

Embodiment 108: The foamable composition according to embodiment 105, wherein: the emulsifier component is polysorbate 60 (Tween 60); the emollient is glycerin and the co-solvent is polyethylene glycol 300; the solvent is propylene glycol; and he compound is deuruxolitinib phosphate in an amount of about 2.5% on a free base basis, of the foamable carrier component.

Embodiment 109: The foamable composition according to embodiment 105, wherein: the emulsifier component is polysorbate 60 (Tween 60); the emollient is glycerin and the co-solvent is polyethylene glycol 300; the solvent is propylene glycol; and the compound is deuruxolitinib phosphate in an amount of about 1.5% on a free base basis, of the foamable carrier component.

Embodiment 110: The foamable composition according to embodiment 96, wherein: the compound or the salt is present in an amount of 1.5% or 2.5%, by weight of the foamable carrier composition, the hydroethanolic mixture is present in an amount from about 80% to about 90%, by weight of the foamable carrier composition, the emollient component is present in an amount from about 1% to about 2%, by weight of the foamable carrier composition, wherein the emollient component comprises about 0.8% to about 1.2% of an emollient and about 0.3% to about 0.6% of a co-solvent, by weight of the foamable carrier composition, the one or more C₁₆₋₁₈ fatty alcohols are cetyl alcohol and stearyl alcohol; the cetyl alcohol is present in an amount from about 2% to about 2.5%, by weight of the foamable carrier composition, the stearyl alcohol is present in an amount of from about 0.6% to about 0.9%, by weight of the foamable carrier composition, the emulsifier component is present in an amount from about 1% to about 2%, by weight of the foamable carrier composition, and the solvent is present in an amount from about 4% to about 6%, by weight of the foamable carrier composition, wherein the hydroethanolic mixture is a mixture of ethanol and water, the ethanol is present in amount ranging from about 55% to about 65% of the hydroethanolic mixture, and the water is present in an amount ranging from about 35% to about 45% of the hydroethanolic mixture.

Embodiment 111: The foamable composition according to embodiment 110, wherein: the emulsifier component is polysorbate 60 (Tween 60); the emollient is myristyl lactate and the co-solvent is transcutol-P, the solvent is propylene glycol; and the compound is ruxolitinib phosphate in an amount of about 2.5% on a free base basis, of the foamable carrier component.

Embodiment 112: The foamable composition according to embodiment 110, wherein: the emulsifier component is polysorbate 60 (Tween 60); the emollient is myristyl lactate and the co-solvent is transcutol-P; the solvent is propylene glycol; and the compound is ruxolitinib phosphate in an amount of about 1.5% on a free base basis, of the foamable carrier component.

Embodiment 113: The foamable composition according to embodiment 110, wherein: the emulsifier component is polysorbate 60 (Tween 60); the emollient is myristyl lactate and the co-solvent is transcutol-P; the solvent is propylene glycol; and the compound is deuruxolitinib phosphate in an amount of about 2.5% on a free base basis, of the foamable carrier component.

Embodiment 114: The foamable composition according to embodiment 110, wherein: the emulsifier component is polysorbate 60 (Tween 60); the emollient is myristyl lactate and the co-solvent is transcutol-P, the solvent is propylene glycol; and the compound is deuruxolitinib phosphate in an amount of about 1.5% on a free base basis, of the foamable carrier component.

Embodiment 115: The foamable composition according to embodiment 96, wherein: the emollient component comprises PEG 300 in an amount of about 2% and glycerin in an amount of about 0.5% by weight of the foamable carrier component.

Embodiment 116: The foamable composition according to embodiment 110, wherein: the emollient component comprises myristyl lactate in an amount of about 1%, by weight of the foamable carrier component and transcutol-P in an amount of about 0.5% by weight of the foamable carrier component.

Embodiment 117: The foamable composition according to any one of embodiments 96-101, wherein the pH of the foamable composition ranges from about 5.0 to about 8.0.

Embodiment 118: The foamable composition according to any one of embodiment 117, wherein the pH of the foamable composition ranges from about 5.0 to about 6.0.

Embodiment 119: The foamable composition according to embodiment 117, wherein the pH is adjusted by addition of trolamine to the foamable carrier composition.

Embodiment 120: The foamable composition according to embodiment 118, wherein the pH is adjusted by addition of trolamine to the foamable carrier composition.

Embodiment 121: A foam produced by expelling the foamable composition according to any one of embodiments 96-101 from a pressurized container.

Embodiment 122: The foam according to embodiment 121, wherein the foamable composition is aerosolized.

Embodiment 123: A method for treating an inflammatory or autoimmune skin or hair disease in a human patient in need thereof comprising administering to a body surface area affected by the disease of the patient a foam produced according to embodiment 121.

Embodiment 124: The method of embodiment 123, wherein the inflammatory or autoimmune skin or hair disease is alopecia.

Embodiment 125: The method according to embodiment 124, wherein the alopecia is alopecia areata.

Embodiment 126: The method according to embodiment 125, wherein the alopecia areata is mild to moderate.

Embodiment 127: The method according to embodiment 125, wherein the alopecia areata is severe.

Embodiment 128: The method according to embodiment 125, wherein the alopecia areata is acute.

Embodiment 129: The method according to embodiment 125, wherein the alopecia areata is chronic.

Embodiment 130: The method according to embodiment 123, wherein the inflammatory or autoimmune skin or hair disease is seborrheic dermatitis.

### EXAMPLES

The presently claimed subject matter will be described in greater detail by way of specific examples. The following examples are offered for illustrative purposes and are not intended to limit the presently claimed subject matter in any manner. Those of skill in the art will readily recognize a variety of non-critical parameters, which can be changed or modified to yield essentially the same results.

### Example 1: Solubility of Ruxolitinib in Organic Media

Under pre-formulation studies, the solubility of ruxolitinib phosphate was evaluated in organic media over the course of three days at ambient temperature. Table 1 presents the reported and calculated solubilities.

TABLE 1
Ruxolitinib phosphate solubility in
organic media (no propellant added).

| | | Reported | |
|---|---|---|---|
| | | Solubility % | Calculated |
| Organic Media | | w/w, ruxolitinib | Solubility, % |
| (neat) | Function | phosphate | w/w in base form |
| Oleyl alcohol | Surfactant, | 0.068 | 0.052 |
| | emulsifier, | | |
| | emollient, | | |
| | thickener | | |
| 1,3-Dimethyl-2- | Solvent | 0.22 | 0.168 |
| Imadolidinone | | | |
| (DMI) | | | |
| DIPA | Emollient | <0.01 | <0.01 |
| Propylene | Solvent, | 2.179 | 1.651 |
| Glycol (PG) | permeation | | |
| | enhancer | | |
| PEG400 | Solvent | 1.117 | 0.846 |
| PEG 300 | Solvent | 1.543 | 1.169 |
| Octadodecabol | Emollient | 0.029 | 0.022 |
| (ODD) | | | |
| Ethyl Oleate | Solvent, | <0.01 | <0.01 |
| | emollient | | |
| Isopropyl | Penetration | <0.01 | <0.01 |
| Myristate (IPM) | enhancer, | | |
| | emollient | | |
| Light Mineral | Emollient | <0.01 | <0.01 |
| Oil (LMO) | | | |
| GTCC | Permeation | 0.02 | 0.015 |
| | enhancer | | |
| Transcutol-P | Permeation | 1.223 | 0.927 |
| | enhancer, | | |
| | solvent | | |
| Polysorabte 80 | Emulsifier | 1.214 | 0.920 |
| Polysorbate 20 | Emulsifier | 1.263 | 0.957 |
| Span 20 | Emulsifier | 1.157 | 0.877 |
| Oleth-3 | Emulsifier | 0.677 | 0.513 |
| Etocas 35 | Emulsifier, | 0.962 | 0.729 |
| | solvent | | |
| Castor Oil | Emollient | 0.037 | 0.028 |
| MW, salt | — | 404.4 | — |
| MW, base | — | — | 306.4 |
| CF to base form | — | — | 0.758 |

### Example 2: Ruxolitinib Emulsion/Foamable Carrier Component/Base

Table 2 provides a ruxolitinib emulsion or foam base manufactured according to the below procedures:

TABLE 2
Ruxolitinib emulsion or foam base.

| Ingredient | % w/w |
|---|---|
| Ruxolitinib phosphate | 0.6596 |
| (equiv to 0.50% Ruxolitinib) | |
| Benzyl alcohol | 0.50 |
| Butylated hydroxytoluene | 0.10 |
| Cetyl alcohol | 2.50 |
| Citric acid anhydrous | 0.10 |
| Edetate disodium | 0.10 |
| Glycerin | 7.05 |
| Glyceryl monostearate | 1.05 |
| Hydroxyethyl cellulose | 0.45 |
| Mineral Oil | 27.08 |
| Medium-chain triglycerides | 12.44 |
| Polyoxyl 20 cetostearyl ether | 2.45 |
| Propylene glycol | 15.00 |
| Sodium hydroxide, 20% aqueous solution | Approx 0.27 |
| Trisodium citrate dihydrate | 0.25 |
| Water, Purified | 30.00 |
| Total | 100.00 |

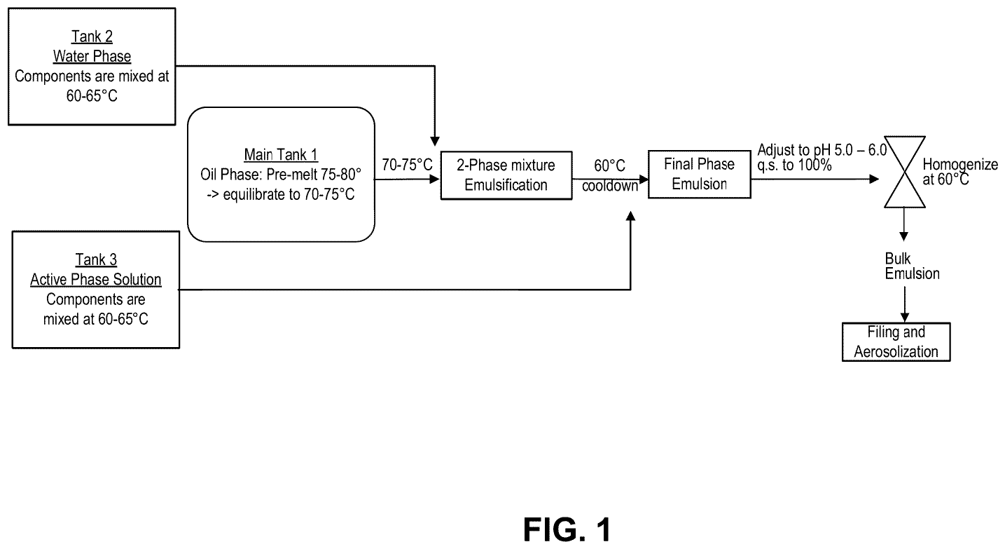

The manufacturing procedure included the preparation of an active phase (Table 3), a water phase (Table 4), an oil phase (Table 5), and a final phase-emulsion (Table 6). FIG. 1 graphically illustrates a proposed manufacturing process described in Tables 3-6.

TABLE 3
Active Phase.
Active Phase

| | |
|---|---|
| 1 | To a tank (Tank III), dispense 80% of propylene glycol at |
| | ambient temperature, and start stirring. |
| 2 | While stirring, slowly add ruxolitinib phosphate and continue |
| | stirring the mixture until ruxolitinib phosphate is dissolved. |
| | Note: To accelerate dissolution of ruxolitinib phosphate, |
| | the tank (Tank III) can be heated to 60° to 65° C. |
| 3 | Upon dissolution, cool the tank (Tank III) to 55°-60° C. |

TABLE 4
Water Phase - with 10% extra quantity
Water Phase - with 10% extra quantity

| | |
|---|---|
| 4 | To a tank (Tank II), dispense the amount of purified |
| | water and start stirring. |
| 5 | While stirring, add the citric acid, sodium citrate, |
| | disodium edetate, and mix until clear and dissolved. |
| 6 | To the tank (Tank II), sprinkle Natrosol 250 HX and |
| | continue stirring until Natrosol is fully hydrated and |
| | the resulting gel solution appears clear and speck-free. |
| 7 | Add the glycerin to the tank (Tank II) and continue |
| | stirring until uniform and a clear gel solution results. |
| 8 | Heat up and maintain the tank (Tank II) to 60° to 65° C. |
| 9 | Add a quantity sufficient (QS) with water to compensate |
| | for any loss due to evaporation. |
| 10 | Note that the required quantity of stock water phase |
| | required for the formulation. |

TABLE 5
Oil Phase.
Oil Phase

| | |
|---|---|
| 11 | To a main tank (Tank I), dispense all the oil phase ingredients. |
| 12 | Heat the main tank (Tank I) to 75°-80° C. while stirring, all |
| | the material to melt until clear and uniform oil phase results. |
| | Adjust the bulk temperature to 70°-75° C. and maintain until use. |

TABLE 6
Final Phase - Emulsion.
Final Phase - Emulsion

| | |
|---|---|
| 13 | To tank (Tank I) at 70°-75° C. while stirring, transfer |
| | slowly the required quantity of the Water Phase from the |
| | tank (Tank II) (60° to 65° C.). |
| 14 | Once the tank (Tank I) equilibrates to 70°-75° C., |
| | continue with stirring for another 10-15 minutes. |
| 15 | Cool the tank (Tank I) to 60° C. and maintain with stirring. |
| 16 | At 60° C., transfer the Active Phase (Tank III) into |
| | Tank I in moderate pace with gentle mixing (i.e., |
| | preventing any splurge of the active solution) and |
| | continue stirring until fully blended with the emulsion. |
| | Using the reserved pre-warmed propylene glycol (approx. |
| | 60° C.) rinse Tank III and add the rinsings back to Tank I. |
| | Continue with stirring until uniform. |
| 17 | At 55°-60° C. check the pH of the emulsion and adjust to |
| | pH 5-6 using 20% aqueous solution of sodium hydroxide. |
| 18 | Reconcile the yield and QS with water to 100%. |
| 19 | At 55°-60° C., pass the emulsion through a homogenizer, |
| | e.g., a Silverson homogenizer (L5M model), 2-2.5 minutes |
| | at 5000 rpm-6000 rpm using the emulsor screen (350 g |
| | batch size), taking note of the bulk weight before and |
| | after homogenization. Record the loss incurred from the |
| | homogenization. |
| 20 | Cool down while stirring and stop once the bulk temperature |
| | achieves 30°-35° C. Check any loss due to evaporation, and |
| | if necessary, QS with water to the weight after homogenization |
| | or prior to final mixing. (post homogenization). Seal bulk |
| | container and low to fully set overnight (8-12 hours). |
| | Calculate and record batch container yield and loss due to |
| | processing/homogenization. |
| 21 | Characterize the product, check viscosity, and pH. |

The final product is a blend of two components, the emulsion base, and the propellant (Table 7).

TABLE 7
Ruxolitinib foam composition product
components per unit packaging.

| | | Approx. | Proposed net fill |
|---|---|---|---|
| Main Ingredients | Description | % w/w | specifications, g |
| Emulsion base | Ruxolitinib | 92.5% | 30.5 |
| | emulsion | | |
| Propellant, P75 | Propane, | 7.5% | 2.5 |
| | Isobutane, butane | | |
| Total per | Ruxolitinib | 100.0 | 33.0 |
| unit container | alopecia foam | | |
| | finished product | | |

Table 8 proposes intermediate bulk product specifications of the ruxolitinib emulsion. Table 9 proposes finished product specifications for the ruxolitinib foam composition.

TABLE 8
Proposed intermediate bulk product specifications
of the ruxolitinib emulsion.

| | | | Proposed Bulk |
|---|---|---|---|
| | | | Product |
| Test Type | Test Attribute | Test Method | Specification |
| Analytical | Assay for | — | 90-110% label |
| | ruxolitinib and | | claim |
| | total impurities | | |
| Analytical | Content | Reference USP<905> | RSD ≤2% |
| | uniformity | | |
| Physical | Appearance | Visual | Opaque-white, |
| | | | viscous cream |
| Physio- | Viscosity at | Anton Paar Visco QC | Approx. |
| chemical | 25° C. | 300- RH4, 20 rpm | 7000-12000 mPa's |
| | | and RH5, 10 rpm | |
| Physio- | pH at 25° C. | pH Meter - Seven | pH 5.0-6.0 |
| chemical | | Compact Mettler | |
| | | Toldeo S220 | |
| Physio- | Density at | Anton Paar Density | Approx. |
| chemical | 25° C. | Meter DMA 501 | 0.9400-0.9800 g/ml |

TABLE 9
Proposed finished product specifications for the ruxolitinib foam composition.

| | | | Proposed |
|---|---|---|---|
| Test Type | Test Attribute | Test Method | Specification |
| Analytical | Assay for ruxolitinib | — | 90-110% label claim |
| | and total impurities | | |
| Analytical | Delivered Dose | Per USP <607>, | Complies with the |
| | Uniformity - Metered | pharmaceutical foams | requirement for |
| | dose products | | delivered dose |
| | | | uniformity |
| Physical | Foam appearance, | Visual | White creamy foam, |
| | foam structure and | | full firm well formed |
| | rate of foam collapse | | and stable peak. |
| Physio-chemical | Foam pH at 25° C. | pH Meter - Seven | pH 5.0-6.0 |
| | | Compact Mettler | |
| | | Toldeo S220 | |
| Packaging/Container | Metered dosing | Per USP <603>, | Record |
| closure | properties - number | Topical Asersols | |
| | of discharges, Drug | | |
| | Dose Delivery Profile | | |
| Microbial limits | Antimicrobial | Per USP general | Complies with AET |
| | Efficacy Testing | chapter <51> | requirement for |
| | (AET) | | category 2 products |

### Example 3: Stability of Topical Ruxolitinib Foam Formulations

A total of five base formulations were assessed with ruxolitinib phosphate for chemical stability as well as base physical stability up to 4 weeks.

### Base Vehicle Formulation Details

Five vehicle formulations (i.e., base solutions) were developed and ruxolitinib phosphate was added to all five vehicle base formulations to assess chemical and physical stability. These formulations were not aerosolized. Additional stability may be possible upon the addition of a propellant. Details of the five vehicle base formulations and corresponding active formulations are summarized in Table 10.

TABLE 10
Vehicle base formulation details and corresponding active formulation numbers.

| | | Vehicle | Vehicle | Vehicle | Vehicle | Vehicle |
|---|---|---|---|---|---|---|
| | | Base 1 | Base 2* | Base 3 | Base 4 | Base 5 |
| Ingredients | Function | % w/w | % w/w | % w/w | % w/w | % w/w |
| Water (deionized) | Solvent | 69.28 | 76.37 | 72.44 | 68.10 | 13.91 |
| Citric acid, | pH | 0.05 | 0.11 | 0.11 | 0.10 | 0.11 |
| anhydrous | adjustment | | | | | |
| Sodium Citrate | pH | 0.25 | 0.20 | 0.25 | 0.30 | 0.24 |
| Dihydrate | buffering | | | | | |
| Oleth-10 | Surfactant | 7.53 | — | — | — | — |
| PEG 40 stearate | Thickening | — | — | 3.43 | — | — |
| | agent, | | | | | |
| | viscosity | | | | | |
| | modifier | | | | | |
| Disodium EDTA | Chelating | 0.10 | 0.11 | 0.11 | 0.10 | — |
| | agent | | | | | |
| Polysorbate 80 | Emulsifier | — | — | — | 7.51 | — |
| Polysorbate 20 | Emulsifier | — | 3.95 | — | — | — |
| Sorbitan laurate | Emulsifier | — | 3.58 | — | — | — |
| Ceteareth-20 | Emollient | — | — | — | — | 3.91 |
| Cetyl alcohol | Emollient, | 1.50 | 1.51 | 1.53 | 1.51 | 3.68 |
| | surfactant | | | | | |
| PEG 300 | Solvent | 3.00 | 2.96 | — | 4.99 | — |
| PEG 400 | Solvent | 2.0 | — | 7.03 | 4.97 | — |
| Glycerin | Humectant | — | — | — | −11.73 | |
| BHT | Preservative | 0.11 | 0.11 | 0.10 | — | — |
| Benzyl Alcohol | Solvent, | 0.51 | 0.52 | 0.50 | 0.56 | 0.49 |
| | Preservative | | | | | |
| Emulsifying Wax | Emulsifier | 4.99 | — | — | — | — |
| Glyceryl stearate | Emollient, | — | — | 2.58 | — | 1.01 |
| | Thickening | | | | | |
| | agent | | | | | |
| NaOH 20% to pH | pH | qs | qs | qs | qs | qs |
| 5-6 | adjustment | | | | | |
| Petrolatum/Mineral | Emollient | — | — | 1.09 | 0.99 | 35.42 |
| Oil | | | | | | |
| Medium chain | Emollient | — | — | — | 0.10 | — |
| triglycerides | | | | | | |
| Natrosol 250H | Thickening | — | — | 0.24 | — | — |
| (HEC) | agent, | | | | | |
| | viscosity | | | | | |
| | modifier | | | | | |
| Propylene glycol | Permeation | 10.02 | 9.86 | 9.99 | 10.03 | 9.44 |
| | enhancer, | | | | | |
| | solvent | | | | | |
| Carbopol 981 | Thickening | — | — | — | 0.10 | — |
| | agent, | | | | | |
| | viscosity | | | | | |
| | modifier | | | | | |
| Hydrogenated | Emollient | — | — | — | 0.25 | — |
| Castor Oil | | | | | | |
| TOTAL | — | 100.020 | 99.28 | 99.50 | 99.51 | 99.85 |
| Rux Formulation | — | F176-6-8 | F176-6-3 | F176-6-4 | F176-6-6 | F176-6-7 |
| ID | | | | | | |
| Final pH | — | 5.57 | 5.55 | 5.57 | 5.64 | 5.29 |

*Preliminary formulation tested for chemical stability (F176-6-3) is based on Vehicle Base 2, minus the addition of cetyl alcohol.

### Chemical Stability

Analytical assay data and degradants are summarized below in Table 11. The protocol used for determining the purity was a liquid chromatography (LC) method. The LC method is provided below.

TABLE 11
Stability Data for Ruxolitinib Phosphate at 40° C./75% RH.

| | | | | Ruxolitinib | Total |
|---|---|---|---|---|---|
| Formulation | LC (LC) | | | Phosphate | Impurities |
| ID | (% w/w) | Storage | Weeks | (% LC) | (% area)** |
| F/L 176-3-02 | 0.54 | 40° C. | 0 | 101.3 | <0.2 |
| (Preliminary) | | | 4 | 100.8 | <0.2 |
| 176-6-3* | 0.50 | 40° C. | 0 | 101.5 | <0.2 |
| | | | 4 | 91.1 | <0.2 |
| 176-6-4 | 0.50 | 40° C. | 0 | 102.8 | <0.2 |
| | | | 4 | 100.9 | <0.2 |
| 176-6-6 | 0.50 | 40° C. | 0 | 102.0 | <0.2 |
| | | | 2 | 100.7 | <0.2 |
| | | | 4 | 100.5 | <0.2 |
| 176-6-7 | 0.15 | 40° C. | 0 | 103.6 | <0.2 |
| | | | 4 | 103.6 | <0.2 |
| 176-6-8 | 0.50 | 40° C. | 0 | 103.5 | <0.2 |
| | | | 2 | 102.5 | <0.2 |
| | | | 4 | 102.0 | <0.2 |

**Total impurities data are an estimate only using the method that was not specifically directed for impurities analysis.

Ruxolitinib did not show impurities across all formulations. The potency loss of 10% for formulation 176-6-3, however, is not likely due to chemical degradation. The potency loss is more likely due to physical instability at 40° C. of the formulation (based on Vehicle 2) leading to inhomogeneity of the ruxolitinib in the sample.

Preliminary formulation F176-3-2 is the same base formulation as F176-6-3 with the exception of no addition of cetyl alcohol in F176-3-2, suggesting cetyl alcohol may potentially cause physical instability over time. Formulation F176-3-2 was initially used for the solubility study where cetyl alcohol was omitted and was also analyzed for chemical stability as a comparison. Furthermore, the cetyl alcohol present in formulation F176-6-3 may be further solubilized when the formulation is charged with propellant and this may physically stabilize the formulation further. Assessment of both potency and total impurities for Vehicle base 5 shows stability and is the only base that contains low water.

### Determination of Ruxolitinib Phosphate in Foam Samples by High Performance Liquid Chromatography

This method is for the determination of Ruxolitinib Phosphate in foam formulations that contain Ruxolitinib Phosphate as active pharmaceutical ingredient (API). The method is suitable for the determination of active content in stability screening samples. The method can also be used to estimate the level of major degradants of Ruxolitinib Phosphate.

### Apparatus

Analytical column: Kinetex C18 Column 4.6×100 mm, 2.6 μm, 100 A.

Guard column (optional); Phenomenex Security Guard Gemini C18 4×2 mm ID or equivalent.

HPLC system: Agilent 1260 comprising a pump, 1290 AD detector or 1260 DAD detector, or a multi-wavelength UV detector, an auto sampler and data collection system or equivalent.

5-digit analytical balance and 6-digit microbalance.

Volumetric glass ware

### Preparation of Solutions:

Diluent: 85% Methanol-15% water-0.05% phosphoric acid

To prepare 1 L of diluent, mix 850 mL of methanol, 150 ml of water, and 1 mL of 50% (w/w) phosphoric acid in a solvent bottle. Mix well.

Mobile Phase A: 0.05% H₃PO₄ in water

To make 1 liter, add 1 mL of 50% (w/w) phosphoric acid into 1 L of water. Mix well.

Mobile Phase B: 100% Acetonitrile

### Preparation of Standard Solutions:

Standard Solution 1 (Stdl. 0.1 mg/mL Ruxolitinib Phosphate)

Using a 6-digit micro balance, weigh accurately about 3.3 mg of Ruxolitinib Phosphate reference material into a 25 mL volumetric flask.

Add 5 mL of methanol to dissolve the content and make up to volume with diluent. Mix well.

Conversion factor from Ruxolitinib Phosphate to Ruxolitinib is 0.758.

Standard Solution 2 (Stdl. 0.05 mg/mL Ruxolitinib Phosphate)

Using a 6-digit micro balance, weigh accurately about 3.3 mg of Ruxolitinib Phosphate reference material into a 50 mL volumetric flask.

Add 5 mL of methanol to dissolve the content and make up to volume with diluent. Mix well.

Conversion factor from Ruxolitinib Phosphate to Ruxolitinib is 0.758.

### Resolution Solution (RS):

Weigh accurately about 3.3 mg of Ruxolitinib Phosphate reference material and approximately 0.5 mg of Ruxolitinib Phosphate into a 25 mL volumetric flask.

Add 5 mL of methanol to dissolve and then make up to volume with diluent. Mix well.

### Preparation of Foam Sample for HPLC Analysis:

Foam formulations contain about 0.5% Ruxolitinib base (API added as Ruxolitinib Phosphate)

Shake the Can for 10 seconds and fit a PE tubing to the Can spout.

Discard the first metered dose of foam from the Can.

Tare a 25 mL volumetric flask.

Dispense one metered dose of foam into the tared 25 mL flask and record the sample weight (Wu, mg) at 2 minutes after dispensing the foam.

Add about 5 mL of methanol to the sample flask to disperse the foam sample and immediately make up to volume with diluent.

Sonicate the sample for 10 minutes and mix well.

Equilibrate the sample solution to room temperature and filter a portion through 0.22 μm PTFE syringe filter into 2 mL HPLC vial for HPLC analysis. Discard the first 1-2 mL of filtrate.

### Preparation of Foam Base/Bulk Sample for HPLC Analysis

Weigh accurately about 500 mg of sample into a tared 25 mL volumetric flask.

Add about 5 mL of methanol to disperse the sample and make up to volume with diluent immediately.

Sonicate the sample for 10 minutes and mix well.

Equilibrate the sample solution to room temperature and filter a portion through

0.22 μm PTFE syringe filter into 2 mL HPLC vial for HPLC analysis. Discard the first 1 mL of filtrate.

### HPLC Analysis

Equilibrate the chromatographic system with starting mobile phase composition until a stable baseline is obtained. The following gradient (Table 12) at a flow rate of 1.0-1.5 mL/min is used:

TABLE 12
Gradient for the HPLC analysis

| Time (min) | Flow Rate mL/min | % A | % B |
|---|---|---|---|
| 0 | 1.0 | 90 | 10 |
| 0.5 | 1.0 | 90 | 10 |
| 7 | 1.0 | 78 | 22 |
| 9 | 1.0 | 70 | 26 |
| 11 | 1.0 | 34 | 54 |
| 16 | 1.5 | 0 | 100 |
| 18 | 1.5 | 0 | 100 |
| 18.1 | 1.5 | 90 | 10 |
| 22 | 1.5 | 90 | 10 |

Ruxolitinib retention time is about 9.3 minutes. Using the instrument conditions, equilibrate the chromatographic system with mobile phase. Record the chromatograms of Ruxolitinib Phosphate peak for the standard and sample solutions. The retention time of principal peak in the HPLC chromatogram of the sample should correspond with the retention time of the principal peak produced by Ruxolitinib Phosphate Standard Solution. The retention time difference between sample and standard should not be more than 5%. V spectrum of Ruxolitinib Phosphate in sample and standard is comparable.

### Physical Stability

Physical stability was assessed for each vehicle base formulation. Physical stability at 4 weeks is summarized below in Table 13.

TABLE 13
Physical stability of vehicle bases with ruxolitinib.

| | | |
|---|---|---|
| Vehicle | | |
| Base Number | Formulation ID | Observation |

Vehicle 1

| | | |
|---|---|---|
| Ruxolitinib | F176-6-8 | Creaming, not physically stable. |
| phosphate | | |

Vehicle Base 2

| | | |
|---|---|---|
| Ruxolitinib | F176-6-3 | Additional of cetyl alcohol may |
| phosphate | | potentially cause physical instability |
| | | over time, Preliminary formulation |
| | | F176-3-2 where cetyl alcohol was |
| | | omitted shows to be physical stable. |

Vehicle Base 3

| | | |
|---|---|---|
| Ruxolitinib | F176-6-4 | Physically stable, light emulsion. |
| phosphate | | |

Vehicle Base 4

| | | |
|---|---|---|
| Ruxolitinib | F176-6-6 | Agglomeration appearing, may |
| phosphate | | potentially cause physical instability. |

Vehicle Base 5

| | | |
|---|---|---|
| Ruxolitinib | F176-6-7 | Physically stable, low water |
| phosphate | | emulsion. |

Based on the observations in Table 13, Vehicle bases 3 and 5 are consistently stable. Vehicle base 1 shows the most physical instability where creaming occurred, followed by Vehicle base 4 where some agglomeration of the emulsion was evident.

Vehicle base 2 shows potential physical instability when cetyl alcohol was added (F176-6-3), however, with no addition of ceytl alcohol (F176-3-2) Vehicle base 2 is physically stable.

### Example 4: Stability of 0.5% Ruxolitinib Foam Composition

Ruxolitinib was prepared with the following lipophilic base formulation:

| Ingredient | Function | % w/w |
|---|---|---|
| Water (Deionized) | Solvent | 30.0 |
| Citric acid, Anhydrous | pH adjustment | 0.10 |
| Sodium Citrate Dihydrate | pH buffering | 0.25 |
| Disodium Edetate | Cheleating agent | 0.1 |
| Ceteareth-20 | Emollient | 3.5 |
| Glycerin | Humectant | 6.91 |
| Natrosol 250 HX | Thickening agent, | 0.30 |
| | viscosity modifier | |
| Cetyl Alcohol | Emollient, surfactant | 2.50 |
| Glyceryl monostearate | Emollient, thickening | 1.50 |
| | agent | |
| Light Mineral Oil | Emollient | 26.52 |
| Medium chain triglyceride | Emollient, permeation | 12.19 |
| | enhancer | |
| Benzyl alcohol | Preservative, solvent | 0.50 |
| SUBTOTAL | — | 84.37 |
| Ruxolitinib phosphate | — | |
| Propylene glycol | Permeation enhancer, | 15.00 |
| | solvent | |
| NaOH 20% to pH 5-6 | — | |

The emulsion based was filled in a vial and the corresponding foam product with 6.9% P75 (propane, isobutane, butane) in a Turbiscan crimped vial, which were kept at 40° C. oven to monitor emulsion stability and base-propellant miscibility.

When taken out after about 4 weeks at 40° C., visual examination revealed the sample was already showing signs of phase separation with a soapy layer at the bottom of the respective container.

### Example 5: Topical Ruxolitinib Foam Compositions

Topical foam formulations of ruxolitinib will be prepared based on the formulations in Table 14.

TABLE 14
Foam formulations of ruxolitinib.

| | | Form 1 | Form 2 | Form 3 | Form 4 | Form 5 |
|---|---|---|---|---|---|---|
| Component | Function | (% w/w) | (% w/w) | (% w/w) | (% w/w) | (% w/w) |
| Ruxolitinib | Active | 0.66 | 0.66 | 0.66 | 0.66 | 0.66 |
| (Phosphate salt = | Ingredient | | | | | |
| 0.5% FB) | | | | | | |
| Bezyl alcohol | Preservative, | 0.5 | 0.51 | 0.5 | 0.56 | 0.49 |
| | solvent | | | | | |
| BHT | Preservative | — | 0.11 | 0.1 | — | — |
| Carbopol 981 | Viscosity | — | — | — | 0.1 | — |
| | modifier, | | | | | |
| | Thickening | | | | | |
| | agent, | | | | | |
| | mucoadhesive | | | | | |
| Ceteareth-20 | Emollient | — | — | — | — | 3.91 |
| Cetyl alcohol | Emollient | 0.1 | 1.5 | 1.53 | 1.51 | 3.68 |
| Citric acid, | pH adjustment | 2.5 | 0.05 | 0.11 | 0.1 | 0.11 |
| anhydrous or | | | | | | |
| trisodium citrate | | | | | | |
| dihydrate | | | | | | |
| EDTA | Cheleating | 0.1 | 0.1 | 0.11 | 0.1 | — |
| | agent | | | | | |
| Emulsifiying Wax | Emulsifier | — | 4.99 | — | — | — |
| Glycerin | Humectant | 0.1 | — | — | — | 11.73 |
| Glyceryl | Emollient, | 7.05 | — | 2.58 | — | 1.01 |
| monostearate | Thickening | | | | | |
| | agent | | | | | |
| Hydrogenated Castor | Emollient | | — | — | 0.25 | — |
| Oil | | | | | | |
| Hydroxyethyl | Thickening | 1.05 | — | — | — | — |
| cellulose | agent, | | | | | |
| | viscosity | | | | | |
| | modifier | | | | | |
| Mineral Oil | Emollient | 27.08 | — | 1.09 | 0.99 | 35.42 |
| MCT | Emollient | 12.44 | — | — | — | 19.91 |
| Natrasol 250H (HEC) | Thickening | — | — | 0.34 | — | — |
| | agent, | | | | | |
| | viscosity | | | | | |
| | modifier | | | | | |
| Oleth-10 | Surfactant | — | 7.53 | — | — | — |
| PEG 40 stearate | Thickening | — | — | 3.43 | — | — |
| | agent, | | | | | |
| | viscosity | | | | | |
| | modifier | | | | | |
| PEG 300 | Solvent | — | 3 | — | 4.99 | — |
| PEG 400 | Solvent | — | 2.5 | 7.03 | 4.97 | — |
| Polyoxyl 20 | Emulsifier, | 2.45 | — | — | — | — |
| cetostearyl ether | Surfactant | | | | | |
| Polysorbate 20 | Emulsifier | — | — | — | — | — |
| Polysorbate 80 | Emulsifier | — | — | — | 7.51 | — |
| Propylene glycol | Permeation | 15 | 10.02 | 9.99 | 10.03 | 9.44 |
| | enhancer, | | | | | |
| | solvent | | | | | |
| Sodium Citrate | pH buffering | — | 0.25 | 0.25 | 0.3 | 0.24 |
| Dihydrate | | | | | | |
| Sodium hydroxide, | pH adjustment | 0.27 | q.s to | q.s to | q.s to | q.s to |
| 20% solution | | | pH 5-6 | pH 5-6 | pH 5-6 | pH 5-6 |
| (Approx) | | | | | | |
| Water, Purified | Solvent | q.s to | q.s to | q.s to | q.s to | q.s to |
| | | 100 | 100 | 100 | 100 | 100 |

The foamable formulations provided above can be placed in a pressurized can with a propellant, which when expelled from the container provides a foam.

### Example 6: Additional Topical Foam Formulations

Additional topical foam formulations to be prepared according to FIG. 1 include the following in Table 15.

TABLE 15
Additional foam formulations.

| | | Formulation ID | | | | | | |
|---|---|---|---|---|---|---|---|---|
| | | | | | | | | F176-6-3 |
| | | | | | | | | unstable |
| | | F176-3-01 | F176-3-02 | F176-3-03 | F176-3-04 | F176-3-05 | F176-6-08 | at 40 C. |
| Ingredients | Function | % w/w | % w/w | % w/w | % w/w | % w/w | % w/w | % w/w |
| Active Phase Solution | | | | | | | | |
| Ruxolitinib | | 0.66 | 0.66 | 0.66 | 0.66 | 0.66 | 0.66 | 0.66 |
| Propylene Glycol | Solvent, | 9.98 | 15.30 | 10.01 | 14.14 | 15.14 | 10.02 | 9.86 |
| | permeation | | | | | | | |
| | enhancer | | | | | | | |
| Water Phase Gel | | | | | | | | |
| Water (Deionized) q.s | Solvent | 73.01 | 72.45 | 81.84 | 69.54 | 63.39 | 69.28 | 76.37 |
| to 100 | | | | | | | | |
| Ethanol | Solvent | | | | | | | |
| Citric Acid Anhydrous | pH adjustment | 0.10 | 0.10 | 0.11 | 0.11 | 0.10 | 0.05 | 0.11 |
| Sodium Citrate | pH buffering | 0.10 | 0.21 | 0.10 | 0.20 | 0.31 | 0.25 | 0.20 |
| Dihydrate | | | | | | | | |
| Disodium EDTA | Cheating agent | | | | 3.57 | | 0.10 | 0.11 |
| Natrosol 250H | Thickening | | | | | | | |
| (Hydroxyethyl | agent, viscosity | | | | | | | |
| cellulose) | modifier | | | | | | | |
| Glycerin | Humectant | | | | | | | |
| PEG 40 Stearate | Thickening | 0.19 | 0.11 | 0.10 | 0.11 | | | |
| | agent, viscosity | | | | | | | |
| | modifier | | | | | | | |
| PEG 300 | Solvent | 3.01 | 3.06 | | | 5.09 | 3.00 | 2.96 |
| PEG 400 | Solvent | 2.55 | | | 7.22 | 5.07 | 2.50 | |
| Carbopol 981 | Thickening | | | | | | | |
| | agent, viscosity | | | | | | | |
| | modifier, mucoad | | | | | | | |
| | hesive | | | | | | | |
| Oleth-10 | Surfactant | 7.97 | | | | | 7.53 | |
| Ceteareth-20 | Emmolient | | | | | | | |
| Natrosol 250H | Thickening | | | | | | | |
| (Hydroxyethyl | agent, viscosity | | | | | | | |
| cellulose) | modifier | | | | | | | |
| Oil Phase | | | | | | | | |
| Polysorbate 80 | Emulsifier | | | | | 7.65 | | |
| Polysorbate 20 | Emulsifier | | 4.06 | | | | | 3.95 |
| Sorbitan Laurate | Emulsifier | | 3.54 | | | | | 3.58 |
| Steareth-10 | Emulsifier, | | | 3.00 | | | | |
| | surfactant | | | | | | | |
| Glyceryl Monostearate | Emollient, | | | | 2.64 | | | |
| | Thickening agent | | | | | | | |
| Cetyl alcohol | Emollient | 1.47 | | 1.48 | | | 1.50 | 1.51 |
| Stearyl alcohol | Emmolient | | | | | | | |
| BHT | Preservative | | | | | | 0.11 | 0.11 |
| Benzyl Alcohol | Preservative, | 0.64 | 0.65 | 0.51 | 0.53 | 1.28 | 0.51 | 0.52 |
| | solvent | | | | | | | |
| Emulsifying Wax | Emulsifier | | | | | | 4.99 | |
| Glyceryl Stearate | Emollient, | | | 2.03 | | | | |
| | Thickening agent | | | | | | | |
| Petrolatum/Mineral Oil | Emollient | | | | 1.09 | 1.12 | | |
| Medium Chain | Emollient, | | | | | | | |
| Triglycerides | permeation | | | | | | | |
| | enhancer | | | | | | | |
| Hydrogenated Castor | Emollient | | | | | | | |
| Oil | | | | | | | | |
| pH Adjustment | | | | | | | | |
| NaOH 20% pH | | qs to | qs to | qs to | qs to | qs to | pH to | pH to |
| adjustment | | pH 6-7 | pH 6-7 | pH 6-7 | pH 6-7 | pH 6-7 | 5-6 | 5-6 |

| | | Formulation ID | | | | | |
|---|---|---|---|---|---|---|---|
| | | | | | Hydroethanolic | | |
| | | | | | Foam formulations | | |
| | | F176-6-4 | F176-6-6 | F176-6-7 | | Ex 1* | Ex 2* |
| Ingredients | Function | % w/w | % w/w | % w/w | Range | % w/w | % w/w |
| Active Phase Solution | | | | | | | |
| Ruxolitinib | | 0.66 | 0.66 | 0.66 | 0.1-1.0% | 1.00 | 1.00 |
| Propylene Glycol | Solvent, | 9.99 | 10.03 | 9.44 | 2-20% | 15.00 | 15.00 |
| | permeation | | | | | | |
| | enhancer | | | | | | |
| Water Phase Gel | | | | | | | |
| Water (Deionized) q.s | Solvent | 72.44 | 68.10 | 13.91 | 30.00-60% | 30.00 | 30.0 |
| to 100 | | | | | | | 0 |
| Ethanol | Solvent | | | | 40.00-65% | 40.00 | 40.00 |
| Citric Acid Anhydrous | pH adjustment | 0.11 | 0.10 | 0.11 | | | |
| Sodium Citrate | pH buffering | 0.25 | 0.30 | 0.24 | | | |
| Dihydrate | | | | | | | |
| Disodium EDTA | Cheating agent | 0.11 | 0.10 | | | | |
| Natrosol 250H | Thickening | 0.34 | | | | | |
| (Hydroxyethyl | agent, viscosity | | | | | | |
| cellulose) | modifier | | | | | | |
| Glycerin | Humectant | | | 11.73 | | | |
| PEG 40 Stearate | Thickening | 3.43 | | | | | |
| | agent, viscosity | | | | | | |
| | modifier | | | | | | |
| PEG 300 | Solvent | | 4.99 | | 2-20% | 4.00 | |
| PEG 400 | Solvent | 7.03 | 4.97 | | 2-20% | | 4 |
| Carbopol 981 | Thickening | | 0.10 | | | | |
| | agent, viscosity | | | | | | |
| | modifier, mucoad | | | | | | |
| | hesive | | | | | | |
| Oleth-10 | Surfactant | | | | | | |
| Ceteareth-20 | Emmolient | | | 3.91 | | | |
| Natrosol 250H | Thickening | 0.34 | | | | | |
| (Hydroxyethyl | agent, viscosity | | | | | | |
| cellulose) | modifier | | | | | | |
| Oil Phase | | | | | | | |
| Polysorbate 80 | Emulsifier | | 7.51 | | | | |
| Polysorbate 20 | Emulsifier | | | | | | |
| Sorbitan Laurate | Emulsifier | | | | | | |
| Steareth-10 | Emulsifier, | | | | | | |
| | surfactant | | | | | | |
| Glyceryl Monostearate | Emollient, | | | | | | |
| | Thickening agent | | | | | | |
| Cetyl alcohol | Emollient | 1.53 | 1.51 | 3.68 | 0.5-5% | 5.00 | 5.00 |
| Stearyl alcohol | Emmolient | | | | 0.5-5% | 5.00 | 5.00 |
| BHT | Preservative | 0.10 | | | | | |
| Benzyl Alcohol | Preservative, | 0.50 | 0.56 | 0.49 | | | |
| | solvent | | | | | | |
| Emulsifying Wax | Emulsifier | | | | | | |
| Glyceryl Stearate | Emollient, | 2.58 | | 1.01 | | | |
| | Thickening agent | | | | | | |
| Petrolatum/Mineral Oil | Emollient | 1.09 | 0.99 | 35.42 | | | |
| Medium Chain | Emollient, | | | 19.91 | | | |
| Triglycerides | permeation | | | | | | |
| | enhancer | | | | | | |
| Hydrogenated Castor | Emollient | | 0.25 | | | | |
| Oil | | | | | | | |
| pH Adjustment | | | | | | | |
| NaOH 20% pH | | pH to | pH to | pH to | pH to | pH to | pH to |
| adjustment | | 5-6 | 5-6 | 5-6 | 5-6 | 5-6 | 5-6 |

* Theoretical

### Example 7: Further Topical Foam Formulation

According to the present disclosure, a further topical foam formulation can include:
from 40%-65% Ethanol
from 30%-60% Water
from 0.5%-5% Stearyl Alcohol
from 0.5%-5% Cetyl Alcohol
from 2%-20% Propylene glycol or PEG300 or PEG400 or other solubilizer

The above further topical foam formulation can be prepared according to the manufacturing procedures outlined in Tables 3-6 above.

The foamable formulations provided above can be placed in a pressurized can with a propellant, which when expelled from the container provides a foam.

### Example 8: In Vivo Testing

One or more of the above-identified topical foam formulations will be examined in an in vivo mouse model. The C3H/HeJ mouse model has been used for in vivo AA research. An example using the C3H/HeJ mouse model can be found in U.S. Pat. No. 9,198,911 and in Xing, et al., “Alopecia areata is driven by cytotoxic T lymphocytes and is reversed by JAK inhibition”, Nat Med, 1043-1049 (2014)). The in vivo testing would be carried out using the C3H/HeJ mouse model.

### Example 9: Proposed Phase 2 in Participants with Mild to Moderate Alopecia Areata

A phase 2, randomized, double-blind, placebo-controlled, dose-ranging study of the efficacy and safety of ruxolitinib phosphate foam or deuruxolitinib phosphate foam in participants with mild to moderate Alopecia Areata (AA) is proposed with about 180 participants.

Participants to be included in the study include: (1) men and women 18 to 65 years of age; (2) a history of AA for ≥2 years; and (3) patchy alopecia with baseline SALT score ≤50%.

The primary endpoint will be a portion of participants achieving a Severity of Alopecia Tool (SALT) score of ≤20. The secondary endpoints include proportion of participants achieving SALT≤10, SALT reduction≤50, and patient reported outcomes (PRO) endpoints.

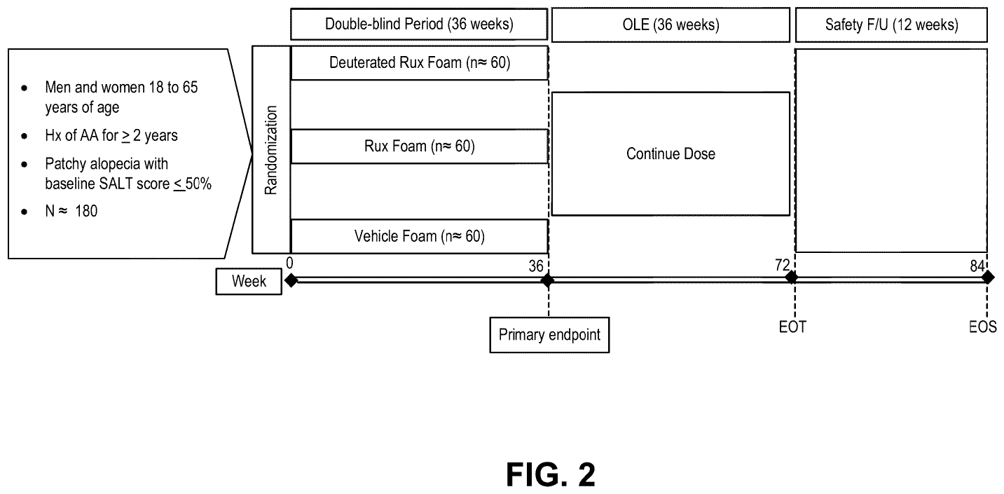

FIG. 2 outlines the proposed study treatment schedule. For the double-blind period of the first 36 weeks, approximately 180 participants will be randomized into three treatment groups: (1) deuruxolitinib phosphate foam (n≈60); (2) ruxolitinib phosphate foam (n≈60); and (3) vehicle foam (n≈60). At the end of the double-blind period, the primary endpoint will be assessed. An open label extension (OLA) will continue for another 36 weeks with the continued dosing scheme. Finally, a long-term safety follow-up (Safety F/U) arm will conclude for an additional 12 weeks.

### Example 10: Proposed A Phase 2 in Participants with Severe Alopecia Areata

A phase 2, randomized, double-blind, placebo-controlled, study of the efficacy and safety of adjunctive ruxolitinib phosphate foam with oral deuruxolitinib phosphate in participants with severe AA is proposed.

Participants to be included in the study include: (1) men and women 18 to 65 years of age; (2) a history of AA for ≥2 years; and (3) alopecia with baseline SALT score >50% for greater than 6 months.

The primary endpoint will be a portion of participants achieving a Severity of Alopecia Tool (SALT) score of ≤20. The secondary endpoints include proportion of participants achieving SALT≤10, and PRO endpoints.

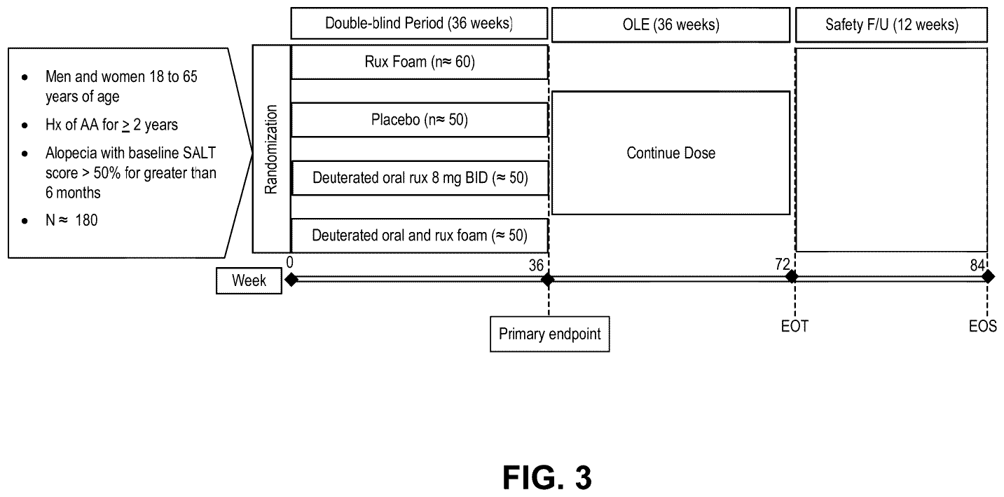

FIG. 3 outlines the proposed study treatment schedule. For the double-blind period of the first 36 weeks, approximately 180 participants will be randomized into four treatment groups: (1) ruxolitinib phosphate foam (n≈50); (2) placebo (n≈50); (3) oral deuruxolitinib phosphate 8 mg (on a free base basis) BID (n≈50); and (4) oral deuruxolitinib phosphate 8 mg (on a free base basis) and ruxolitinib phosphate foam (n≈50). At the end of the double-blind period, the primary endpoint will be assessed. An open label extension (OLA) will continue for another 36 weeks with the continued dosing scheme. Finally, a long-term safety follow-up (Safety F/U) arm will conclude for an additional 12 weeks.

### Example 11: Ruxolitinib Foamable Compositions

### Materials and Methods

### Synthesis of Intermediate 1

Intermediate 1 was prepared by the procedure in WO2022/03603, which is incorporated herein by reference in its entirety.

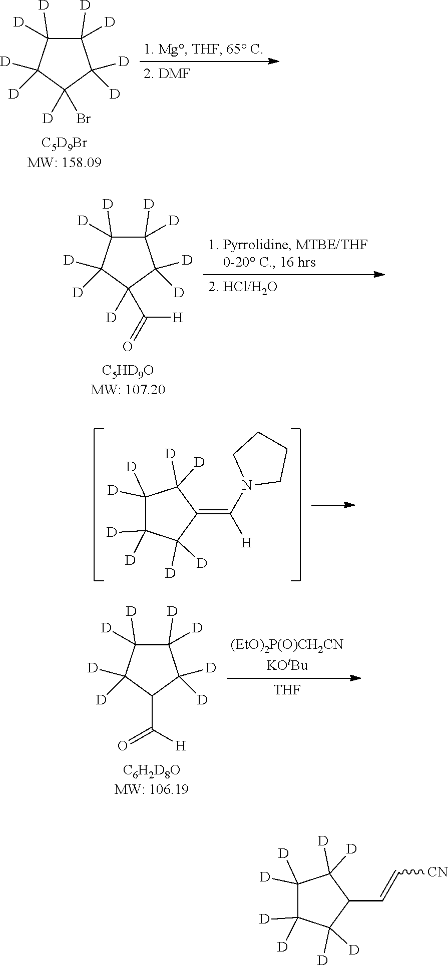

### Step 1: Preparation of Cyclopentane-d9-1-carbaldehyde

Into an 250 mL 3-neck round bottom flask fitted with overhead stirring, condenser, thermocouple and nitrogen inlet was charged magnesium (3.54 g, 145 mmol) and THF (30.0 mL), followed by 1,2-dibromoethane (0.185 mL, 2.147 mmol). The slurry was warmed to 65° C., and then a a solution of 1-bromocyclopentane-1,2,2,3,3,4,4,5,5-d9 (20.0 g, 127 mmol) in THF (58.0 mL) was added portionwise via syringe over 36 minutes. The addition was exothermic with foaming and refluxing solution. When foaming slowed more bromocyclopentane-d9 solution was added. Maximum internal temperature was 72.7° C. Following the addition the reaction was held at 66-67° C. for 2 hours. After completion of the reaction as determined by HPLC, the heating was stopped, and the reaction mixture cooled to 0-5° C. DMF (10.53 mL) was added neat via syringe over 14 minutes. The maximum internal temperature was 7.6° C. The reaction mixture was allowed to stir in ice bath for 10 minutes, then warmed to room temperature. The reaction mixture was stirred for a total of 2 hours following DMF addition. Into a 500 mL round bottom with stir bar was charged 2M hydrochloric acid (100.0 mL, 200 mmol) and then chilled to to 0-5° C. The reaction mixture was added in portions via large plastic pipet. The reaction solution was decanted away from the unreacted turnings. Next, MTBE was added (75 mL). The organic layer was then separated and then back extracted with MTBE (50 mL). The organic fractions were combined and washed with brine (50 mL), then dried over magnesium sulfate. The organic fractions were then filtered to remove magnesium sulfate and then the solid rinsed with THF and filtered. The organic solution was then concentrated in vacuo to 35 mL (60.6 g). The yield was determined by 1H NMR (63%).

### Step 2: Enamine Formation and Hydrolysis

A solution of cyclopentane-d9-1-carbaldehyde (8.85 g, 83 mmol) (step 1) and 6M hydrochloric acid (0.885 mL, 5.31 mmol) was chilled to 0-5° C. in an ice bath, and then pyrrolidine (13.65 mL, 165 mmol) was added in portions. The reaction mixture was stirred in ice for 5 minutes then allowed to warm to room temperature and stirred overnight. After 24.5 hours, the starting aldehyde was consumed as determined by 1H NMR. The reaction mixture was chilled in an ice bath, and then 6M hydrochloric acid (31.0 mL, 186 mmol) was added to the reaction in portions. The reaction mixture was stirred 5 minutes in the ice bath, then the ice bath was removed and the reaction stirred for 3.5 hours. The pH of the reaction mixture was adjusted to ˜8 with 6.38 g 6M KOH. The reaction mixture was partitioned, then the aqueous fraction was back extracted with MTBE (50 mL). The organic fractions were dried over magnesium sulfate and then filtered. The magnesium sulfate was rinsed with MTBE (19 mL), then the solution was concentrated in vacuo to 45 mL) to give 6.1 g of product (crude yield: 69%).

### Step 3: Horner-Wadsworth-Emmons Reaction

Into an oven dried 100 mL, 3-neck round bottom flask fitted with stir bar, septa, thermocouple and nitrogen inlet was charged diethyl (cyanomethyl)phosphonate (3.20 mL, 19.78 mmol) and anhydrous THF (12.0 mL), then the solution was chilled to 0-5° C. Potassium tert-butoxide (19.33 mL, 19.33 mmol) was added via syringe over 14 minutes. Internal temperature went as high as 6.0° C. Following the base addition, the reaction mixture was stirred in the ice bath for 1 hour. Next, the cyclopentane-2,2,3,3,4,4,5,5-d8-1-carbaldehyde (1.91 g, 17.99 mmol) (step 2) solution was charged via syringe over 22 minutes. Internal temperature went as high as 8.2° C. The reaction mixture was stirred for 5 min. in the ice bath then allowed to warm to room temperature. Consumption of the aldehyde was complete after 2 hours. The reaction was quenched with 25 mL 25% (w/w) NaCl solution, then partitioned. The aqueous fraction was back extracted with MTBE (25 mL). The organic fractions were combined and then passed through a silica gel plug. The solvent was then removed in vacuo to give 2.205 g based on 1H NMR wt %. (yield: 94.8%).

### Synthesis of Deuruxolitinib Phosphate

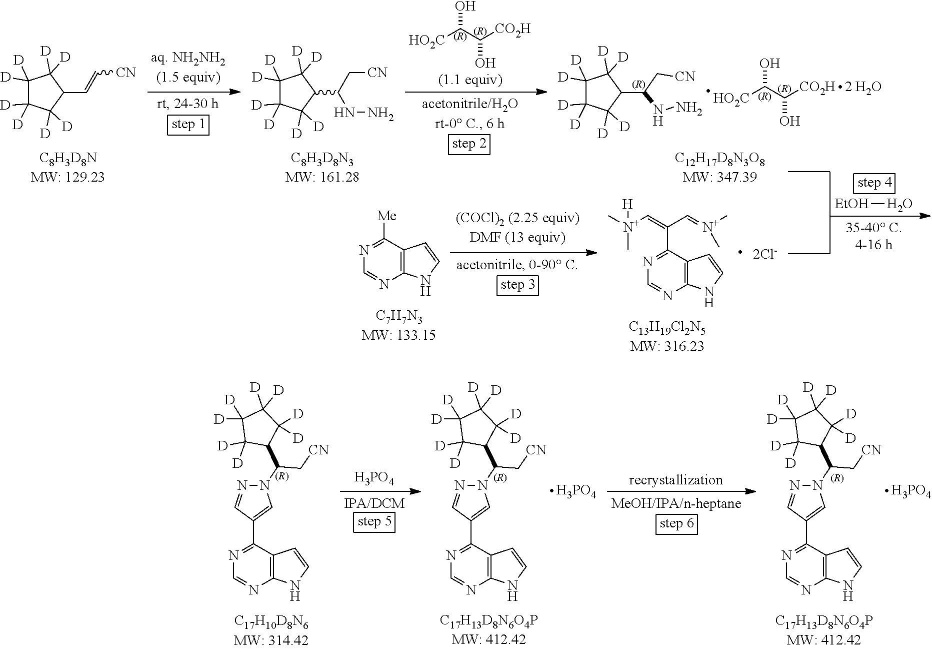

### Step 1. 1(E)-3-(Cyclopentyl-2,2,3,3,4,4,5,5-d8) acrylonitrile

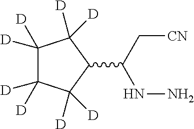 [SMILES: [2H]C1([2H])C(C(CC#N)NN)C([2H])([2H])C([2H])([2H])C1([2H])[2H]]

1(E)-3-(Cyclopentyl-2,2,3,3,4,4,5,5-d8) acrylonitrile (232.2 g, 1204 mmol) (Intermediate 1; also available from Ambinter, AMB38162499, Reg. No. 1513884-14-4) was charged into a 1000 mL round bottom flask equipped with stir bar, addition funnel, thermocouple and nitrogen inlet and then chilled to 2.7° C. Hydrazine hydrate (Aldrich, Lot #SHBK4663; 176 ml, 1806 mmol) was added dropwise, while maintaining the internal temperature of less of 5° C. After 47 hours, the reaction was complete by 1H NMR. The reaction was diluted with reaction with dichloromethane (DCM) (467 ml, 7258 mmol). The reaction was diluted with brine (156 mL and then separated. The aqueous fraction was extracted with DCM (156 ml, 2425 mmol), and then the organic fractions were combined and concentrated. Acetonitrile (ACN) (156 ml, 2987 mmol) was then added and then concentrated. The addition of acetonitrile and concentration was then repeated. The cloudy/slightly turbid solution was filtered through a celite pad. Acetonitrile (156 ml, 2987 mmol) was then added and again concentrated. After holding overnight, the solvent was concentrated in vacuo to yield 337.12 of a solution and 174.6 g (86%) of the product which was used as is in the next step.

### Step 2. (R)-3-(cyclopentyl-2,2,3,3,4,4,5,5-d8)-3-hydrazineylpropanenitrile L-tartrate dihydrate dihydrate

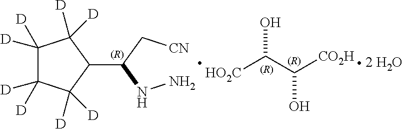

A 5 L, 3 neck round bottom equipped with overhead stirring, septa, thermocouple, 1 L addition funnel and nitrogen inlet was charged with acetonitrile (655 mL), water (655 mL), and (2R,3R)-2,3-dihydroxysuccinic acid (179 g, 1191 mmol) (Alfa Aesar, Lot #U21F057) and then stirred until a solution formed. Stir until solids go into solution. Next, a solution was prepared of 3-(cyclopentyl-2,2,3,3,4,4,5,5-d8)-3-hydrazineylpropanenitrile (174.6 g, 1083 mmol) (step 1, 218 mL, 4174 mmol) and water (218 mL, 1.21E+04 mmol) and then added dropwise to the first solution (40% added over 24 minutes, maintaining temperature at 15.8° C., then remaining solution dropwise over 49 minutes). As the second solution is added, the salt product precipitates out of solution first in large plates but then becomes more voluminous. The slurry was stirred for 2.5 hours at ambient temperature (18-22° C.), then chilled to 1.5° C. and held in ice bath for 2.5 hours. In order to isolate the product, the chilled slurry was filter and then the filter cake washed in portions with a wash solution with ACN (1029 mL) and water (54 mL). The solid was then dried in vacuo overnight to isolate 137.6 g of white crystalline salt. Submit to analytical for analysis (salt chiral purity: 99.12%, yield: 36.6% (73.2%).

### Step 2a: Reslurry of (R)-3-(cyclopentyl-2,2,3,3,4,4,5,5-d8)-3-hydrazineylpropanenitrile L-rartrate dihydrate

A 5 L 4 neck round bottom flask was equipped with with overhead stirring, addition funnel and nitrogen inlet and then charged with 3-(cyclopentyl-2,2,3,3,4,4,5,5-d8)-3-hydrazineylpropanenitrile L-tartrate dihydrate (step 2, 274.7 g, 791 mmol). A solution of CAN (825 mL) and water (275 mL) was then added to the flask and the resultant slurry stirred for 2 hours. ACN then was added (1650.0 mL) dropwise over approximately 2 hours and then the slurry stirred overnight at room temperature. After 18 hours, the slurry was filtered and then rinsed with a wash solution of 5% water/ACN by gently agitating the filter cake. The solids were dried in vacuo overnight to give 263.2 grams (96% yield, chiral purity 99.65%).

### Step 3: (E)-3-(Dimethyliminio)-N,N-dimethyl-2-(7H-pyrrolo[2,3-d]pyrimidin-4-yl) prop-1-en-1-aminium chloride

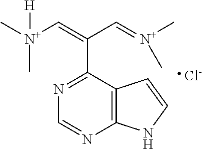

(E)-N-(3-(Dimethylamino)-2-(7H-pyrrolo[2,3-d]pyrimidin-4-yl) allylidene)-N-methylmethanaminium chloride hydrochloride (125 g, 395 mmol) (which can be prepared as described in U.S. Pat. No. 11,905,292 (Example 7), which is incorporated herein by reference in its entirety) and water (125 mL) were charged into a 500 mL round bottom flask. The resultant solution was chilled to 0-10° C. The pH of the solution was adjusted to 7-9 with 30% sodium hydroxide (48.2 g, 362 mmol) added portionwise over 20 minutes. Activated charcoal (25.0 g, 2081 mmol) (EMD, Norit SX-2, Lot #UF28AZEMS) was added along with water (125 mL). The slurry was stirred for 4 hours at room temperature. The slurry was then filtered through a celite pad into a 3 L round bottom flask. The celite pad was rinsed with water (188 mL) and then with ethanol (375 mL). The filtrate was yellow and carried forward into the next step.

### Step 4. Deuruxolitinib

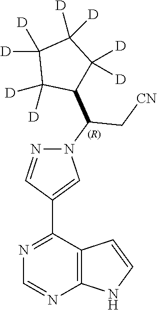

To the solution from step 3 was added ethanol (469 mL) of ethanol (943 mL) and 3-(cyclopentyl-2,2,3,3,4,4,5,5-d8)-3-hydrazineylpropanenitrile (2R,3R)-2,3-dihydroxysuccinate dihydrate (145 g, 417 mmol) (step 2a), followed by more ethanol (469 mL). The solution was stirred overnight at room temperature. After 16.5 hours, the reaction mixture was transferred to a 3 L round bottom flask and then concentrated to 1500 mL volume. Water (438 mL) was then charged to the flask and then the solution concentrated to 1250 mL volume. Next, dichloromethane (438 mL) was charged to the flask, and the solution chilled in an ice bath. The pH of the solution was adjusted with 30% sodium hydroxide (106 g, 794 mmol) to a target pH of 5-7. DCM (125 ml, 1943 mmol) was then charged, and the mixture stirred for 5 minutes. The organic layer was then separated, and the aqueous fraction was extracted with DCM (250 ml, 3886 mmol). The organic fractions were combined and then washed with water (1000 ml, 5.55E+04 mmol), then stirred for 10 minutes then settle for 5 minutes. This was repeated 3 times. All the organic fractions were combined, and polish filtered through a celite pad into a tared 2 L round bottom flask. The solution was then concentrated to a foam by rotary evaporation, then dried in vacuo for 7 hours. An orange foam was recovered (128.4 grams; yield by 1H NMR 94%).

### Step 5. Deuruxolitinib Phosphate

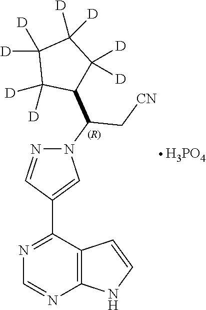

To a 1 L 4-neck round bottom flask containing (R)-3-(4-(7H-pyrrolo[2,3-d]pyrimidin-4-yl)-1H-pyrazol-1-yl)-3-(cyclopentyl-2,2,3,3,4,4,5,5-d8) propanenitrile (225 g, 716 mmol) (step 4) was outfitted with overhead stirring, nitrogen inlet, condenser, addition funnel and thermocouple. IPA (2700 mL) was charged in portions and then warm to 60-62° C. A solution of phosphoric acid (58.8 mL, 859 mmol) and IPA (675 ml, 8761 mmol) was added dropwise to the refluxing solution over 1 hour 34 minutes. A solid precipitated out of solution. The slurry was held at reflux temperature for 15 minutes, then cooled to a temperature of 33.4° C. over 2.5 hours. The slurry was then chilled to 0-5° C. and stirred for 2 hours. The slurry was filtered and then rinsed with the filtrate, then ice cold IPA (900 mL). The filter cake was rinsed at room temperature with n-heptane (900 mL) to displace residual IPA, then dried under house vacuum. The filter cake was dried overnight in 45-50° C. vacuum oven under nitrogen and house vacuum. A light yellow solid was isolated (265.2 g, yield 90%: purity 99.92%; chiral purity: 99.72%).

### Step 6: Recrystallization of deuruxolitinib phosphate

To a 3 L, round bottom with Claisen adaptor, stir bar, thermocouple, septa, condenser and nitrogen inlet was charged (R)-3-(4-(7H-pyrrolo[2,3-d]pyrimidin-4-yl)-1H-pyrazol-1-yl)-3-(cyclopentyl-2,2,3,3,4,4,5,5-d8) propanenitrile phosphate (132.1 g, 320 mmol) to the round bottom flask, then charged methanol (1463 mL), then charcoal (26.4 g, 2201 mmol), then an additional methanol (264 mL). Warm slurry to 55-60° C., and stir for 3 hours at 60° C. The slurry was filtered through a pressed 1″ celite pad, then filtered through a second celite pad. The filtered charcoal was rinsed with methanol (1064 mL), and then warmed to 55-60° C. The methanol wash was filtered through a celite pad into a 5 L 4-neck round bottom. In a round bottom with overhead stirring, short path distillation head, thermocouple and 1 L addition funnel, the solution was distilled at an internal temperature to 67° C. by atmospheric distillation. IPA (1064 mL) was added to dropwise over 19 minutes. A white solid precipitate was produced. IPA was added at such a rate to break up the large clumps of precipitated salt clinging to the side of the flask and improve stirring. The internal temperature was set to 72° C., then n-heptane (661 mL) was added dropwise over 36 minutes while matching the addition and distillation rates. N-heptane (2642 mL) was added in portions, matching the distillation rate as closely as possible. The distillation was completed until a temperature of 68-72° C. The slurry was cooled and then stirred under nitrogen overnight. The salt was filtered 16 hours after distillation was complete, and then rinsed a solution of IPA (132 mL) and n-heptane (661 mL), then n-heptane (500 mL). After drying under house vacuum, the salt was dried in a 45-50° C. vacuum oven to constant weight (approximately 23 hours) Yield 127.2 g (96%).

### Manufacturing Procedures for Foamable Carrier Components and Foamable Compositions

### Manufacturing Procedure (Method A1):

| Step# | MANUFACTURING PROCEDURE |
|---|---|
| 1 | Dispensed all raw materials except ethanol then stir |
| 2 | Heat up to 70° c. to dissolve fatty alchohols with stirring then |
| | cool down |
| 3 | At 40 to 50° C. qs with water then add in the ethanol |
| 4 | Allowed to stir until uniform and cooled to ambient |
| 5 | Leave stirring overnight, reconcile yield and qs with ethanol |
| | the next day |
| 6 | The mixture is reheated |
| 7 | Cool down to determine cloud point - approx. 31° C. |

### Manufacturing Procedure (Method A2):

| Step# | MANUFACTURING PROCEDURE |
|---|---|
| 1 | Tare the bottle incl mag bar. |
| 2 | Dispense PG then all the solid ingredients incl fatty alcohol |
| 3 | Water added then stirred while heating to 50° C. |
| 4 | — |
| 5 | Heating at 55-57° C., Ethanol added |
| 6 | Examine visually |
| 7 | Allowed to cool down |
| 8 | Check yield qs to 96.04 with Ethanol |

### Manufacturing Procedure (Method A3):

| Step# | MANUFACTURING PROCEDURE- Stability 268-5 |
|---|---|
| 1 | Tare the bottle incl mag bar. |
| 2 | Dispense PG/PEG 400 then the surfactants |
| 3 | Add WATER then manually agitate (swirl) bottle |
| 4 | Add the fatty alcohols |
| 5 | Heating is started to the mixture then ETHANOL is added |
| 6 | Heating continued until FA fully dissolved |
| 7 | Cool down, the active substance added at 50° C. |
| 8 | Add Ethanol the last at low temp (40° C.), stir until dissolved. |
| 9 | Check yield qs with Ethanol |
| 10 | Check and adjust pH 5.5 to 6.0 |
| Step# | MANUFACTURING PROCEDURE- Stability 268-6 |
| 1 | Tare the bottle incl mag bar. |
| 2 | Dispense PG/PEG400 then the surfactants |
| 3 | Water added then manually agitated (swirled) |
| 4 | The fatty alcohols are added, heating commenced until dissolved |
| 5 | Allowed to cool down then add the active ingredient. |
| | Stir until dissolved. Reconcile qs with water any loss |
| | from evaporation |
| 6 | Add Ethanol last at low temp (40 C.), stir until no |
| | particulate is left un-dissolved |
| 7 | Check yield qs with Ethanol |
| 8 | Adjust pH 5.5 to 6.0° C. |

### Manufacturing Procedure (Method A4):

| Step# | MANUFACTURING PROCEDURE |
|---|---|
| 1 | Record the tare weight of main container incl mag bar. |
| 2 | To the tared container dispense PG then, record the quantity |
| | to the batch document |
| 3 | Dispense the 2 surfactants one after the other then record |
| | the respective quantity to the doc. |
| 4 | Add the required WATER then record the quantity to the |
| | batch document |
| 5 | Manually agitate the mixture by swirling the container |
| 6 | Add the fatty alcohols one after the other record the |
| | quantity added for each material |
| | note: should be dissolved between 60-65° C. |
| 7 | Once clear and fully dissolved cooldown to 40° C. (the |
| | solution should remain clear and uniform) |
| 8 | At 40° C. check the weight and compensate any lost |
| | water due to evaporation |
| | Actual gross weight =_{——}_{——}_{——}g Qs water |
| | added =_{——}_{——}_{——}g |
| 9 | At 40° C. add the required quantity of Ethanol then |
| | record the quantity to the batch document |
| 10 | Check the pH and adjust within pH 5.0 to 6.0 using 20% |
| | NaOH solujtion |
| | Record initial pH =_{——}_{——}_{——}, Adjusted final |
| | pH =_{——}_{——}_{——} |
| | Quantity NaOH 20% added =_{——}_{——}_{——}g, |
| | Equivalent neat NaOH added =_{——}_{——}_{——}g, Equivalent |
| | water from the NaOH solution =_{——}_{——}_{——}g |
| 11 | Cooldown with stirring, maintain at 38-40° C. if |
| | required for filling-packaging |
| 12 | Calculate actual batch yield: |
| | Actual net/Theoretical net by input per document × |
| | 100 =_{——}_{——}_{——}% |
| 13 | Inspect and characterise the product visually. |

### Manufacturing Procedure (Method A5):

| Step# | MANUFACTURING PROCEDURE |
|---|---|
| 1 | Record the tare weight of main container incl mag bar. |
| 2 | To the tared container dispense PG then add the emollient |
| | material, record the quantity to the batch document |
| 3 | Dispense the 2 surfactants one after the other then |
| | record the respective quantity to the doc. |
| 4 | Add the required WATER then record the quantity to the |
| | batch document. Manually agitate the mixture swirling |
| | the container. |
| 5 | Add the fatty alcohols one after the other record the |
| | quantity added for each material |
| 6 | Transfer the container to a heating plate and start |
| | heating while stirring to dissolve the fatty alcohols. |
| | Note: should be dissolved between 65-70° C. |
| 7 | At 40° C. check the weight and add lost water due |
| | to evaporation. |
| | Actul gross =_{——}_{——}_{——}g Qs water added =_{——}_{——}_{——}g |
| 8 | At 40° C. add the required quantity of Ethanol then |
| | record the quantity to the batch document |
| | Add the required amount of the active ingredient. |
| | Allow to stir until dissolved. Record the temp of |
| | dissolution_{——}_{——}_{——}° C. |
| 9 | Add the required amount of the active ingredient. |
| | Allow to stir until dissolved. Record the temp of |
| | dissolution_{——}_{——}_{——}° C. |
| 10 | At the same temperature add the Glycerin + an emollient |
| | material either PPG15 stearyl ether/Oleic acid/DIPA. |
| | Continue stirring until homogeneous. |
| 11 | Check the pH and adjust within pH 5.0 to 6.0 using 20% |
| | NaOH solution |
| | Record initial pH =_{——}_{——}_{——}, Adjusted final |
| | pH =_{——}_{——}_{——} |
| | Weight of NaOH 20% solution added =_{——}_{——}_{——}g |
| 12 | Cool down with stirring, maintain at 38-40° C. if |
| | required to complete up to packaging |
| 13 | Check the final weight add Ethanol if necessary to |
| | compensate any loss. |
| | Check Final gross weight_{——}_{——}_{——}g Equivalent Net |
| | weight:_{——}_{——}_{——}g |
| 14 | Calculate actual batch yield: Actual net/Theoretical |
| | net by input per document × 100 =_{——}_{——}_{——}% |
| 15 | Inspect and characterise the product visually. |

### Manufacturing Procedure (Method A6):

| Step# | MODIFIED MANUFACTURING PROCEDURE |
|---|---|
| 1 | Record the tare weight of main container incl mag bar. |
| 2 | Prepare the Oil phase in the main container: |
| | If included, dispense Myristyl lactate and Transcutol-P and |
| | dissolve with swirling or applying low heating to main container |
| | Once dissolved, add in/weigh the fatty alcohols and/or Span |
| | 20 and PEG 300, if included any included |
| | Heat up the oil phase to 70° C. (75° C. max) and allow with |
| | stirring for 10 mins |
| 3 | Prepare the Water phase in a separate beaker: |
| | To a beaker dispense PG, Tween 60, Water and Glycerin. |
| | Heat up to 60° C. |
| | Add water required and then record the respective quantity |
| | to the doc. |
| | Continue heating until homogeneous_{——}_{——}_{——}° C. Set aside |
| | until oil phase ready. |
| | Describe appearance of W phase before use and record the |
| | temperature_{——}_{——}_{——}° C._{—} |
| | Check the gross weight of WP and qs with water to make up |
| | for any loss due to evaporation |
| 4 | When the Oil phase is at 65-70° C. transfer required water |
| | phase to the oil phase while stirring. |
| 5 | Allow the Final Phase to emulsify stirring for 10 mins |
| | maintaining temp between 65-70° C. (max 75° C.). Describe |
| | appearance of emulsion, record the temperature eg 60° C. |
| 6 | While stirring cool down to 40° C. |
| 7 | At 40° C. check the weight and add qs water to make up |
| | for loss due to evaporation |
| 8 | At 40° C. add the ¾ quantity of Ethanol then record the |
| | quantity to the batch document |
| 9 | Add the required amount of the active ingredient. Allow |
| | to stir until dissolved. |
| | Record the temp ° C. |
| 10 | Add the remaining ¼ of alcohol while rinsing the sides of |
| | the main container. Continue stirring until fully dissolved. |
| 11 | Check the pH and adjust within pH 5.0 to 6.0 using 20% |
| | NaOH solution/Trolamine |
| | Record initial pH =_{——}_{——}_{——}Weight of alkalising |
| | agent_{——}_{——}_{——}g to final pH = |
| 12 | Cool down with stirring, maintain at 38-40° C. if required |
| | to complete up to packaging |
| 13 | Calculate actual batch yield: Actual net/Theoretical net |
| | by input per document × 100 =_{——}_{——}_{——}% |
| 14 | Visually check and characterise the product |

### Manufacturing Procedure (Method B)

Water Phase (WP), Stock solution 25% excess quantity-Tank 2

Prepared WP stock container Tank-2, weighed and recorded the WP-Tank 2 tare weight.

To Tank 2, dispensed all the WP ingredients according to the batch document.

Recorded actual quantity dispensed for each raw material.

When all ingredients have been weighed in, checked weight and recorded the actual Gross weight of Tank 2.

Transferred Tank 2 to a heating element then, started heating to 65° C. to 70° C. with stirring.

Continued stirring for further 10 minutes, while maintaining 65° C. to 70° C. temperature and continued until required for the next step.

Prior use of the WP-Stock, checked the gross weight of Tank 2, and if necessary qs with purified water to make up any weight loss due to evaporation. Recorded the Gross weight of WP-Stock after qs.

Recorded the net amount WP-stock taken and transferred to the main Tank-1, obtained the Gross weight after taking amount transferred to the main Tank 1.

Set aside Tank 2 discard any residual WP-stock left unused.

### Oil Phase—Main Tank 1

Prepared the oil phase main container-Tank 1, weighed, and recorded the tare weight of main Tank 1.

To Tank 1, dispensed all oil phase ingredients according to the batch document.

Recorded actual quantity dispensed for each raw material.

When all ingredients have been weighed in, checked weight, and recorded the actual Gross weight of Tank 1.

Transferred Tank 1 to a second heating element then, started heating to 70° C.-75° C. with stirring.

Continued to stir for further 10 minutes, while maintaining 70° C. to 75° C. temperature and continued until required for the next step.

When ready for the next step, obtained Gross weight of the main-Tank 1, recorded the gross weight i.e., prior adding the WP.

### Emulsion/Final Phase—Tank 1

From the Water Phase Stock/Tank 2 took out the required quantity and added to the oil phase in Tank 1 while stirring and heating to 75° C. Recorded the net amount WP added to Tank 1.

Continued stirring Tank 1 for further 10 minutes maintaining 70° C. to 75° C. temperature.

After 10 minutes, cooled down the emulsion to 50° C. to 55° C.

At 50° C. to 55° C., added partial amount of ethanol to Tank 1 then resumed stirring, recorded the partial amount ethanol added.

Prepared for the weighing of the active material-weighing is done under laminar flow cabinet: (1) Added the last portion of ethanol to Tank 1 while carefully rinsing around the sides of the main container and to fully solubilize the drug at low heating approx. 50° C. (2) Obtained gross weight of Tank 1 after complete addition of the active and of ethanol. Allowed the active to blend in the mixture by gentle stirring. (3) As soon as fully dissolved and prior to pH check, obtained the actual gross weight of Tank 1 and reconciled against the gross weight by input. Added ethanol to compensate for any loss due to evaporation. Recorded the final gross weight of Tank 1 after final qs with ethanol. (4) Resumed gentle stirring in Tank 1 with temperature approximately 50° C. or up to 60° C., if the bulk starts to become cloudy and show less fluidity.

When the drug has fully solubilized in Tank 1, checked, and recorded the initial pH then sparingly added triethanolamine (TEA) to adjust to target pH 5.5 (5.0 to 6.0 pH range). Recorded the amount of TEA added and the final pH of the bulk.

Resumed stirring in Tank 1 at 50° C. then obtained the final weight and calculated for % yield. Allowed to fully cool down with stirring, stop at 30° C. to 35° C.

Sampled the bulk (foam base) from top and bottom.

### Sealed Tank 1.

Test method for measuring collapse of foam at 32° C.

Maintained a temp 32° C. to mimic the biological temperature of an adult scalp.

A water bath is used as the main equipment, which has been calibrated to provide constant temperature of 32° C.

A glass crucible is acclimatized while afloat the 32° C. water and which is where the foam is discharged (approx. 0.7 g to 1.0 g weight) as a scalp substitute. Total time for the foam to liquefy maintaining 32° C. was recorded. It was noted, film like residue sometimes persist lengthening the collapse time.

Prior to test, the aluminum can sample was pre-acclimatized to 25° C., a calibrated digital thermometer was immersed to the bath to monitor the temperature displayed on water bath dial. The timer was triggered from discharge of the foam to the glass crucible.

### IVPT Procedure:

IVPT test conditions are summarized below (Test Condition A):

IVPT Test Conditions

| | |
|---|---|
| Skin type: | Human abdominal skin from elective surgery |
| Thickness (μm): | 500 ± 100 |
| No. skin donors: | 3 |
| Receptor solution: | Phosphate buffered solution |
| | pH 7.4 + 0.01% Brij |
| No. formulations: | 10 test formulations |
| | 1 control formulation (Opzelura ®) |
| | 5 placebo formulations |
| No. replicates: | 4 (per active formulation); |
| | 1 (per placebo) |
| No. skin blanks: | 1 (per skin donor) |
| Dose amount: | 10 mg/cm² |
| Flow rate: | 6 μL/min |
| RS collection times | Every 3 hours for 24 hours |

Skin tissue procedures

| | |
|---|---|
| Extraction fluid: | 90:10 v/v acetonitrile: water |
| Residual drug: | Per DOC-0096; discard |
| Stratum corneum: | Per DOC-0096; discard. |
| Separate dermis/epidermis? | Yes |

### IVPT Test Conditions B:

IVPT Test Conditions

| | |
|---|---|
| Skin type: | Human abdominal skin from elective surgery |
| Thickness (μm): | 500 ± 100 |
| No. skin donors: | 3 |
| Receptor solution: | Phosphate buffered solution |
| | pH 7.4 + 0.01% Brij |
| No. formulations: | 10 test formulations |
| | 1 control formulation (Opzelura ®) |
| | 5 placebo formulations |
| No. replicates: | 4 (per active formulation); |
| | 1 (per placebo) |
| No. skin blanks: | 1 (per skin donor) |
| Dose amount: | 10 mg/cm² |
| Flow rate: | 6 μL/min |
| RS collection times | Every 3 hours for 24 hours |

Skin tissue procedures

| | |
|---|---|
| Extraction fluid: | 90:10 v/v acetonitrile: water |
| Residual drug: | Per DOC-0096; discard |
| Stratum corneum: | Per DOC-0096; discard. |
| Separate dermis/epidermis? | Yes |
| Extraction procedure | According to DOC-0096. |

The concentration of ruxolitinib and deuruxolitinib detected in the receptor solution and skin layers was quantified using a calibration range optimized for the analysis of the samples generated during the ex vivo skin permeation and penetration experiments. The following parameters were calculated, where possible, for each replicate according to the table below:

| Parameter | Definition | Units |
|---|---|---|
| AUC | Cumulative amount of API permeated into the | ng/cm²; |
| | receptor solution over the duration of the | |
| | experiment. | |
| PF | The maximal rate of absorption, or peak flux. | ng/cm²/hr |
| Epidermis | Total API recovered from the epidermis | ng; |
| Dermis | Total API recovered from the dermis | |

Significant differences between IVPT results were determined by Tukey's HSD test (α=0.05).

### LC-MS/MS:

### Solutions:

Solution and stocks prepared were as follow:

Receptor Solution: Phosphate buffered saline (PBS)+0.01% Brij98

Extraction Fluid: 90/10 Acetonitrile/water (v/v)

Stock Solution Diluent: 90/10 DMSO/water (v/v)

Spike Diluent: 70/30 Ethylene glycol/water (v/v)

Diluent X: 50/50 Methanol/water (v/v)

Mobile phase A (MPA): 0.1% Formic acid (v/v) in water

Mobile phase B (MPB): Methanol

Solution and stocks prepared were as follow:

An internal standard of the following compound (compound A) at a concentration of 25 ng/ml in methanol was used as an internal standard:

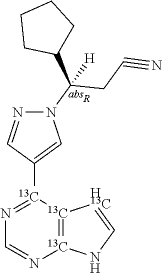

### Receptor Solution Method

Standards/QCs: Diluted the STD and QC Spiking Solutions by 20-fold into Receptor Solution and mix well.

All samples/blanks/STD/QC: Added a ⅙th volume of ISWS (or methanol for blanks) to all samples. (e.g., if the Receptor Solution final volume is 0.600 mL, add 0.100 mL ISWS). Mixed well and store at 5° C.

### Tissue (Extraction Fluid) Method

Sample extract dilutions (if needed): Mixed samples well and centrifuged. Serially diluted samples using the Extraction Fluid. Typical dilution factors employed are 50-fold and 2500-fold. Mixed well after each dilution step.

Standards/QCs: Diluted the STD and QC Spiking Solutions by 20-fold into Extraction Fluid and mixed well.

All samples/blanks/STD/QC: Combined 0.0500 mL ISWS (or methanol for blanks), 0.0500 mL each prepared sample, and 0.300 mL of Diluent X. Mixed well and store at 5° C.

Note: This procedure assumes that these samples have already been homogenized, centrifuged, and transferred to a 96 well plates.

### Analytical Method:

Method parameters are identical for both APIs, but separation was required as there is significant crosstalk between Ruxolitinib and Ruxolitinib-d8 MRM channels. Parameters marked with an * may be adjusted to achieve optimal peak intensities, retention times, etc.

TABLE 16
UHPLC-MS/MS system method parameters

| UHPLC-MS/MS System | Waters Acquity I-Class UPLC | | | |
|---|---|---|---|---|
| | Waters Xevo TQ-XS Triple | | | |
| | Quadrupole Mass Spectrometer | | | |
| | Waters TargetLynx Software | | | |
| Mobile Phase A | 0.1% Formic Acid in Water | | | |
| Mobile Phase B | 100% Methanol | | | |
| Purge solution | 100% Methanol | | | |
| Wash solution | 80:20 Water:Methanol | | | |
| Injection Volume | 2 | | | |
| (μL) * | | | | |
| Guard Column | Acquity UPLC BEH C18 1.7 um Guard Column | | | |
| Column | Acquity UPLC BEH C18 1.7 um 2.1 × 50 mm | | | |
| Flow Rate (initial) | 0.400 | | | |
| (mL/min) | | | | |
| % B (initial) * | 10 | | | |
| Run Time (min.) | 1.85 | | | |
| Column | 40 | | | |
| Temperature (° C.) | | | | |
| Autosampler | 10 | | | |
| Temperature (° C.) | | | | |
| Divert Valve sequence | To start, flow diverted to waste. At 30 | | | |
| | seconds, flow diverted to LC and at 108 | | | |
| | seconds, diverted back to waste. | | | |
| | Time | Flow rate | | |
| | (min) | (mL/min.) | % B | Curve |
| LC Time Program | Initial | 0.400 | 10.0 | Initial |
| | 0.05 | 0.400 | 10.0 | 6 |
| | 0.20 | 0.400 | 45.0 | 6 |
| | 1.00 | 0.400 | 55.0 | 6 |
| | 1.25 | 0.400 | 90.0 | 6 |
| | 1.50 | 0.400 | 90.0 | 6 |
| | 1.51 | 0.400 | 10.0 | 6 |
| | 1.85 | 0.400 | 10.0 | 6 |

### MS Parameters—

| Polarity | Positive |
|---|---|
| Capillary (kV) | 1.00 |
| Cone (V) | 30 |
| Source Temperature (° C.) | 150 |
| Desolvation Temperature (° C.) | 500 |
| Cone Gas Flow (L/Hr) | 150 |
| Desolvation Gas Flow (L/Hr) | 1000 |
| Collision Gas Flow (mL/Min) | 0.15 |
| Nebuliser Gas Flow (Bar) | 7.00 |
| Dwell time (ms) | 52 |

MRM table

| Compound | Q1 m/z * | Q3 m/z * | CE * |
|---|---|---|---|
| Ruxolitinib | 307.1700 | 159.0300 | 50 |
| Ruxolitinib** | 307.1700 | 186.0800 | 38 |
| Ruxolitinib D8 | 315.2000 | 158.9500 | 50 |
| Ruxolitinib D8** | 315.2000 | 186.1000 | 38 |
| Compound A** | 311.2000 | 190.1000 | 38 |

**Transition used in quantitation.

Note: Curve Type: Quadratic, weighting: 1/×2, acceptance criteria: accuracy #20%, precision ≤20

### Test Procedure for Evaluation of Foam Formulations on Human Alopecia Areata (AA) Lesional Skin Organ Cultures

Patient biopsies for this study were obtained after informed, written patient consent under the ML Biobank approval (2019-297-f-S) and processed for further analysis under ethics committee study approval (healthy scalp skin donors and alopecia areata patients: 2020-954-f-S). The study was conducted according to the Declaration of Helsinki principles.

Skin punch biopsies measuring 4 mm were obtained from acute (consisting of two lesional and two non-lesional samples) and chronic AA patients (comprising two lesional samples). The samples were cultured at 37° C. within a 5% CO2 atmosphere, using a minimal medium of William's E media and RPMI 1640 in equal proportions (provided by Gibco, Life Technologies). This medium was further supplemented with 2 mM of L-glutamine (supplied by Gibco), 10 ng/ml hydrocortisone (offered by Sigma Aldrich), 10 μg/ml insulin (distributed by Sigma Aldrich) and a 1% mixture of penicillin/streptomycin (procured from Gibco). The medium thus prepared is referred to as Williams Complete/RPMI Media (WCM+RPMI). Each skin punch biopsy was topically treated on a daily basis with either Placebo foam (Vehicle) or API foam. The foam was consistently weighed at the time of each topical treatment. Photographs were captured at the beginning, midpoint, and conclusion of the skin organ culture. The skin punch biopsies were weighed before the start of the organ culture. The skin punch biopsies were weighed before the start of the organ culture. Note: Acute samples are presented in the report.

### Methods and Red-Out Parameters

Masson Fontana for hair cycle staging Cryosections (7 μm) were fixed with an Ethanol-Acetic acid mixture (2:1) for 10 minutes at −20° C. Post-fixation, slides were sequentially washed with TBS and distilled water. The samples were then incubated in a 5% Ammonia-based Silver Nitrate solution for 40 minutes at 56° C. in absence of light. The slides were washed again with distilled water and incubated in 5% Sodium thiosulfate for 1 minute at room temperature. After a further wash in tap water, sections were counterstained with hematoxylin, washed, dehydrated, and embedded in Eukitt.

### Ki-67/TUNEL

Hair matrix keratinocyte proliferation and apoptosis for hair cycle staging Cryosections of 7 μm underwent fixation with 4% PFA for 10 minutes and a wash in PBS, followed by incubation with the equilibration buffer for 5 minutes, TdT-Enzyme for 60 minutes at 37° C., and stop buffer for 10 minutes, each at room temperature. These steps concluded with a PBS rinse. Next, slides were blocked using a 5% NGS/PBS solution for 20 minutes, then treated overnight at 4° C. with mouse anti-human Ki-67 antibody in 2% NGS/PBS solution. The following day, slides were washed with PBS, and incubated with fluorescent-labelled Anti-Digoxigenin antibody for 30 minutes and with the secondary antibody “Goat anti-mouse rhodamine red” in 2% NGS/PBS solution for 45 minutes, both at room temperature. Afterwards, slides were rinsed with PBS, counterstained with DAPI, and given a final PBS wash before being mounted using fluoromount.

### Hair Cycle Staging=Hair Growth Assessment −1 (Ex Vivo Skin Organ Culture):

Hair cycle staging was performed at the end of the culture. The hair cycle stage of each hair follicle was determined according to several established microscopic parameters (Oh et al., J Invest Dermatol 2016). It was determined using Ki-67/TUNEL immunohistology and Masson Fontana histochemistry.

TABLE 17
Hair growth assessment.

| Hair cycle stage | Anagen I | Anagen II | Anagen III | Anagen IV | Anagen V + VI |
|---|---|---|---|---|---|
| Hair matrix | absent | forming out | newly | prominent, | thick matrix, |
| shape and | | of SHG | formed, 4-5 | envelops the | envelops DP |
| thickness | | | cell layers | DP | |
| | | | thick, | | |
| | | | encloses at | | |
| | | | least 60% of | | |
| | | | DP | | |
| Dermal | condensed, | small, ball- | enlarged, | oval in | large, onion- |
| papilla (DP) | ball-like | shaped with | oval-shaped | shape, | shapec |
| shape | | wide stalk | with wide | increased in | |
| | | | stalk; | size | |
| | | | increased | | |
| | | | extracellular | | |
| | | | volume | | |
| Epithelial | CTS trail, | CTS trail, | CTS trail | CTS trail | absent |
| strand | SHG: | SHG: wide | still visible | still visible | |
| | triangular or | strand, | | below DP | |
| | crescent- | crescent- | | | |
| | shaped, wraps | shaped, | | | |
| | around DP | partially | | | |
| | | covers DP | | | |
| Pigmentation | melanin | unpigmented, | not | visible jn the | Hair shaft |
| | clumps in DP | few melanin | pigmented | hair shaft | fully |
| | and CTS trail | inclusions in | | | pigmented |
| | | DP | | | |
| Proliferation | / | localized | proliferating | proliferating | prominent |
| | | proliferation | cells in the | cells in the | proliferating |
| | | in SHG | ORS | hair matrix | matrix cells |
| | | | | and ORS | |
| Hair length/ | entirely above | entire HF | hair shaft | bulb located | bulb is |
| hair shaft | the dermal/ | resides in | starts to | in the | located deep |
| | adipose | dermis | form, IRS | adipose | in the |
| | junction | | visible | layer; hair | adipose |
| | | | | shaft fully | layer; hair |
| | | | | mature, tip | shaft is |
| | | | | reached the | mature and |
| | | | | sebaceous | emerges |
| | | | | gland | from the |
| | | | | | epidermis |

Abbreviations: HF-hair follicle; DP-dermal papilla; ORS-outer root sheath; CTS-connective tissue sheath; and SHG-secondary hair germ.

### Hair Cycle Staging=Hair Growth Assessment −2 (Ex Vivo Skin Organ Culture)

TABLE 18
Hair growth assessment - 2

| Hair cycle stage | Telogen | Early catagen | Mid catagen | Late catagen |
|---|---|---|---|---|
| Hair matrix | absent | matrix volume loss, | further loss in | matrix |
| shape and | | reduced to two- | volume, | disappears |
| thickness | | three cell layers | one-two cell | |
| | | | layers, only | |
| | | | partially | |
| | | | surrounding the | |
| | | | DP | |
| Dermal papilla | very compact, | condensed, | even more | condensed, |
| (DP) shape | well-rounded | almond-shape | condensed, | ball-shape |
| | | | almond-shape | |
| Epithelial | CTS trail, | absent | pleat-like, ruffled | short with |
| strand | maximally | | appearance, | smooth |
| | shortened, | | apoptotic cells | outlines, |
| | unpigmented | | visible, thickened | apoptotic |
| | SHG: short strand | | vitreous | cells visible, |
| | | | membrane | CTS trail |
| | | | | (“dermal |
| | | | | streamer”) |
| Pigmentation | no pigmentation | loss of melanin in | no pigmentation | melanin |
| | | precortex and | | clumps along |
| | | proximal shaft, | | CTS |
| | | melanin | | |
| | | incontinence in | | |
| | | DP | | |
| Proliferation | no, but dispersed | / | / | / |
| | proliferating cells | | | |
| | in SHG | | | |
| Hair length/ | entirely above the | not changed, | Brush-like club | shortened |
| hair shaft | dermal/adipose | lower HF rests | hair residing | epithelial |
| | junction, serrated | below | above the | strand, about |
| | club hair | dermal/adipose | dermal/adipose | half the |
| | | junction, club hair | junction | length as in |
| | | formation starts | | mid catagen |

Abbreviations: HF-hair follicle; DP-dermal papilla; ORS-outer root sheath; CTS-connective tissue sheath; and SHG-secondary hair germ.

### Standardized Measurements and Testing for Topical Foam Treatment

To standardize the procedure of the topical treatment of the foam, we used a 2 μl sample of liquefied foam, which weighed precisely 1.52 mg. The foam was discharged into a 3.5 cm dish for a precise weight measurement. Employing a 10 μl tip, we removed the foam from the dish twice for each application during the evaluation. A careful record of the weight change in the foam-filled dish was kept.

This procedure was performed ten times each for the Placebo foam, API-1 foam, and API-2 foam.

TABLE 19
Testing of measure amount for the application procedure

| | Placebo (mg) | API 1 (mg) | API 2 (mg) |
|---|---|---|---|
| Test application 1 | 1.65 | 1.36 | 2.26 |
| Test application 2 | 1.48 | 1.8 | 1.06 |
| Test application 3 | 1.11 | 1.55 | 1.18 |
| Test application 4 | 1.21 | 1.17 | 0.99 |
| Test application 5 | 1.01 | 1.27 | 1.07 |
| Test application 6 | 1.21 | 1.19 | 1.11 |
| Test application 7 | 1.29 | 1.07 | 1.09 |
| Test application 8 | 1.17 | 0.99 | 1.11 |
| Test application 9 | 1.15 | 1.26 | 1.07 |
| Test application 10 | 0.99 | 0.99 | 0.93 |
| Average | 1.227 | 1.265 | 1.187 |

Placebo and API-1 (ruxolitinib) was examined in acute AA lesional skin organ culture

TABLE 20
Skin punch donor information

| Donor | Age | Sex | Localisation | Groups | Experiment |
|---|---|---|---|---|---|
| 1-1 | 24 | Female | Scalp | #1-4 | chemokine/cytokine |
| | | | | | array and tissue |
| | | | | | embedding in |
| | | | | | Cryomatrix for |
| | | | | | analysis |

TABLE 21
Treatment groups

| Groups | Treatment in WCM + RPMI (50:50) |
|---|---|
| #1 | Placebo foam (lesional) |
| #2 | API-1 foam (lesional) |
| #3 | Placebo foam (non-lesional) |
| #4 | API-1 foam (non-lesional) |

### Synthesis of (R)-3-(4-(7H-pyrrolo[2,3-d]pyrimidin-4-yl-4,4a,5,7a-13C4)-1H-pyrazol-1-yl)-3-cyclopentylpropanenitrile (internal LC/MS standard)

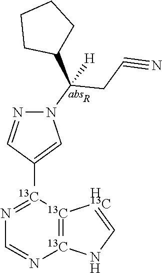

This standard was made by the scheme below, wherein the stars indicate that the carbon atoms are ¹³C labeled (M+H⁺ of 311.3; ee 99.5%). The ¹³C labeled diethyl maloanate was available from Sigma-Aldrich (catalog no. 488771; reg. no. 53051-81-3). (3R)-3-Cyclopentyl-3-[4-(4,4,5,5-tetramethyl-1,3,2-dioxaborolan-2-yl)-1H-pyrazol-1-yl]propanenitrile was available from Sigma-Aldrich (catalog no. AMBH99C03807, reg. no. 1146629-84-6).

In the final step, a 50 ml round bottom fitted with stir bar, condenser and 3-way valve was connected to nitrogen and charged with 4-chloropyrrolo[2,3-d]pyrimidine (0.272 g, 0.00177 mmol), (3R)-3-cyclopentyl-3-[4-(4,4,5,5-tetramethyl-1,3,2-dioxaborolan-2-yl)-1H-pyrazol-1-yl]propanenitrile (0.757 g, 0.00240 mmol) and 1,4-Dioxane (0.0654 mmol) to give a homogeneous solution. Water (0.283 mmol) and sodium hydrogenecarbonate (0.766 g, 0.00912 mmol) were added, and then the solution was degassed with nitrogen.

Tetrakis (triphenylphosphine) palladium (0) (171 mg, 0.000148 mmol) was added under nitrogen, and the solution warmed to Degas 4× backfilling with nitrogen each time to 100° C. and allowed to stir overnight. After 17 hours, the reaction was complete. The mixture was diluted with 35 ml EtOAc and 20 ml 20% brine and then stirred until all solids were in solution. The aqueous layer was extracted with 2×25 ml EtOAc. The organic layers were then combined organics and dried over magnesium sulfated, then filtered and concentrated. The product was then purified by flash chromatography (50% to 100% EtOAc/hexane w/CH₂Cl₂ loading). The desired fractions were combined and concentrated in vacuo to yield a yellow foam. Combine and concentrate desired fractions. Place on high vacuum pump to yield a yellow foam (484 mg; M+H⁺ of 311.3; HPLC purity 97.3%; ee 99.5%).

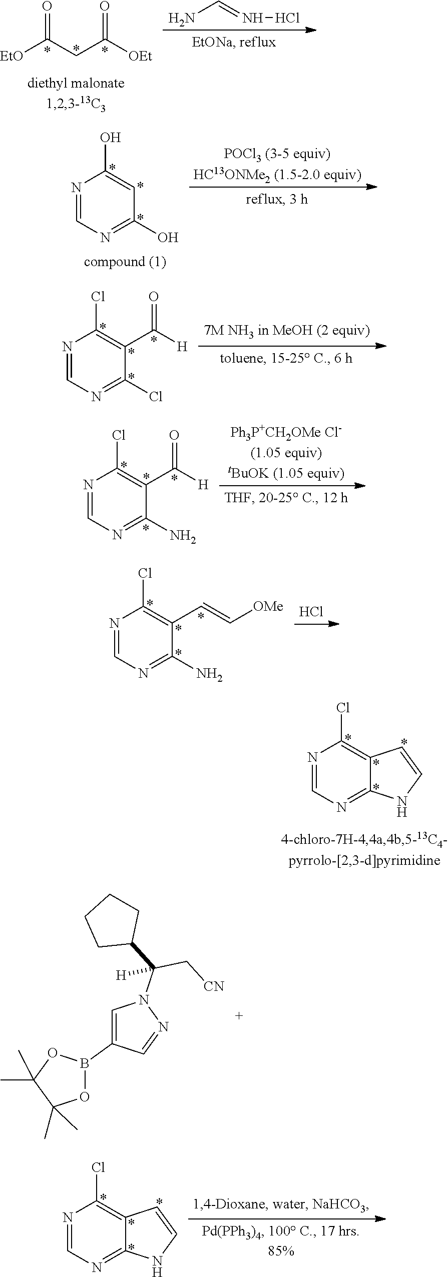

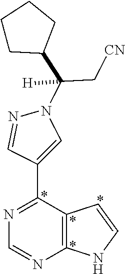

### Example 11A: Solubility of Ruxolitinib Phosphate in Hydroethanolic Formulations

Ruxolitinib phosphate was found to have good solubility in a mixture of ethanol and water with loading levels reaching ˜4% on a free base basis (w/w) in mixtures of 60:40 or 40:60 ethanol:water (see Table 22).

TABLE 22
Solubility in neat ethanol-water solutions

| | | Content | | Ruxolitinib | Equivalent | pH with | Analytical |
|---|---|---|---|---|---|---|---|
| | Ethanol:Water, | Ethanol:Water | | phosphate | Ruxolitinib | drug in | result- |
| Sample, | (without drug), | (with drug), | | added, | (CF 0.753), | (no pH | Ruxolitinib |
| F/L | % ratio | % w/w | | % w/w | % w/w | adjustment) | % w/w (% RSD) |
| 213-2-01 | 100:0 | 96.1 | 0.0 | 3.9 | 2.9 | 3.79 | 0.613 (1.1) |
| 213-2-02 | 80:20 | 79.4 | 17.4 | 3.2 | 2.4 | 3.91 | 1.821 (1.7) |
| 213-2-03 | 60:40 | 58.4 | 36.3 | 5.3 | 4.0 | 3.39 | 2.637 (0.3) |
| 213-2-04 | 40:60 | 38.8 | 55.4 | 5.8 | 4.4 | 3.13 | 2.269 (1.3) |
| 213-2-05 | 20:80 | 20.1 | 76.8 | 3.1 | 2.3 | 2.74 | 0.995 (1.2) |

Based on the good solubility of ruxolitinib phosphate in hydroethanolic mixtures, the solubility of ruxolitinib phosphate in a hydroethanolic base formulation was studied. Utilizing Method A (Materials and Methods), the formulations in Table 23 were prepared. As can be seen in Table 23, approximately 3% (w/w) of ruxolitinib phosphate on a free base basis dissolved in the base having 60% ethanol with and without the addition of additional propylene glycol (PG). PG was added as a penetration enhancer to facilitate better skin permeation of the API from application of the foam.

TABLE 23
Solubility of Ruxolitinib in foamable carrier components.

| | Formulation | | | |
|---|---|---|---|---|
| | 213-2-07 | | 213-2-08 | |
| | (nil PG) | | (contains PG) | |
| | Final pH | | | |
| | 6.41 | | 6.16 | |
| | Required, | Actual | Required, | Actual |
| Ingredients | % w/w | % w/w | % w/w | % w/w |
| Water | 35.60 | 35.40 | 35.60 | 35.25 |
| Propylene glycol | — | | 5.00 | 4.93 |
| Polysorbate 60 | 0.40 | 0.40 | 0.40 | 0.38 |
| Laureth-4 | 1.00 | 1.00 | 1.00 | 0.99 |
| Ethanol | 60.00 | 59.40 | 51.80 | 51.39 |
| Ruxolitinib phosphate | 3.00 | 3.77 | 3.00 | 3.86 |
| Fatty alcohols- | — | — | 3.20 | 0.00 |
| Cetyl alcohol, 2.20 and | | | | |
| Stearyl alcohol, 1.00 | | | | |
| TOTAL | 100.00 | 100.00 | 100.00 | 96.80 |
| Ratio Ethanol:water | 63:37 | 63:37 | 59:41 | 59:41 |
| (E:W) | | | | |
| Equiv. Ruxolitinib free | 2.26 | 2.9 | 2.26 | 2.9 |
| base CF = 0.758) | | | | |
| Analytical result, % w/w | — | 2.92 (0.5) | — | 3.09 (1.3) |
| solubility Ruxolitinib | | | | |
| (% RSD) | | | | |

### Foamability and Miscibility

Next, the foamability and miscibility of the formulation (213-2-08) with PG (with and without ruxolitinib phosphate) was tested using P75 propellant (the foamable compositions were prepared using the manufacturing method in Method A) (Table 24). The base without ruxolitinib phosphate was shown to be miscible with P75 and resulted in a well-formed foam which did not collapse after 2 minutes. While the base with 3% (w/w) on a free base basis of ruxolitinib phosphate did show inhomogeneity and phase separation, the base did form a well-formed foam, just needing further optimization for miscibility of the API.

TABLE 24
Formulations for miscibility and foamability evaluation

| | PRODUCT | | | |
|---|---|---|---|---|
| | PLACEBO Sample | | 3.0% RUXOLITINIB FOAM | |
| | Sample, Formulation/Lot | | | |
| | 268-3-01 | | 268-3-02 | |
| | Required | Actual | Required | Actual |
| INGREDIENTS | % w/w | % w/w | % w/w | % w/w |
| Purified Water | 35.44 | 35.42 | 35.44 | 36.41 |
| Laureth-4 | 1.00 | 1.00 | 1.00 | 0.98 |
| Polysorbate 60 | 0.40 | 0.41 | 0.40 | 0.4 |
| Propylene glycol | 5.00 | 5.00 | 5.00 | 4.89 |
| Cetyl alcohol | 2.20 | 2.20 | 2.20 | 2.15 |
| Stearyl alcohol | 1.00 | 1.00 | 1.00 | 0.98 |
| Ethanol, g | 51.00 | 51.01 | 51.00 | 49.88 |
| Ruxolitinib phosphate | — | — | 3.960 | 3.87 |
| NaOH 20% ag. | — | — | qs | 0.44 |
| solution | | | | |
| TOTAL Net | 96.04 | 96.04 | 100.00 | 100.00 |
| Total (Ethanol + Water) | 86.44 | 86.43 | 86.44 | 86.29 |
| (E:W) % Ratio | 59:41 | 59:41 | 59:41 | 58:42 |
| Miscibility with P75 | — | Miscible/ | — | Oily droplets |
| propellant in clear | | homogeneous, | | forming a blob, |
| aerosolized bottle | | no separation | | inhomogeneous |
| | | 6.3% P75 | | 6.7% P75 |
| Foam appearance, | — | Well-formed | — | Well-formed |
| ambient | | foam | | foam |
| Foam collapse, | — | Little to no | — | Little to no |
| minutes at ambient | | change in | | change in |
| condition | | appearance | | appearance |
| | | after 2 mins. | | after 2 mins. |

### Impact of Fatty Alcohols on Foamability

Based on these results, the impact of the fatty alcohols in the formulation was investigated as to their impact on foam appearance and foam collapse. Generally, foams used for application to the scalp will ideally have a foam collapse rate that is at least that of ROGAINE®, which has a collapse rate of about 2.3 minutes. For example, the foam collapse rate will be higher (e.g., ≥3 minutes) such as ≥5 minutes. This is because ROGAINE is known to collapse too quickly, resulting in the formulation draining off of the scalp to the face or shoulders.

Accordingly, the impact of stearyl alcohol and cetyl alcohol on the appearance and collapse rate of the foam formed using P75 was explored using the placebo formulations (Table 25) (prepared using manufacturing Method A). These results showed that stearyl alcohol is critical to formation of a well-formed foam with good collapse rate (see Tables 26 and 27). When stearyl alcohol was removed, a runny liquid or a unstable flattening foam was obtained, while the foams with 0.5% or 1% stearyl alcohol formed well-formed foams (Tables 26 and 27).

TABLE 25
Screening the quantity of fatty alcohols in placebo formulations.

| | PRODUCT | | | | |
|---|---|---|---|---|---|
| | 3% Ruxolitinib Foam - Reference | | PLACEBOS-screening fatty alcohols | | |
| | Sample ID | | | | |
| | 268-3-02 | | 268-4-01 | 268-4-02 | 268-4-03 |
| | Required, | Actual, | Required | Required | Required |
| Ingredients | % w/w | % w/w | % w/w | % w/w | % w/w |
| Purified Water | 35.44 | 36.41 | 36.10 | 36.30 | 35.85 |
| Laureth-4 | 1.00 | 0.98 | 1.00 | 1.00 | 1.00 |
| Polysorbate 60 | 0.40 | 0.4 | 0.40 | 0.40 | 0.40 |
| Propylene glycol | 5.00 | 4.89 | 5.00 | 5.00 | 5.00 |
| Cetyl alcohol | 2.20 | 2.15 | 1.10 | 1.10 | 2.20 |
| Stearyl alcohol | 1.00 | 0.98 | 0.50 | nil | nil |
| Ethanol, g | 51.00 | 49.88 | 51.94 | 52.32 | 51.59 |
| Ruxolitinib | 3.960 | 3.87 | nil | nil | nil |
| phosphate | | | | | |
| NaOH 20% aq soln | qs | 0.44 | qs | qs | qs |
| Total Net | 100.00 | 100.00 | 96.04 | 96.04 | 96.04 |
| Total (Ethanol + | 86.44 | 86.29 | 88.04 | 88.62 | 87.44 |
| Water) | | | | | |
| (E:W) % ratio | 59:41 | 58:42 | 59:41 | 59:41 | 59:41 |

TABLE 26
Foam appearance at ambient temperature

| | Cetyl | Stearyl | |
|---|---|---|---|
| | alcohol | alcohol | |
| Sample | content, | content | Test result/observations, |
| ID | % w/w | % w/w | ambient |
| 268-4-01 | 1.1 | 0.56 | Foam well formed with larger |
| | | | particle/bubble, stable no foam |
| | | | collapse within 2 minutes. |
| 268-4-02 | 1.1 | nil | Liquefied upon spray, large bubbles, |
| | | | and runny liquid. |
| 268-4-03 | 2.2 | nil | Foam formed but quite unstable |
| | | | flattening and collapsing early from |
| | | | half a minute of being sprayed out. |
| 268-3-02 | 2.2 | 1.0 | Comparator |

TABLE 27
Foam collapse evaluation

| | | Cetyl | Stearyl | Foam |
|---|---|---|---|---|
| Sample, | | alcohol, | alcohol, | collapse, |
| F/L | Product | % w/w | % w/w | minutes |
| 268-3-01 | Placebo foam | 2.2% | 1.0% | 5 |
| 268-3-02 | 3% Ruxolitinib | 2.2% | 1.0% | 5 |
| | foam | | | |
| 268-4-01 | Placebo foam | 1.1% | 0.56% | 2 |
| 268-4-02 | Placebo foam | 1.1 | nil | liquefied |
| 268-4-03 | Placebo foam | 2.2% | nil | 1 |
| Reference | ROGAINE ® Foam | unknown | unknown | 2.3 |
| foam | | | | |

### Example 11B: One Month Stability Studies

Similar foamable compositions having 3% (w/w) and 1.5% (w/w) ruxolitinib phosphate on a free base basis were studied as to their chemical and physical stability over 1 month at 40° C./75% relative humidity (HD) (Table 28 and Table 29) as prepared by manufacturing Method A. Ruxolitinib phosphate was found to be chemically stable in the foamable compositions and the compositions produced well-formed foams.

In addition, the formulations in Table 29 were studied as to their physical stability. The use of PEG400 in the 268-5-01 formulation having 1.5% (w/w) ruxolitinib phosphate on a free base basis was found to undergo phase separation, indicating its lack of suitability in the formulation.

TABLE 28
Short term stability study

| | PRODUCT | | | | | | | |
|---|---|---|---|---|---|---|---|---|
| | ACTIVE FORMULATIONS | | | | | | PLACEBO | |
| | Sample ID/Batch F/L | | | | | | | |
| | 268-5-01 | | 268-5-02 | | 268-5-01 | | 268-5-03 | |
| | Required | Actual | Required | Actual | Required | Actual | Required | Actual |
| INGREDIENTS | % w/w | % w/w | % w/w | % w/w | % w/w | % w/w | % w/w | % w/w |
| Ro Water | 35.04 | 36.18 | 35.04 | 36.04 | 35.04 | 36.47 | 35.44 | 35.15 |
| Laureth-4 | 1.00 | 0.99 | 1.00 | 1.00 | 1.00 | 0.99 | 1.00 | 1.00 |
| Polysorbate 60 | 0.40 | 0.39 | 0.40 | 0.40 | 0.40 | 0.40 | 0.40 | 0.40 |
| Propylene glycol | 5.00 | 4.92 | — | — | 2.50 | 4.87 | 2.50 | 2.51 |
| PEG 400 | — | — | 5.00 | 4.93 | — | — | 2.50 | 2.53 |
| Cetyl alcohol | 2.20 | 2.16 | 2.20 | 2.16 | 2.20 | 2.14 | 2.20 | 2.22 |
| Stearyl alcohol | 1.00 | 0.97 | 1.00 | 0.99 | 1.00 | 0.96 | 1.00 | 1.01 |
| Ethanol, g | 51.00 | 50.04 | 51.00 | 50.10 | 51.00 | 49.95 | 51.00 | 51.14 |
| Ruxolittinib PO4 | 3.958 | 3.90 | 3.958 | 3.98 | 3.958 | 3.85 | — | — |
| NAOH 20% aq. | 0.40 | 0.45 | 0.40 | 0.40 | 0.40 | 0.38 | qs | 0.05 |
| solution | | | | | | | | |
| Glacial acetic acid | — | — | — | — | — | — | qs | 0.03 |
| TOTAL NET | 100.00 | 100.00 | 100.00 | 100.00 | 100.00 | 100.00 | 96.04 | 96.04 |
| Ethanol:Water | 59:41 | 58:42 | 59:41 | 58:42 | 59:41 | 58:42 | 59.41 | 59:41 |
| ratio | | | | | | | | |
| Equiv. | 3.00 | 2.96 | 3.00 | 3.02 | 3.00 | 2.92 | -0- | -0- |
| Ruxolitinib, | | | | | | | | |
| % w/w | | | | | | | | |

Chemical and physical stability evaluation

| | | | | | | | | |
|---|---|---|---|---|---|---|---|---|
| Ruxolitinib % LC | — | 101.2 (0.2) | — | 101.9 (0.4) | — | 98.2 (0.0) | — | NA |
| (w/w), Ave (% RSD) | | | | | | | | |
| Total Impurities, | — | 0.11 | — | 0.15 | — | 0.10 | — | ND |
| (% area)_{—} | | | | | | | | |
| Foam appearance, | — | Well- | — | Well- | — | Well- | — | — |
| ambient | | formed | | formed | | formed | | |
| Foam collapse, | — | No test | — | 7 mins | — | 6 mins | — | — |
| 32° C. surface | | done | | | | | | |
| (25° C. samples) | | | | | | | | |

NA = not applicable;
ND = not detected

TABLE 29
Short term stability study

| | PRODUCT | | | | | |
|---|---|---|---|---|---|---|
| | ACTIVE FORMULATIONS | | | | PLACEBO | |
| | Sample ID | | | | | |
| | 268-6-01 | | 268-6-02 | | 268-5-03 | |
| | pH 6.02 | | pH 5.38 | | pH 5.48 | |
| | Required | Actual | Required | Actual | Required | Actual |
| INGREDIENTS | % w/w | % w/w | % w/w | % w/w | % w/w | % w/w |
| Purified Water | 35.85 | 36.16 | 35.85 | 26.16 | 35.44 | 35.15 |
| Laureth-4 | 1.00 | 1.01 | 1.00 | 1.01 | 1.00 | 1.00 |
| Polysorbate 60 | 0.40 | 0.40 | 0.40 | 0.41 | 0.40 | 0.40 |
| Propylene glycol | 5.00 | 5.00 | — | — | 2.50 | 2.51 |
| PEG 400 | — | — | 5.00 | 5.00 | 2.50 | 2.53 |
| Cetyl alcohol | 2.20 | 2.19 | 2.20 | 2.20 | 2.20 | 2.22 |
| Stearyl alcohol | 1.00 | 1.00 | 1.00 | 1.00 | 1.00 | 1.01 |
| Ethanol, g | 52.17 | 52.06 | 52.17 | 52.05 | 51.00 | 51.14 |
| Ruxolitinib Phosphate | 1.979 | 1.9802 | 1.979 | 1.987 | — | — |
| NaOH 20% aq solution | 0.40 | 0.20 | 0.40 | 0.19 | qs | 0.05 |
| Glacial acetic acid | — | — | — | — | qs | 0.03 |
| TOTAL net | 100.00 | 100.00 | 100.00 | 100.00 | 96.04 | 96.04 |
| (Ethanol:Water) ratio | 59:41 | 59:41 | 59:41 | 59:41 | 59.41 | 59:41 |
| Equiv. Ruxolitinib, | 1.500 | 1.501 | 1.500 | 1.506 | -0- | -0- |
| % w/w | | | | | | |
| Chemical stability, | — | 1.517 (0.4) | — | 1.519 (0.4) | — | NA |
| Ruxolitinib % LC (w/w), | | ambient; | | ambient; | | |
| Ave (% RSD at 1 month, | | 1.515 (0.2) | | 1.527 (0.1) | | |
| ambient and 40° C. | | at 40° C. | | at 40° C. | | |
| Total Impurities, | — | 0.05; 0.05 | — | 0.05; 0.05 | — | ND |
| (% area) 1 month, | | | | | | |
| ambient and 40° C. | | | | | | |
| Foam appearance, | — | Well | — | Well | — | NA |
| ambient | | formed | | formed | | |
| | | stable | | stable | | |
| | | foam | | foam | | |
| Foam collapse, 32° C. | — | 8 | — | 9 | — | 7 |
| samples 25° C.), minutes | | | | | | |
| Miscibility/homogeneity | — | Uniform, | — | 2-phase | — | — |
| | | miscible | | separation | | |

N/A = not applicable;
ND = not detected

### Example 11C: Further Investigation of Stearyl Alcohol

The levels of stearyl alcohol and cetyl alcohol were further investigated as to their impact on foam appearance and collapse rate. As noted above, a higher collapse rate than ROGAINE is preferred for a foam to be applied to the scalp.

The study was designed as follows:

| | | Cetyl | Stearyl |
|---|---|---|---|
| | | alcohol | alcohol |
| | | (C16) | (C18) |
| Samples | | content, | content, |
| code | SAMPLE Placebo, F/L | % w/w | % w/w |
| A-1 | 268-3-02/268-3-02 (Reference 1) | 2.20 | 1.00 |
| A-2 | 268-7-01/268-7-01 | 2.20 | 0.50 |
| A-3 | 268-7-02/268-7-02 | 2.20 | 0.25 |
| B-1 | 268-4-01/268-4-01 (Reference-2) | 1.10 | 0.56 |
| B-2 | 268-7-03/268-7-03 | 1.10 | 0.28 |
| B-3 | 268-7-04/268-7-04 | 1.10 | 0.14 |

The additional formulations were prepared and studied as shown in Table 30 below (as prepared by manufacturing Method A). The formulation showing best foam collapse (i.e., ˜2.0 mins.) demonstrated in placebo foam 268-7-01/268-7-01 contains fatty alcohols at 2.2% w/w cetyl alcohol and 0.5% w/w of stearyl alcohol. A quick break foam is expected in hydroethanolic formulations composed of 2.2% (w/w) cetyl alcohol and between 0.5 to 0.75% (w/w) of stearyl alcohol.

TABLE 30
Levels of fatty alcohols in placebo formulations

| | Formulation | | | | | | | |
|---|---|---|---|---|---|---|---|---|
| | 268-7-01 | | 268-7-02 | | 268-7-03/ | | 268-7-04 | |
| | (C16:C18) ratio | | | | | | | |
| | (2.20:0.50) | | (2.20:0.25) | | (1.10:0.25) | | (1.10:0.125) | |
| | Req'd | Actual, | Req'd | Actual, | Req'd | Actual, | Req'd | Actual, |
| Ingredients | % w/w | % w/w | % w/w | % w/w | % w/w | % w/w | % w/w | % w/w |
| Propylene glycol | 5.00 | 5.00 | 5.00 | 5.00 | 5.00 | 5.00 | 5.00 | 5.02 |
| Laureth-4 | 1.00 | 1.00 | 1.00 | 1.00 | 1.00 | 1.00 | 1.00 | 1.01 |
| Polysorbate 60 | 0.40 | 0.40 | 0.40 | 0.40 | 0.40 | 0.40 | 0.40 | 0.41 |
| Cetyl alcohol | 2.20 | 2.22 | 2.20 | 2.20 | 1.10 | 1.10 | 1.10 | 1.10 |
| Stearyl alcohol | 0.50 | 0.50 | 0.25 | 0.26 | 0.25 | 0.25 | 0.125 | 0.12 |
| Purified water | 35.65 | 35.66 | 35.71 | 35.71 | 36.20 | 36.20 | 36.25 | 36.24 |
| Ethanol, g | 51.29 | 51.26 | 51.48 | 51.47 | 52.09 | 52.09 | 52.17 | 52.15 |
| Ruxolitinib | nil | 0 | nil | 0 | nil | 0 | nil | 0 |
| phosphate | | | | | | | | |
| NaOH 20% aq | qs | 0 | qs | 0 | qs | 0 | qs | 0 |
| soln | | | | | | | | |
| TOTAL net | 96.04 | 96.04 | 96.04 | 96.04 | 96.04 | 96.04 | 96.04 | 96.04 |
| % w/w | | | | | | | | |
| TOTAL (E + W) | 86.94 | 86.92 | 87.19 | 87.18 | 88.29 | 88.29 | 88.42 | 88.39 |
| (E:W) % ratio | 59:41 | 59:41 | 59:41 | 59:41 | 59:41 | 59:41 | 59:41 | 59:41 |
| Foam appearance, | — | | Broad base, | | — | | Flat almost | |
| 25° C. | | | well-formed | | | | liquid foam i.e., | |
| | | | foam | | | | (C16:C18) | |
| | | | | | | | at (1.1:0.125) % | |
| Foam collapse | 2 | | 1.5. | | 1.5 | | 1 | |
| time (32° C./oven), | | | | | | | | |
| mins. | | | | | | | | |

### Example 11D: Addition of Emollients to the Hydroethanolic Foamable Compositions

Because hydroethanolic foams contain high amounts of ethanol, the skin can become dry upon evaporation of ethanol. Therefore, the use of emollients in the hydroethanolic formulations was explored to protect the skin surface from this drying effect.

Several emollients were explored as to their compatibility in hydroethanolic (HE) mixtures having 60% ethanol by weight. The following emollients were found to be miscible in the HE mixtures: PEG-6 caprylic capric glycerides (Glycerox 767), glycerin, glyceryl caprylate, glyceryl caprate, diisopropyl adipate (DIPA), isostearaic acid, oleic acid, PPG-15 stearyl ether, and glyercol monolaurate. Myristyl lactate was also later explored in the formulations with co-solvent present.

Several formulations were prepared to study physical stability, foam appearance and collapse time using manufacturing Method B (Materials and Methods). As shown in Table 31, the use of glycerin, PPG-15 stearyl ether, oleic acid, and DIPA were explored as mixtures. DIPA is a polar oil ester with emollient properties and is miscible with glycerin. However, Table 31 shows that the formulation with a combination of DIPA and glycerin resulted in aggregation as oily droplets. Other emollient combinations did not show this phase separation.

TABLE 31
Emollient combinations

| | | Formulation Lots | | | |
|---|---|---|---|---|---|
| | | 268-3-02 | 268-9-01 | 268-9-02 | 268-9-03 |
| | | Emollient materials | | | |
| | | | Glycerin &, | | |
| | | Base | PPG 15 | Glycerin & | Glycerin & |
| | | formulation | stearyl ether | DIPA | Oleic acid |
| INGREDIENTS | | % w/w | Actual % w/w | Actual % w/w | Actual % w/w |
| Propylene glycol | | 5.00 | 5.00 | 5.01 | 5.01 |
| Laureth-4 | | 1.00 | 1.03 | 1.00 | 1.02 |
| Polysorbate 60 | | 0.40 | 0.44 | 0.40 | 0.41 |
| Ro Water | | 32.57 | 32.54 | 32.57 | 32.56 |
| Cetyl alcohol | | 2.20 | 2.20 | 2.20 | 2.20 |
| Stearyl alcohol | | 1.00 | 1.00 | 1.00 | 1.00 |
| Ethanol, g | | 46.87 | 46.85 | 46.86 | 46.84 |
| Ruxolitinib phosphate | | 3.960 | nil | nil | nil |
| (CF = 0.758) | | | | | |
| EMOLLIENT | Glycerin | 5.00 | 1.00 | 1.00 | 1.02 |
| | PPG 15 Stearyl | | 4.00 | — | — |
| | Ether | | | | |
| | Oleic acid, refined | | — | — | 4.00 |
| | Diisopropyl adipate | | — | 4.00 | — |

| | | | | |
|---|---|---|---|---|
| 20% NaOH aq solution, to pH | 2.00 | NIL | NIL | NIL |
| 5.0-6.5 | | | | |
| TOTAL NET | 100.00 | 94.04 | 94.04 | 94.04 |
| Sample % content propellant, | — | 4.4% P75 | — | 4.7% P75 |
| P75 | | | | |
| Miscibility examination | — | a | Uniform clear | a |
| through a glass aerosol bottle | | | but oil blob at | |
| | | | the bottom (b) | |
| Foam Appearance, 25° C. | — | Flat coarse | Well-formed | Flat, coarse, |
| | | growing to | stable foam | and liquefied |
| | | larger bubbles | | |
| Foam collapse time, | — | — | 6 minutes | — |
| 32° C./oven method, sample | | | | |
| 25° C. pre-acclimatised | | | | |

Legend:
a—Immiscible. Clear but with 2-phase separation observed, appear hazy upon shaking.
(b)—Immiscible. Visual examination of the sample revealed small oil globules at the bottom. Remains clear when shaken.

Other formulations with different emollient combinations were also prepared (Table 32).

TABLE 32
Other formulations with different emollient combinations

| | | Formulation Lot | | | | |
|---|---|---|---|---|---|---|
| | | 268-10-04/ | 268-9-05/ | 268-9-08 | 268-9-09 | 268-9-10 |
| | | Emollient materials, % w/w ratio | | | | |
| | | | PEG | PS11 | | Myristyl |
| | | PPG15SE:Trans- | 300:Glycerin | E:Trans- | DIPA:Oleic | lactate:Trans- |
| | | cutol-P (4:1) | (4:1) | cutol (4:1) | acid (4:1) | cutol-P (2:3) |
| | | Actual, | Actual, | Actual, | Actual, | Actual, |
| INGREDIENTS | | % w/w | % w/w | % w/w | % w/w | % w/w |
| Propylene glycol | | 4.87 | 4.95 | 5.00 | 4.99 | 5.00 |
| Laureth-4 | | 0.98 | 1.04 | 1.00 | 1.00 | 1.00 |
| Polysorbate 60 | | 0.42 | 0.40 | 0.41 | 0.41 | 0.40 |
| Purified Water | | 31.58 | 32.25 | 32.54 | 32.54 | 32.57 |
| Cetyl alcohol | | 2.13 | 2.18 | 2.20 | 2.20 | 2.20 |
| Stearyl alcohol | | 0.99 | 1.00 | 1.00 | 1.00 | 1.00 |
| Ethanol 100HG | | 45.55 | 47.23 | 46.83 | 46.83 | 46.87 |
| Ruxolittinib Phosphate, A/S | | 3.84 | 3.95 | 3.96 | 3.98 | 4.026 |
| Diethylene glycol monoethyl | | 0.98 | — | 1.02 | — | 3.00 |
| ether/Transcutol-P | | | | | | |
| EMOLLIENT/S | PPG15 stearyl ether/ | 3.91 | — | — | — | — |
| | Crodamol PS15E | | | | | |
| | Super Refined Oleic acid | — | — | — | 1.01 | — |
| | Arlamol PS11E Pharma | — | — | 4.06 | — | — |
| | (propoxylate) | | | | | |
| | PEG 300 super refined | — | 4.02 | — | — | — |
| | DIPA/Crodamol DA | — | — | — | 4.02 | — |
| | Glycerin | — | 1.01 | — | — | — |
| | Myristyl lactate/SP | — | — | — | — | 2.01 |
| | Crodamol ML | | | | | |

| | | | | | |
|---|---|---|---|---|---|
| NaOH as 20% aq soln, to pH 5.0- | 2.57 | 1.98 | 2.00 | 2.03 | 2.00 |
| 6.0 | | | | | |
| Acetic acid | 2.19 | nil | nil | nil | nil |
| TOTAL NET | 100.00 | 100.00 | 100.00 | 100.00 | 100.00 |
| Equivalent % Ruxolitinib free | 2.91 | 2.99 | 3.00 | 3.02 | 3.05 |
| base | | | | | |
| Final pH of base | 6.17 | 5.17 | 5.20 | 5.20 | 5.10 |
| Miscibility, visual examination | Immiscible, | Miscible, | Immiscible, hazy | Miscible, | Miscible, |
| through a glass aerosol bottle | layer on top, | uniform clear, | on shaking (c) | uniform (c) | uniform (c) |
| | small oil | No oil blob (c) | | | |
| | blobs (a) | | | | |
| Foam appearance, ambient | — | Well-formed | — | Flat, liquid | Flat coarse |
| (Error! Reference source not | | stable foam | | | bubbles |
| found.) | | | | | growing i.e., |
| | | | | | post foaming |
| Foam collapse, mins at | Not tested | >5 < 10 minutes | Not tested | Not tested | Not tested |
| 32° C./oven method, sample pre- | | to collapse, | | | |
| acclimatised to 25° C. | | 28-38° C. | | | |

Legend:
(a)—Immiscible. Clear but with 2-phase separation observed, appear hazy upon shaking;
(b)—Immiscible. Careful visual examination of the sample revealed minute oil globules at the bottom. Also, remaining clear when shaken; and
(c)—Miscible, Clear, uniform and no separation nor oil globules/particulates afloat. Remains clear when sample is shaken.

Of the five formulations from Table 32, the following formulations were found to result in a homogeneous foam showing no phase separation. The samples also showed good base miscibility resulting in transparent uniform foam product.
268-9-05/268-9-05, PEG 300+Glycerin (4%: 1%) w/w;
268-9-09/268-9-09, DIPA+Oleic acid (4%: 1%) w/w; and
268-9-10/268-9-10, Myristyl lactate & Transcutol-P (2%: 3%) w/w.

Ruxolitinib phosphate was found to maintain its chemical stability in these three formulations for one month at 40° C./75% RH, indicating the API is stable in the presence of these emollients.

The 2 remaining formulations in Table 32 are eliminated due to inhomogeneity/phase separation with the appearance of hazy layer on the top and turning cloudy upon agitating the glass container.
268-9-04/268-9-06 ie, PPG 15 SE+Transcutol-P (4%: 1%) and
268-9-08/268-9-08 ie, PS 11 E+Transcutol-P (4%: 1%).

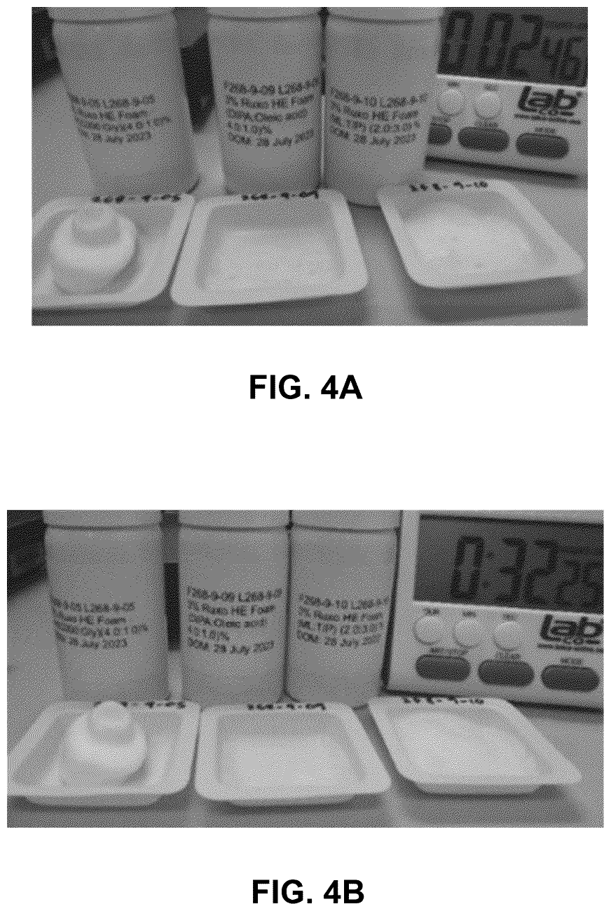

FIG. 4 shows the foam appearance after 0-2 minutes and then after 32 minutes. That is, FIG. 4A are the foam appearances of formulations, 268-9-05, 268-9-09 and 268-9-10 at time 0 to 2 minutes; FIG. 4B are the foam appearances of formulations, 268-9-05, 268-9-09 and 268-9-10 at time 32 minutes.

The best of the formulations was 268-9-05 which contains glycerin as the emollient and PEG 300 as a solvent and penetration enhancer. PEG300 replaces the PEG400 in the earlier formulations due to the lack of compatibility of PEG400 in the formulations having the API. While the foam quality of 268-10 was not good, the miscibility of the emollient, myristyl lactate (ML) was good with optimization needed as to the levels of ML and trancutol-P, transcutol-P being a solvent/penetration enhancer.

### Example 11E: Investigation of the Emulsification System in High HE Formulations

The use of non-ionic emulsifiers in the HE formulations having emollients was investigated. Specifically, sorbitan monolaurate (Span 20) (HLB 8.8) and polyethylene glycol sorbitan monostearate (Tween 60) (HLB 14.9) were substituted for laureth-4 in the HE formulations (Table 33, prepared by manufacturing Method B).

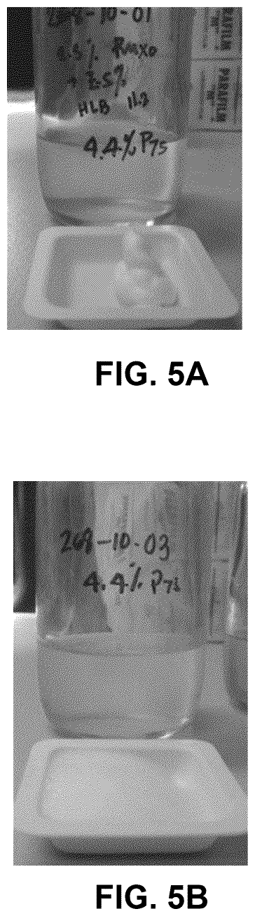

As shown below, the 268-10-01 formulation with Tween 60/Span 20 and mixture of glycerin and PEG300 was found to form a well-formed and stable foam in HE formulations having at 60:40 ethanol:water ratio (Table 34 and FIG. 5A, directed to the 268-10-01 formulation; FIG. 5B directed to the 268-10-03 formulation).

TABLE 33
Non-ionic emulsifiers in the HE formulations

| | Formulation Lot | | | |
|---|---|---|---|---|
| | 268-10-01 | | 268-10-03 | |
| | Req'd | Actual, | Req'd | Actual |
| INGREDIENTS | % w/w | % w/w | % w/w | % w/w |
| Propylene glycol, super refined | 5.00 | 4.94 | 5.00 | 5.00 |
| Sorbitan laurate (HLB 8.8) | 1.00 | 1.01 | 1.00 | 1.01 |
| Polysorbate 60 (HLB14.9) | 0.70 | 0.70 | 0.70 | 0.71 |
| Purified Water | 29.00 | 28.75 | 29.90 | 29.89 |
| Cetyl alcohol | 2.20 | 2.18 | 2.20 | 2.20 |
| Stearyl alcohol | 1.00 | 1.00 | 1.00 | 1.00 |
| Ethanol 100HG | 52.50 | 52.10 | 54.45 | 54.42 |
| Ruxolittinib Phosphate | 3.300 | 3.26 | 3.300 | 3.31 |
| (CF = 0.758) | | | | |
| Glycerin | 1.00 | 0.98 | 1.00 | 1.01 |
| Super Refined PEG300 | 4.00 | 3.96 | — | — |
| Myristyl lactate | — | — | 1.00 | 1.01 |
| Mono-ethanolamine 99% | 0.30 | 0.25 | 0.45 | 0.44 |
| 30% aq. KOH to pH 5-6 | | 0.86 | | — |
| TOTAL net | 100.00 | 100.00 | 100.00 | 100.00 |
| Total E + W | 81.50 | 80.85 | 84.35 | 84.31 |
| Ethanol Input | 52.50 | 52.10 | 54.45 | 54.42 |
| Water Input | 29.00 | 28.75 | 29.90 | 29.89 |
| % Ethanol | 64.0 | 64.0 | 65.0 | 65.0 |
| % Water | 36.0 | 36.0 | 35.0 | 35.0 |
| Equiv Ruxolitinib conc as free | 2.50 | 2.47 | 2.50 | 2.51 |
| base | | | | |
| pH | — | 5.75 | — | 5.04 |
| Propellant AP75 | — | 4.4% | — | 4.4% |
| Emollients | Glycerin:PEG 300 | | Glycerin:Myristyl | |
| | (1:4) % w/w | | lactate (1:1) % w/w | |
| Miscibility of propellant to foam | — | Uniform, | — | Uniform, |
| base (glass aerosol bottle) | | yellow tinge | | Yellow |
| | | with slight | | tinge, |
| | | haziness | | transparent |
| | | | | than 268- |
| | | | | 10-01 |
| Foam appearance, ambient | — | Well- | — | Flat, |
| | | formed and | | coarse, |
| | | stable | | liquified |
| Foam collapse, 37° C. test (oven)- | — | 0.5 min (no | — | — |
| indicative | | test at | | |
| | | 32° C.) | | |

TABLE 34
Non-ionic emulsifiers in the HE formulations

| | 268-3-02 | | |
|---|---|---|---|
| Formulation Lot | Reference | 268-10-01 | 268-10-03 |
| Equiv. % Ruxolitinib | 2.97 | 2.47 | 2.51 |
| free base | | | |
| pH | 6.4 | 5.75 | 5.04 |
| Fatty alcohol | 2.2:1.0 | 2.2:1.0 | 2.2:1.0 |
| ratio (C16:C18) | | | |
| Surfactants-binary | Tween 60:Laureth-4 | Tween 60:Span 20 | Tween 60:Span 20 |
| | (1.4% total), | (1.7% total), | (1.7% total), |
| | HLB 11.3 | HLB11.2 | HLB 11.2 |
| Emollient | nil | 5% total (PEG300 | 2% total (Myristyl |
| | | and Glycerin, 4:1) | lactate and |
| | | | Glycerin, 1:1) |
| High % Ethanol | (58:42) | (64:36) | (65:35) |
| (E:W) ratio | | | |
| P75% content | 4.4% | 5.1% | 4.5% |
| Foam appearance, | Well formed, | Well-formed | Flat, coarse |
| ambient | stable | stable | liquefied |

Further investigation into the use of a non-ionic emulsification system was investigated, along with modification of the stearyl alcohol levels (Table 35; manufacturing Method B). Specifically, laureth-4 was replaced with Tween 60/Span 20 or Tween 60 alone and stearyl alcohol was modified to 0.75% instead of the previous 1% levels. 2.5% (w/w) of ruxolitinib phosphate on a free base basis was used.

The best foam structure, ambient is that of 268-13-02 (60:40) E:W ratio, although slightly long foam collapse (>6 minutes). Note single surfactant Tween 60 at 1.33% and HLB 14.9.

With a slightly high ethanol in 268-13-03 (61:39, E:W) ratio, the foam collapse is reduced to 2 mins. This formulation contains a binary surfactant system-Tween 60 and Span 20, total 1.4% with HLB 12.18. There is, however, an immiscibility issue noticing a 1 mm layer on surface of this sample-possibly an excess propellant at 5%.

TABLE 35
Additional formulations of the non-ionic emulsification systems

| | Formulation Lot | | | | | |
|---|---|---|---|---|---|---|
| | 268-13-01 | | 268-13-02/ | | 268-13-03 | |
| | | Actual | | Actual | | Actual |
| INGREDIENTS | % w/w | % w/w | % w/w | % w/w | % w/w | % w/w |
| WATER PHASE | | | | | | |
| Purified Water | 35.30 | 35.29 | 34.30 | 34.29 | 33.35 | 33.39 |
| Polysorbate 60 | 0.80 | 0.80 | 1.35 | 1.33 | 0.80 | 0.79 |
| Propylene glycol | 5.00 | 5.01 | 5.00 | 5.02 | 5.00 | 4.98 |
| Sub-total | 41.10 | 41.09 | 40.65 | 40.65 | 39.15 | 39.16 |
| OIL PHASE | | | | | | |
| Cetyl alcohol | 2.20 | 2.20 | 2.20 | 2.20 | 2.20 | 2.20 |
| Stearyl alcohol | 0.75 | 0.75 | 0.75 | 0.75 | 0.75 | 0.76 |
| Sorbitan laurate | 0.60 | 0.65 | — | — | 0.60 | 0.60 |
| Sub-total | 3.55 | 3.59 | 2.95 | 2.95 | 3.55 | 3.56 |
| FINAL | | | | | | |
| Ethanol, g | 50.80 | 50.74 | 51.85 | 51.88 | 53.00 | 52.77 |
| Ruxolitinib | 3.30 | 3.31 | 3.30 | 3.31 | 3.3000 | 3.29 |
| Phosphate | | | | | | |
| Triethanolamine | 1.25 | 1.26 | 1.25 | 1.22 | 1.00 | 1.21 |
| 99% | | | | | | |
| Sub-total | 55.35 | 55.31 | 56.40 | 56.40 | 57.30 | 57.27 |
| TOTAL NET | 100.00 | 100.00 | 100.00 | 100.00 | 100.00 | 100.00 |
| Ruxolitinib free | 2.50 | 2.51 | 2.5014 | 2.51 | 2.50 | 2.4976 |
| base, | | | | | | |
| HLB | 12.2 | 12.08 | 14.9 | 14.9 | 12.20 | 12.18 |
| pH (initial/adjusted) | pH 5-6 | 5.66 | pH 5-6 | 5.59 | — | 3.23/5.18 |
| Ethanol input | 50.8 | 86.02 | 51.85 | 51.88 | 53.00 | 52.77 |
| Water Input | 35.3 | 50.74 | 34.30 | 34.29 | 33.35 | 33.39 |
| TotalL E + W | 86.10 | 35.29 | 86.15 | 86.17 | 86.35 | 86.16 |
| % ratio E | 59.0 | 59.0 | 60.0 | 60.0 | 61.38 | 61.25 |
| % ratio W | 41.0 | 41.0 | 40.0 | 40.0 | 38.62 | 38.75 |

| | | | |
|---|---|---|---|
| Miscibility P75 with | Immiscible base- | Miscible. Clear, | Immiscible, 1 mm |
| the foam base in | P75, Clear layer on | uniform, homogeneous | layer almost |
| glass aerosol bottle | the surface- PS (hazy | both at still or | unnoticed, slightly |
| | when shaken) | when shaken. | hazy when shaken. |
| Foam appearance - | Liquid at first | Well-formed tight | Well-formed, slightly |
| ambient | then grows to coarse, | particles, stable | airy foam, stable |
| | big particles but | | |
| | flat foam | | |
| Foam collapse, | Not tested | >>6 mins | 2 minutes |
| 32° C. (water bath) | | | |

Further investigation was made into the HE formulations having glycerin/PEG300 or myristyl laurate/transcutol-P as emollients (Tables 36 and 37). Further optimization of the myristyl laurate/transcutol-P compositions was made as to the levels the emollients and solvent (Table 38). The formulations in Table 38 were found to form well-formed and tight foams of fine particles or bubbles resulting in longer collapse rates. However, higher ethanol ratios were necessary to form miscible formulations (268-15-03/268-15-04). Trancutol-P is a stabilizer for myristyl lactate, allowing the emollient to stay in solution without precipitation. The formulations were prepared by manufacturing Method B.

TABLE 36
Additional formulations under investigation.

| | Formulation Lot | | | |
|---|---|---|---|---|
| | 268-14-01 | | 268-14-03 | |
| | Emollients | | | |
| | PEG 300, Glycerin and | | PEG 300, Glycerin and | |
| | Myristyl lactate | | Myristyl lactate | |
| | (3, 0.75, 0.25) | | (1.8, 0.25, 0.5) | |
| | | Actual | | Actual |
| INGREDIENTS | % w/w | % w/w | % w/w | % w/w |
| A. WATER PHASE | | | | |
| Propylene glycol | 5.00 | 5.02 | 5.00 | 5.00 |
| Polysorbate 60 HLB 14.9 | 0.70 | 0.41 | 0.50 | 0.51 |
| Ro Water | 32.57 | 32.60 | 31.69 | 31.62 |
| Glycerin | 0.75 | 0.78 | 0.25 | 0.25 |
| Subtotal Water Phase | 38.72 | 38.80 | 37.44 | 37.38 |
| B. OIL PHASE | | | | |
| Super Refined PEG 300 | 3.00 | 2.98 | 1.80 | 1.78 |
| Myristyl lactate | 0.25 | 0.26 | 0.50 | 0.50 |
| Cetyl alcohol | 1.10 | 1.09 | 1.10 | 1.10 |
| Stearyl alcohol | 0.50 | 0.50 | 0.25 | 0.25 |
| Sorbitan laurate HLB 8.6 | 1.00 | 0.62 | 0.40 | 0.40 |
| Subtotal Oil Phase | 5.45 | 5.46 | 4.05 | 4.02 |

Phase 3/Active + Solvent

| | | | | |
|---|---|---|---|---|
| Ruxolitinib Phosphate | 3.960 | 3.97 | 3.30 | 3.28 |
| (CF = 0.758) | | | | |
| Ethanol, g | 51.47 | 51.19 | 54.71 | 54.13 |
| Ethanolamine | 0.40 | 0.58 | 0.50 | 1.19 |
| Subtotal Active Phase | 55.83 | 55.743 | 58.51 | 58.60 |
| TOTAL NET | 100.00 | 100.00 | 100.00 | 100.00 |
| Equiv % w/w Ruxolitinib | 3.00 | 3.01 | 2.50 | 2.49 |
| Total Ethanol and Water | 84.04 | 83.79 | 86.40 | 85.75 |
| E-W ratio | 61.24 | 61.10 | 63.32 | 63.13 |
| | 38.76 | 38.90 | 36.68 | 36.87 |

| | | |
|---|---|---|
| Miscibility of P75 to foam | No visible layer or separation | Miscible, clear no haze at still |
| base in glass aerosol bottle | but slight haze when shaken | and when shaken. No separation |
| Foam appearance - | Liquid froth when sprayed, | Liquid frothy, big bubbles, |
| ambient | grows to coarse flat foam | grows to coarse flat foam |
| Foam collapse, 32° C. | No test conducted | No test conducted |

TABLE 37
Additional formulations under investigation.

| | Formulation Lot: | | | | | | | |
|---|---|---|---|---|---|---|---|---|
| | 268-14-02 | | 268-14-04 | | 268-14-05 | | 268-14-06 | |
| | Emollient/s | | | | | | | |
| | | | PEG 300; Myristyl | | | | | |
| | 0.8% Myristyl | | lactate, Transcutol-P | | 0.42% Myristyl | | 0.41% Myristyl | |
| | lactate only | | (2.5, 1.0, 01.5) | | lactate only | | lactate only, | |
| | (63:37, E:W) | | (59:41, E:W) | | (63:37, E:W) | | (59:41, E:W) | |
| INGREDIENTS | % w/w | Actual % w/w | % w/w | Actual % w/w | % w/w | Actual % w/w | % w/w | Actual % w/w |
| A. WATER PHASE | | | | | | | | |
| Propylene glycol | 5.00 | 4.94 | 5.00 | 5.03 | 5.00 | 5.00 | 5.00 | 4.99 |
| Polysorbate 60 HLB | 0.50 | 0.51 | 0.70 | 0.71 | 0.50 | 0.50 | 0.70 | 0.70 |
| 14.9 | | | | | | | | |
| Purified Water | 32.33 | 31.98 | 33.13 | 33.02 | 32.29 | 32.25 | 35.12 | 35.03 |
| Transcutol-P | — | — | 1.50 | 1.50 | — | — | — | — |
| Subtotal Water Phase | 37.83 | 37.43 | 40.33 | 40.26 | 37.79 | 37.75 | 40.82 | 40.72 |
| B. OIL PHASE | | | | | | | | |
| Super Refined PEG300 | — | — | 2.50 | 2.49 | — | — | — | — |
| Myristyl lactate | 0.80 | 0.82 | 1.00 | 1.01 | 0.40 | 0.42 | 0.40 | 0.41 |
| Cetyl alcohol | 1.10 | 1.09 | 2.20 | 2.18 | 1.10 | 1.10 | 2.20 | 2.18 |
| Stearyl alcohol | 0.25 | 0.25 | 1.00 | 0.99 | 0.25 | 0.25 | 0.75 | 0.75 |
| Sorbitan laurate HLB 8.6 | 0.40 | 0.41 | 1.00 | 1.29 | 0.40 | 0.43 | 1.00 | 0.99 |
| Subtotal OilPhase | 2.55 | 2.57 | 7.70 | 7.96 | 2.15 | 2.20 | 4.35 | 4.34 |
| Phase 3/Active + Solvent | | | | | | | | |
| Ruxolittinib Phosphate | 3.30 | 3.28 | 3.30 | 3.29 | 3.300 | 3.34 | 3.300 | 3.30 |
| Ethanol 100HG | 55.82 | 55.08 | 47.67 | 47.27 | 55.76 | 55.72 | 50.53 | 50.25 |
| Ethanolamine | 0.50 | 1.64 | 1.00 | 1.24 | 1.00 | 1.00 | 1.00000 | 1.39 |
| Subtotal | 59.62 | 60.01 | 51.97 | 51.80 | 60.06 | 60.05 | 54.83 | 54.94 |
| TOTAL NET | 100.00 | 100.00 | 100.00 | 100.02 | 100.00 | 100.00 | 100.00 | 100.00 |
| Equivalent % | 2.5014 | 2.49 | 2.50 | 2.49 | 2.50 | 2.53 | 2.50 | 2.50 |
| Ruxolitinib | | | | | | | | |
| Total Ethanol and | 88.15 | 87.06 | 80.80 | 80.29 | 88.05 | 87.96 | 85.65 | 85.28 |
| Water | | | | | | | | |
| E:W ratio | 63:37 | 63:37 | 59:41 | 59:41 | 63:37 | 63:37 | 59:41 | 59:41 |

| | | | | |
|---|---|---|---|---|
| Miscibility P75 with the | Miscible, clear no haze | Visible yellowish clear | Miscible, clear no haze at | Visible clear layer on |
| foam base in glass | at still and when shaken. | layer on surface ie phase | still and when shaken. | surface ie phase |
| aerosol bottle | No phase separation | separation. Hazy when | No phase separation | separation. Hazy when |
| | | bottle is shaken | | bottle is shaken |
| Foam appearance - | Slightly coarse foam | Liquid when spayed | Coarse liquid when | Liquid when spayed |
| ambient | growing larger | growing throughout to a | sprayed then grows | growing throughout to a |
| | throughout but quickly | flat slightly coarse but | larger throughout but | flat slightly coarse but |
| | breaks down/ collapses. | stable post foam | quickly breaks down/ | stable post foam, similar |
| | | | collapses to a liquid | to 268-14-04 |
| | | | again. | |
| Foam collapse, 32° C. | N/A | N/A | N/A | N/A |
| (H2O bath) | | | | |

TABLE 38
Additional formulations under investigation.

| | Formulation Lot | | | | | | | |
|---|---|---|---|---|---|---|---|---|
| | 268-15-01 | | 268-15-02 | | 268-15-03 | | 268-15-04 | |
| | Details, Emollient & E:W content | | | | | | | |
| | Myristyl lactate + | | Myristyl lactate + | | Myristyl lactate + | | Myristyl lactate + | |
| | Transcutol-P ((1:0.50) %; | | Transcutol-P ((1:0.50) %; | | Transcutol-P ((1:0.50) %; | | Transcutol-P ((1:0:1.0) %; | |
| | (55:45, E:W) ratio; Single | | (56:44, E:W); Single | | (59:41, E:W); Single | | (59:41, E:W); Single | |
| | surfactant (Tween 60) | | surfactant (Tween 60) | | surfactant (Tween 60) | | surfactant (Tween 60) | |
| INGREDIENTS | % w/w | Actual % w/w | % w/w | Actual % w/w | % w/w | Actual % w/w | % w/w | Actual % w/w |
| Water PHASE | | | | | | | | |
| Propylene glycol | 5.00 | 5.02 | 5.00 | 5.34 | 5.00 | 4.98 | 7.50 | 7.47 |
| Polysorbate 60 | 1.35 | 1.39 | 1.35 | 1.47 | 1.35 | 1.37 | 1.35 | 1.34 |
| (HLB14.9) | | | | | | | | |
| Ro Water | 38.09 | 38.16 | 36.40 | 37.09 | 34.71 | 34.58 | 33.48 | 33.42 |
| Sub total | 44.44 | 44.574 | 42.75 | 43.91 | 41.06 | 40.94 | 42.33 | 42.24 |
| OIL PHASE | | | | | | | | |
| Cetyl alcohol | 2.20 | 2.19 | 2.20 | 2.17 | 2.20 | 2.21 | 2.20 | 2.19 |
| Stearyl alcohol | 0.75 | 0.75 | 0.75 | 0.73 | 0.75 | 0.76 | 0.75 | 0.76 |
| Transcutol-P (HB 4.2) | 0.50 | 0.52 | 0.50 | 0.57 | 0.50 | 0.56 | 1.00 | 0.99 |
| Myristyl laurate | 1.00 | 1.00 | 1.00 | 0.98 | 1.00 | 1.03 | 1.00 | 0.99 |
| Subtotal | 4.45 | 4.452 | 4.45 | 4.45 | 4.45 | 4.57 | 4.95 | 4.94 |
| ACTIVE + SOLVENT | | | | | | | | |
| Ethanol | 46.56 | 46.42 | 48.25 | 47.22 | 49.94 | 49.84 | 48.17 | 48.29 |
| Ruxolitinib Phosphate | 3.300 | 3.33 | 3.30 | 3.25 | 3.300 | 3.33 | 3.300 | 3.30 |
| Ethanolamine 99% | 1.25 | 1.23 | 1.25 | 1.17 | 1.250 | 1.32 | 1.250 | 1.24 |
| Subtotal | 51.11 | 50.973 | 52.80 | 51.643 | 54.490 | 54.493 | 52.720 | 52.825 |
| TOTAL net | 100.00 | 100.00 | 100.00 | 100.00 | 100.00 | 100.00 | 100.00 | 100.00 |
| Total E + W | 84.65 | 84.58 | 84.65 | 84.31 | 84.65 | 84.42 | 81.65 | 81.71 |
| Ethanol input | 46.56 | 46.42 | 48.25 | 47.22 | 49.94 | 49.84 | 48.17 | 48.29 |
| Water input | 38.09 | 38.16 | 36.40 | 37.09 | 34.71 | 34.58 | 33.48 | 33.42 |
| % Ethanol | 55.00 | 54.88 | 57.00 | 56.00 | 59.00 | 59.03 | 59.00 | 59.10 |
| % Water | 45.00 | 45.12 | 43.00 | 44.00 | 41.00 | 40.97 | 41.00 | 40.90 |
| Equiv Ruxolitinib | 2.5014 | 2.52 | 2.50 | 2.47 | 2.50 | 2.52 | 2.50 | 2.50 |
| freebase | | | | | | | | |

| | | | | |
|---|---|---|---|---|
| Miscibility P75 with the | Visible clear layer on | Visible clear layer on | Miscible, clear no haze | Miscible, clear no haze |
| foam base in glass | surface i.e., phase | surface i.e., phase | at still and when shaken. | at still and when shaken. |
| aerosol bottle | separation. Hazy when | separation. Hazy when | Uniform, no phase | Uniform, no phase |
| | bottle is shaken | bottle is shaken | separation | separation |
| Foam appearance - | Well formed, light | Well formed creamy | Well formed creamy | Well formed creamy |
| ambient | foam with tight | foam with tight | foam with tight | foam with tight |
| | particulate/bubbles, | particulates/bubbles, | particulates/bubbles, | particulates/bubbles, |
| | stable light foam | stable creamy foam | stable creamy foam | stable creamy foam |
| Foam collapse, 32° C. | No test performed | No test performed | No test performed | No test performed |
| (H2O bath) | | | | |

Further HE formulations were prepared having high ethanol content (E:W of 60:40), having glycerin/PEG300 or myristyl lactate/transcutol-P, Tween 60 as the emulsifier, and either (a) no API (placebo) (Tables 39 and 40), (b) 1.5% (w/w) or 2.5% (w/w) on free base basis of ruxolitinib phosphate (Tables 41 and 42), or (c) 1.5% (w/w) or 2.5% (w/w) on free base basis of deuruxolitinib phosphate (Tables 43 and 44). The formulations were made by manufacturing Method B.

TABLE 39
Placebo formulations

| | | Formulation Lot | | | | | | | | | |
|---|---|---|---|---|---|---|---|---|---|---|---|
| | | 268-16-01 | | 268-16-19 | | 268-16-20 | | 268-16-04 | | 268-16-05 | |
| | | PRODUCT DESCRIPTION | | | | | | | | | |
| | | | | | | | | PLACEBO HE | | PLACEBO HE | |
| | | PLACEBO HE | | PLACEBO HE | | PLACEBO HE | | FOAM 1% | | FOAM 1% | |
| | | FOAM NIL | | FOAM 5% | | FOAM 2.5% | | Emollient + 0.5% | | Emollient + 1% | |
| | | Emollient | | Emollient | | Emollient | | Penetration enhancer | | Penetration enhancer | |
| | | | % w/w | | % w/w | | % w/w | | % w/w | | % w/w |
| ITEM | | % w/w | By | % w/w | By | % w/w | By | % w/w | By | % w/w | By |
| # | Ingredients | Required | Input | Required | Input | Required | Input | Required | Input | Required | Input |
| 1 | Water | 34.30 | 34.30 | 32.96 | 32.94 | 33.96 | 33.95 | 35.19 | 35.19 | 34.98 | 35.01 |
| 2 | Tween 60 (Polysorbate | 1.35 | 1.35 | 1.35 | 1.36 | 1.35 | 1.35 | 1.35 | 1.35 | 1.35 | 1.35 |
| | 60) | | | | | | | | | | |
| 3 | Propylene Glycol | 5.00 | 5.00 | 5.00 | 5.00 | 5.00 | 5.00 | 5.00 | 5.00 | 5.00 | 5.01 |
| 4 | Glycerin | — | — | 1.00 | 1.01 | 0.50 | 0.51 | — | — | — | — |
| 5 | Polyethylene Glycol 300 | — | — | 4.00 | 4.01 | 2.00 | 2.01 | — | — | — | — |
| 6 | Myristyl Lactate | — | — | — | — | — | — | 1.00 | 1.01 | 1.00 | 1.00 |
| 7 | Diethylene Glycol | — | — | — | — | — | — | 0.50 | 0.51 | 1.00 | 1.04 |
| | Monoethyl Ether | | | | | | | | | | |
| | (Transcutol-P) | | | | | | | | | | |
| 8 | Cetyl Alcohol | 2.20 | 2.20 | 2.20 | 2.20 | 2.20 | 2.20 | 2.20 | 2.20 | 2.20 | 2.20 |
| 9 | Stearyl Alcohol | 0.75 | 0.75 | 0.75 | 0.76 | 0.75 | 0.75 | 0.75 | 0.75 | 0.75 | 0.75 |
| 10 | Ethanol | 51.85 | 51.84 | 49.44 | 49.43 | 50.94 | 50.93 | 50.72 | 50.69 | 50.42 | 50.34 |
| | TOTAL | 95.45 | 95.44 | 96.70 | 96.71 | 96.70 | 96.70 | 96.71 | 96.70 | 96.70 | 96.70 |
| | Total emollients | 0% | | 4% PEG 300 | | 2% PEG 300 | | 1% Myristyl | | 1% Myristyl | |
| | | | | 1% Glycerin | | 0.5% Glycerin | | lactate | | lactate | |
| | Total Permeation | 0% | | 0% | | 0% | | 0.5% Transcutol | | 1% Transcutol | |
| | enhancer | | | | | | | | | | |
| | Alkalizing agent, TEA | 0% | | 0% | | 0% | | 0% | | 0% | |
| | 99% | | | | | | | | | | |
| | pH of the base, no | 6.19 | | 6.72 | | 6.69 | | 5.02 | | 5.03 | |
| | adjustment | | | | | | | | | | |

TABLE 40
Placebo formulations

| | | Formulation Lot | | | | | | | | | |
|---|---|---|---|---|---|---|---|---|---|---|---|
| | | 268-16-01 | | 268-16-19 | | 268-16-20 | | 268-16-04 | | 268-16-05 | |
| | | PRODUCT DESCRIPTION | | | | | | | | | |
| | | | | | | | | PLACEBO HE | | PLACEBO HE | |
| | | PLACEBO HE | | PLACEBO HE | | PLACEBO HE | | FOAM 1% | | FOAM 1% | |
| | | FOAM NIL | | FOAM 5% | | FOAM 2.5% | | Emollient + 0.5% | | Emollient + 1% | |
| | | Emollient | | Emollient | | Emollient | | Penetration enhancer | | Penetration enhancer | |
| | | | % w/w | | % w/w | | % w/w | | % w/w | | % w/w |
| | | % w/w | By | % w/w | By | % w/w | By | % w/w | By | % w/w | By |
| ITEM# | Ingredients | Required | Input | Required | Input | Required | Input | Required | Input | Required | Input |
| 1 | Water | 34.30 | 34.30 | 32.96 | 32.94 | 33.96 | 33.95 | 35.19 | 35.19 | 34.98 | 35.01 |
| 2 | Tween 60 (Polysorbate | 1.35 | 1.35 | 1.35 | 1.36 | 1.35 | 1.35 | 1.35 | 1.35 | 1.35 | 1.35 |
| | 60) | | | | | | | | | | |
| 3 | Propylene Glycol | 5.00 | 5.00 | 5.00 | 5.00 | 5.00 | 5.00 | 5.00 | 5.00 | 5.00 | 5.01 |
| 4 | Glycerin | — | — | 1.00 | 1.01 | 0.50 | 0.51 | — | — | — | — |
| 5 | Polyethylene Glycol | — | — | 4.00 | 4.01 | 2.00 | 2.01 | — | — | — | — |
| | 300 | | | | | | | | | | |
| 6 | Myristyl Lactate | — | — | — | — | — | — | 1.00 | 1.01 | 1.00 | 1.00 |
| 7 | Diethylene Glycol | — | — | — | — | — | — | 0.50 | 0.51 | 1.00 | 1.04 |
| | Monoethyl Ether | | | | | | | | | | |
| | (Transcutol-P) | | | | | | | | | | |
| 8 | Cetyl Alcohol | 2.20 | 2.20 | 2.20 | 2.20 | 2.20 | 2.20 | 2.20 | 2.20 | 2.20 | 2.20 |
| 9 | Stearyl Alcohol | 0.75 | 0.75 | 0.75 | 0.76 | 0.75 | 0.75 | 0.75 | 0.75 | 0.75 | 0.75 |
| 10 | Ethanol | 51.85 | 51.84 | 49.44 | 49.43 | 50.94 | 50.93 | 50.72 | 50.69 | 50.42 | 50.34 |
| | TOTAL | 95.45 | 95.44 | 96.70 | 96.71 | 96.70 | 96.70 | 96.71 | 96.70 | 96.70 | 96.70 |
| | Total emollients | 0% | | 4% PEG 300 | | 2% PEG 300 | | 1% Myristyl | | 1% Myristyl | |
| | | | | 1% Glycerin | | 0.5% Glycerin | | lactate | | lactate | |
| | Total Permeation | 0% | | 0% | | 0% | | 0.5% Transcutol | | 1% Transcutol | |
| | enhancer | | | | | | | | | | |
| | Alkalizing agent, TEA | 0% | | 0% | | 0% | | 0% | | 0% | |
| | 99% | | | | | | | | | | |
| | pH of the base, no | 6.19 | | 6.72 | | 6.69 | | 5.02 | | 5.03 | |
| | adjustment | | | | | | | | | | |

TABLE 41
Ruxolitinib phosphate formulations

| | | Formulation Lot | | | |
|---|---|---|---|---|---|
| | | 268-16-06 | 268-16-07 | 268-16-08 | 268-16-09 |
| | | PRODUCT DESCRIPTION | | | |
| | | | | 2.5% | 1.5% |
| | | 2.5% | 1.5% | Ruxolitinib | Ruxolitinib |
| | | Ruxolitinib | Ruxolitinib | Foam 5% | Foam 5% |
| | | Foam (NE) | Foam (NE) | Emollient | Emollient |
| | | % w/w | % w/w | % w/w | % w/w |
| ITEM# | Ingredients | Required | Required | Required | Required |
| 1 | Water | 34.30 | 34.83 | 32.46 | 32.99 |
| 2 | Tween 60 (Polysorbate 60) | 1.35 | 1.35 | 1.35 | 1.35 |
| 3 | Propylene Glycol | 5.00 | 5.00 | 5.00 | 5.00 |
| 4 | Glycerin | — | — | 1.00 | 1.00 |
| 5 | Polyethylene Glycol | — | — | 4.00 | 4.00 |
| | (PEG 300) | | | | |
| 6 | Myristyl Lactate | — | — | — | — |
| 7 | Diethylene Glycol Monoethyl | — | — | — | — |
| | Ether (Transcutol-P) | | | | |
| 8 | Cetyl Alcohol | 2.20 | 2.20 | 2.20 | 2.20 |
| 9 | Stearyl Alcohol | 0.75 | 0.75 | 0.75 | 0.75 |
| 10 | Ethanol 100HG | 51.85 | 52.64 | 48.69 | 49.48 |
| 11 | Ruxolitinib Phosphate, A/S | 3.30 | 1.98 | 3.30 | 1.98 |
| 12 | Triethanolamine 99% USP | 1.25 | 1.25 | 1.25 | 1.25 |
| 1 | Water | 34.30 | 34.83 | 32.46 | 32.99 |
| 2 | Tween 60 (Polysorbate 60) | 1.35 | 1.35 | 1.35 | 1.35 |
| 3 | Propylene Glycol | 5.00 | 5.00 | 5.00 | 5.00 |
| 4 | Glycerin | — | — | 1.00 | 1.00 |
| 5 | Polyethylene Glycol | — | — | 4.00 | 4.00 |
| | (PEG 300) | | | | |
| | TOTAL | 100.00 | 100.00 | 100.00 | 100.00 |
| | Total emollients | 0% | 0% | 4% PEG 300 | 4% PEG 300 |
| | | | | 1% Glycerin | 1% Glycerin |
| | Total Permeation enhancer | 0% | 0% | 0% | 0% |
| | pH of the base, spec 5-6 | 5.55 (n = 2) | 5.72 | 5.69 | 5.60 |

TABLE 42
Ruxolitinib phosphate formulations

| | | Formulation Lot | | | | | |
|---|---|---|---|---|---|---|---|
| | | 268-16-10 | 268-16-11 | 268-16-12 | 268-16-13 | 268-16-14 | 268-16-15 |
| | | PRODUCT DESCRIPTION | | | | | |
| | | 2.5% | 1.5% | 2.5% | 1.5% | 2.5% | 1.5% |
| | | Ruxolitinib | Ruxolitinib | Ruxolitinib | Ruxolitinib | Ruxolitinib | Ruxolitinib |
| | | Foam 2.5% | Foam 2.5% | Foam 1% | Foam 1% | Foam 1% | Foam 1% |
| ITEM | | Emollient | Emollient | Emollient | Emollient | Emollient | Emollient |
| # | Ingredients | % w/w Req'd | % w/w Req'd | % w/w Req'd | % w/w Req'd | % w/w Req'd | % w/w Req'd |
| 1 | Purified Water | 33.46 | 33.99 | 34.71 | 35.21 | 34.47 | 35.01 |
| 2 | Tween 60 (Polysorbate 60) | 1.35 | 1.35 | 1.35 | 1.35 | 1.35 | 1.35 |
| 3 | Propylene Glycol | 5.00 | 5.00 | 5.00 | 5.00 | 5.00 | 5.00 |
| 4 | Glycerin | 0.50 | 0.50 | — | — | — | — |
| 5 | Polyethylene Glycol 300 | 2.00 | 2.00 | — | — | — | — |
| 6 | Myristyl Lactate | — | — | 1.00 | 1.00 | 1.00 | 1.00 |
| 7 | Diethylene Glycol Monoethyl Ether | — | — | 0.50 | 0.50 | 1.00 | 1.00 |
| | Transcutol-P) | | | | | | |
| 8 | Cetyl Alcohol | 2.20 | 2.20 | 2.20 | 2.20 | 2.20 | 2.20 |
| 9 | Stearyl Alcohol | 0.75 | 0.75 | 0.75 | 0.75 | 0.75 | 0.75 |
| 10 | Ethanol 100HG | 50.19 | 50.98 | 49.94 | 50.76 | 49.68 | 50.46 |
| 11 | Ruxolitinib Phosphate, A/S | 3.30 | 1.98 | 3.30 | 1.98 | 3.30 | 1.98 |
| 12 | Triethanolamine 99% USP | 1.25 | 1.25 | 1.25 | 1.25 | 1.25 | 1.25 |
| | TOTAL | 100.00 | 100.00 | 100.00 | 100.00 | 100.00 | 100.00 |
| | Total emollients | 2% PEG 300 | 2% PEG 300 | 1% Myristyl | % Myristyl | % Myristyl | % Myristyl |
| | | 0.5% Glycerin | 0.5% Glycerin | lactate | lactate | lactate | lactate |
| | Total Permeation enhancer | 0% | 0% | 0.5% Trans- | 0.5% Trans- | 1% Trans- | 1% Trans- |
| | | | | cutol-P | cutol-P | cutol-P | cutol-P |
| | pH of the base, spec 5-6 | 5.70 | 5.78 | 5.80 | 5.64 | 5.62 | 5.68 |

TABLE 43
Deuruxolitinib phosphate formulations

| | | Formulation Lot | | | |
|---|---|---|---|---|---|
| | | 268-18-01 | 268-18-07 | 268-18-03 | 268-18-08 |
| | | PRODUCT DESCRIPTION | | | |
| | | 1.5% | 2.5% | 1.5% | 2.5% |
| | | Deuterated | Deuterated | Deuterated | Deuterated |
| | | Ruxolitinib | Ruxolitinib | Ruxolitinib | Ruxolitinib |
| | | FOAM (NE) | FOAM (NE) | FOAM (HE) | FOAM (HE) |
| | | % w/w | % w/w | % w/w | % w/w |
| ITEM# | Ingredients | Required | Required | Required | Required |
| 1 | Water | 34.90 | 34.29 | 32.98 | 32.51 |
| 2 | Tween 60 (Polysorbate 60) | 1.35 | 1.35 | 1.35 | 1.35 |
| 3 | Propylene Glycol | 5.00 | 5.00 | 5.00 | 5.00 |
| 4 | Glycerin | — | — | 1.00 | 1.00 |
| 5 | Polyethylene Glycol | — | — | 4.00 | 4.00 |
| | (PEG 300) | | | | |
| 6 | Myristyl Lactate | — | — | — | — |
| 7 | Diethylene Glycol Monoethyl | — | — | — | — |
| | Ether (Transcutol-P) | | | | |
| 8 | Cetyl Alcohol | 2.20 | 2.20 | 2.20 | 2.20 |
| 9 | Stearyl Alcohol | 0.75 | 0.75 | 0.75 | 0.75 |
| 10 | Ethanol 100HG | 52.55 | 51.88 | 49.47 | 48.71 |
| 11 | Deuruxolitinib Phosphate, | 2.051 | 3.281 | 2.051 | 3.281 |
| | A/S | | | | |
| 12 | Triethanolamine 99% USP | 1.20 | 1.25 | 1.20 | 1.20 |
| | TOTAL | 100.00 | 100.00 | 100.00 | 100.00 |
| | Total emollients | 0% | 0% | 4% PEG 300 | 4% PEG 300 |
| | | | | 1% Glycerin | 1% Glycerin |
| | Total Permeation enhancer | 0% | 0% | 0% | 0% |

TABLE 44
Deuruxolitinib phosphate formulations

| | | Formulation ID | | | | | |
|---|---|---|---|---|---|---|---|
| | | 268-18-04 | 268-18-09 | 268-18-05 | 268-18-10 | 268-18-06 | 268-18-11 |
| | | PRODUCT DESCRIPTION | | | | | |
| | | 1.5% | 2.5% | 1.5% | 2.5% | 1.5% | 2.5% |
| | | Deuterated | Deuterated | Deuterated | Deuterated | Deuterated | Deuterated |
| | | Ruxolitinib | Ruxolitinib | Ruxolitinib | Ruxolitinib | Ruxolitinib | Ruxolitinib |
| ITEM | | FOAM (LE) | FOAM (LE) | FOAM (E-1T) | FOAM (E-1T) | FOAM (E-0.5T) | FOAM (E-0.5T) |
| # | Ingredients | % w/w Req'd | % w/w Req'd | % w/w Req'd | % w/w Req'd | % w/w Req'd | % w/w Req'd |
| 1 | Water | 33.98 | 33.49 | 35.00 | 34.49 | 35.20 | 34.74 |
| 2 | Tween 60 (Polysorbate 60) | 1.35 | 1.35 | 1.35 | 1.35 | 1.35 | 1.35 |
| 3 | Propylene Glycol | 5.00 | 5.00 | 5.00 | 5.00 | 5.00 | 5.00 |
| 4 | Glycerin | 0.50 | 0.50 | — | — | — | — |
| 5 | Polyethylene Glycol (PEG | 2.00 | 2.00 | — | — | — | — |
| | 300) | | | | | | |
| 6 | Myristyl Lactate | — | — | 1.00 | 1.00 | 1.00 | 1.00 |
| 7 | Diethylene Glycol Monoethyl | — | — | 1.00 | 1.00 | 0.50 | 0.50 |
| | Ether (Transcutol-P) | | | | | | |
| 8 | Cetyl Alcohol | 2.20 | 2.20 | 2.20 | 2.20 | 2.20 | 2.20 |
| 9 | Stearyl Alcohol | 0.75 | 0.75 | 0.75 | 0.75 | 0.75 | 0.75 |
| 10 | Ethanol 100HG | 50.97 | 50.23 | 50.45 | 49.73 | 50.75 | 49.98 |
| 11 | Deuruxolitinib Phosphate, | 2.051 | 3.281 | 2.051 | 3.281 | 2.051 | 3.281 |
| | A/S | | | | | | |
| 12 | Triethanolamine 99% USP | 1.20 | 1.20 | 1.20 | 1.20 | 1.20 | 1.20 |
| | TOTAL | 100.00 | 100.00 | 100.00 | 100.00 | 100.00 | 100.00 |
| | Total emollients | 2% PEG 300 | 2% PEG 300 | 1% Myristyl | 1% Myristyl | 1% Myristyl | 1% Myristyl |
| | | 0.5% Glycerin | 0.5% Glycerin | lactate | lactate | lactate | lactate |
| | Total Permeation enhancer | 0% | 0% | 1.0% Transcutol- | 1.0% Transcutol- | 0.5% Transcutol- | 0.5% Transcutol- |
| | | | | P | P | P | P |

### Example 11F: Stability of Formulations in Tables 45-51

As provided in Table 45, analytical test results at timepoints zero and 3-months reported Ruxolitinib is stable to 3 months 25° C./60% RH and 40° C./75% RH. Monitoring of samples in glass aerosol bottles at every timepoint reveal physical inhomogeneity in certain samples at 1 month 40° C./75% RH. The inhomogeneity has not impacted the chemical stability, the samples easily redisperse upon container agitation prior to use.

TABLE 45
Stability formulations in Tables 46-51.

| | Appearance at | T = 1 month, |
|---|---|---|
| Stability Samples | ambient, T = 0 | 40° C./75% RH |
| 268-16-06/268-16-06 | Clear | Clear uniform, same |
| | | appearance after shaking |
| 268-16-07/268-16-07 | Clear | Clear uniform, same |
| | | appearance after shaking |
| 268-16-08/268-16-08 | Hazy | Hazy layer on surface; |
| | | slight haze after shaking |
| 268-16-09/268-16-09 | Hazy | Hazy layer on surface; |
| | | slight haze after shaking |
| 268-16-10/268-16-10 | Hazy | Hazy layer on surface; |
| | | slight haze after shaking |
| 268-16-11/268-16-11 | Hazy | Hazy layer on surface; |
| | | slight haze after shaking |
| 268-16-12/268-16-12 | Clear | Clear uniform, same |
| | | appearance after shaking |
| 268-16-13/268-16-13 | Clear | Clear uniform, same |
| | | appearance after shaking |
| 268-16-14/268-16-21 | Clear | Clear uniform, same |
| | | appearance after shaking |
| 268-16-15/268-16-22 | Clear | Clear uniform, same |
| | | appearance after shaking |

### Example 11G: Investigation into Propellants to Improve Stability

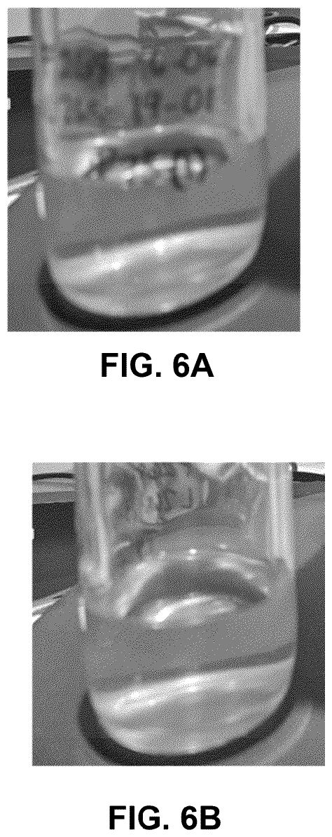

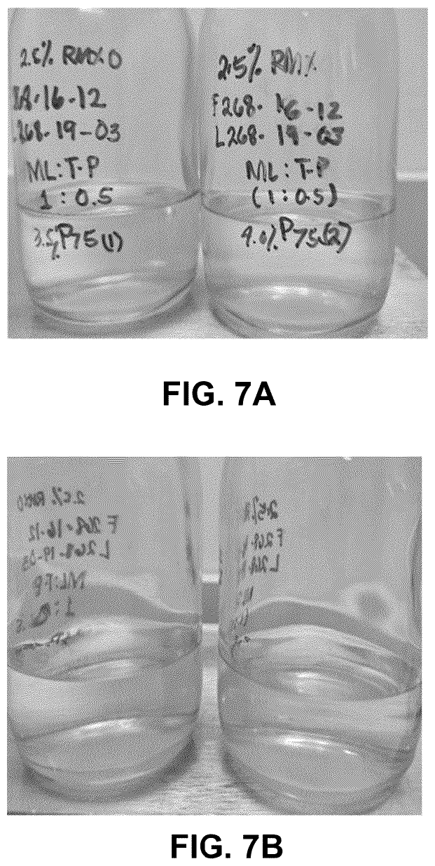

Base formulations with 2.5% (w/w) of ruxolitinib phosphate on free base basis were manufactured as described below and in the Tables 46 and 47 below, as prepared by manufacturing Method B. FIGS. 6A and 6B show the appearance of the foamable compositions of F268-16-06 Lot 268-19-01 with 4.18% Gas P75 with front and rear view, respectively. FIGS. 7A and 7B show the appearance of the foamable compositions from F268-16-12 Lot 268-19-03, 1% emollient & Transcutol-P foams, with 3.5% and 4.0% P75 propellant, respectively.
a) 268-19-01, nil emollient (NE)
b) 268-19-02, 5% emollient (HE)
c) 268-19-03, 1% emollient with Transcutol-P (1-0.5T)

Samples were filled in glass aerosol bottles and gassed with P45 (low to medium pressure HC propellant) and/or P75 (high pressure HC propellant) and screen miscibility.

The miscibility of the base 268-19-01 (no emollient) with a medium pressure propellant P45 and with high pressure P75 propellant was screened in the following 5 samples:
i) Foam samples with increasing P45 levels i.e., 3%, 4% and 5%
ii) A foam sample gassed with 3.2% P45 and topped up with 4.2% P75
ii) A foam sample gassed with 4.18% P75

All 5 samples showed no layer formed on the foam surface indicative that base is miscible with propellants P45 and P75. All 5 samples however, showed fine white precipitates present at the bottom of the bottle. The two-lead formulations—i.e., the 3.0% P45 and the 4.18% P75 samples-showed foam clarity while at rest and when agitated, ambient and/or at 40° C. Both samples showed minimal (hardly visible) precipitation and when reexamined after 10 days at ambient resulted in the same observations. The precipitation suspected to be due to limited solubility of the fatty alcohols in alcoholic media, can therefore be resolved by reducing the levels of cetyl and/or stearyl alcohol in the HE formulations. P75 is the preferred propellant for the Ruxolitinib HE formulation, 4% P75 as optimal level and possibly at reduced levels of the fatty alcohols, a more physically stable ruxolitinib foam can be achieved.

The formulations in Table 47 were investigated:
i) Lot 268-19-02, 5% emollient (HE) and
ii) Lot 268-19-03, 1% emollient with Transcutol-P (1-0.5T)

Samples of the above two formulations with respective emollients are filled in glass aerosol bottles and gassed with propellant P75 each at levels 3.5% and 4.0%. The 268-16-08 Lot 268-19-02 foam samples gassed with 3.5% and 4.0% P75 both appeared slightly hazy and showing 2-mm clear layer on top surface indicating immiscibility. The foams turned more hazy upon agitation, no precipitates were observed at the bottom in any of the 2 samples.

The F 268-16-12 Lot 268-19-03 foam samples also gassed with 3.5% and 4.0% P75, both samples appeared clear and miscible. In both samples there was no layer formation on the foam surface and there was no precipitation observed. The foams remained fluid and clear, homogeneous, and miscible upon agitation.

P75 is an excellent propellant for F268-16-12 L268-19-03 formulation containing myristyl lactate and Transcutol-P at 3.5% to 4.0% P75. The foams appeared clear and fully miscible, no layer on the surface and no precipitate were observed present, the product solution remained clear upon agitation. F268-16-08 L268-19-02, 5% emollient samples gassed with 3.5% or 4.0% of P75 propellant resulted in clear layer formed on the surface of each foam.

TABLE 46
Additional formulations investigated.

| | | FORMULATION & LOT NUMBER | | | |
|---|---|---|---|---|---|
| | | 268-16-06/268-16-06 | | 268-16-06/Lot 268-19-01 | |
| | | Rationale/Iteration | | | |
| | | Reference | | Clear layer investigation | |
| | | Date of Manufacture (DOM) | | | |
| Item | | 23 Nov. 2023 | | 4 Apr. 2024 | |
| No | Raw Material Trade Name | % w/w | Batch % w/w | % w/w | Batch % w/w |

A. WATER PHASE (WP)

| | | | | | |
|---|---|---|---|---|---|
| 1 | Water | 34.30 | 34.32 | 34.30 | 34.41 |
| 2 | SP Tween 60 MBAL | 1.35 | 1.35 | 1.35 | 1.36 |
| 3 | Propylene glycol, BP/USP | 5.00 | 5.01 | 5.00 | 5.02 |
| | Sub-total, Water Phase | 40.65 | 40.68 | 40.65 | 40.78 |

B. OIL PHASE (OP)

| | | | | | |
|---|---|---|---|---|---|
| 4 | Cetyl alcohol 98-100% | 2.20 | 2.20 | 2.20 | 2.20 |
| 5 | Stearyl alcohol 98-100% | 0.75 | 0.75 | 0.75 | 0.75 |
| 6 | MBAL Span 20 | — | — | — | — |
| | Sub-total, Oil Phase | 2.95 | 2.95 | 2.95 | 2.949 |

C. FINAL PHASE (Active + Solvent)

| | | | | | |
|---|---|---|---|---|---|
| 7 | Ethanol 96.5% | 51.85 | 51.82 | 51.85 | 51.77 |
| 8 | Ruxolitinib Phosphate | 3.30 | 3.32 | 3.30 | 3.293 |
| 9 | Triethanolamine 99% USP, | 1.25 | 1.22 | 1.25 | 1.21 |
| | pH 5.0 to 6.0 | | | | |
| | Sub-total, Final Phase | 56.40 | 56.37 | 56.40 | 56.27 |
| | TOTAL qs to | 100.00 | 100.00 | 100.00 | 100.00 |
| | Ruxolitinib Freebase | 2.501 | 2.52 | 2.501 | 2.496 |
| | (F = 0.758) | | | | |
| | Total E + W | 86.15 | 86.15 | 86.15 | 86.17 |
| | % ratio E:W | 60:40 | 60:40 | 60:40 | 60:40 |

TABLE 47
Additional formulations investigated.

| | | REFERENCE FORMULATION & LOT # | | | |
|---|---|---|---|---|---|
| | | F 268-16-08/Lot 268-19-02 | | F 268-16-12/Lot 268-19-03 | |
| | | Date of Manufacture (DOM) | | | |
| | | 8 Apr. 2024 | | 10 Apr. 2024 | |
| Item # | Raw Material Trade Name | % w/w | Batch % w/w | % w/w | Batch % w/w |

A. WATER PHASE (WP)

| | | | | | |
|---|---|---|---|---|---|
| 1 | Water | 32.46 | 32.79 | 34.71 | 34.71 |
| 2 | SP Tween 60 MBAL | 1.35 | 1.35 | 1.35 | 1.35 |
| 3 | Propylene glycol, | 5.00 | 4.98 | 5.00 | 5.00 |
| | BP/USP | | | | |
| 4 | Glycerin | 1.00 | 1.00 | — | — |
| | Sub-total, Water Phase | 39.81 | 40.11 | 41.06 | 41.065 |

B. OIL PHASE (OP)

| | | | | | |
|---|---|---|---|---|---|
| 4 | SP Crodamol ML MBAL | — | — | 1.00 | 1.00 |
| 5 | Transcutol-P | — | — | 0.50 | 0.51 |
| 6 | Cetyl alcohol 98-100% | 2.20 | 2.19 | 2.20 | 2.20 |
| 7 | Stearyl alcohol 98-100% | 0.75 | 0.75 | 0.75 | 0.75 |
| | PEG -6/KOLLISOLV 300 | 4.00 | 3.98 | — | — |
| | Sub-total, Oil Phase | 6.95 | 6.921 | 4.45 | 4.45 |

C. FINAL PHASE (Active + Solvent)

| | | | | | |
|---|---|---|---|---|---|
| 8 | Ethanol 100 HG | 48.69 | 48.44 | 49.94 | 49.93 |
| 9 | Ruxolitinib | 3.30 | 3.30 | 3.30 | 3.31 |
| | Phosphate, A/S | | | | |
| 10 | Triethanolamine 99% USP | 1.25 | 1.23 | 1.25 | 1.24 |
| | Sub-total, Final Phase | 53.24 | 52.97 | 54.49 | 54.48 |
| | TOTAL qs to | 100.00 | 100.00 | 100.00 | 100.00 |
| | Equiv Ruxolitinib | 2.501 | 2.501 | 2.501 | 2.509 |
| | free base, CF = 0.578 | | | | |
| | Ethanol + Water by Input | 81.15 | 81.23 | 84.65 | 84.65 |
| | Water by Input | 60.00 | 59.64 | 59.00 | 58.99 |
| | Total E:W ratio | 40.00 | 40.36 | 41.00 | 41.01 |

The formulation containing 5% PEG 300 and Glycerin as emollients and showing physical instability might also be related to the levels of fatty alcohols in the formulations. To investigate this, the following study was conducted: (1) Manufacture 2 formulations with reduced fatty alcohols, Table 48, prepared by manufacturing Method B. (2) Fill base in glass aerosol bottles, gas with propellant P75 at 4.0% level, evaluate miscibility.

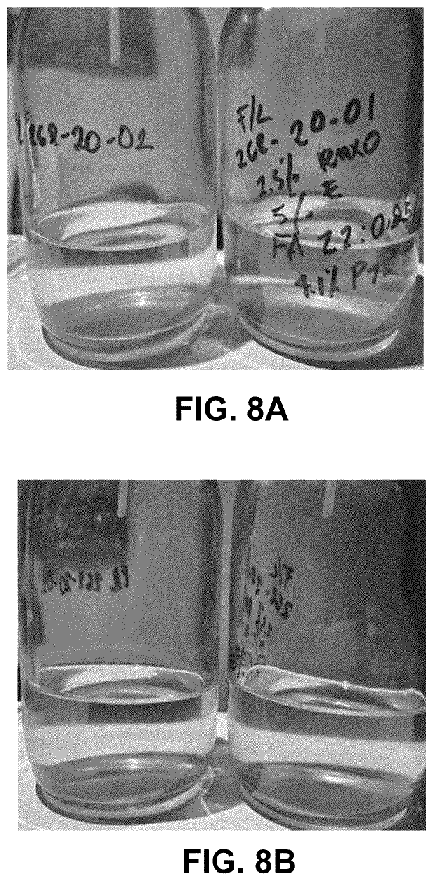

FIGS. 8A and 8B show the appearance of the foamable compositions. That is, in FIG. 8A shows F/L 268-20-02 (with (1.1:0.5) % w/w (C16:C18)), front view (left side) and F/L 268-20-01 (with 2.20:0.25) % w/w (cetyl alcohol:stearyl alcohol)), front view (right side). FIG. 8B shows F/L 268-20-02, back view (left side) and F/L 268-20-01, back view (right side). Both samples 268-20-01 and 268-20-02 appeared clear, practically transparent, no layer formed, and no precipitates present in the foams of 4.0% P75 propellant. Both variations in fatty alcohol levels resulted in fully miscible foam products, FIG. 8.

P75 is excellent propellant for Ruxolitinib foam formulation containing PEG-300 and Glycerin as emollients with 4% as the optimum level for the propellant P75.

By reducing the level of fatty alcohols in the ruxolitinib phosphate HE foams with 5% emollient, 268-20 01 and 268-20-02, precipitation is prevented to occur.

TABLE 48
Additional formulations investigated.

| | | FORMULATION & LOT # | |
|---|---|---|---|
| | | F268-20-01 and L268-20-01 | F268-20-02 and L268-20-02 |
| | | RATIONALE/ITERATION | |
| | | (2.20:0.25) of (C16:C18)% ratio | (1.10:0.50) of (C16:C18)% ratio |
| | | Date of Manufacture (DOM) | |
| Item | Raw Material | 24 Apr. 2024 | 24 Apr. 2024 |

| | | | | | |
|---|---|---|---|---|---|
| No | Trade Name | Equiv weight/ | Batch | Equiv weight/ | Batch |

| | | | | | | |
|---|---|---|---|---|---|---|
| Batch Size, g | % w/w | batch, g | % w/w | % w/w | batch, g | % w/w |

A. WATER PHASE (WP)

| | | | | | | | |
|---|---|---|---|---|---|---|---|
| 1 | Water | 32.66 | 32.63 | 32.55 | 33.00 | 33.28 | 32.99 |
| 2 | SP Tween 60 MBAL | 1.35 | 1.35 | 1.35 | 1.35 | 1.37 | 1.36 |
| 3 | Propylene glycol, | 5.00 | 5.01 | 5.00 | 5.00 | 5.05 | 5.00 |
| | BP/USP | | | | | | |
| 4 | Glycerin | 1.00 | 1.02 | 1.02 | 1.00 | 1.02 | 1.01 |
| | Sub-total, Water Phase | 40.01 | 40.01 | 39.92 | 40.35 | 40.71 | 40.36 |

B. OIL PHASE (OP)

| | | | | | | | |
|---|---|---|---|---|---|---|---|
| 5 | Cetyl alcohol 98-100% | 2.20 | 2.200 | 2.20 | 1.10 | 1.100 | 1.09 |
| 6 | Stearyl alcohol 98- | 0.25 | 0.260 | 0.26 | 0.50 | 0.500 | 0.50 |
| | 100% | | | | | | |
| 7 | PEG -6/KOLLISOLV | 4.00 | 4.00 | 3.99 | 4.00 | 4.02 | 3.99 |
| | 300 | | | | | | |
| | Sub-total, Oil Phase | 6.45 | 6.460 | 6.445 | 5.60 | 5.620 | 5.572 |

C. FINAL PHASE (Active + Solvent)

| | | | | | | | |
|---|---|---|---|---|---|---|---|
| 8 | Ethanol 96.5% | 48.99 | 49.27 | 49.16 | 49.50 | 49.97 | 49.55 |
| 9 | Ruxolitinib Phosphate | 3.30 | 3.3073 | 3.30 | 3.30 | 3.315 | 3.29 |
| 10 | Triethanolamine 99% | 1.25 | 1.18 | 1.18 | 1.25 | 1.24 | 1.23 |
| | USP, pH 5.0 to 6.0 | | | | | | |
| | Sub-total, Final Phase | 53.54 | 53.76 | 53.64 | 54.05 | 54.53 | 54.06 |
| | TOTAL | 100.00 | 100.23 | 100.00 | 100.00 | 100.86 | 100.00 |
| | Ruxolitinib Freebase | 2.501 | — | 2.501 | 2.501 | — | 2.491 |
| | (F = 0.758) | | | | | | |
| | Total E + W by Input | 81.65 | | 81.71 | 82.50 | | 82.54 |
| | (E:W) ratio | 60:40 | | 60:40 | 60:40 | | (60:40) |

### Example 11H: In-Vitro Permeation Testing (IVPT)

The foam formulations were expelled into a glass container with a lid and was left at ambient conditions (lid off) for 5 minutes to allow the propellant to dissipate. The lid was then placed back onto the container (to prevent evaporation of ethanol) and the container was placed into an oven heated to 37° C. for 10 minutes. Once the foam was liquid, it was dosed using a pipette.

The control formulation (ruxolitinib phosphate cream, 1.5%) was applied using a syringe.

IVPT experiments were performed on 10 batches of the foam formulations containing ruxolitinib (n=4) (Tables 41 and 42), 1 control formulation (ruxolitinib cream 1.5%) (n=4) and 5 placebo formulations (n=1) (Tables 39 and 40) on 3 skin donors using Test Conditions A (see Materials and Methods).

Further IVPT experiments were performed on 10 batches of the foam formulation containing deuruxolitinib phosphate (n=4) (Tables 43 and 44), 1 control formulation (ruxolitinib cream 1.5%) (n=4) and 5 placebo formulations (n=1) (Tables 39 and 40) on 3 skin donors using Test Conditions B (see Materials and Methods).

The control formulation was the commercially available ruxolitinib cream 1.5%.

Deuruxolitinib phosphate was prepared as described in the Materials and Methods section.

### Results:

### 1.5% Ruxolitinib Phosphate Foams:

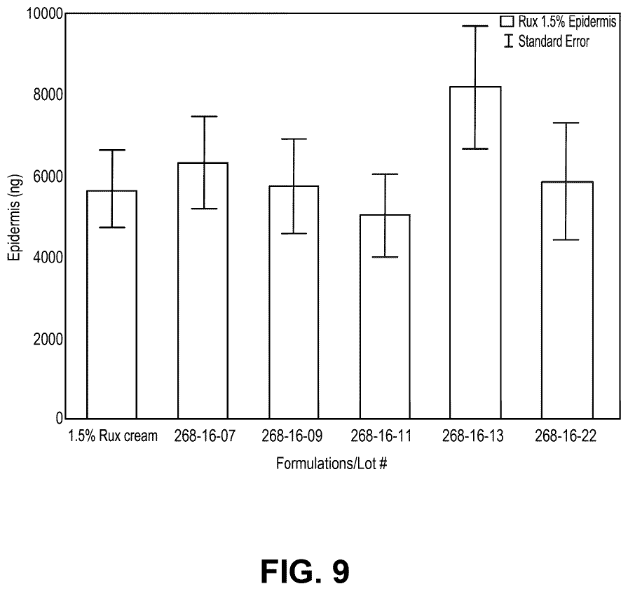

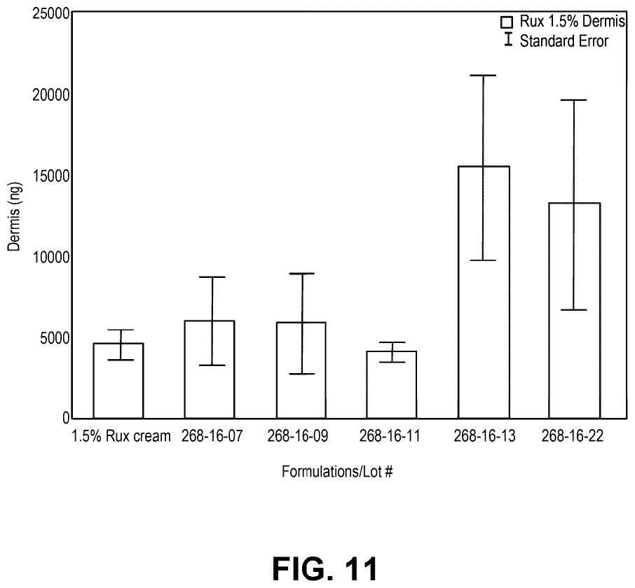

The mean cumulative amount (ng) of ruxolitinib recovered from the epidermis and dermis is presented in FIGS. 9 and 11, respectively.

As a general trend, lot 268-16-13 resulted in the most ruxolitinib being recovered from the epidermis and dermis with mean cumulative amounts of 8140.91 ng and 15435.83 ng, respectively, while lot 268-16-11 delivered the least ruxolitinib with mean cumulative amounts of 5031.00 ng and 4072.00 ng from the epidermis and dermis, respectively. However, there were no significant differences in the amounts of ruxolitinib recovered from the epidermis and dermis of all the formulations tested.

### 2.5% Ruxolitinib Phosphate Foams:

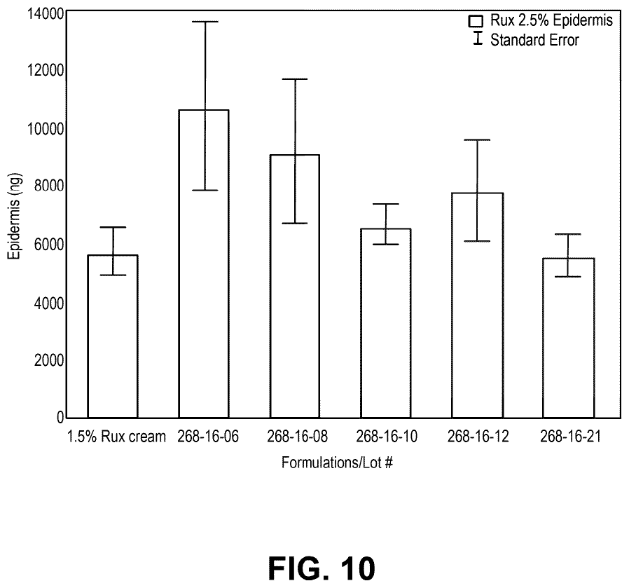

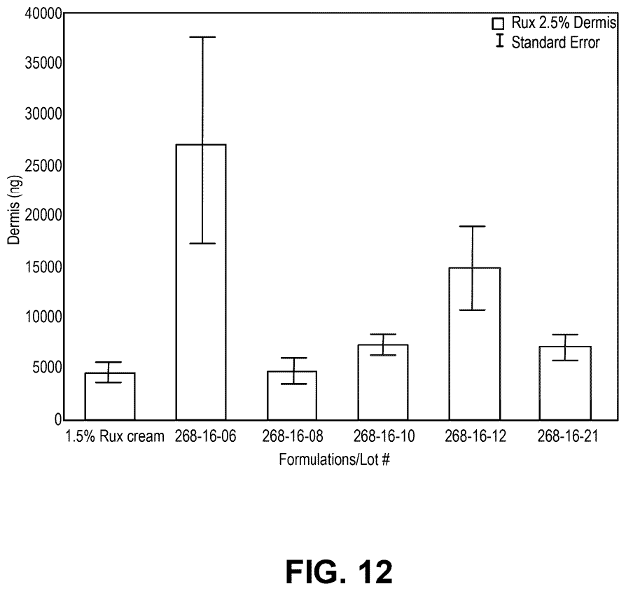

The mean cumulative amount (ng) of ruxolitinib recovered from the epidermis and dermis is presented in FIGS. 10 and 12, respectively.

There were no significant differences in the mean cumulative amounts of ruxolitinib recovered from the epidermis between all the tested formulations; however, 268-16-06 resulted in significantly more (p<0.02) ruxolitinib being recovered from the dermis compared to lot 268-16-08 and ruxolitinib cream 1.5%.

### 1.5% Deuruxolitinib Phosphate Foams:

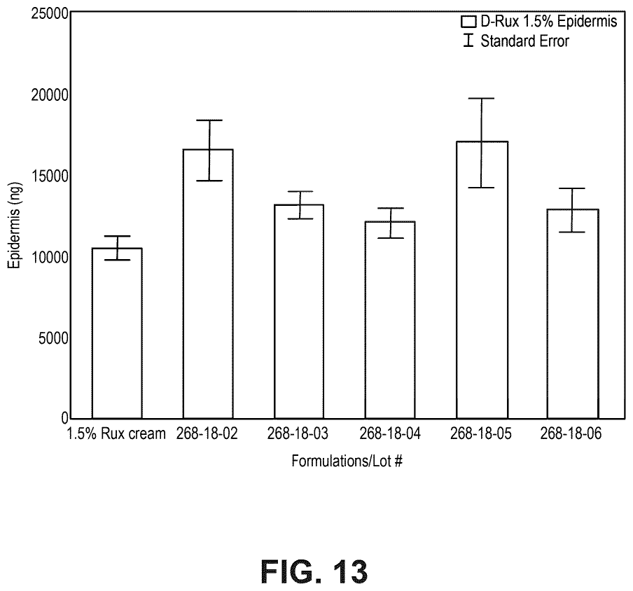

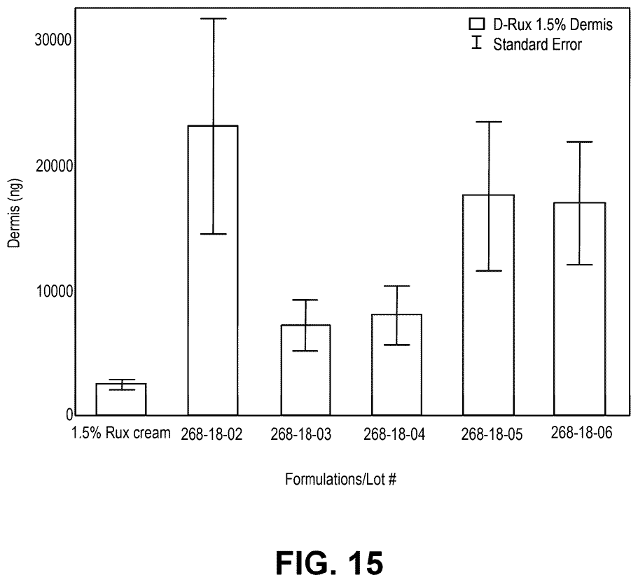

The mean cumulative amount (ng) of ruxolitinib recovered from the epidermis and dermis is presented in FIGS. 13 and 15, respectively.

There were no significant differences in the mean cumulative amounts of deuterated ruxolitinib and ruxolitinib recovered from the epidermis of all the 1.5% formulations tested. However, lots 268-16-02, 268-16-06 and 268-16-05 resulted in more (p<0.01) deuterated ruxolitinib being recovered from the dermis compared to the amount of ruxolitinib recovered from the control formulation, with 268-16-02 resulting in the most deuterated ruxolitinib (23076 ng) being recovered from the dermis of all the 1.5% formulations.

### 2.5% Deuruxolitinib Phosphate Foams:

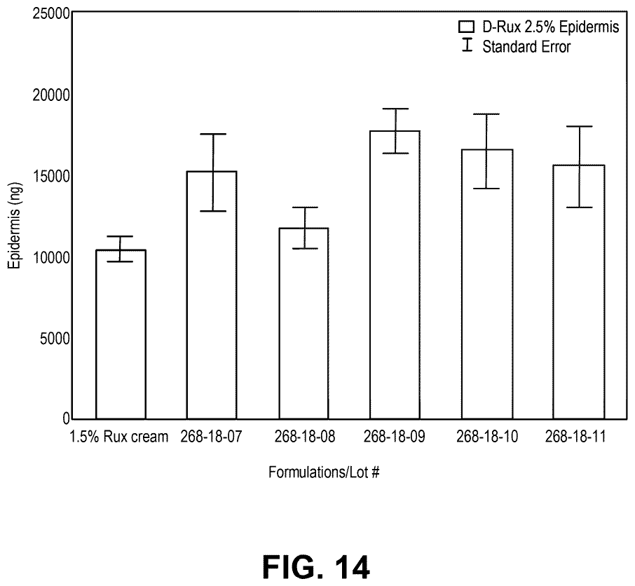

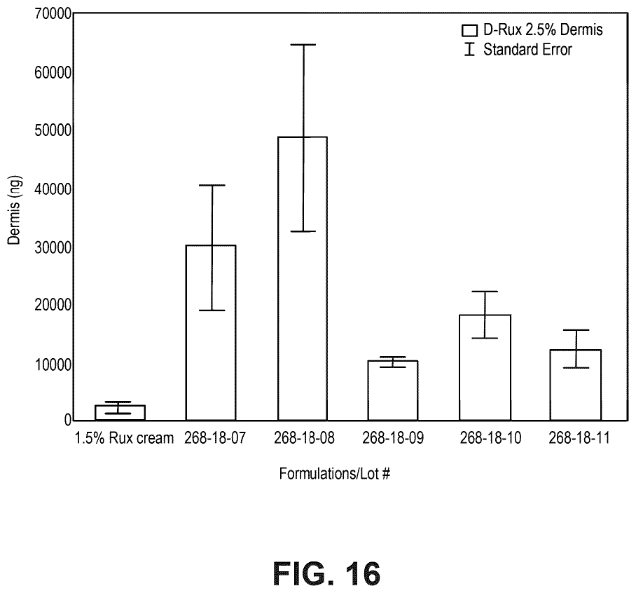

The mean cumulative amount (ng) of ruxolitinib recovered from the epidermis and dermis is presented in FIGS. 14 and 16, respectively.

Compared to the amount of ruxolitinib recovered from the control formulation, batch 268-16-09 had more (p<0.05) deuterated ruxolitinib recovered from the epidermis. There were no other significant differences in the amount of deuterated ruxolitinib and ruxolitinib recovered from the epidermis between all remaining 2.5% formulations and the control formulation. Batch 268-16-08 resulted in the highest mean cumulative amount (48666 ng) of deuterated ruxolitinib recovered from the dermis compared to the remaining 2.5% formulations as well as ruxolitinib cream (1.5%); however, all foam formulations resulted in significantly more (p<0.01) deuterated ruxolitinib being recovered from the dermis than that of ruxolitinib recovered from ruxolitinib cream (1.5%).

### Example 11I: Evaluation of Foam Formulations in Acute AA Lesional Skin Organ Cultures

Using the procedures described in the Materials and Methods section, the foam formulations were evaluated ex vivo. Skin punch biopsies measuring 4 mm were obtained from acute Alopecia Areata (AA) patients. Each skin punch biopsy was topically treated with either placebo foam (vehicle; Table 39, Lot 268-16-01) or ruxolitinib phosphate foam (Table 41, Lot 268-16-07, hereinafter referred to API-1). The foam was consistently weighed at the time of each topical treatment. Photographs were captured at the beginning, midpoint, and conclusion of the skin organ culture. The skin punch biopsies were weighed before the start of the organ culture.

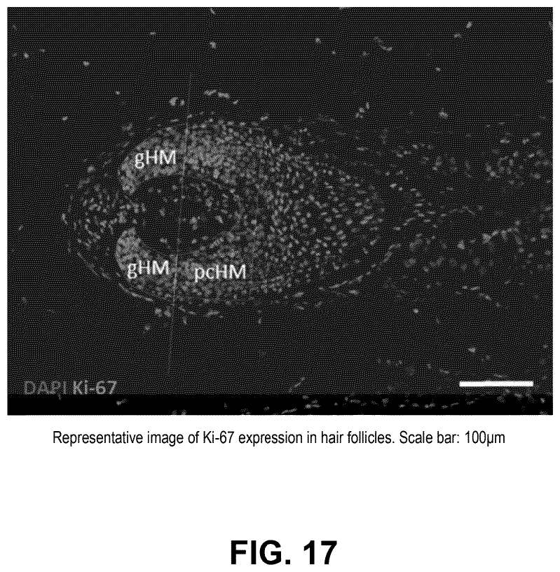

Proliferation of germinative hair matrix keratinocytes is directly correlated to anagen, as catagen induction results in decreased proliferation (Langan et al., Exp Dermatol 2015). The hair matrix is the part of the hair follicle where matrix keratinocytes proliferate to form the hair shaft of growing hair (Martel et al., Anatomy, Hair Follicle, 2022). Increase in proliferation amongst anagen HFs suggests that the treatment may have direct effects on hair matrix keratinocyte proliferation. Ki-67 is a marker of cell proliferation. Skin sections were stained with an antibody against Ki-67 (Cell signaling, Cat. 9449) followed by a secondary goat anti-mouse IgG conjugated to Rhodamine Red (Jackson ImmunoResearch Labs, 115-295-062). The germinative hair matrix keratinocyte proliferation is calculated as the percentage of Ki-67 positive nuclei. FIG. 17 shows a representative image of Ki-67 expression in hair follicles (scale bar: 100 μm).

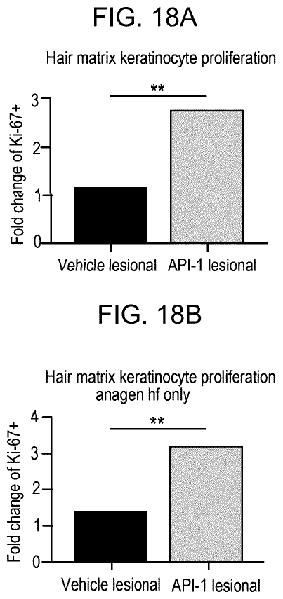

Treating ex vivo biopsy of alopecia areata lesional skin with vehicle and API-1 foam. Samples treated with the API-1 foam showed an increase in proliferation of hair matrix keratinocytes for all and anagen hair follicles in alopecia areata lesional skin (FIG. 18). FIG. 18 shows the expression in hair matrix, fold change to normal non-lesional skin: A) hair matrix proliferation by Ki-67 expression for all hair follicles and B) hair matrix prolifertation by Ki-67 expression for anagen cycle hair follicles only (Mann Whitney t-test for significance). This suggests that the treatment may have direct effects on hair matrix keratinocyte proliferation and thus formation of hair shaft for a growing hair follicle in the context of AA disease.

### Example 11J: MHC-I Expression

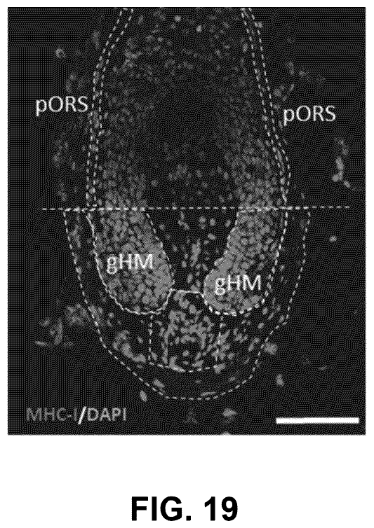

Anagen hair follicles (HFs) exhibit immune privilege (IP) in the lower cycling portion. Collapse of HF IP is an essential prerequisite for the development of alopecia areata (AA) (Bertolini et al., Exp Dermatol 2020). Molecules of the primary classes of major histocompatibility complex (MHC-I) are important IP markers, which are found downregulated in normal HF homeostasis, while upregulated in IP collapse. Increased MHC-I expression skin sections were stained with a monoclonal mouse antibody against human MHC-I (Santa Cruz, Cat. Sc-32235, clone W6/32) followed by a secondary goat anti-mouse IgG conjugated to Rhodamine (Jackson ImmunoResearch Labs, 115-295-062). MHC-I expression was assessed by fluorescence intensity in the follicular proximal outer root sheath (pORS) and germinative hair matrix (gHM) areas. FIG. 19 shows a representative image of MHC-I expression (scale bar: 100 μm).

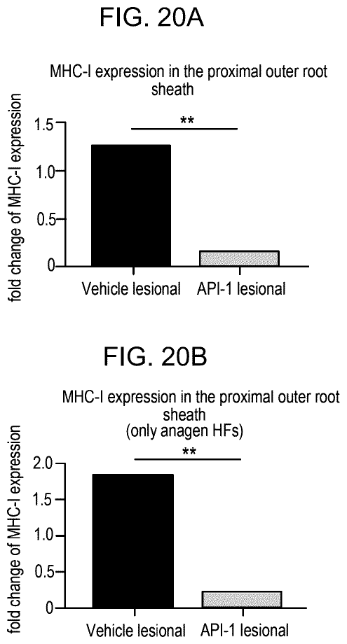

Treating ex vivo biopsy of alopecia areata lesional skin with vehicle and API-1. Samples treated with API-1 showed a decrease in MHC-I expression in outer root sheath for all hair follicles and also anagen hair follicles (FIG. 20). In FIG. 20, MHC-I expression in outer root sheath, fold change to normal non-lesional skin: A) MHC-I expression in outer root sheath for all hair follicles; and B) MHC-I expression in outer root sheath for anagen hair follicles only (Mann Whitney t-test for significance). This suggests that the treatment may have direct effects on decreasing immune privilege collapse and thus development of alopecia areata.

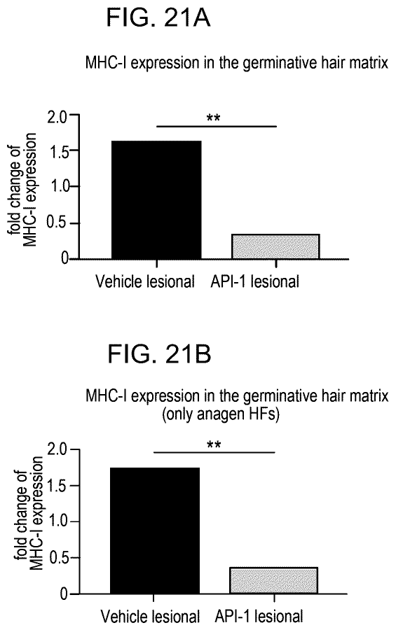

Samples treated with API-1 foam also showed a decrease in MHC-I expression in germinative hair matrix for all hair follicles and also anagen hair follicles (FIG. 21). In FIG. 21, MHC-I expression in outer root sheath, fold change to normal non-lesional skin: A) MHC-I expression in outer root sheath for all hair follicles; and B) MHC-I expression in outer root sheath for anagen hair follicles only (Mann Whitney t-test for significance). This suggests that the treatment may have direct effects on decreasing immune privilege collapse and thus development of alopecia areata.

### Conclusions

The application of a ruxolitinib topical foam to acute AA lesional punches indicate an increase in keratinocyte matrix proliferation. This has a direct correlation to the formation of the hair shaft for growing hair follicles for acute alopecia areata. The decrease in MHC-I expression in both the root sheath and germinative hair matrix indicates a decrease in the immune response for the progression of alopecia.

### Example 12: Sixth Month Stability on Ruxolitinib Foamable Compositions

From Tables 40-42 above, placebo foam and ruxolitinib foam formulations were tested for longer term stability (out to 6 months). HPLC data from the 6-month stability testing results are provided in Tables 49 and 50 below.

TABLE 49
Placebo foam formulations stability HPLC results

| | | | | | sample | P75 | | | % LC | Total |
|---|---|---|---|---|---|---|---|---|---|---|
| SAMPLE | | Formulation/ | % Label | Timepoint, | can, | content, | Storage | % w/w | average, | Impurities, |
| NAME | DESCRIPTION | Lot # | claim | month | # | % w/w | ° C. | Average | (% RSD) | % area |
| Placebo | Nil Emollient | 268-16-01/ | 0 | 0 | 1 | 5.08 | ambient | ND | NA | ND |
| Foam | (NE) | 268-16-01 | | 1 | 9 | 5.15 | 40 | ND | NA | ND |
| | | | | 3 | 7 | 5.06 | 40 | ND | NA | ND |
| | | | | 6 | 11 | 5.00 | 40 | ND | NA | ND |
| Placebo | 5% Emollient | 268-16-19/ | 0 | 0 | 1 | 4.92 | ambient | ND | NA | ND |
| Foam | (HE) | 268-16-19 | | 1 | 9 | 5.07 | 40 | ND | NA | ND |
| | | | | 3 | 7 | 5.11 | 40 | ND | NA | ND |
| | | | | 6 | 6 m evaluation not required | | | | | |
| Placebo | 2.5% Emollient | 268-16-20/ | 0 | 0 | 1 | 5.43 | ambient | ND | NA | ND |
| Foam | (LE) | 268-16-20 | | 1 | 9 | 5.04 | 40 | ND | NA | ND |
| | | | | 3 | 7 | 5.04 | 40 | ND | NA | ND |
| | | | | 6 | 11 | 5.00 | 40 | ND | NA | ND |
| Placebo | 1% Emollient, | 268-16-04/ | 0 | 0 | 1 | 5.53 | ambient | ND | NA | ND |
| Foam | 0.5% Transcutol | 268-16-16 | | 1 | 9 | 4.97 | 40 | ND | NA | ND |
| | (1E-0.5T) | | | 3 | 7 | 4.89 | 40 | ND | NA | ND |
| | | | | 6 | 11 | 5.20 | 40 | ND | NA | ND |
| Placebo | 1% Emollient, | 268-16-05/ | 0 | 0 | 1 | 5.02 | ambient | ND | NA | ND |
| Foam | 1% Transcutol | 268-16-05 | | 1 | 9 | 5.3 | 40 | ND | NA | ND |
| | (1E-1T) | | | 3 | 7 | 4.98 | 40 | ND | NA | ND |

| | |
|---|---|
| 6 | 6 m evaluation not required |

TABLE 50
Ruxolitinib foam formulations stability HPLC results

| | | | | | sample | P75 | | | % LC | | Total |
|---|---|---|---|---|---|---|---|---|---|---|---|
| SAMPLE | | Formulation/ | % Label | Timepoint, | can, | content, | Storage | % w/w | average, | | Impurities, |
| NAME | DESCRIPTION | Lot # | claim | month | # | % w/w | ° C. | Average | (% RSD) | | % area |
| 2.5% | Nil Emollient | 268-16-06/ | 2.5 | 0 | 1, 22 | 5.05, 5.26 | ambient | 2.533 | 101.3 | (0.3) | ND |
| Ruxolitinib | (NE) | 268-16-06 | | 1 | 6 | 5.02 | 5 | 2.503 | 101.1 | (0.5) | ND |
| Foam | | | | | 15 | 4.98 | 40 | 2.515 | 100.6 | (0.7) | ND |
| | | | | 3 | 13 | 5.02 | 25 | 2.521 | 100.8 | (0.1) | ND |
| | | | | | 11 | 4.99 | 40 | 2.522 | 100.9 | (0.6) | ND |
| | | | | 6 | 17 | 5.4 | 25 | 2.546 | 101.9 | (0.0) | ND |
| | | | | | 7 | 5.1 | 40 | 2.551 | 102.0 | (0.3) | ND |
| | | | | | 23S-U | 5.0 | 40 | 2.566 | 102.6 | (0.2) | ND |
| | | | | | 24S-I | 5.0 | | 2.556 | 102.2 | (0.3 | ND |
| 1.5% | Nil Emollient | 268-16-07/ | 1.5 | 0 | 1, 22 | 5.13, 4.90 | ambient | 1.524 | 101.6 | (0.6) | ND |
| Ruxolitinib | (NE) | 268-16-07 | | 1 | 15 | 4.99 | 40 | 1.504 | 100.2 | (0.0) | ND |
| Foam | | | | 3 | 12 | 5.05 | 25 | 1.512 | 100.8 | (0.4) | ND |
| | | | | | 11 | 5.15 | 40 | 1.511 | 100.7 | (0.2) | ND |
| | | | | 6 | 16 | 5.0 | 25 | 1.524 | 101.6 | (0.2) | ND |
| | | | | | 7 | 5.0 | 40 | 1.522 | 101.5 | (0.2) | ND |
| 2.5% | 5% Emollient | 268-16-08/ | 2.5 | 0 | 1, 22 | 4.97, 4.88 | ambient | 2.54 | 101.6 | (0.4) | ND |
| Ruxolitinib | (HE) | 268-16-08 | | 1 | 15 | 4.88 | 40 | 2.514 | 100.6 | (0.6) | ND |
| Foam | | | | 3 | 13 | 5.05 | 25 | 2.523 | 100.9 | (0.3) | ND |
| | | | | | 11 | 4.97 | 40 | 2.553 | 102.1 | (0.3) | ND |
| | | | | 6 | 6 m evaluation not required | | | | | | |
| 1.5% | 5% Emollient | 268-16-09/ | 1.5 | 0 | 1, 22 | 5.3, 5.37 | ambient | 1.546 | 103.0 | (0.3) | ND |
| Ruxolitinib | (HE) | 268-16-09 | | 1 | 15 | 4.83 | 40 | 1.52 | 101.3 | (0.1) | ND |
| Foam | | | | 3 | 13 | 5.03 | 25 | 1.527 | 100.8 | (0.9) | ND |
| | | | | | 11 | 4.94 | 40 | 1.539 | 102.6 | (0.2) | ND |
| | | | | 6 | 6 m evaluation not required | | | | | | |
| 2.5% | 2.5% Emollient | 268-16-10/ | 2.5 | 0 | 1, 22 | 4.89.5.01 | ambient | 2.556 | 102.3 | (0.5) | ND |
| Ruxolitinib | (LE) | 268-16-10 | | 1 | 15 | 5.05 | 40 | 2.531 | 101.2 | (0.7) | ND |
| Foam | | | | 3 | 13 | 4.84 | 25 | 2.523 | 100.9 | (0.5) | ND |
| | | | | | 11 | 4.99 | 40 | 2.536 | 101.4 | (0.2) | ND |
| | | | | 6 | 18 | 5.0 | 5 | 2.539 | 101.5 | (0.6) | ND |
| | | | | | 17 | 5.0 | 25 | 2.551 | 102.1 | (0.3) | ND |
| | | | | | 7 | 5.1 | 40 | 2.556 | 102.3 | (0.0) | ND |
| | | | | | 23S-U | 5.0 | 40 | 2.563 | 102.5 | (0.2) | ND |
| | | | | | 24S-I | 4.9 | | 2.567 | 102.7 | (0.63) | ND |
| 1.5% | 2.5% Emollient | 268-16-11/ | 1.5 | 0 | 1, 22 | 4.98, 4.89 | ambient | 1.515 | 101.0 | (0.1) | ND |
| Ruxolitinib | (LE) | 268-16-11 | | 1 | 15 | 4.95 | 40 | 1.504 | 100.3 | (0.4) | ND |
| Foam | | | | 3 | 13 | 5.02 | 25 | 1.509 | 100.6 | (0.2) | ND |
| | | | | | 11 | 4.99 | 40 | 1.517 | 101.1 | (0.4) | ND |
| | | | | 6 | 17 | 5.1 | 25 | 1.505 | 100.3 | (1.1) | ND |
| | | | | | 7 | 4.9 | 40 | 1.524 | 101.6 | (0.3 | ND |
| 2.5% | 1% Emollient, | 268-16-12/ | 2.5 | 0 | 1, 22 | 4.94, 5.02 | ambient | 2.566 | 102.7 | (0.1) | ND |
| Ruxolitinib | 0.5% T-P | 268-16-12 | | 1 | 15 | 5.07 | 40 | 2.588 | 103.5 | (0.6) | ND |
| Foam | (1E-0.5T) | | | 3 | 13 | 5.19 | 25 | 2.554 | 102.1 | (0.3) | ND |
| | | | | | 11 | 5.28 | 40 | 2.553 | 102.1 | (0.2) | ND |
| | | | | 6 | 17 | 5.1 | 25 | 2.56 | 102.4 | (0.0) | ND |
| | | | | | 7 | 5.0 | 40 | 2.589 | 103.6 | (0.5) | ND |
| | | | | | 23S-U | 5.0 | 40 | 2.591 | 103.6 | (0.4) | ND |
| | | | | | 24S-I | no test sample available | | | | | |
| 1.5% | 1% Emollient, | 268-16-13/ | 1.5 | 0 | 1, 22 | 4.96, 5.0 | ambient | 1.517 | 101.1 | (0.1) | ND |
| Ruxolitinib | 0.5% T-P | 268-16-13 | | 1 | 15 | 5.13 | 40 | 1.491 | 99.4 | (0.2) | ND |
| Foam | (1E-0.5T) | | | 3 | 13 | 5.2 | 25 | 1.507 | 100.5 | (0.5) | ND |
| | | | | | 11 | 4.94 | 40 | 1.512 | 100.8 | (0.2) | ND |
| | | | | 6 | 17 | 5.2 | 25 | 1.512 | 100.8 | (0.4) | ND |
| | | | | | 7 | 5.1 | 40 | 1.522 | 101.5 | (0.3) | ND |
| | | | | | 23S-U | 5.0 | 40 | 1.521 | (101.4 | (0.1) | ND |
| | | | | | 24S-I | 5.0 | | 1.518 | 101.2 | (0.0) | ND |
| 2.5% | 1% Emollient, | 268-16-14/ | 2.5 | 0 | 1, 22 | 5.06, 5.18 | ambient | 2.553 | 102.1 | ((0.5) | ND |
| Ruxolitinib | 1% T-P | 268-16-21 | | 1 | 15 | 5.02 | 40 | 2.519 | 100.8 | (0.2) | ND |
| Foam | (1E-1T) | | | 3 | 14 | 5.15 | 5 | 2.533 | 101.3 | (0.1) | ND |
| | | | | | 13 | 5.04 | 25 | 2.527 | 101.1 | (0.1) | ND |
| | | | | 6 | 11 | 5.06 | 40 | 2.505 | 100.2 | (0.2) | ND |
| 1.5% | 1% Emollient, | 268-16-15/ | 1.5 | 0 | 1, 22 | 5.24, 5.04 | ambient | 1.518 | 101.2 | (0.1) | ND |
| Ruxolitinib | 1% T-P | 268-16-22 | | 1 | 15 | 4.88 | 40 | 1.491 | 99.4 | (0.4) | ND |
| Foam | (1E-1T) | | | 3 | 13 | 4.88 | 25 | 1.509 | 100.6 | (0.6) | ND |
| | | | | | 11 | 5.16 | 40 | 1.514 | 101.0 | (0.0) | ND |

| | |
|---|---|
| 6 | 6 m evaluation not required |

From Table 50, the HPLC results show ruxolitinib foam formulations are chemically stable achieving 90%-110% LC to 6-months study timepoint. In some embodiments, the ruxolitinib foam formulations are stable at a temperature ranging from 5° C. to 40° C. for about 6 months.

Additional stability studies were conducted to determine the stability of the pH of the formulations. Six-month stability data are provided below in Table 51, where timepoints 0, 1, 3, and 6-months are provided with the recorded pH of the formulations with SU=Scratched can, Upright position; SI-Scratched can, Inverted position.

TABLE 51
Six-month pH stability

| | | | Foam samples pH reading 25° C./timepoint, month/s | | | |
|---|---|---|---|---|---|---|
| | | | | | | 6-months- |
| Samples | | | | | | 25° C./40° C./ |
| | Formulation/ | Base pH | | 1-month, | 3-month, | 40° C.-SU/ |
| Product name | Lot | 40° C. | Time 0 | (5° C./40° C.) | (25° C./40° C. | 40° C.-SI |
| Placebo Foam, | 268-16-01/ | 6.19 | 6.12 | NC | NC | 5.80 (40° C.) |
| NE | 268-16-01 | | | | | |
| Placebo Foam, | 268-16-19/ | 6.72 | 6.58 | NC | NC | NC |
| HE | 268-16-19 | | | | | |
| Placebo Foam, | 268-16-20/ | 6.69 | 6.34 | NC | NC | 6.40 (40° C.) |
| LE | 268-16-20 | | | | | |
| Placebo Foam, | 268-16-04/ | 5.02 | 5.27 | NC | NC | 5.21 (40° C.) |
| 1E-0.5T | 268-16-16 | | | | | |
| Placebo Foam, | 268-16-05/ | 5.03 | 5.25 | NC | NC | NC |
| 1E-1T | 268-16-05 | | | | | |
| 2.5% | 268-16-06/ | 5.55 | 5.38 | 5.62/5.36 | 5.59/5.34 | 5.44/5.51/5.47/5.5 |
| Ruxolitinib | 268-16-06 | | | | | |
| Foam, NE | | | | | | |
| 1.5% | 268-16-07/ | 5.72 | 5.51 | 5.74/5.74 | 5.58/5.59 | 5.85/5.81/—/— |
| Ruxolitinib | 268-16-07 | | | | | |
| Foam, NE | | | | | | |
| 2.5% | 268-16-08/ | 5.69 | 5.57 | 6.01/6.07 | 5.77/5.60 | NC |
| Ruxolitinib | 268-16-08 | | | | | |
| Foam, HE | | | | | | |
| 1.5% | 268-16-09/ | 5.60 | 5.53 | 5.94/5.95 | 5.76/5.71 | NC |
| Ruxolitinib | 268-16-09 | | | | | |
| Foam, HE | | | | | | |
| 2.5% | 268-16-10/ | 5.70 | 5.85 | 5.94/5.90 | 5.97/5.77 | 5.82/5.78/5.81/5.71 |
| Ruxolitinib | 268-16-10 | | | | | |
| Foam, LE | | | | | | |
| 1.5% | 268-16-11/ | 5.78 | 5.73 | 6.02/5.97 | 5.77/5.94 | 5.86/5.85/—/— |
| Ruxolitinib | 268-16-11 | | | | | |
| Foam, LE | | | | | | |
| 2.5% | 268-16-12/ | 5.80 | 5.80 | 5.92/5.87 | 5.81/5.65 | 5.88/5.55/5.52/— |
| Ruxolitinib | 268-16-12 | | | | | |
| Foam, 1E-0.5T | | | | | | |
| 1.5% | 268-16-13/ | 5.64 | 5.53 | 5.82/5.73 | 5.58/5.46 | 5.60/5.37/5.30/5.38 |
| Ruxolitinib | 268-16-13 | | | | | |
| Foam, 1E-0.5T | | | | | | |
| 2.5% | 268-16-14/ | 5.62 | 5.42 | 5.75/5.67 | 5.62/5.31/ | NC |
| Ruxolitinib | 268-16-21 | | | | 5.70@ 5° C. | |
| Foam, 1E-1T | | | | | | |
| 1.5% | 268-16-15/ | 5.68 | 5.62 | 5.97/5.94 | 5.63/5.71 | NC |
| Ruxolitinib | 268-16-22 | | | | | |
| Foam, 1E-1T | | | | | | |

18/670394

### Example 13: Ruxolitinib and Deuruxolitinib Foamable Formulations for Alopecia Areata (AA) Evaluation

Two foamable formulations of 1.5% ruxolitinib phosphate (Formulation 268-16-07, referred to herein as “API-1”) and 1.5% deuruxolitinib phosphate (Formulation 268-18-01, referred to herein as “API-2”) were formulated as a topical foam for the testing on an alopecia areata model. In alopecia areata (AA), one of the factors leading to the AA indication is the role of immune cells. That is, hair follicles have an immune privilege where the antigen presenting molecules do not result in an inflammatory response. AA is an inflammatory condition where the immune privilege is not observed resulting in an inflammatory condition leading to hair loss. AP-1 and AP-2 topical ruxolitinib foams were applied topically to excised skin punched in an ex vivo study to examine, e.g., the immune response, toxicity (melanocyte clumping), and keratinocyte proliferation.

For tests performed below, skin punch biopsies measuring 4 mm were obtained from acute (consisting of two lesional and two non-lesional samples) and chronic AA patients (comprising two lesional samples). The samples were cultured at 37° C. within a 5% CO₂ atmosphere, using a minimal medium of William's E media and RPMI 1640 in equal proportions (provided by Gibco, Life Technologies). This medium was further supplemented with 2 mM of L-glutamine (supplied by Gibco), 10 ng/ml hydrocortisone (offered by Sigma Aldrich), 10 μg/ml insulin (distributed by Sigma Aldrich) and a 1% mixture of penicillin/streptomycin (procured from Gibco). The medium thus prepared is referred to as Williams Complete/RPMI Media (WCM+RPMI). Each skin punch biopsy was topically treated on a daily basis with either Placebo foam (Vehicle) or API foam. The foam was consistently weighed at the time of each topical treatment. Photographs (not shown) were captured at the beginning, and conclusion of the skin organ culture. The skin punch biopsies were weighed before the start of the organ culture.

Standard measurements and testing for topical foam treatment: To standardize the procedure of the topical treatment of the foam, a 2 μl sample of liquefied foam, which weighed precisely 1.52 mg, was discharged into a 3.5 cm dish for a precise weight measurement. Employing a 10 μl tip, the foam was removed from the dish twice for each application during the evaluation. A record of the weight change in the foam-filled dish was kept. This procedure was performed ten times each for the Placebo foam, API-1 foam, and API-2 foam.

Experimental Layout Acute Alopecia Areata Patients: 4 experimental groups, 4 mm punch per group

TABLE 52
Groups for Donor 1-1 of acute alopecia areata patient

| Group | Treatment in WCM + RPMI (50:50) |
|---|---|
| #1 | Placebo foam (lesional) |
| #2 | API foam (lesional) |
| #3 | Placebo foam (non-lesional) |
| #4 | API foam (non-lesional) |

TABLE 53
Groups for Donor 4-2 of acute alopecia areata patient

| Group | Treatment in WCM + RPMI (50:50) |
|---|---|
| #1 | Placebo foam (lesional) |
| #2 | API foam (lesional) |
| #3 | Placebo foam (non-lesional) |
| #4 | API foam (non-lesional) |

Experimental Layout Chronic Alopecia Areata Patients: 2 experimental groups,

4 mm punch per group

TABLE 54
Groups for all chronic donors of chronic alopecia areata patients

| Group | Treatment in WCM + RPMI (50:50) |
|---|---|
| #1 | Placebo foam (lesional) |
| #2 | API foam (lesional) |

### Example 13A: Hair Follicle Dystrophy (Masson Fontana)—Acute AA

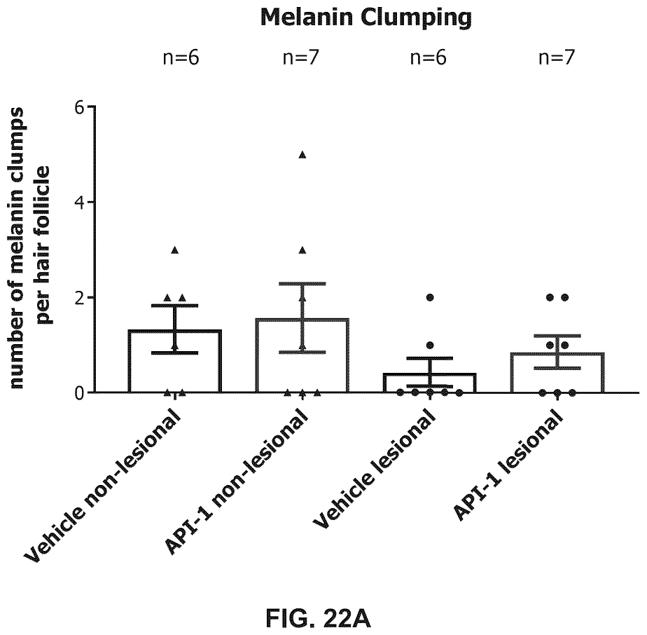

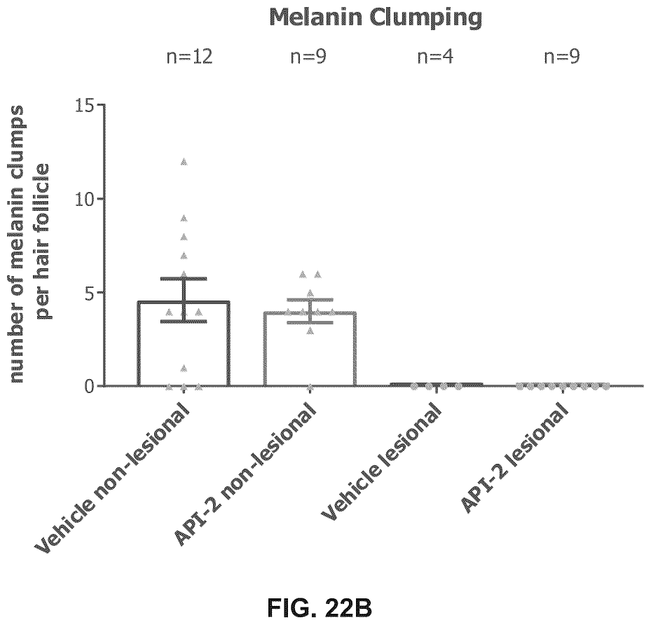

Hair follicle dystrophy (Masson Fontana) relates to the hair follicle pigmentation. Melanin also functions as in indication of hair follicle cytotoxicity and dystrophy. Signs of dystrophy, ectopic melanin clumping and melanin leakage, as well as tissue detachment were evaluated in hair follicle at the end of the experiment. In FIGS. 22A (Donor 1-1) and 22B (Donor 4-2), these figures graphically illustrate the melanin clumping, as the number of melanin clumps per hair follicle for the vehicle non-lesional and lesional and AP-1 and AP-2 non-lesional and lesional. In these figures, n=12-4 HFs/group from N=2 acute AA patients. GraphPad Prism 9; Mean±SEM; D'Agostino & Pearson omnibus normality test, n too small to determine Gaussian distribution, Kruskal-Wallis test, Dunn's multiple comparison test with selected groups: vehicle lesional vs API lesional, vehicle lesional vs vehicle non-lesional or vehicle non-lesional vs API non-lesional n.s.

Based on FIGS. 22A and 22B, melanin clumping was not significantly affected by the application of API-1 or API-2 indicating minimal to no contribution of the application to hair follicle dystrophy.

### Example 13B: Hair Growth Assessment (Ki-67/Tunel)—Acute AA

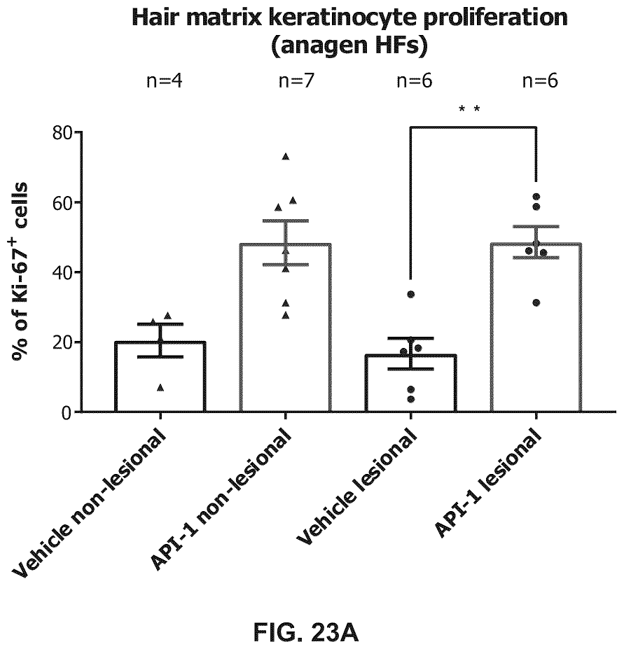

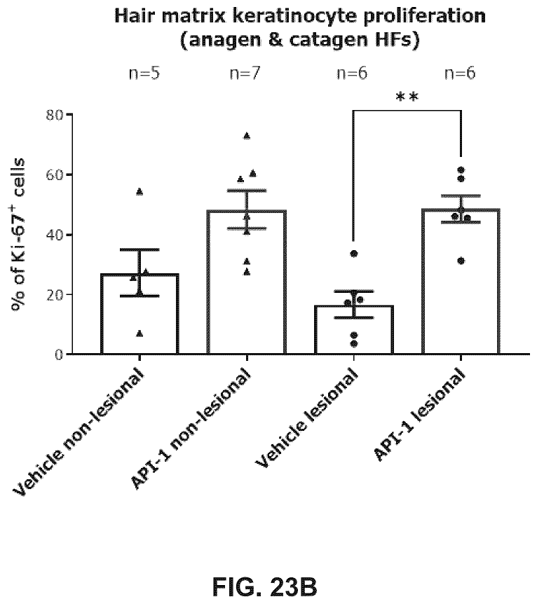

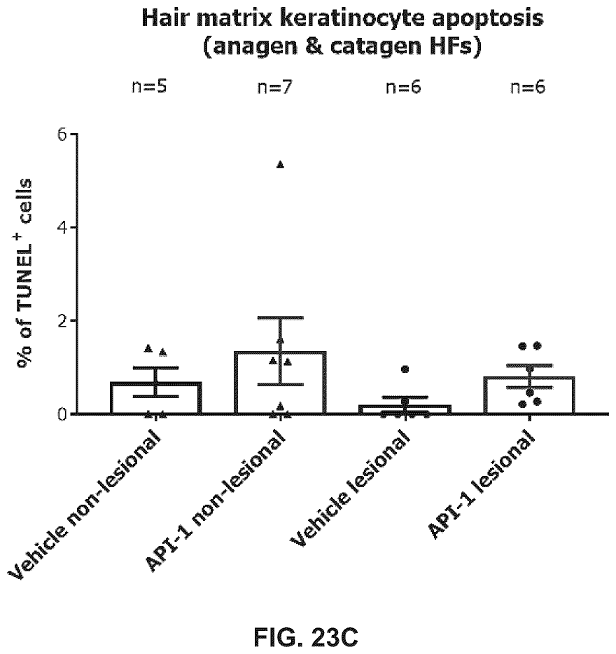

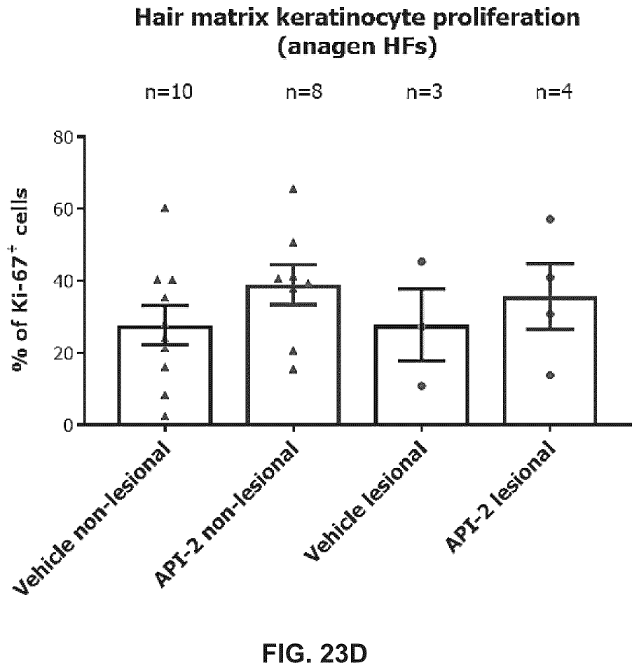

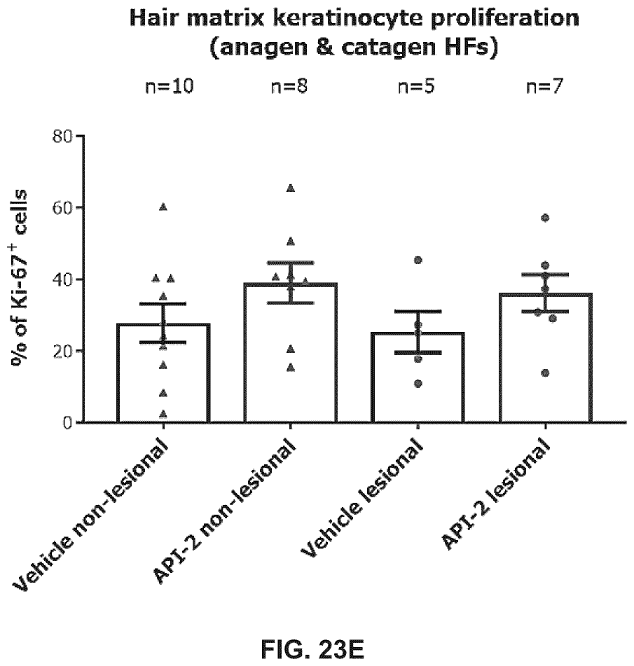

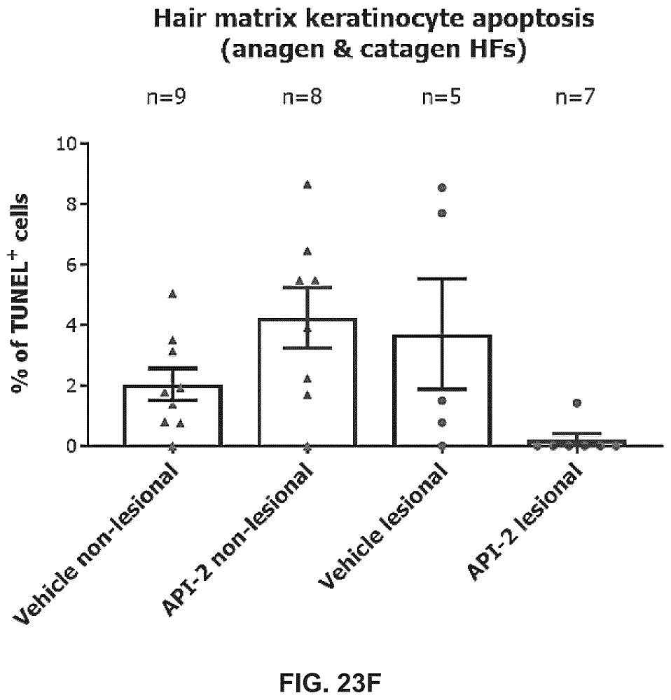

Proliferation of germinative hair matrix keratinocytes directly correlates to anagen, as catagen induction results in decreased proliferation (Langan et al., Exp Dermatol 2015). Apoptosis of hair matrix keratinocytes is used as an additional indicator of anagen/catagen transition. Sections from cultured skin were stained with an antibody against Ki-67 (Abcam, Cat. ab 15580) followed by a secondary goat anti-rabbit IgG conjugated to Rhodamine (Jackson ImmunoResearch Labs, 111-025-144). Hair matrix proliferation is analyzed in the germinative hair matrix (gHM) while apoptosis in the gHM and precortical hair matrix cells pcHM, using TUNEL (TdT-mediated dUTP-biotin nick-end labelling) (Langan et al., Exp Dermatol 2015), with fluorescent-labelled anti-digoxigenin antibody (TUNEL kit, from Cell Signaling). Although Ki-67/TUNEL was attempted in all culture skin samples, staging was only successful in HFs in biopsies from acute AA donors. The loss of immune privilege results in the reduction of germative hair matrix keratinocyte. In FIGS. 23-A-23C (Donor 1-1) and 23D-23F (Donor 4-2), the hair matrix keratinocyte proliferation (anagen hair follicles (HFs)), the hair matrix keratinocyte proliferation (anagen and catagen HFs), and the hair matrix keratinocyte apoptosis (anagen and catagen HFs) are summarized.

As provided in FIGS. 23-A-23C (Donor 1-1) and 23D-23F (Donor 4-2), n=3-10 anagen HFs/group or n=10-5 anagen & catagen HFs/group from N=2 acute AA patients. GraphPad Prism 9; Mean±SEM; D'Agostino & Pearson omnibus normality test, n too small to determine Gaussian distribution, Kruskal-Wallis test, Dunn's multiple comparison test with selected groups: vehicle lesional vs API lesional, vehicle lesional vs vehicle non-lesional or vehicle non-lesional vs API non-lesional, ** p<0.01.

Based on FIGS. 23-A-23C (Donor 1-1) and 23D-23F (Donor 4-2), acute AA samples exposed to API-1 showed an increase in the number and percentage of keratinocyte cells for both lesional and non-lesional samples. The cell counts of Ki-67 positive cells (indication of keratinocyte) indicated an increase in the number of keratinocyte cells compared to samples received the vehicle application. An increase in keratinocyte proliferation indicates the possible restoration of the hair follicle immune privilege.

### Example 13C: Hair Cycle Staging (I)—Acute AA

Using immunohistology and Masson Fontana histochemistry were used to assess hair cycle. Anagen is the growth phase where primary hair growth occurs. Catagen is the transition phase, which is also known as the regression phase, which signifies the start of the degradation of the hair follicle. Dystrophic signifies the lack of recovery of the follicle before the transition to the catagen-telogen stage. This readout was not evaluated in the chronic samples.

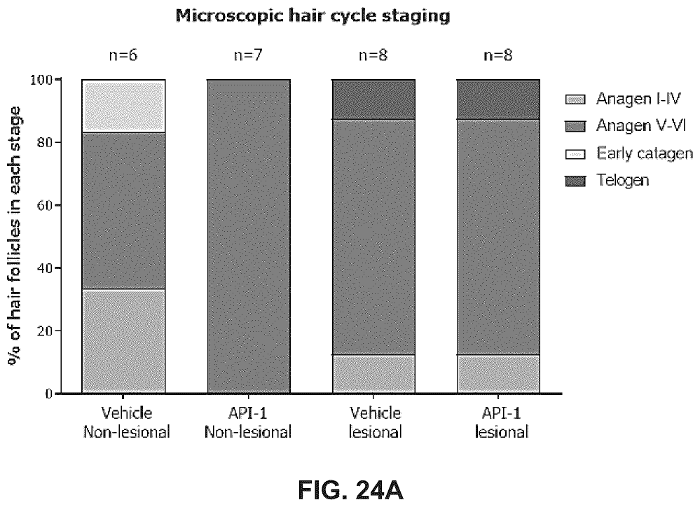

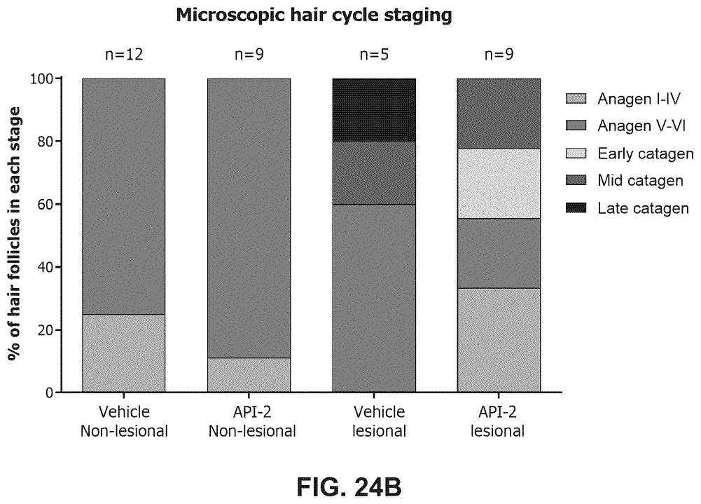

In FIGS. 24A (Donor 1-1) and 24A (Donor 4-2), the macroscopic hair cycle staging is summarized as the % of hair follicles in each stage. API-1 and API-2 both maintain hair follicles in the anagen phase in acute samples. The hair cycle was not changed in the acute lesional samples. As provided in the FIGS. 24A (Donor 1-1) and 24A (Donor 4-2), n=5-12 HFs/group from N=2 acute AA patients. The hair cycle staging was performed by calculating the percentage of HFs in each hair cycle phase. GraphPad Prism 9; statistic not applicable.

Based on FIGS. 24A (Donor 1-1) and 24A (Donor 4-2), in the acute alopecia samples API-1 and API-2 both increase the anagen phase in non-lesional hair follies. The anagen phase signifies the growth phase of the hair follicle. The alopecia samples may require longer treatment; however, this is difficult to test in an ex vivo model.

### Example 13D: Major Histocompatibility Complex I (MHC-I) and Major Histocompatibility Complex II (MCH-II) Expression—Acute AA

Anagen hair follicles (HFs) exhibit immune privilege (IP) in the bulb. Collapse of HF IP is a prerequisite for the development of alopecia areata (AA) (Bertolini et al., Exp Dermatol 2020). Molecules of the primary classes of major histocompatibility complex (MHC-I) are important IP markers, which are found downregulated in normal HF homeostasis, while upregulated in IP collapse. Cryosections of cultured biopsies were stained with a monoclonal mouse antibody against human MHC-I (Santa Cruz, Cat. Sc-32235, clone W6/32) followed by a secondary goat anti-mouse IgG conjugated to Rhodamine (Jackson ImmunoResearch Labs, 115-295-062). MHC-I expression was assessed by fluorescence intensity in the following regions of interest (ROIs): follicular proximal outer root sheath (pORS), dermal papilla stalk (DPst), dermal cup (DC) and germinative hair matrix (gHM) areas, represented in the image on the right. Since hair follicle staging was only successful in biopsies from acute AA donors, the sections from chronic AA donors were spared from MHC-I and used for more suitable immunohistofluorescence studies. MHC-I expression is shown as relative fluorescence, normalized to vehicle-treatment.

Major histocompatibility complex II (MHC-II) molecules are important IP markers, which are found downregulated in normal HF homeostasis, while upregulated in IP collapse. MHC class II molecules are physiologically mostly found on professional antigen-presenting cells (APCs) and they present fragments of extracellular antigens to stimulated CD4+ T lymphocytes, but can also be ectopically expressed in keratinocytes upon IFNg stimulation (Bertolini et al., Exp Dermatol 2020). Cryosections of cultured biopsies were stained with a monoclonal mouse antibody against human MHC-II (Invitrogen, Cat. MA1-25914, clone CD3/43) followed by a secondary goat anti-mouse IgG conjugated to Rhodamine (Jackson ImmunoResearch Labs, 115-295-062). MHC-II expression was assessed, using ImageJ software, by measuring IF intensity in the follicular proximal outer root sheath (pORS) and counting number of positive cells here and in the bulbar connective tissue sheath (bCTS) and perifollicular tissue (PFT) (areas represented on the image on the right). Similar to MHC-I, biopsies from chronic AA donors were not stained for MHC-II. MHC-II expression is shown either as relative fluorescence, normalized to vehicle-treatment or as cell positive counts normalized to area (per 100 μm²).

The loss of immune privilege was evaluated by the upregulation of MHC-I and MHC-II in the hair follicle. The expression was evaluated in acute samples since hair follicle staging was only successful in biopsies from acute samples.

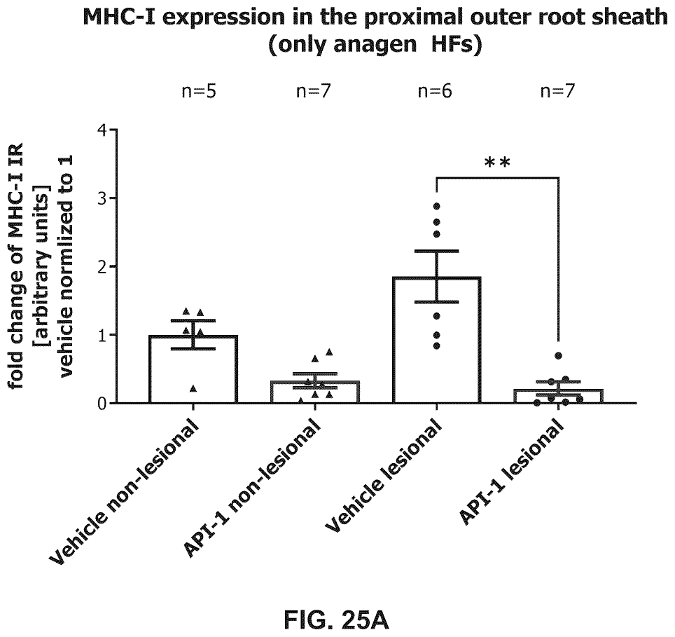

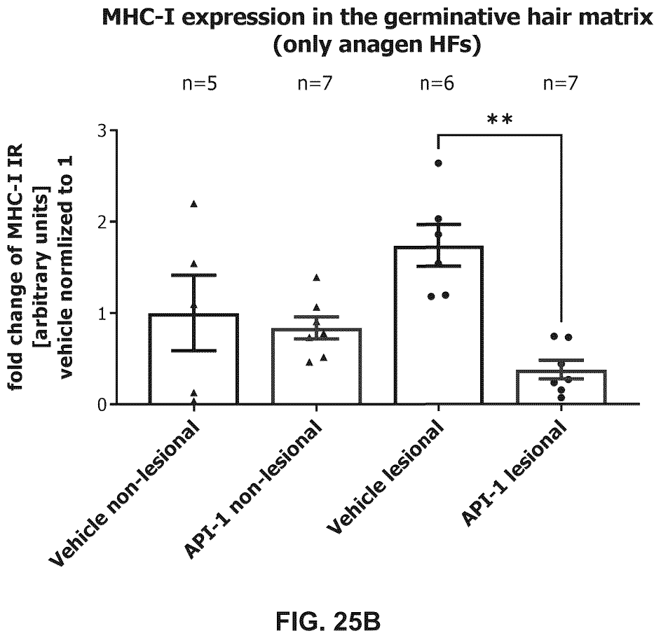

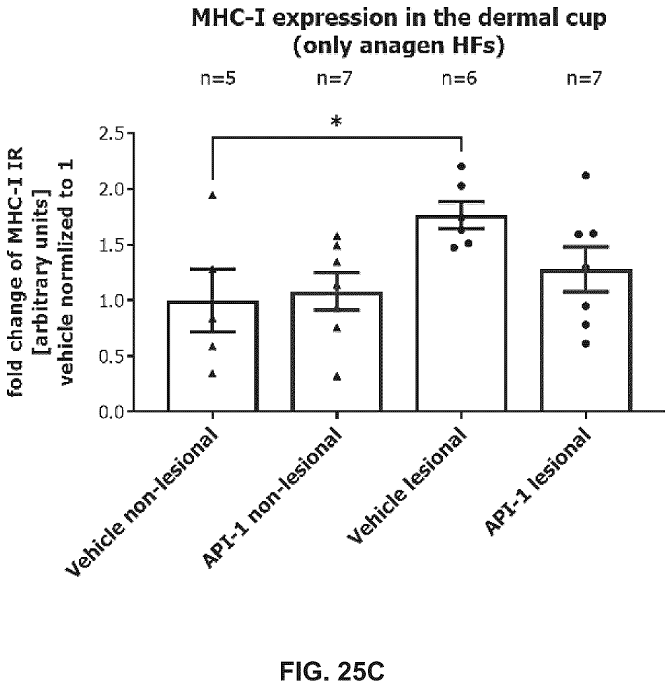

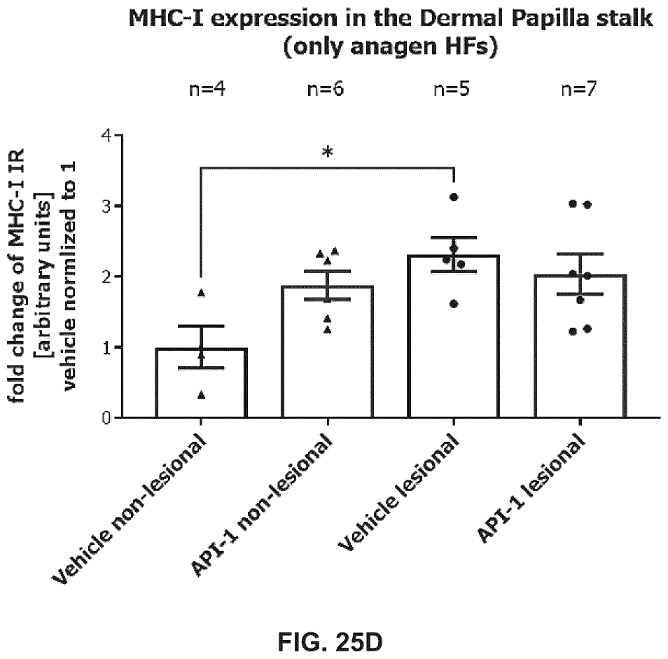

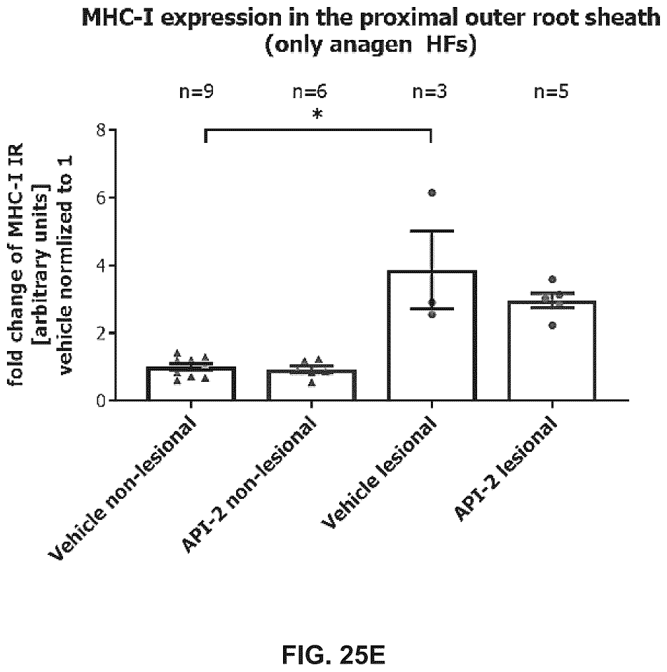

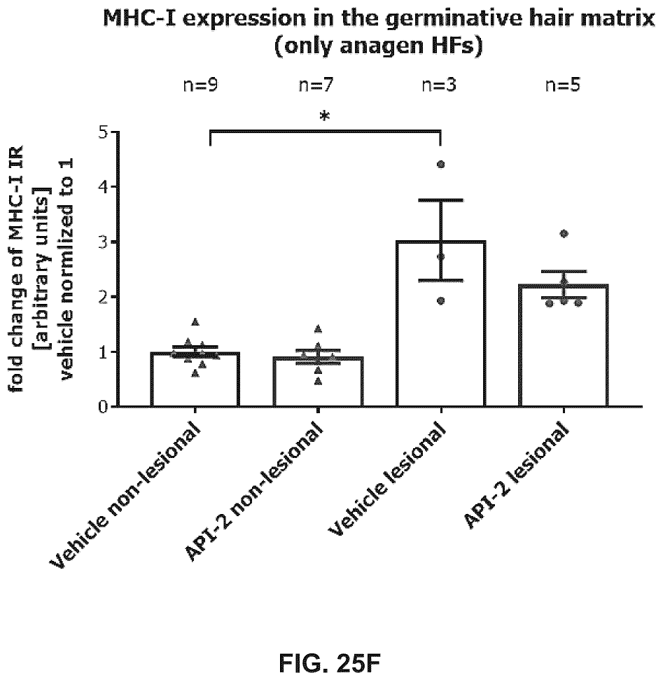

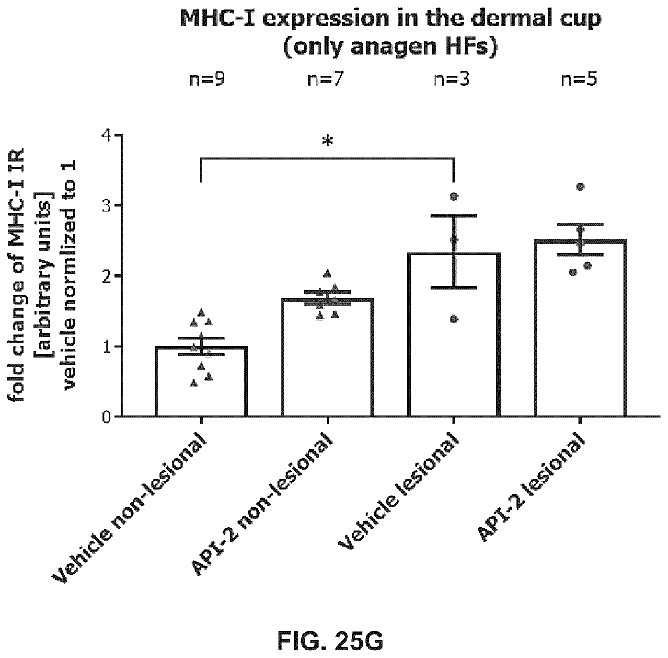

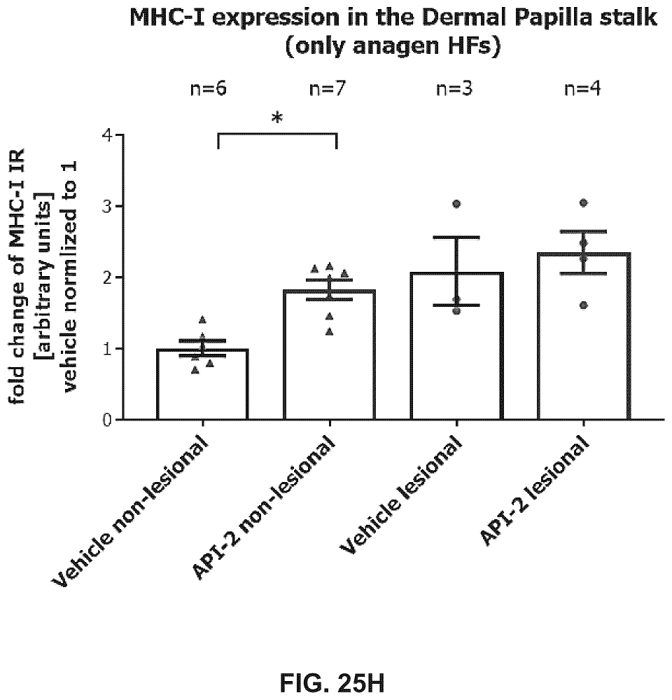

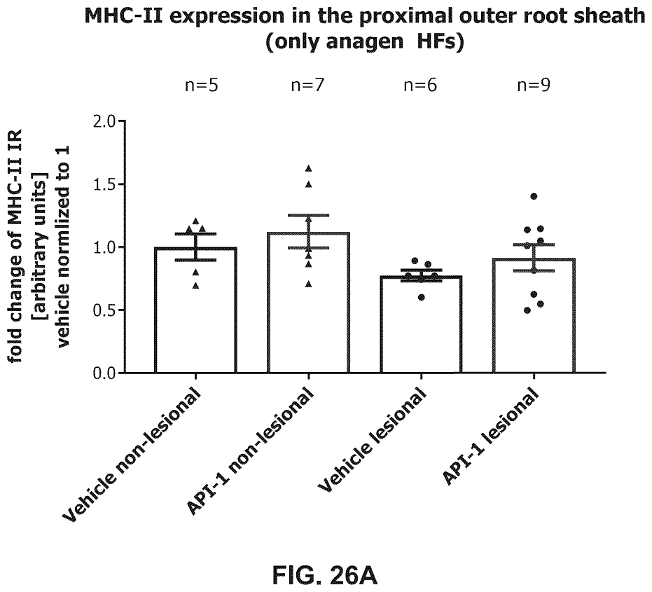

In FIGS. 25A-D (Donor 1-1) and 25E-H (Donor 4-2), the MHC-I expression is shown in the proximal outer root sheath, the germinative hair matrix, the dermal cup, and the dermal papilla stalk, respectively for each donor and only for anagen hair follicles (HFs). For these figures, n=3-9 HFs/group from N=2 acute AA patients; GraphPad Prism 9; Mean±SEM; D'Agostino & Pearson omnibus normality test, n too small to determine Gaussian distribution, Kruskal-Wallis test, Dunn's multiple comparison test with selected groups: vehicle lesional vs API lesional, vehicle lesional vs vehicle non-lesional or vehicle non-lesional vs API non-lesional, *p<0.05

From FIGS. 25A-D (Donor 1-1) and 25E-H (Donor 4-2), the application of API-1 indicated a reduction of MHC-I which indicated a restoration of the immune privilege for the proximal outer root sheath, germinative hair matrix, and dermal cup of acute AA samples. For API-2, there was a tendentially decrease in MHC I for the proximal outer root sheath and germinative hair matrix. This indicates API-1 robustly and API-2 tendentially restored the hair bulb immune privilege and tolerance to CD8 positive T cells.

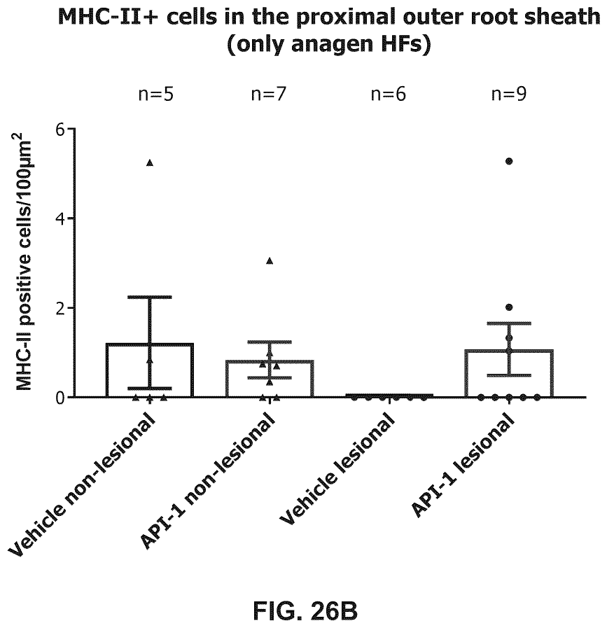

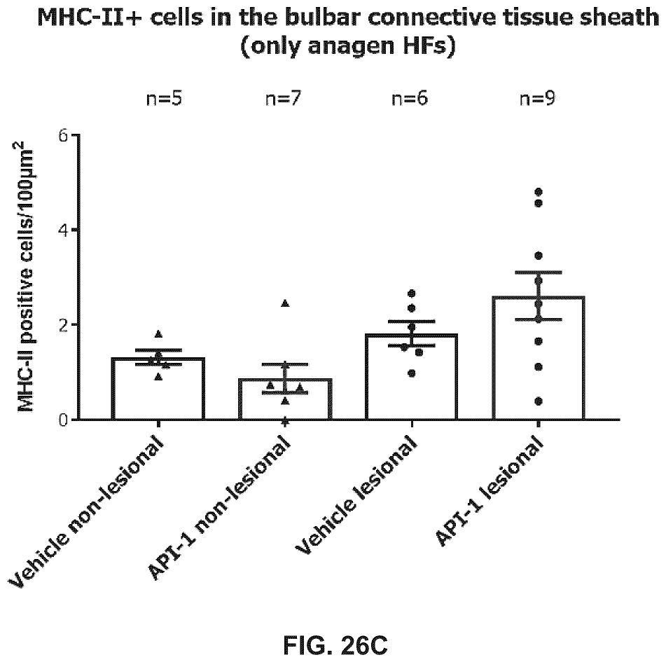

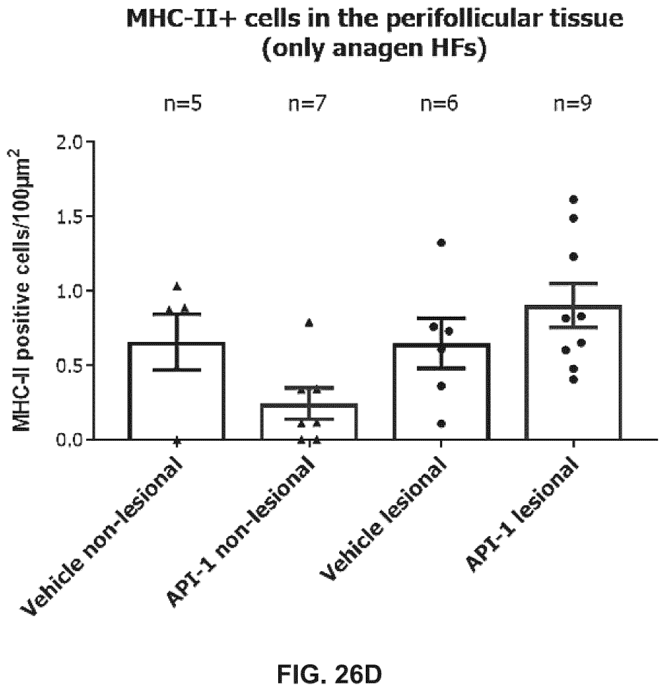

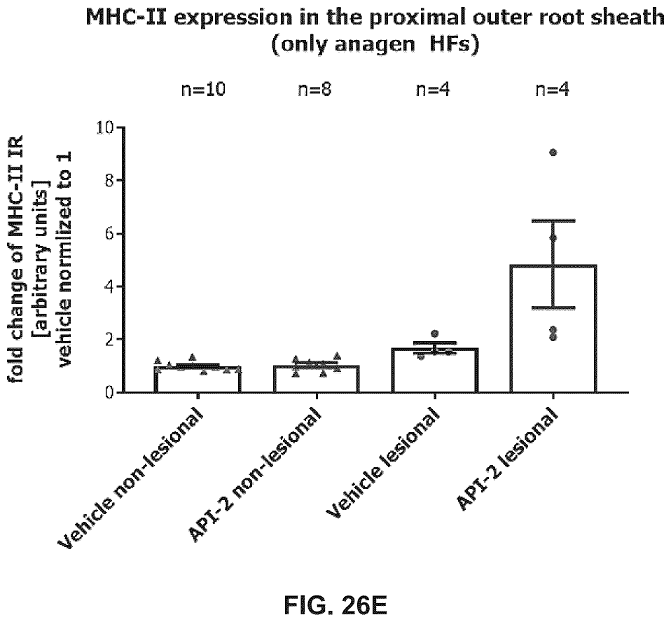

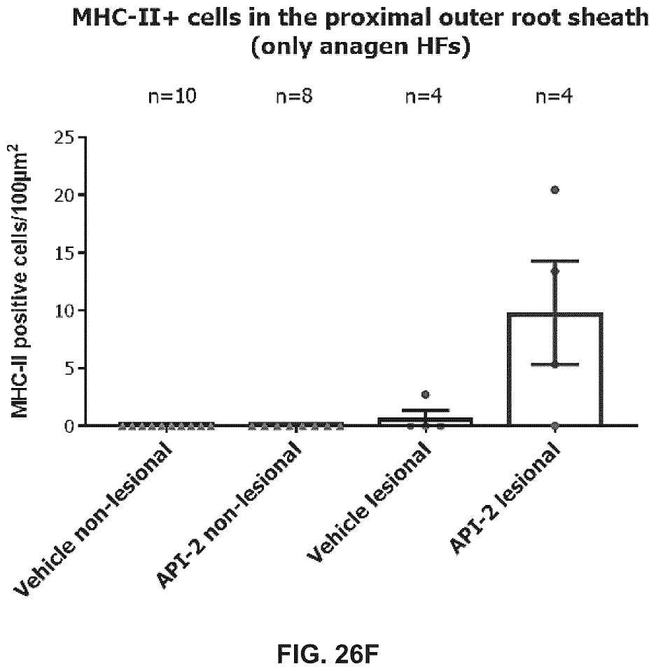

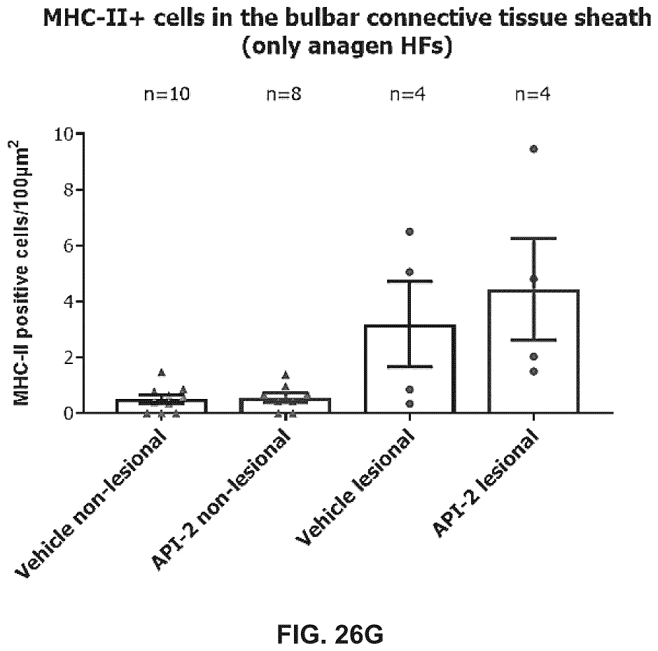

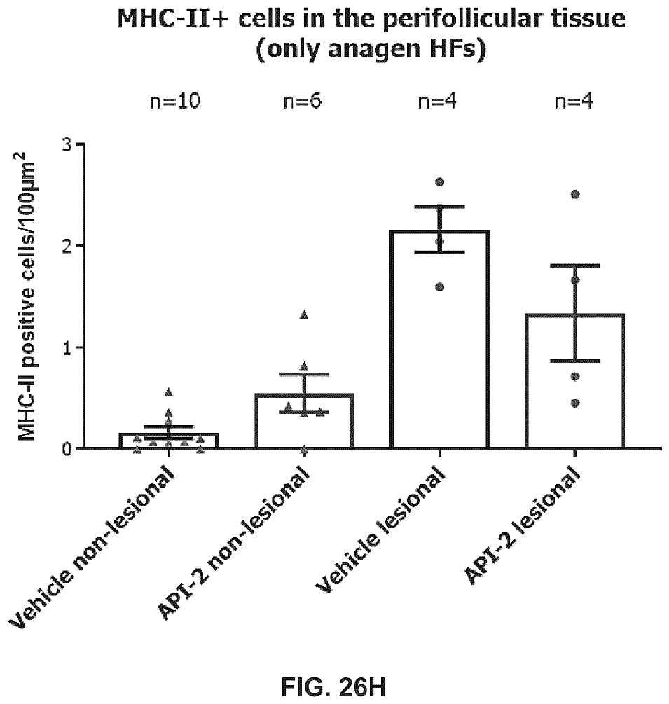

In FIGS. 26A-D (Donor 1-1) and 26E-H (Donor 4-2), the MHC-II expression is shown in the proximal outer root sheath, the germinative hair matrix, the dermal cup, and the dermal papilla stalk, respectively for each donor and only for anagen hair follicles (HFs). The number of MHC II cells were tendentially increased for both API-1 and API-2. According to literature, pre-administration of ruxolitinib before the stimulation of IFNg may be able to restore immune privilege in the hair follicles. Of note, the duration of treatment may not have been adequate to observe a change in the MHC II expression.

### Example 13E: CD8/CD69 Immunofluorescence—Acute AA

T cell number and activation, particularly from CD8+ effector T cells, are typical hallmarks of AA (Bertolini et al., Exp Dermatol 2020) and hence cultured biopsies, from acute AA donors, were stained with antibodies against the human CD8 (Abcam, Cat 196193, Alexa flour 647 conjugated, clone EP1150Y) and CD69 (Biolegend, Cat 310906, PE conjugated clone MOPC-21). After image acquisition, T cell number and activation was assessed by counting total CD8+ and CD8+CD69+ cells, respectively, and potential NK cells by CD8-CD69+ cells counting, in two regions of interest, ROIs: hair follicle (HF) epithelium as well as connective tissue sheath, CTS, and perifollicular tissue. The CTS and perifollicular dermis were defined as an area of 100-200 μm surrounding the HF epithelium, excluding the following structures: glands, blood vessels, damaged tissue and epithelium from neighbouring HFs. A minimum of 6 HFs per condition were evaluated. The number of total CD8+ cells, CD8+CD69+ and CD8-CD69+ is shown normalized to area (per 100 μm²) and the CD8+CD69+ T-cells also as percent of CD8+.

The number and activation of T-cells and more specifically, CD8 positive effector T cells are hallmarks of alopecia areata. Here, CD8 positive, CD8 positive CD69 positive, and CD8 negative CD69 positive cells were examined.

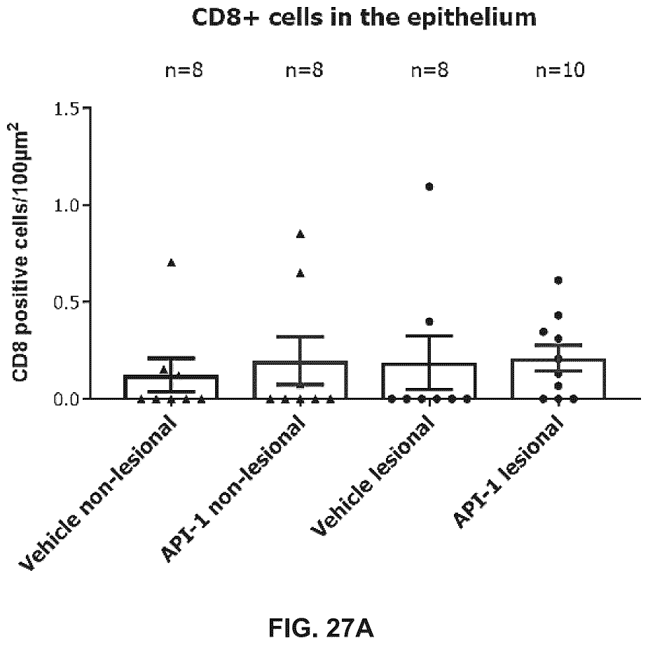

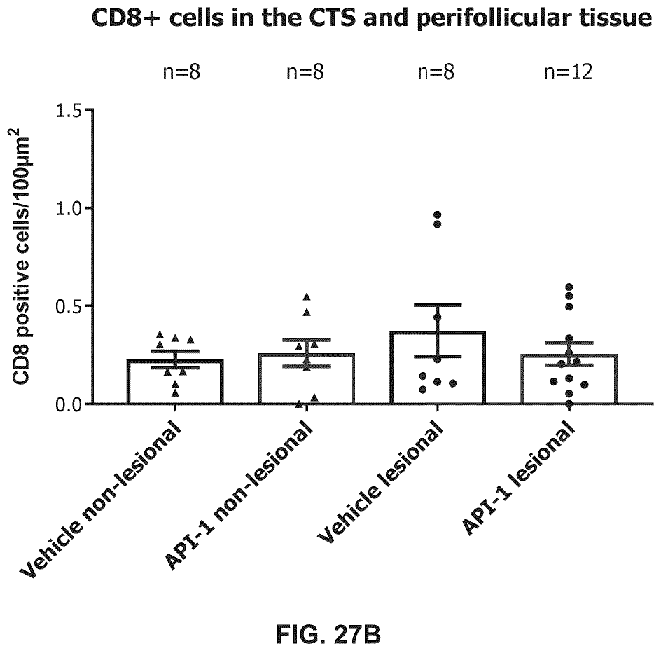

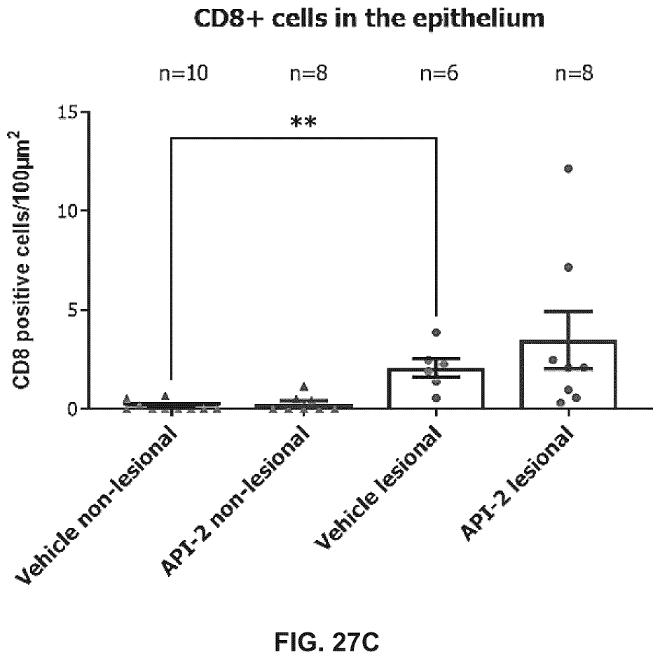

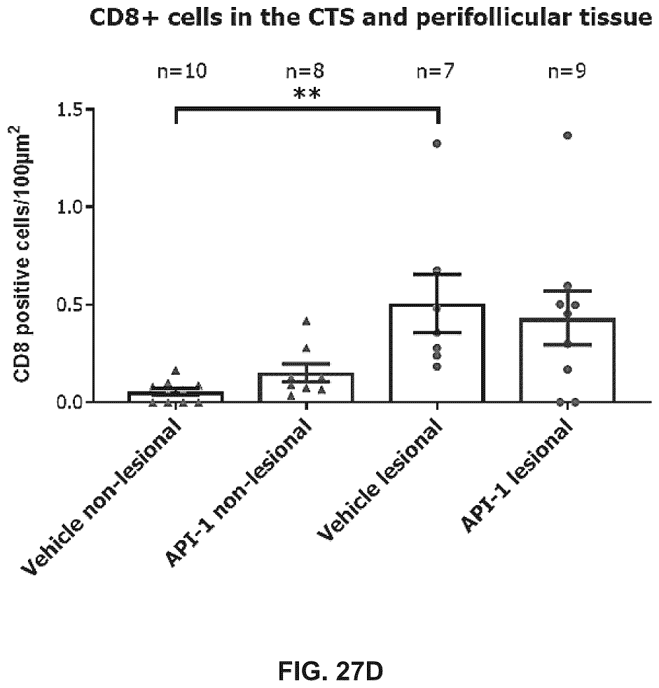

In FIGS. 27A and 27 B (Donor 1-1) and 27C and 27D (Donor 4-2), CD8+ cells in the epithelium, in the CTS and perifollicular tissue were examined. Data shown here for the connective tissue sheath and perifollicular tissue are a combination of data points deriving from two different stainings (CD8/CD69 and CD8/NKG2D). If possible, data were averaged for the hair follicles present in both stainings. For the epithelium, data were only available from the CD8/CD69 staining as in the CD8/NKG2D staining strong staining background in the epithelium prohibited the analysis. In FIGS. 27A and 27 B (Donor 1-1) and 27C and 27D (Donor 4-2), n=6-12 HFs/group from N=2 acute AA patients. GraphPad Prism 9; Mean±SEM; D'Agostino & Pearson omnibus normality test, no Gaussian distribution, Kruskal-Wallis test, Dunn's multiple comparison test with selected groups: vehicle lesional vs API lesional, vehicle lesional vs vehicle non-lesional or vehicle non-lesional vs API non-lesional, p *<0.01

From FIGS. 27A and 27 B (Donor 1-1) and 27C and 27D (Donor 4-2), API-I tendentially decreased in CD8 positive cells for the perifollicular tissue. API-2 had a slight decrease in CD8 positive for the perifollicular tissue. This may indicate a restoration in the immune privilege.

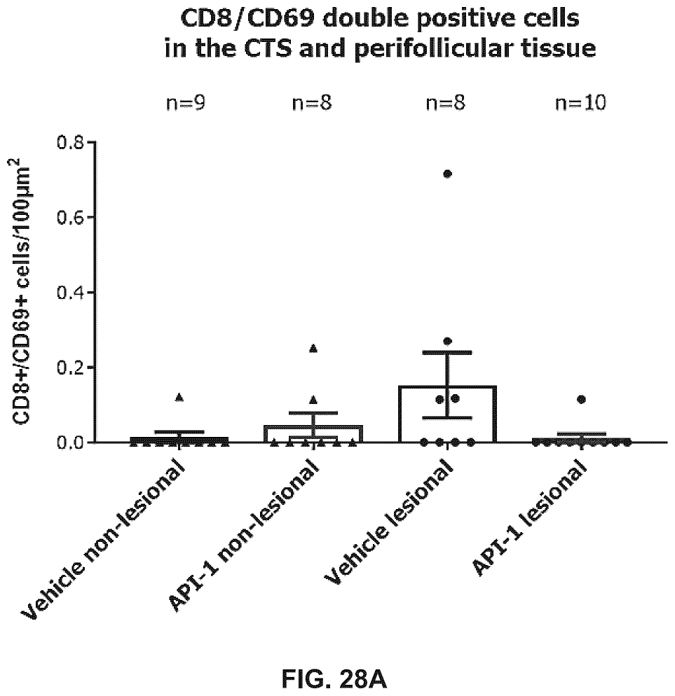

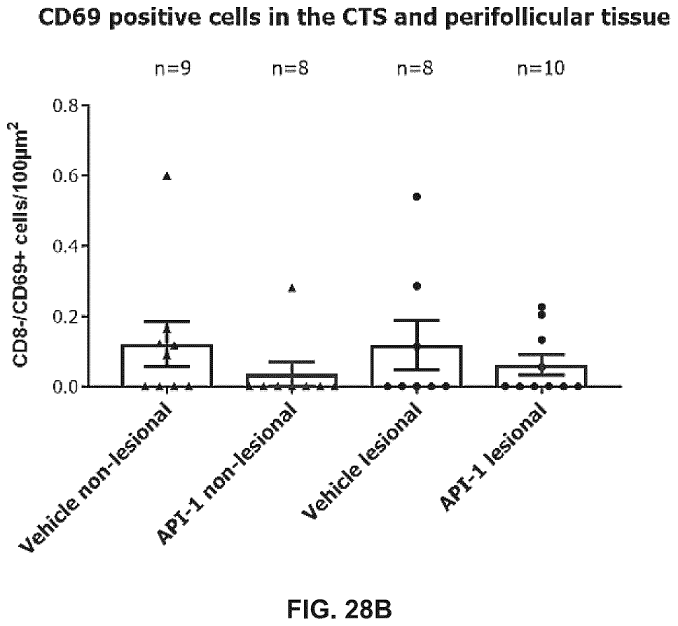

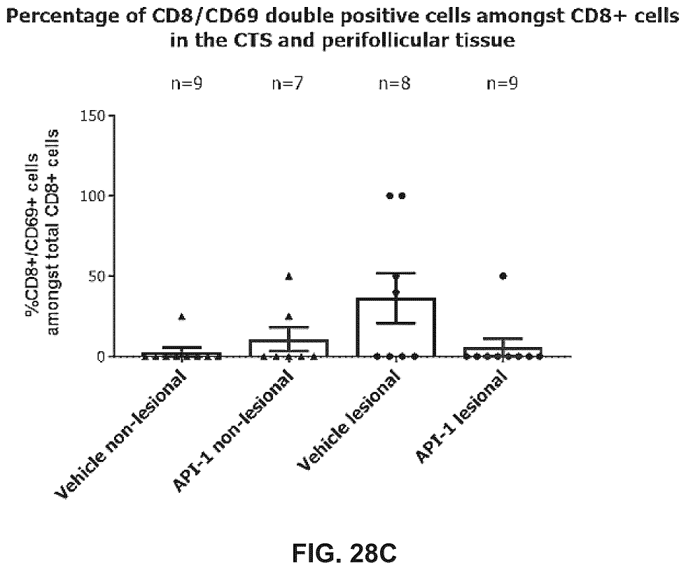

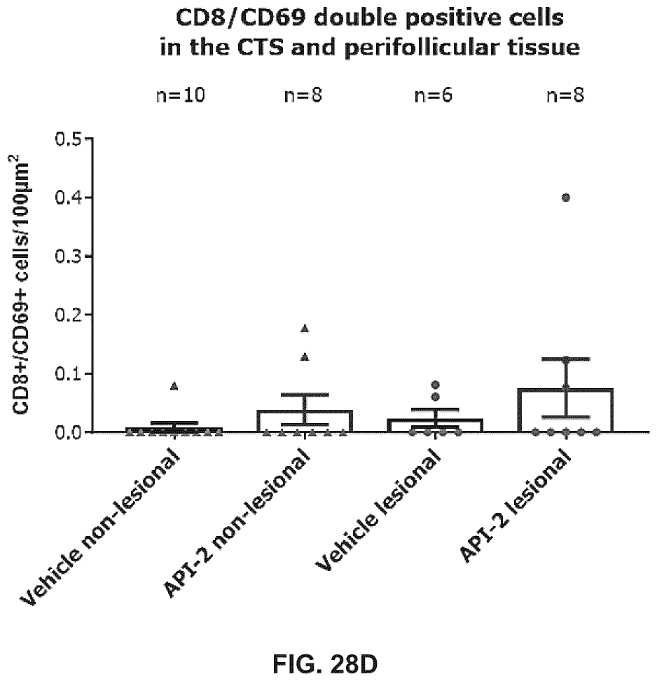

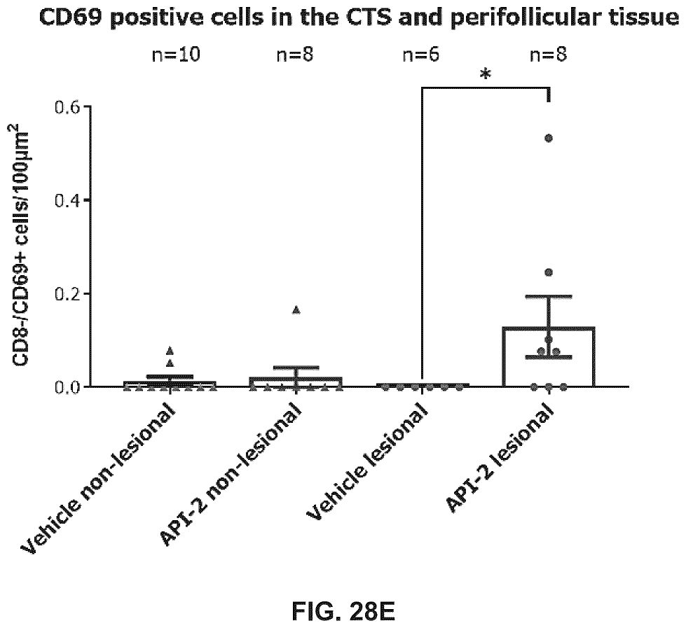

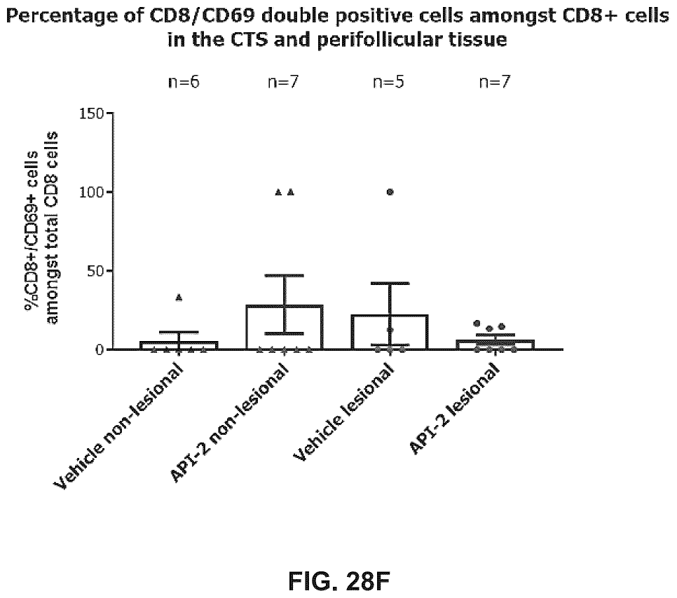

In FIGS. 28A-C (Donor 1-1) and 28D-F (Donor 4-2), CD8/CD69 double positive cells in the CTS and perifollicular tissue, CD69 positive cells and perifollicular tissue, and the percentage of CD8/CD69 double positive cells amongst CD8+ cells in the CTS and perifollicular tissue were examined. From those figures, n=5-10 HFs/group from N=2 acute AA patients. One data point represents the number per area of a hair follicle. GraphPad Prism 9; Mean±SEM; D'Agostino & Pearson omnibus normality test, n too small to determine Gaussian distribution, Kruskal-Wallis test, Dunn's multiple comparison test with selected groups: vehicle lesional vs API lesional, vehicle lesional vs vehicle non-lesional or vehicle non-lesional vs API non-lesional, n.s. Due to 0 for some data points in the vehicle, the evaluation of the percentage was not possible for these HFs. Additionally, data is only shown for the connective tissue sheath and perifollicular tissue, as only very few CD8+CD69+ cells could be detected in the hair follicle epithelium.

From FIGS. 28A-C (Donor 1-1) and 28D-F (Donor 4-2), CD69 is an activation marker of T-cells. API-1 indicated a decrease in dual positive CD8/CD69 cells for the connective tissue perifollicular cells and the number of CD69 (CD8 negative) cells. API-2 demonstrated a reduction in the percentage of dual positive CD8/CD69 cells compared to total CD8 cells in the perifollicular tissue. The inhibition of the activation marker was observed for API-1 and API-2 for the percentage of dual positive cells over the total CD8 cells.

### Example 13F: CD8/NKG2D Immunofluorescence—Acute AA

NKG2D+CD8+ effector T cells are described as the main drivers of AA (Bertolini et al., Exp Dermatol 2020) and hence cultured biopsies were stained with antibodies against the human CD8 (Abcam, Cat 196193, Alexa flour 647 conjugated, clone EP1150Y) and NKG2D (BD Bioscience Cat 552866 Clone 1D11). After image acquisition, T cell number and activation was assessed by counting total CD8+ and CD8+NKG2D+ cells, respectively, and potential NK or gdT cells by CD8-NKG2D+ cells counting, particularly in the connective tissue sheath, CTS, and perifollicular tissue. A minimum of 5 HFs per condition were evaluated. The number of total CD8+ cells, CD8+NKG2D+ and CD8-NKG2D+ is shown normalized to area (per 100 μm²) and the CD8+NKG2D+ T-cells also as percent of CD8+.

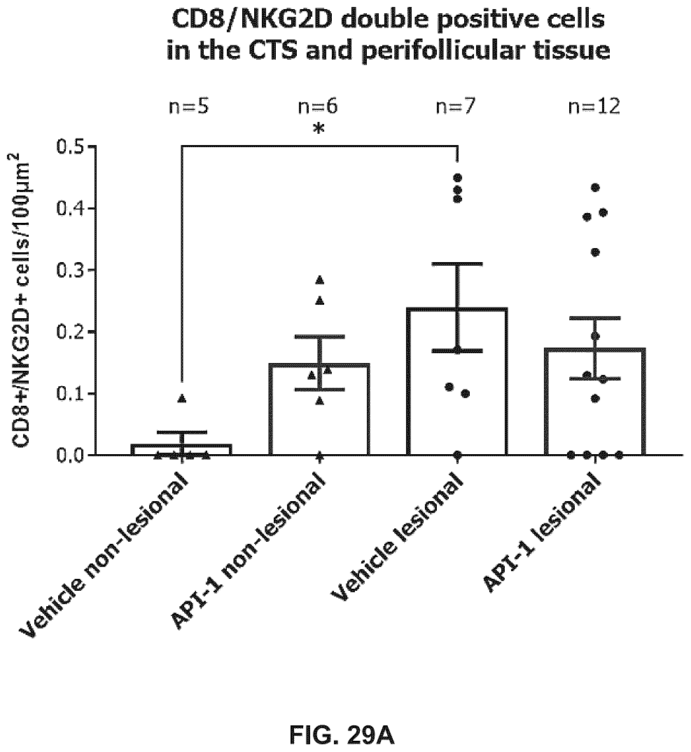

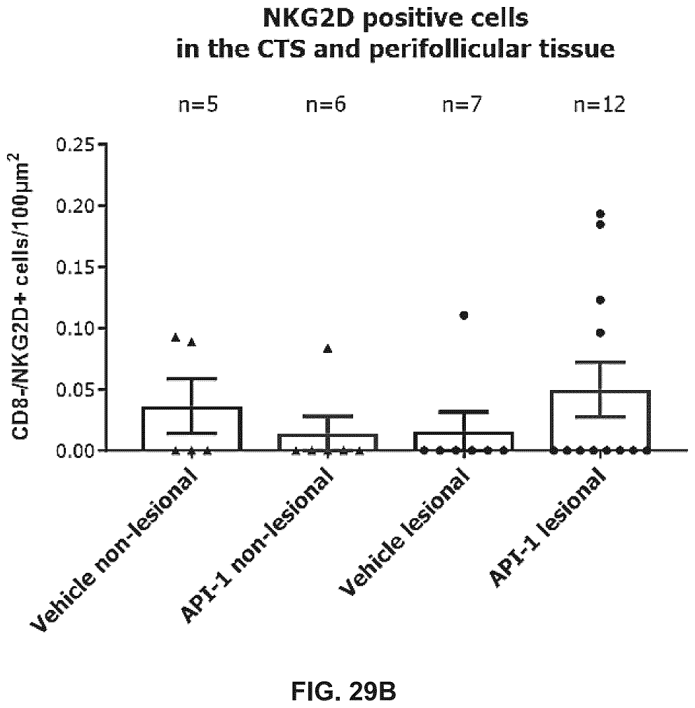

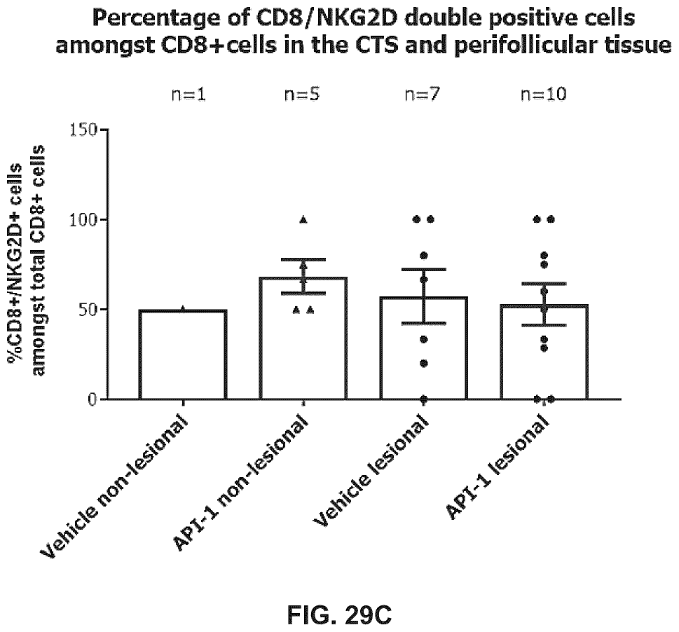

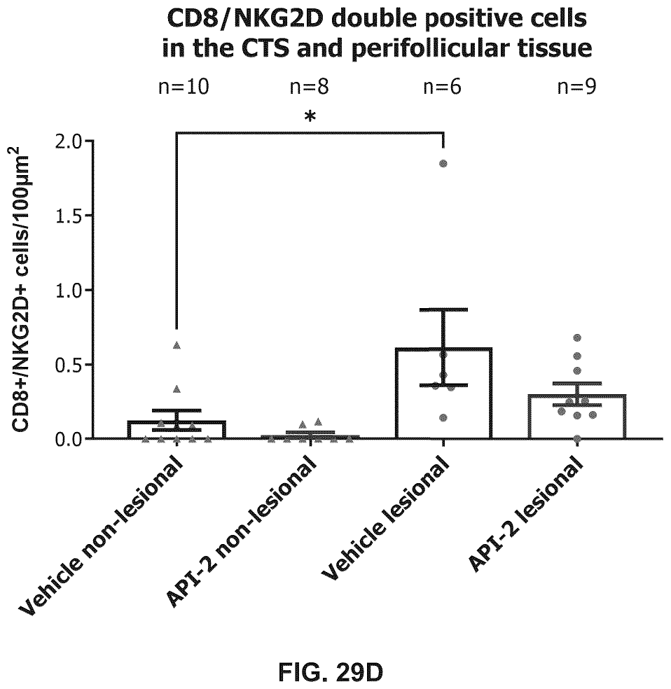

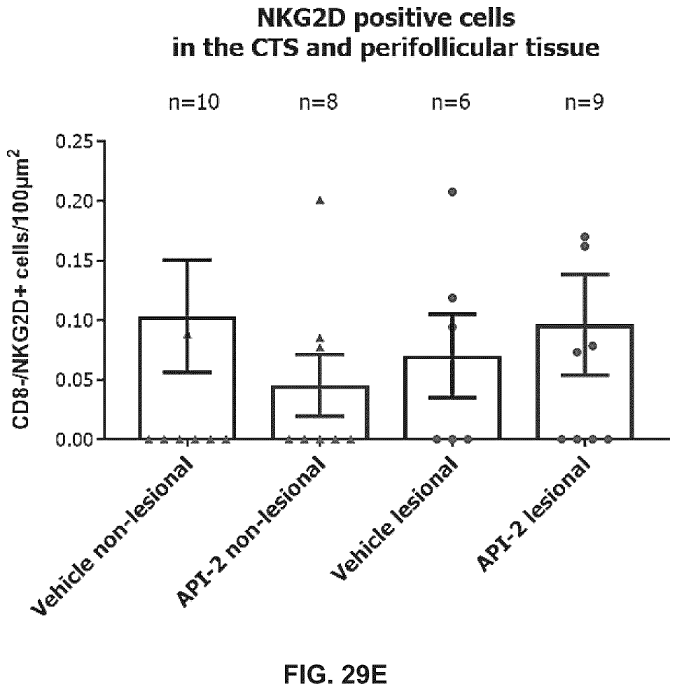

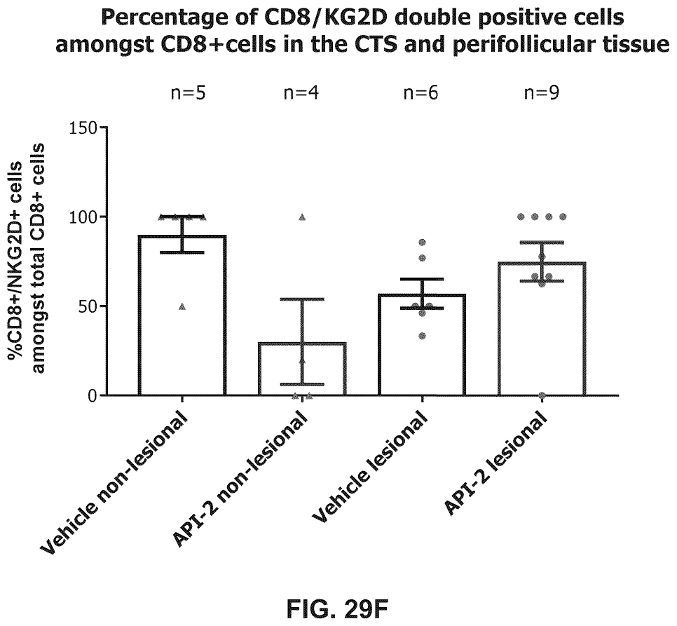

NKG2D positive CD8 positive effector T cells were referenced as a main driver of alopecia areata. Immunofluorescence-stained cells were used to count CD8 positive, CD8 positive NKGD positive, and CD8 negative NKG2D positive cells. In FIGS. 29A-C (Donor 1-1) and 29D-F (Donor 4-2), the CD8/NKG2D double positive in the CTS and perifollicular tissue, NKG2D positive cells in the CTS and perifollicular tissue, and the percentage of CD8/NKG2D double positive cells amongst CD8+ cells in the CTS and perifollicular tissues were examined. Only very few CD8+NKG2D+ cells could be detected in the hair follicle epithelium (data not shown). Due to 0 for some data points in the vehicle, the evaluation of the percentage was not possible for these HFs.

From FIGS. 29A-C (Donor 1-1) and 29D-F (Donor 4-2), NKG2D is an activating factor for natural killer cells which are cytotoxic, effector cells of the immune system. A tendentially reduction in dual positive CD8/NKG2D cells in lesional samples compared to vehicle for API-1 and API-2.

### Example 13G: CD3/CD8/Ki-67 Immunofluorescence-Chronic AA

T cell proliferation and activation is leading to T-cell expansion in AA (Bertolini et al., Exp Dermatol 2020). To investigate the effect on CD3+ and CD8+ cell proliferation, tissue sections from chronic AA donors were stained with antibodies against the human CD3 (Biolegend, Cat 300508, PE conjugated, clone RPA-T4) CD8 (Abcam, Cat 196193, Alexa flour 647 conjugated, clone EP1150Y), Ki-67 (Cell Signaling Cat 9449, clone 8D5). After image acquisition, T cell number and activation was assessed by manual counting of total CD3+, total CD3+CD8+, CD3+CD8-Ki-67+ and CD3+CD8+Ki-67+ cells in two in two regions of interest, ROIs: hair follicle (HF) epithelium as well as connective tissue sheath, CTS, and perifollicular tissue. A minimum of 6 HFs per condition, per donor, were evaluated. The numbers of total CD3+ cells, CD3+CD8+, CD3+Ki-67+ and CD3+CD8+Ki-67+ are shown normalized to area (per 100 μm²) and of proliferating T cells (both CD3+Ki-67+ and CD3+CD8+Ki-67+) as a percent of total CD3+ or CD3+CD8+ (respectively).

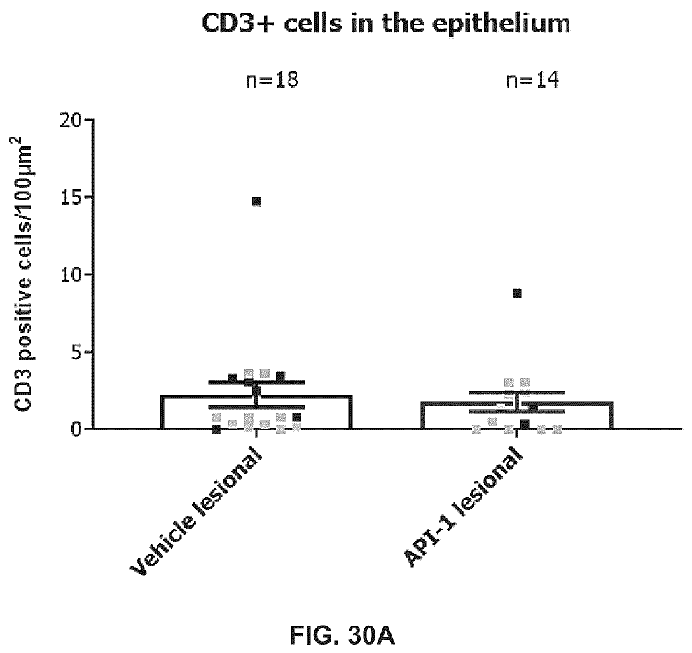

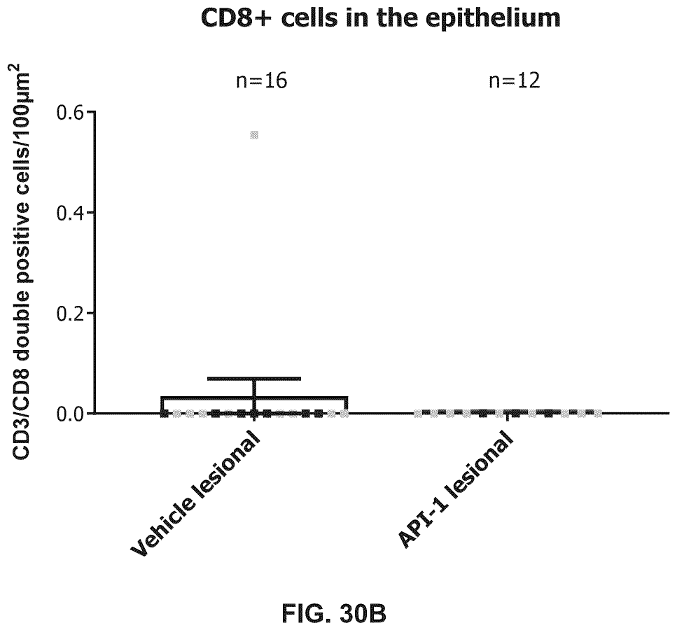

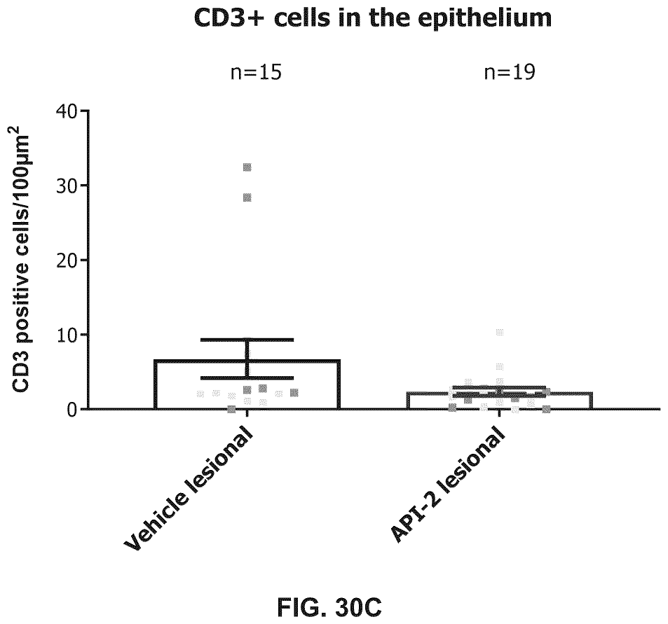

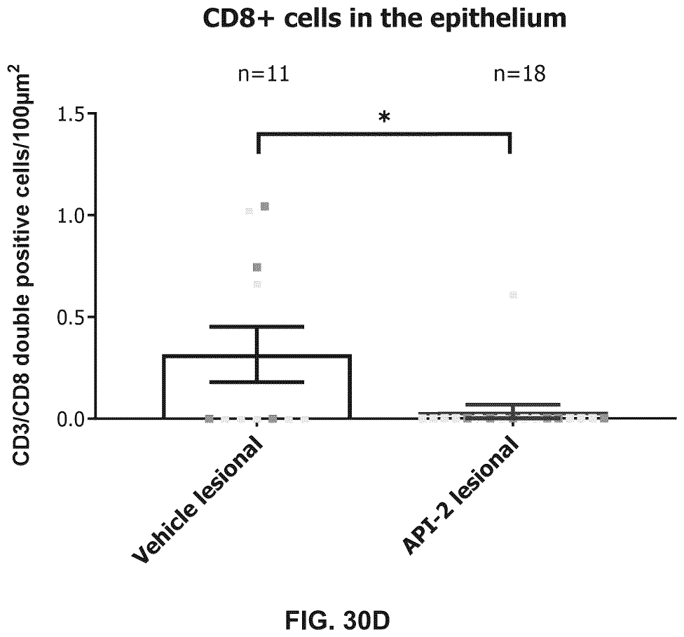

Immunofluorescence staining of CD3/CD8/Ki-67 cells examined T cell proliferation and activation leading to T-cell expansion. The numbers of CD3 positive, CD3 positive CD8 positive, CD3 positive Ki-67 positive, and CD3 positive CD8 positive Ki-67 positive cells were examined. In FIGS. 30A and 30 B (API-1, chronic donor) and 30C and 30D (API-2, chronic donor), CD3+ cells in the epithelium and CD8+ cells in the epithelium were examined. In these figures, n=11-19 HFs/group from N=4 chronic AA patients. One data point represents the number per area of a hair follicle. GraphPad Prism 9; Mean±SEM; D'Agostino & Pearson omnibus normality test, no Gaussian distribution, Mann Whitney test p *<0.05.

From FIGS. 30A and 30 B (API-1, chronic donor) and 30C and 30D (API-2, chronic donor), API-1 tendentially reduced CD3/CD8 dual positive when compared to the vehicle treated lesional samples. API-2 tendentially reduced CD3 positive cells and significantly reduced CD3/CD8 dual positive cells compared to the vehicle. This signifies the reduced activation of an immune response in the epithelium.

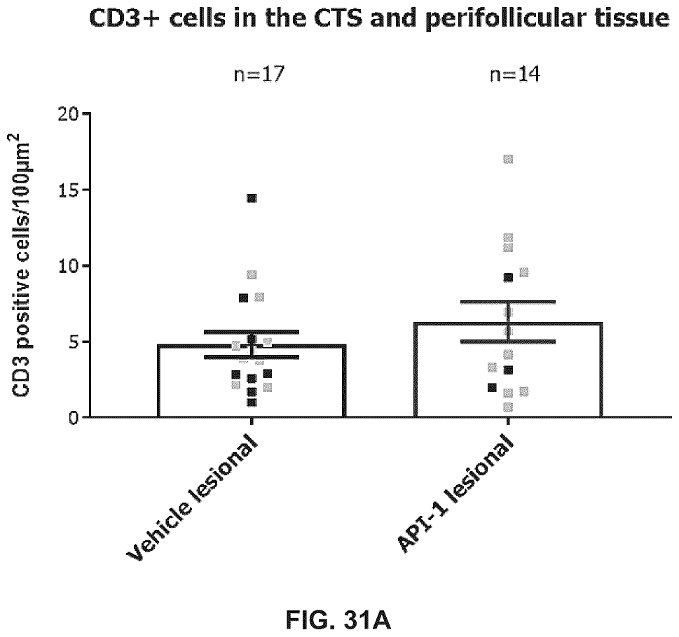

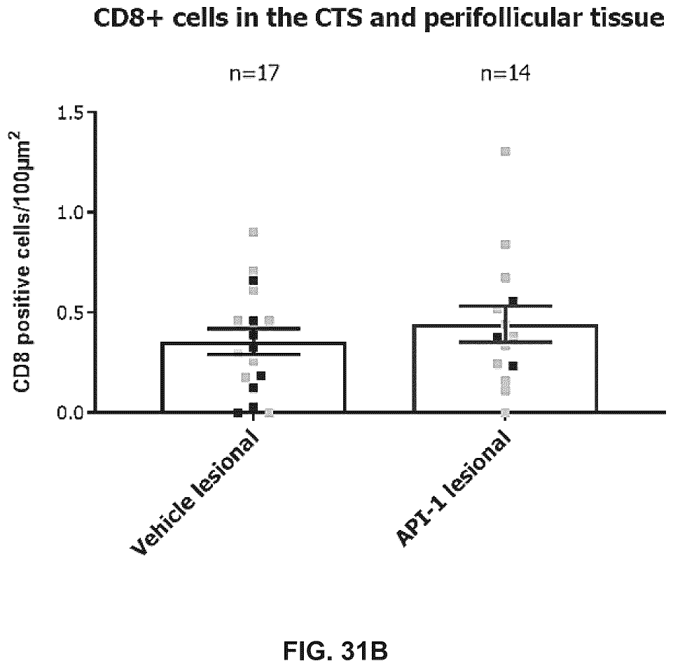

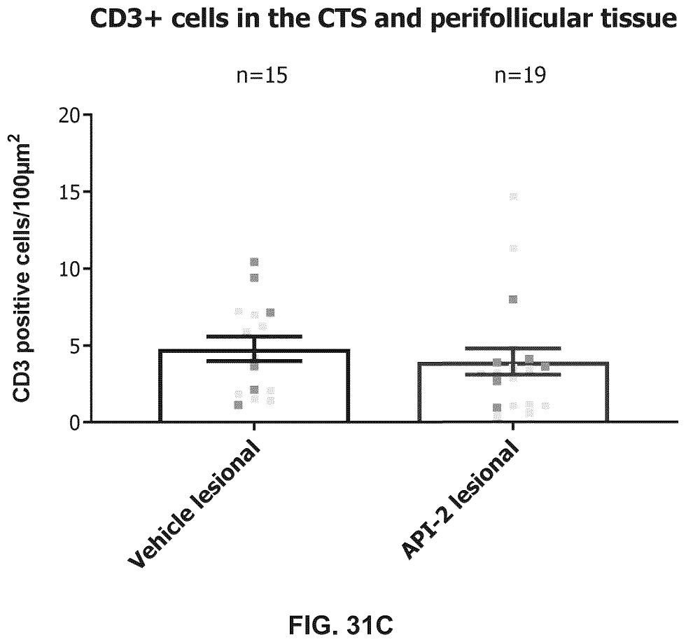

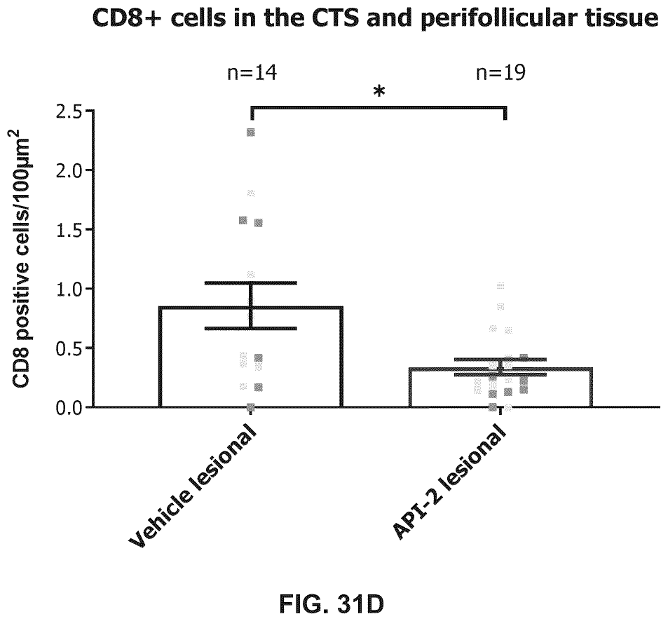

In FIGS. 31A and 31B (API-1, chronic donor) and 31C and 31D (API-2, chronic donor), CD3+ cells in the CTS and perifollicular tissue and CD8+ cells in the CTS and perifollicular tissue were examined. In these figures, n=14-19 HFs/group from N=4 chronic AA patients. One data point represents the number per area of a hair follicle. GraphPad Prism 9; Mean±SEM; D'Agostino & Pearson omnibus normality test, for CD8 API-2: Gaussian distribution, Unpaired t test n.s; for the rest: no Gaussian distribution, Mann Whitney test p *<0.05. Data shown here for the CD3+ cells in the CTS and perifollicular tissue are a combination of data points deriving from two different stainings (CD3/CD69/CD103 and CD3/CD8/Ki-67). Data shown here for the CD8+ cells in the CTS and perifollicular tissue are a combination of data points deriving from two different stainings (CD8/NKG2D and CD3/CD8/Ki-67). If possible, data were averaged for the hair follicles present in both stainings.

From FIGS. 31A and 31B (API-1, chronic donor) and 31C and 31D (API-2, chronic donor), API-2 had a significant reduction in CD8 positive cells in the perifollicular tissue. CD8 can contribute to inflammation.

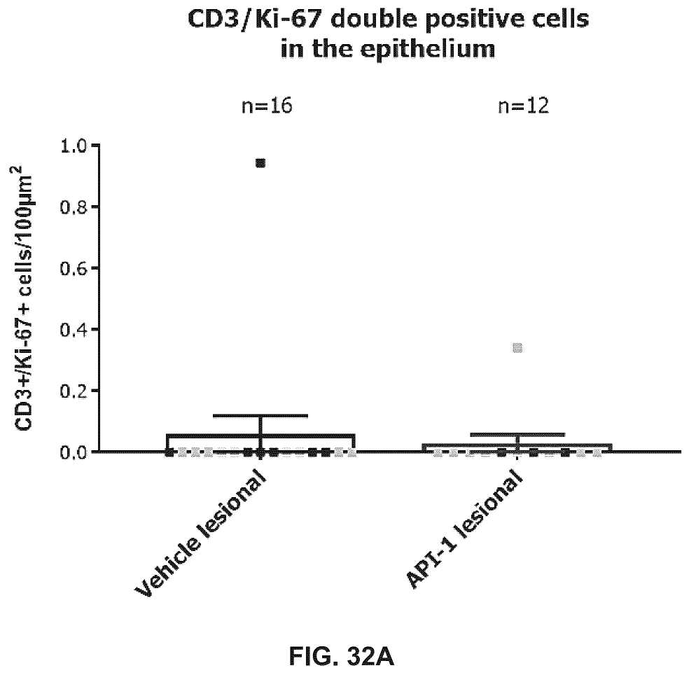

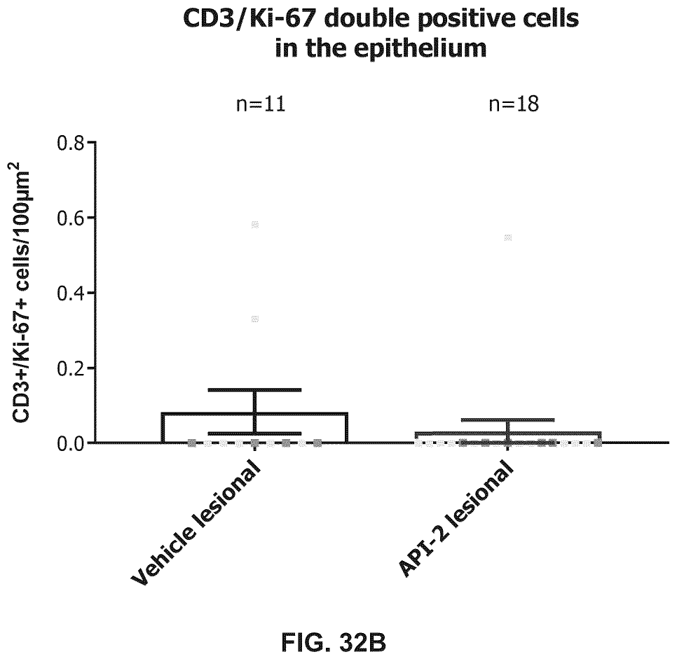

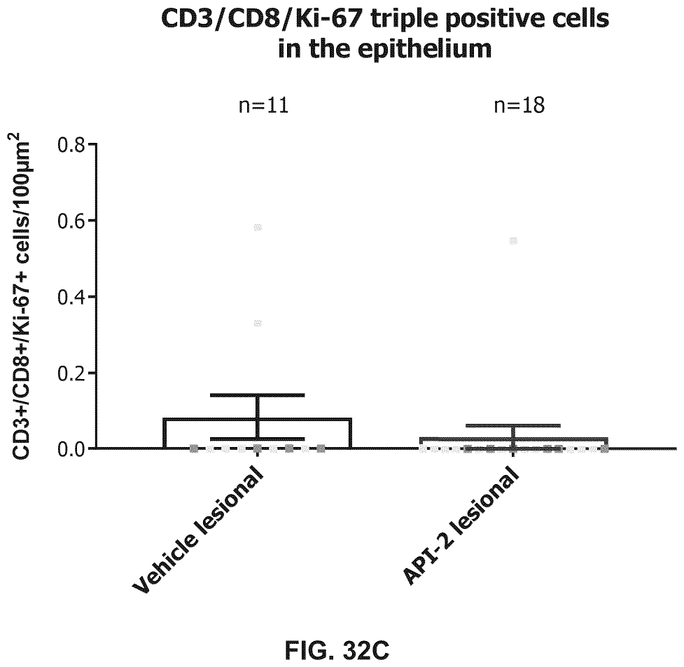

In FIGS. 32A (API-1, chronic donor) and 32B and 32C (API-2, chronic donor), CD3/Ki-67 double positive cells in the epithelium were examined. In these figures, n=11-18 HFs/group from N=4 chronic AA patients, one data point represents the number per area of a hair follicle. GraphPad Prism 9; Mean±SEM; D'Agostino & Pearson omnibus normality test, no Gaussian distribution, Mann Whitney test n.s. CD3/CD8/Ki-67 triple positive cells in the epithelium of donors treated with vehicle or API-1 are not shown, as no cells could be detected.

From FIGS. 32A (API-1, chronic donor) and 32B and 32C (API-2, chronic donor), Ki-67 expression coupled with CD3 and CD8 positive indicators suggest the proliferation of T cells. API-1 slightly reduces CD3/Ki-67 dual positive cells in the epithelium. API-2 tendentially reduced CD3/Ki-67 and CD3/CD8/Ki-67 positive cells in the epithelium. This signifies a reduction in the immune response.

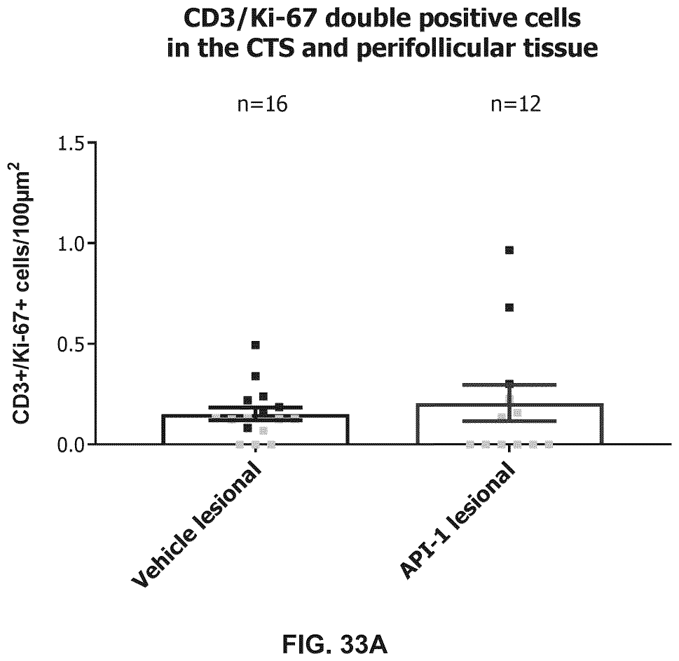

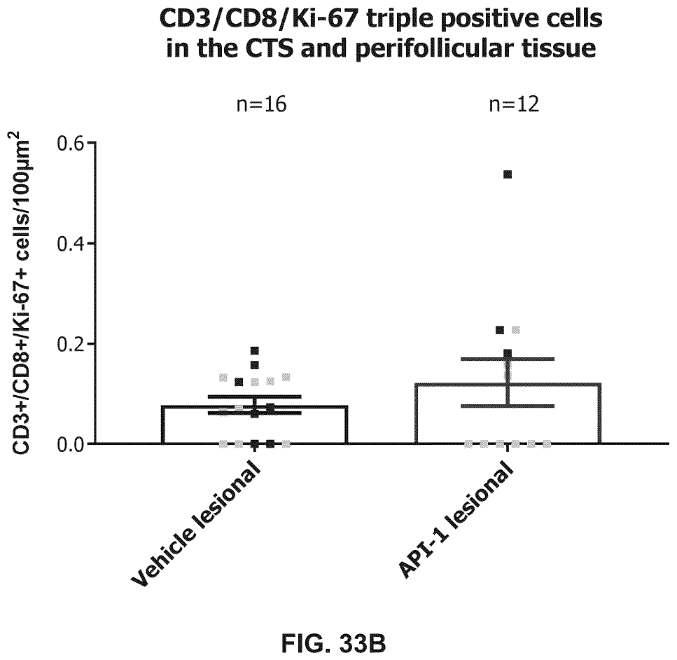

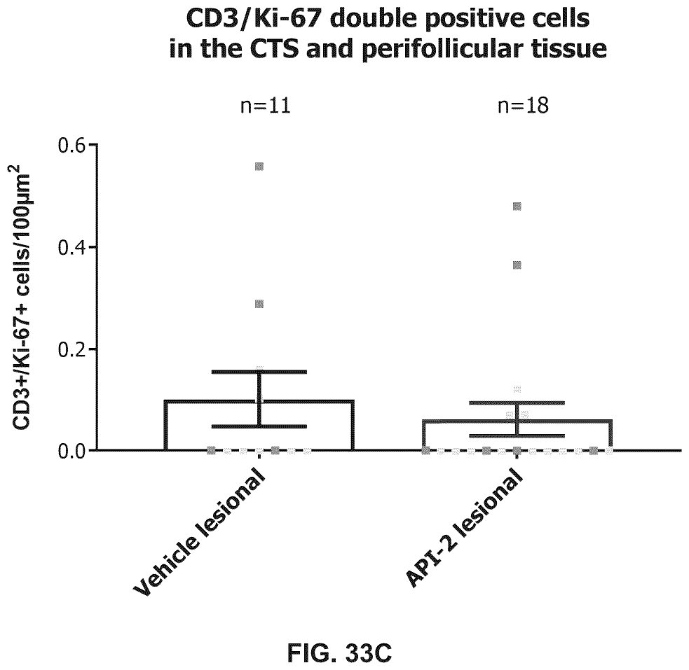

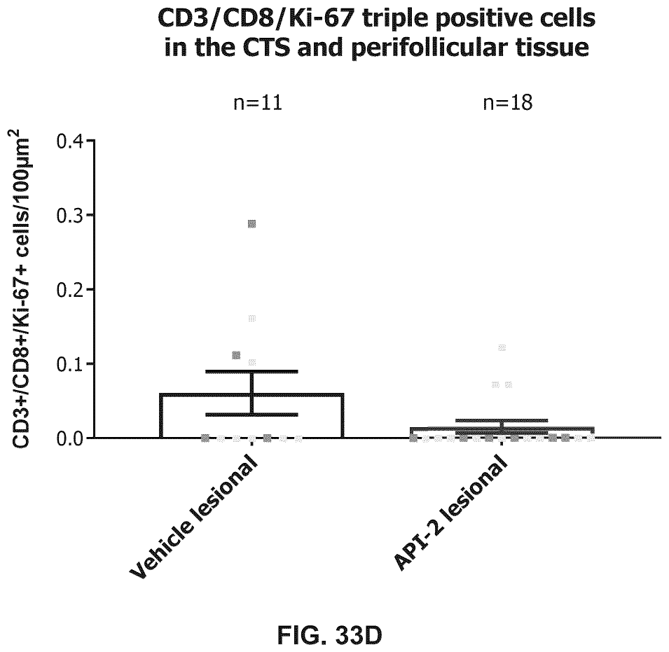

In FIGS. 33A and 33B (API-1, chronic donor) and 33C and 33D (API-2, chronic donor), CD3/Ki-67 double positive cells in the CTS and perifollicular tissue, and CD3/CD8/Ki-67 triple positive cells in the CTS and perifollicular tissue were examined. In these figures, n=11-18 HFs/group from N=4 chronic AA patients, one data point represents the number per area of a hair follicle. GraphPad Prism 9; Mean±SEM; D'Agostino & Pearson omnibus normality test, for donor 2-1 and 6-1: Gaussian distribution, Unpaired t test n.s.; for donor 3-2 and 5-2, no Gaussian distribution, Mann Whitney test n.s.

From FIGS. 33A and 33B (API-1, chronic donor) and 33C and 33D (API-2, chronic donor), Ki-67 expression coupled with CD3 and CD8 positive T cells indicate proliferation. API-2 treated samples compared to vehicle had a reduction in CD3/Ki-67 dual positive and CD3/CD8/Ki-67 positive cells. This signifies a reduced immune response.

More recently tissue resident memory T cells (TRM) have been suggested to play a role in AA (de Jong et al., JCI Insight 2018; Ryan et al., Front Immunol 2021; Tokura et al., Front Immunol 2021) and hence cultured biopsies, from chronic AA donors, were stained with antibodies against the human CD3 (Abcam, Cat A51001, Alexa flour 647 conjugated, clone UCHT1), CD69 (Biolegend, Cat 310906, PE conjugated clone MOPC-21) and CD103 (Thermofisher, cat 14-1038-82 clone B-Ly7) (Tokura et al., Front Immunol 2021). After image acquisition, cell numbers were assessed by manual counting of total CD3+, CD69+ and CD3+CD69+ in two regions of interest. ROIs, (highlighted in the representative image below): hair follicle (HF) epithelium as well as connective tissue sheath, CTS, and perifollicular tissue. Due to the fact that CD103+ cells were rare or even absent in the ROIs, numbers of CD103+ cells are not shown. A minimum of 6 HFs per condition, per donor, were evaluated. The numbers of total CD3+ cells, CD3+CD69+, and CD69+ are shown normalized to area (per 100 μm²) and of CD3+CD69+ T cells also as a percent of total CD3+.

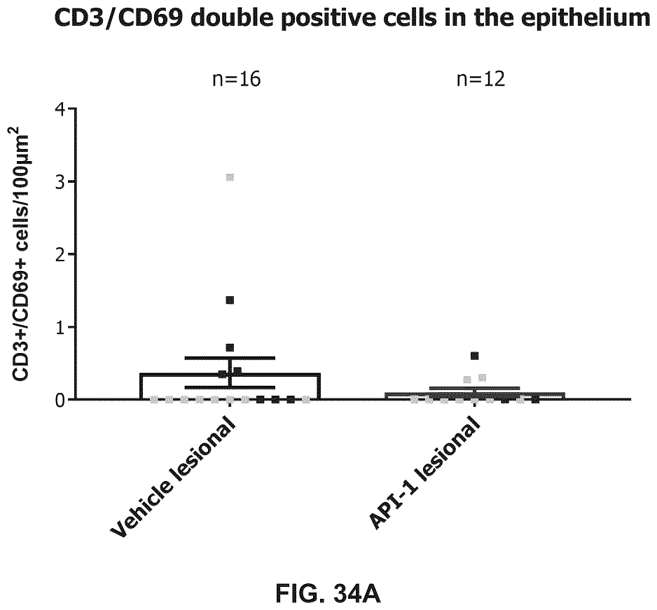

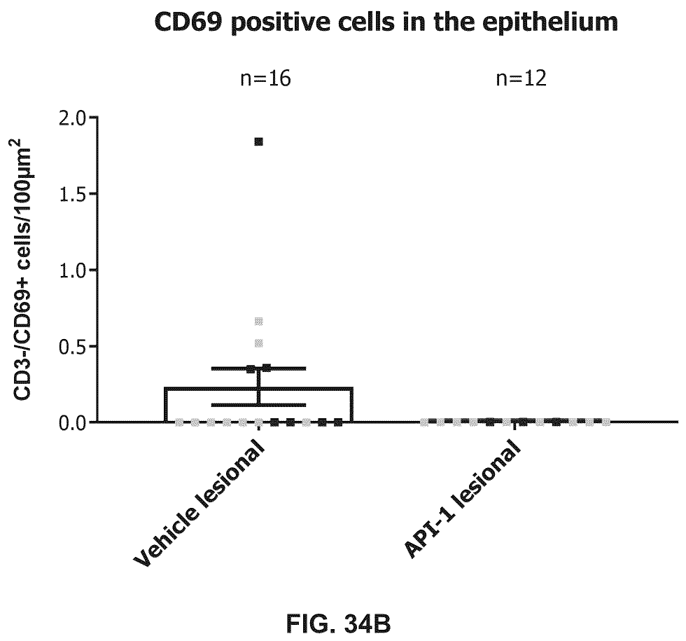

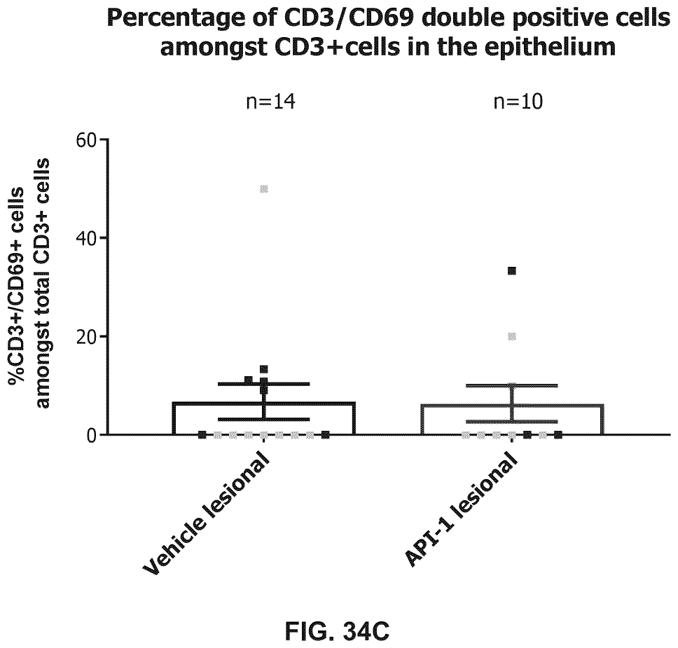

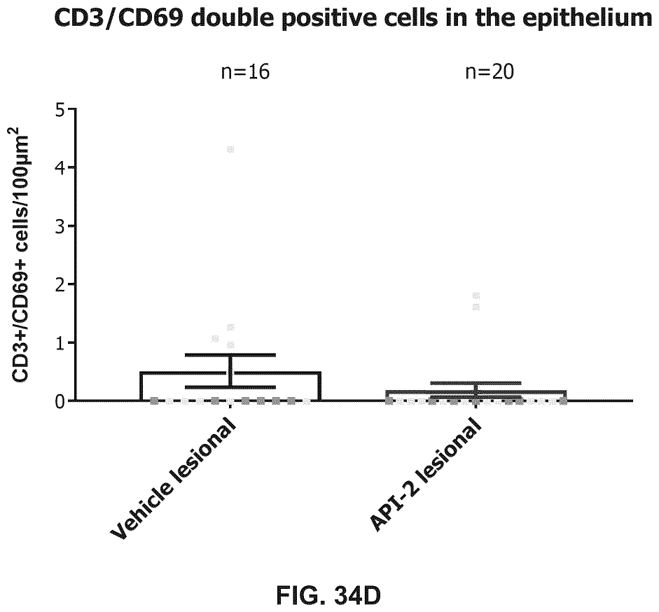

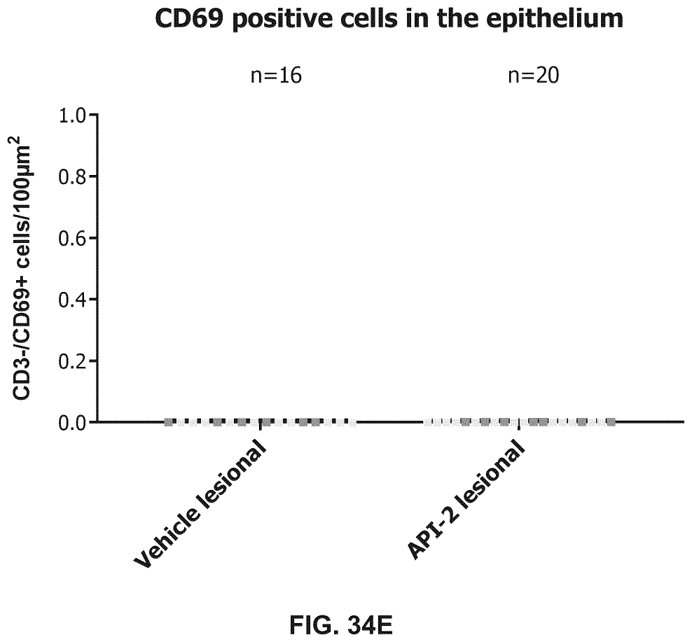

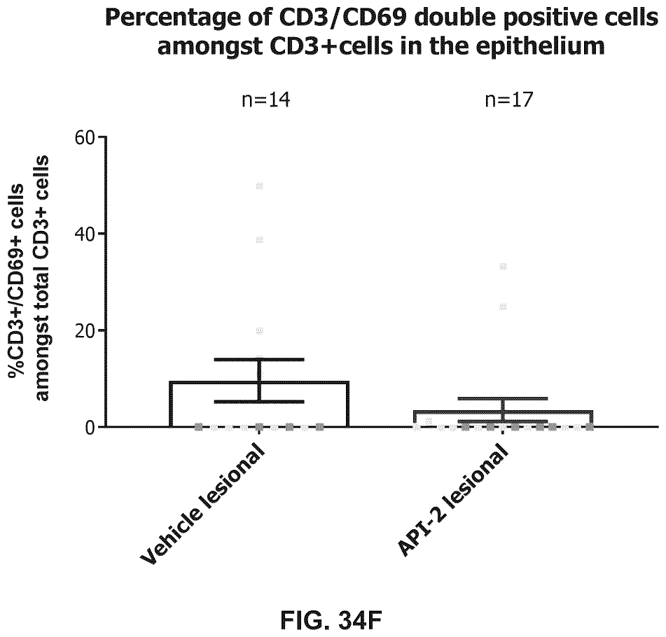

In FIGS. 34A-34C (API-1, chronic donor) and 34D-34F (API-2, chronic donor), CD3/CD69 double positive cells in the epithelium, CD69 positive cells in the epithelium, and the percentage of CD3/CD69 double positive cells amongst CD3+ cells in the epithelium were examined. In these figures, n=12-20 HFs/group from N=4 chronic AA patients. One data point represents the number per area of a hair follicle. GraphPad Prism 9; Mean±SEM; D'Agostino & Pearson omnibus normality test, no Gaussian distribution, Mann Whitney test n.s. Due to 0 for some data points in the vehicle, the evaluation of the percentage was not possible for these HFs.

From FIGS. 34A-34C (API-1, chronic donor) and 34D-34F (API-2, chronic donor), CD69 indicates the activation of T cells. API-1 treated chronic AA lesions had a reduction of CD3/CD69 double positive and CD69 positive cells. This indicates the reduction of activated T cells in the epithelium in chronic AA.

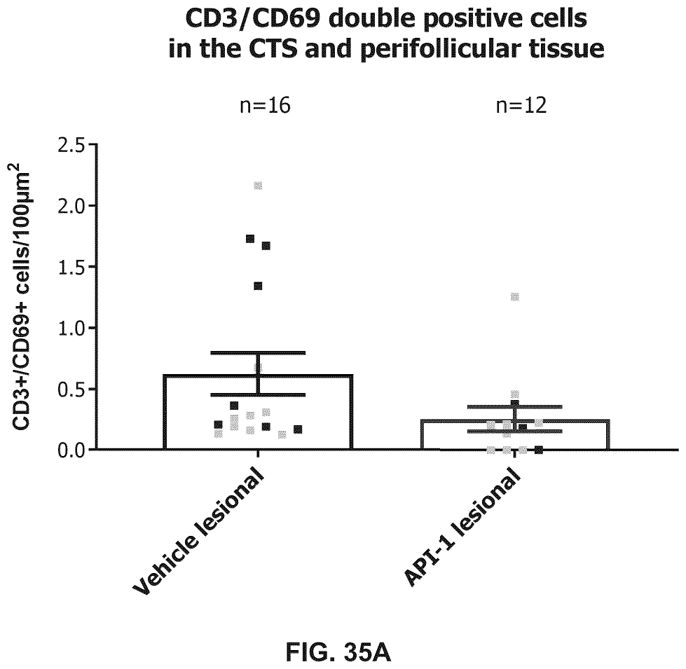

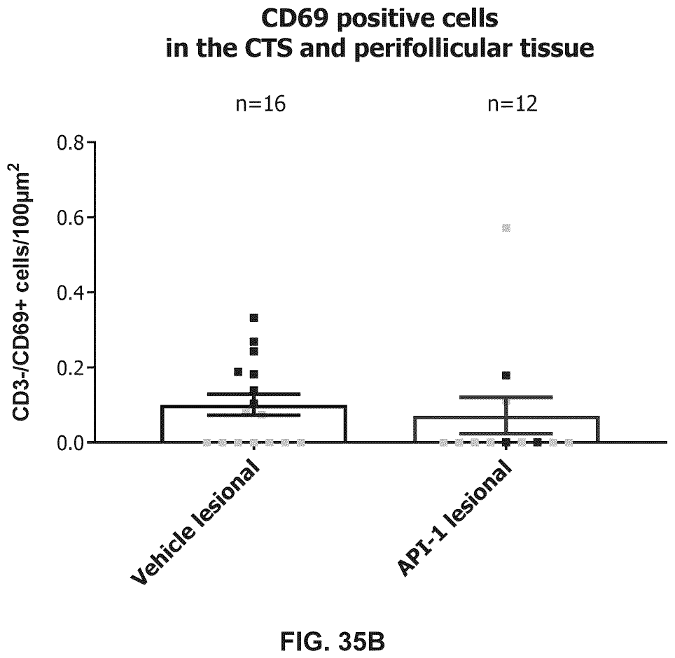

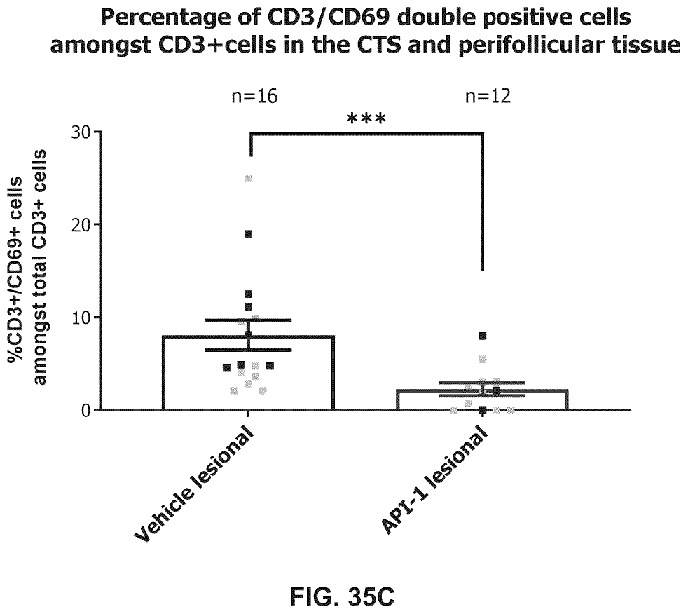

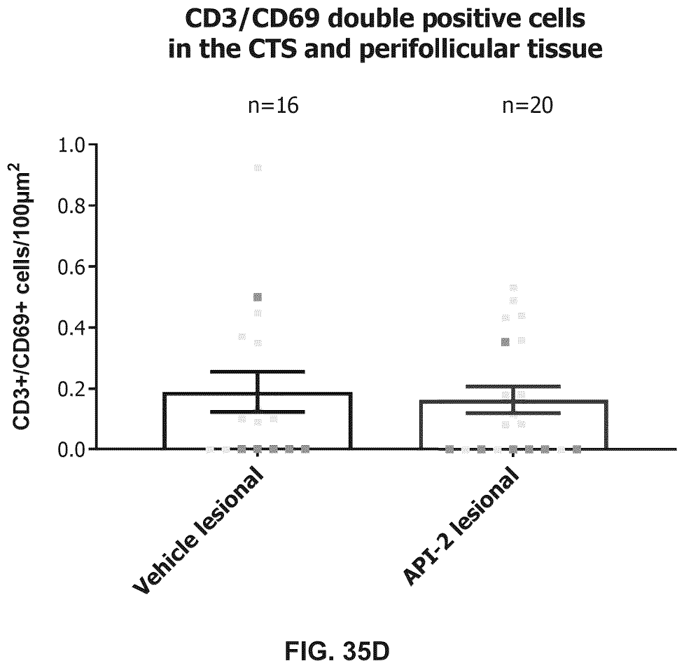

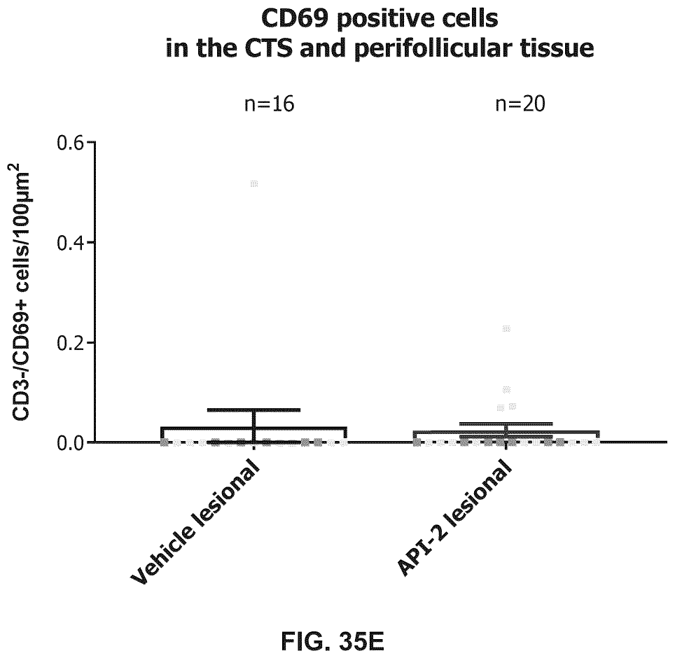

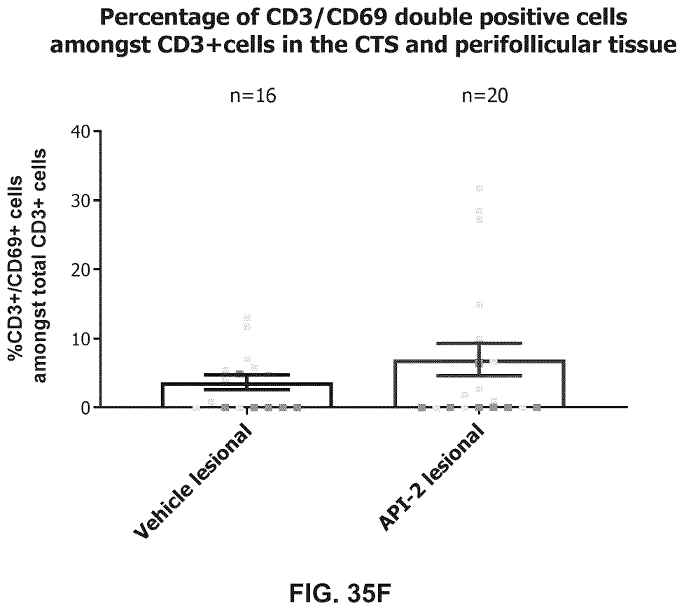

In FIGS. 35A-35C (API-1, chronic donor) and 35D-35F (API-2, chronic donor), CD3/CD69 double positive cells in the CTS and perifollicular tissue, CD69 positive cells in the CTS and perifollicular tissue, and the percentage of CD3/CD69 double positive cells amongst CD3+ cells in the CTS and perifollicular tissue were examined. In these figures, n=12-20 HFs/group from N=4 chronic AA patients. One data point represents the number per area of a hair follicle. GraphPad Prism 9; Mean±SEM; D'Agostino & Pearson omnibus normality test, no Gaussian distribution, Mann Whitney test n.s. Of note, only very few CD103+ cells could be detected in the hair follicle connective tissue sheath and perifollicular tissue (data not shown).

From FIGS. 35A-35C (API-1, chronic donor) and 35D-35F (API-2, chronic donor), CD69 indicates the activation of T cells. API-1 showed a significant decrease in the percentage of CD3/CD69 dual positive cells over the total number of CD3 positive cells in the perifollicular tissue. There was a tendentially reduction for API-2 for CD3/CD69 dual positive and CD69 positive cells in the perifollicular tissue. The reduction of CD69 indicates a reduction of activity of T cells in the samples treated with API compared to the vehicle.

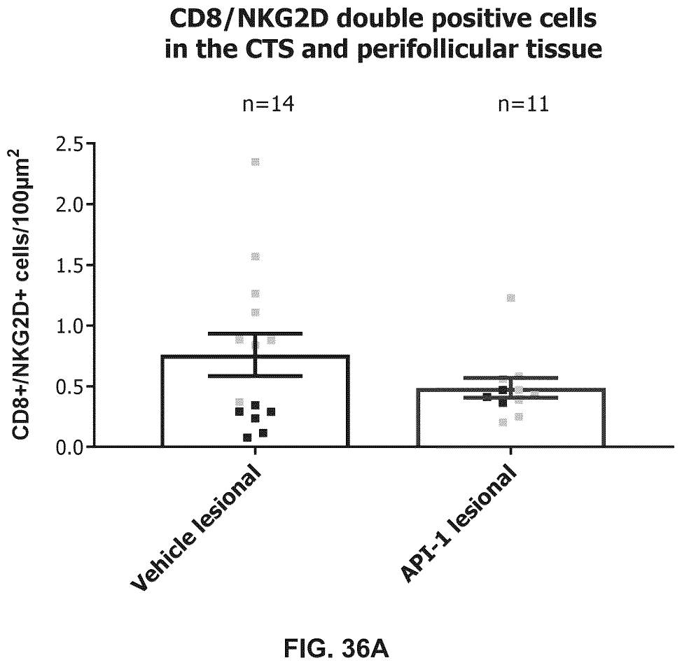

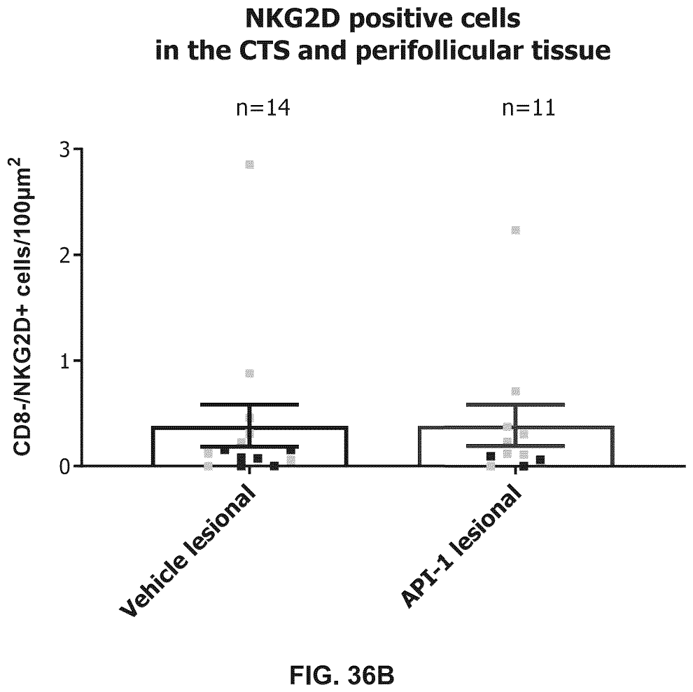

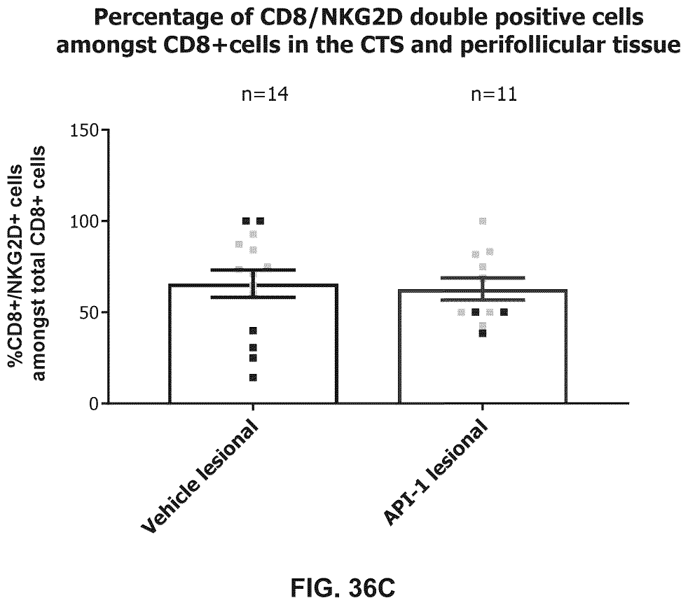

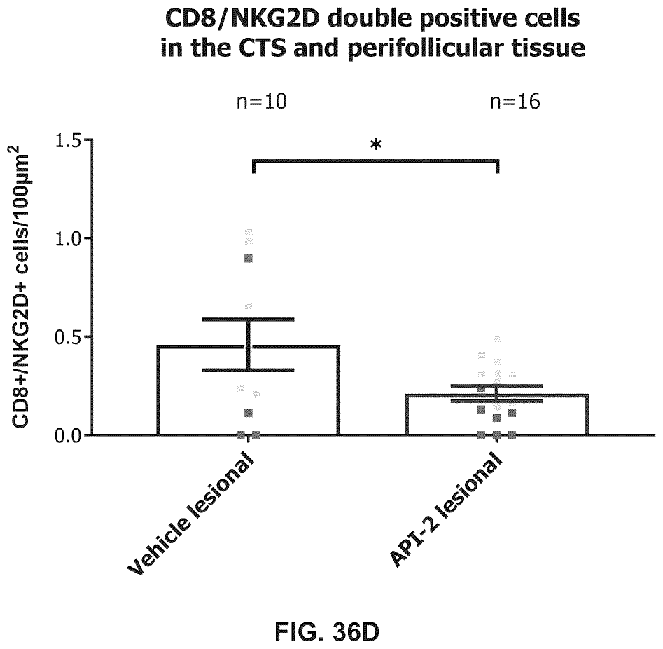

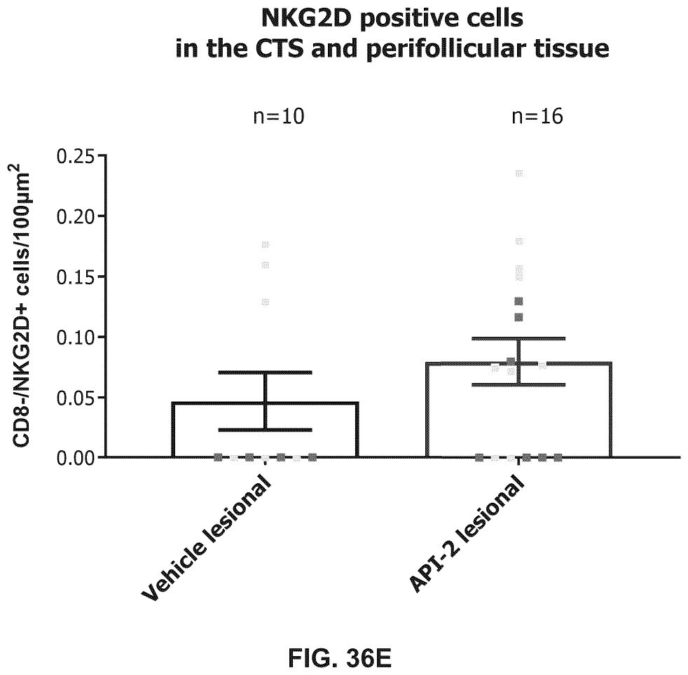

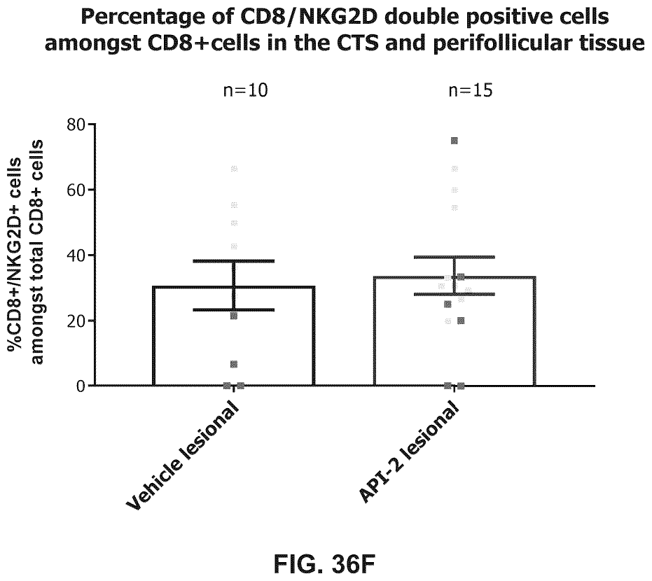

In FIGS. 36A-36C (API-1, chronic donor) and 36D-36F (API-2, chronic donor), CD8/NKG2D double positive cells in the CTS and perifollicular tissue, NKG2D positive cells in the CTS and perifollicular tissue, and the percentage of CD8/NKG2D double positive cells amongst CD8+ cells in the CTS and perifollicular tissue were examined. In these figures, n=10-16 HFs/group from N=4 chronic AA patients. One data point represents the number per area of a hair follicle. GraphPad Prism 9; Mean±SEM; D'Agostino & Pearson omnibus normality test, for CD8/NKG2D double positive cells and for NKG2D positive cells after API-1 treatment: no Gaussian distribution, Mann Whitney test *p<0.05; for percentage of CD8/NKG2D double positive for API-1 and API-2 treatment and for CD8/NKG2D double positive cells and for NKG2D positive cells after API-2 treatment Gaussian distribution, Unpaired t test p *<0.05.

From FIGS. 36A-36C (API-1, chronic donor) and 36D-36F (API-2, chronic donor), NKG2D is an activating receptor which results in the reduction of the threshold for T-cell activation. API-1 tendentially reduced and API-2 was significantly reduced in CD8/NKG2D dual positive cells in the perifollicular tissue in chronic AA.

T cell proliferation and activation, particularly from CD8+ effector T cells, are typical hallmarks of AA (Bertolini et al., Exp Dermatol 2020), however, FoxP3 expressing T-regs can help in resolution of inflammation and restoration of hair follicle immune privilege. Hence cultured biopsies, from chronic AA donors, were stained with antibodies against the human CD3 (Biolegend, Cat 300508, PE conjugated, clone RPA-T4) and FoxP3 (BD bioscience cat 560044, clone 259D/C7). After image acquisition, CD3+FOXP3+ cells could not detected around the hair follicles, therefore, we decided to analyze them in the papillary dermis. T-regs cell number were assessed by manual counting of CD3+FoxP3+ double positive cells in the papillary dermis. From 4 different slides per group one section each was selected for the evaluation. We took 2-4 images from the dermis per section. The number of CD3+FoxP3+ double positive cells are shown normalized to area (per 100 μm²).

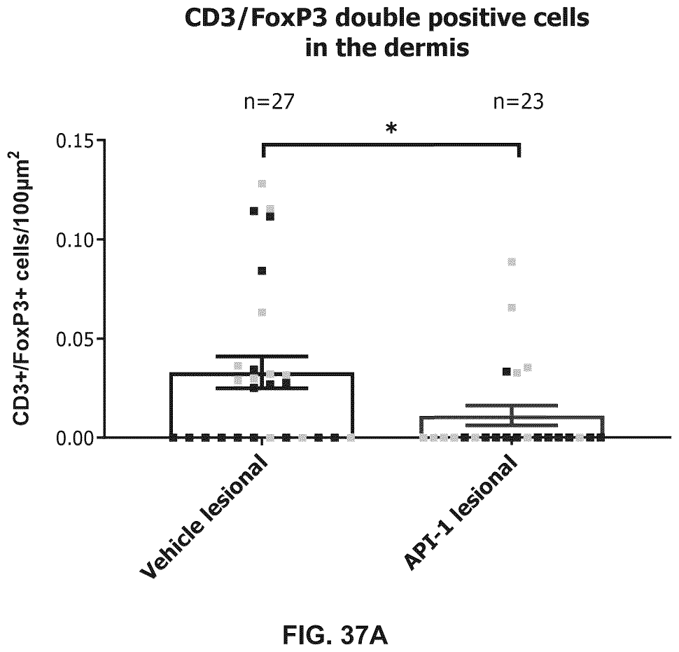

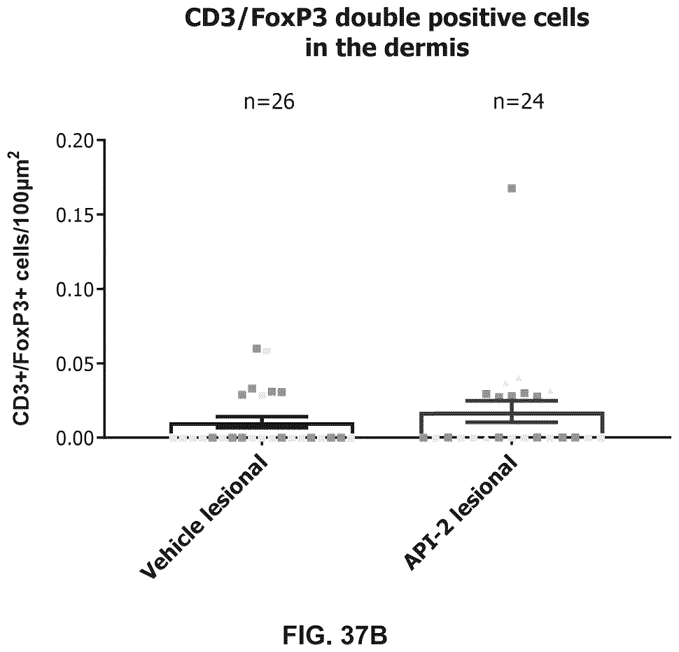

In FIGS. 37A (API-1, chronic donor) and 37B (API-2, chronic donor), CD3/FoxP3 double positive cells in the dermis were examined. In these figures, n=23-27 areas/group from N=4 chronic AA patients. One data point represents the number of cells per area of one image. We have taken 2-4 images from the dermis per section and selected 1 section from four slides per group. GraphPad Prism 9; Mean±SEM; D'Agostino & Pearson omnibus normality test, no Gaussian distribution, Mann Whitney test *p<0.05. Of note, CD3+FOXP3+ cells could not detected around the hair follicles, therefore, the papillary dermis cells were analyzed.

From FIGS. 37A (API-1, chronic donor) and 37B (API-2, chronic donor), FoxP3 indicates the normal regulation of T cells and alopecia areata has shown a deficiency of FoxP3+CD39+ and T regulatory cells. API-1 showed a reduction in CD3/FoxP3 double positive cells and API-2 indicates an increase in CD3/FoxP3 dual positive cells. This may indicate API-2 maybe beneficial in the immune regulation in the hair bulb of chronic alopecia patients.

### Example 13H: Cytokine and Chemikine Release into Culture Medium

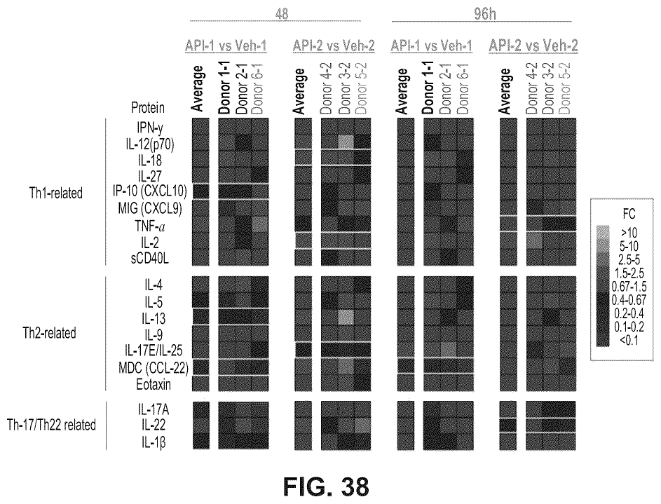

Th1, TH2, Th17/Th22 related cytokines were evaluated in release media. In FIG. 38, the heatmap shows the average fold change of each cytokine secretion into the supernatant after 48 h and 96 h treatment with API-1 or API-2, compared to the vehicle-treated control in lesional skin from acute (Donor 1-1; Donor 4-2) and chronic (Donor 2-1, 6-1, 3-2, 5-2) AA donors. From FIG. 38, API 1 prevented the release of T cell chemoattractant CXCL10 and cytokines for the 48 timepoint. The release impacts the immune response and decrease in the release reduces the potential for the immune progression of AA. There was an observed increase in IL-18 and IL-2 for API-2, however at 96 hours there was a decrease in IL-22 and TNFα secretion.

### Example 131: IFN-γ, CXCL10, CCL22, and IL-17A Production of Pooled Samples

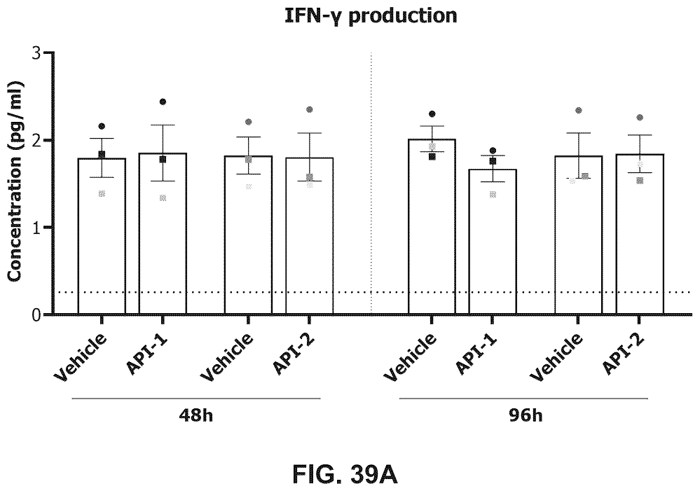

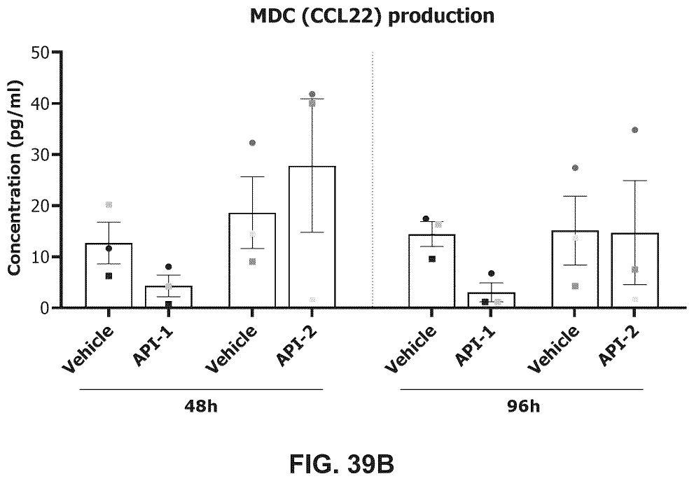

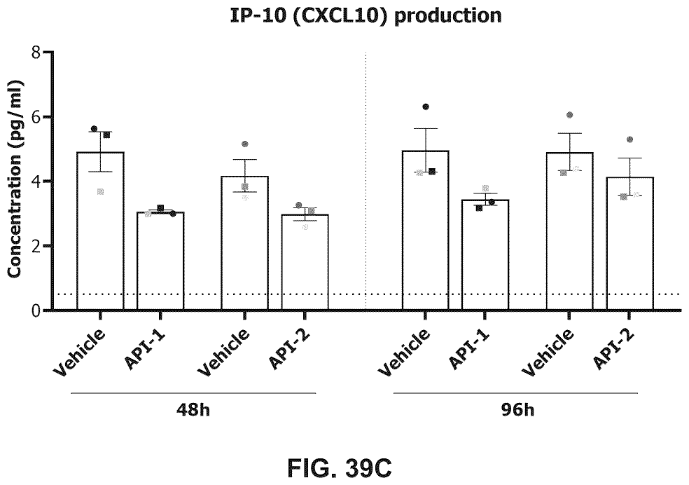

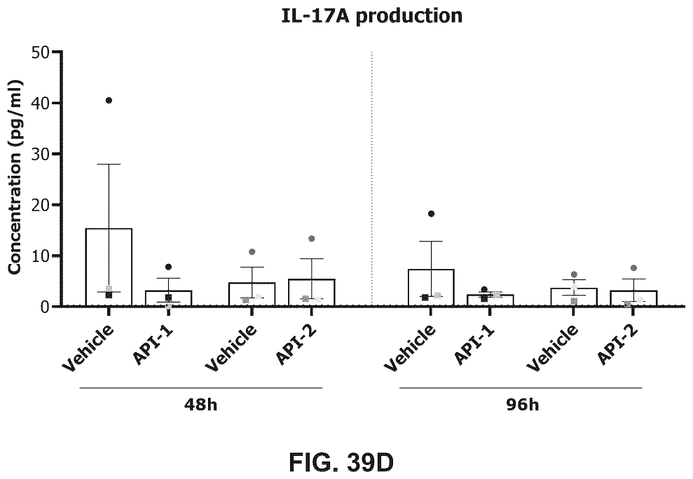

In FIG. 39A, IFN-γ production, 39B MDC (CCL22) production, 39C IP-10 (CXCL10) production, and 39D IL-17A production were examined at 48 hours and 96 hours. From those figures, Mean±SEM cytokine concentration from supernatant of AA lesional skin after 48 h and 96 h treatment ex vivo with API-1, API-2 or vehicle from n=3 independent human donors per API. Dashed lines mark the lower limit of detection (slide 13). GraphPad Prism 9. D'Agostino & Pearson test for normal distribution. Friedman test; Dunn's multiple comparison test for comparison to the vehicle-treatment groups or for comparison API-1 vs API-2, n.s.

From FIGS. 39A-39D, both API-1 and API-2 reduce the release of a T cell chemoattractant CXCL10 and API-1 decreases the secretion of CCL22. CXCL 10 is a protein which is proinflammatory which may lead to an immune response in the hair bulb. CCL22 regulates T cell immunity and the decrease by API-1 indicates the reduction of the auto immune response in the alopecia samples.

### Example 13J: Cytotoxic T Cells/NK Cells

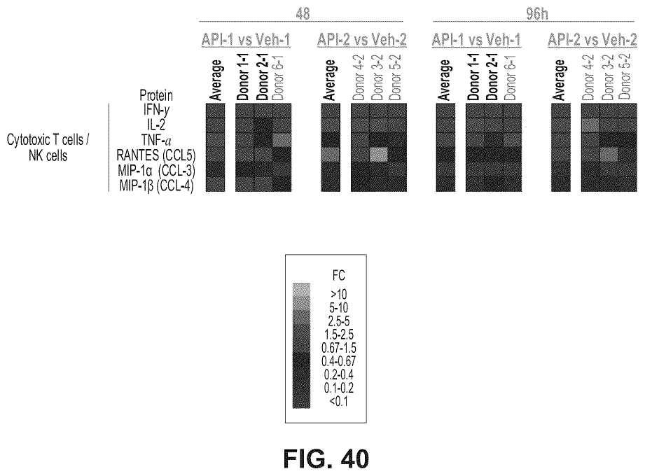

In FIG. 40, the heatmap shows the average fold change of each cytokine secretion into the supernatant after 48 h and 96 h treatment with API-1 or API-2, compared to the vehicle-treated control in lesional skin from acute (Donor 1-1; Donor 4-2) or chronic (Donor 2-1, 6-1, 3-2, 5-2) AA donors.

From FIG. 40, the fold changes indicate the application of treatments API-1 and API-2 decrease the secretions of chemokines associated with the immune response of cytotoxic and cytolytic T cells and natural killer cells (NK) at 48 and 96 hrs.

### Example 13K: Samples for the Secretion of Chemokines

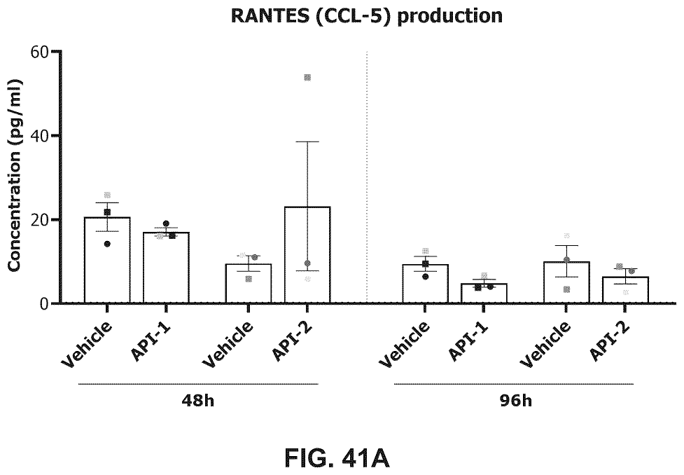

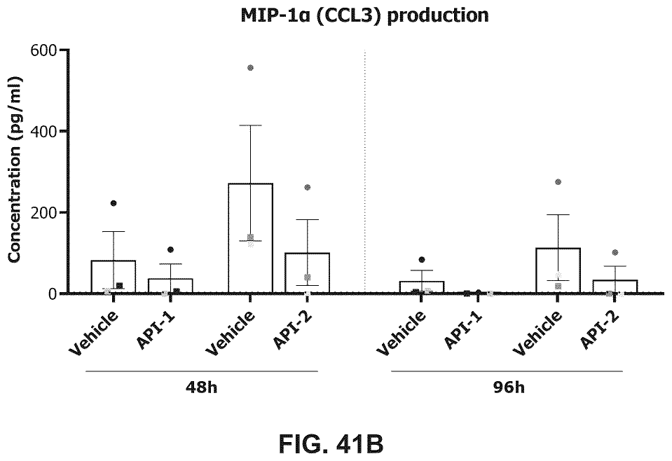

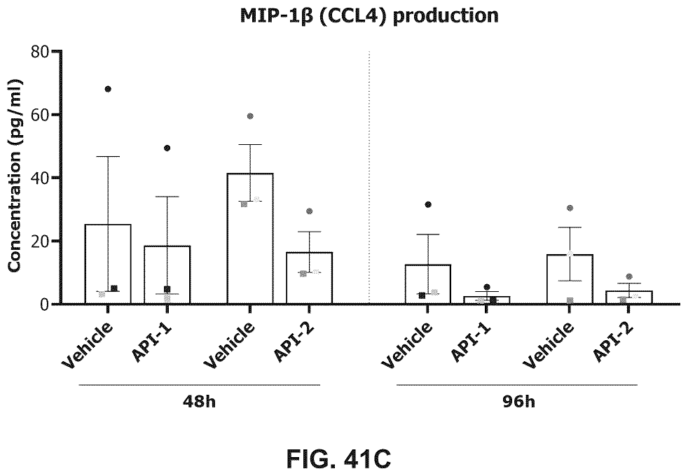

In FIGS. 41A-C, Rantes (CCL-5), MP-1a, and MIP-1B (CCL4) productions were examined. In these figures, Mean±SEM cytokine concentration from supernatant of AA lesional skin after 48 h and 96 h treatment ex vivo with API-1, API-2 or vehicle from n=3 independent human donors per API. Dashed lines mark the lower limit of detection. GraphPad Prism 9. D'Agostino & Pearson test for normal distribution. Friedman test; Dunn's multiple comparison test for comparison to the vehicle-treatment groups or for comparison API-1 vs API-2, n.s.

From FIGS. 41A-C, the plots indicate a decrease in CCL-5 for API-1 at 48 and 96 hours and a decrease for API-1 for 96 hours. Decreases were observed for API-1 and API-2 for CCL3 and CCL4 for both timepoints. This impact NK cells which are a type of white blood cells and lymphocyte that is a part of the immune system.

### Example 13L: Heatmap of Samples to Examine Chemotactic Cytokines

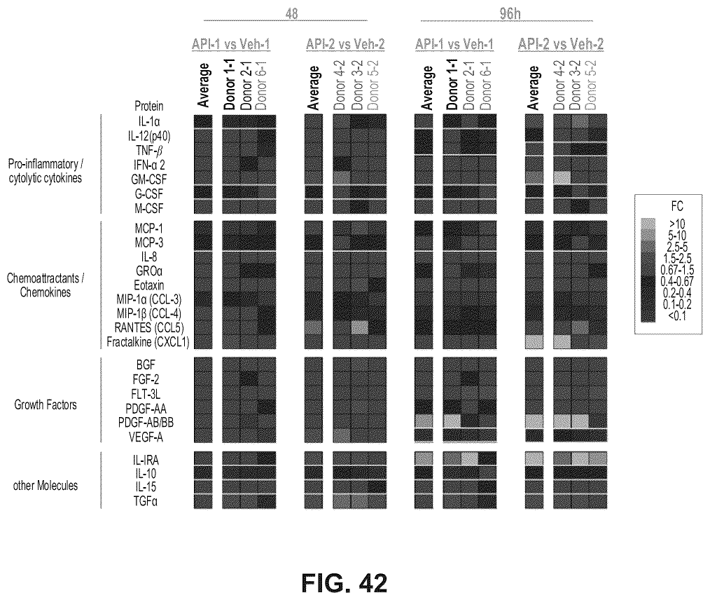

In FIG. 42, the heatmap shows the average fold change of each cytokine secretion into the supernatant after 48 h and 96 h treatment with API-1 or API-2, compared to the vehicle-treated control in lesional skin from acute (Donor 1-1; Donor 4-2) or chronic (Donor 2-1, 6-1, 3-2, 5-2) AA donors.

From FIG. 42, API-1 and API-2 decreased the release of MCP-1, MCP-3, CCL3, CCL4, IL-1α, and IL-10 which are involved in the immune response.

### Example 13M: API-1 and API-2 Conclusions from Chemotactic Cytokines

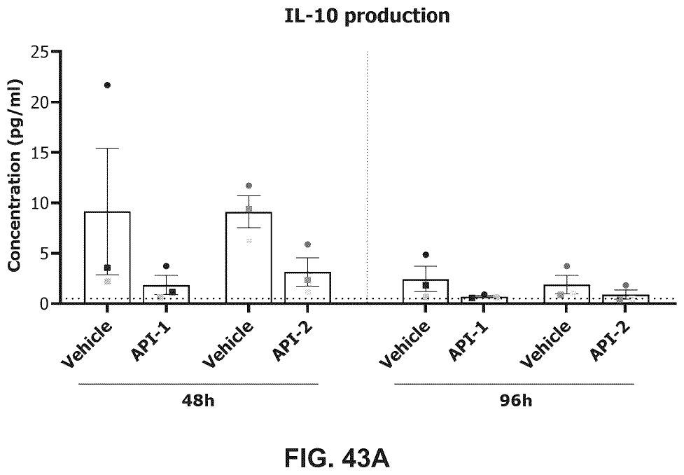

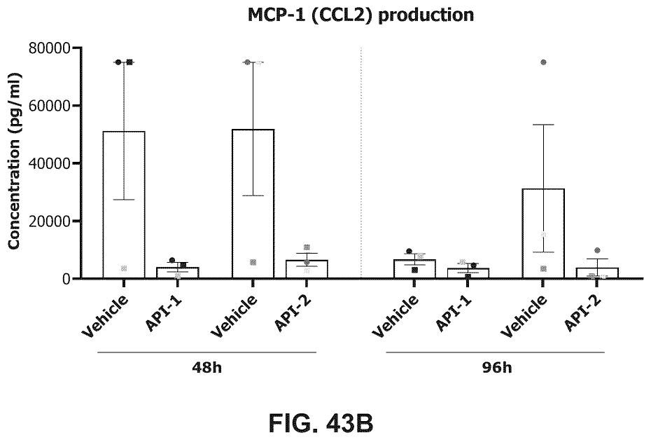

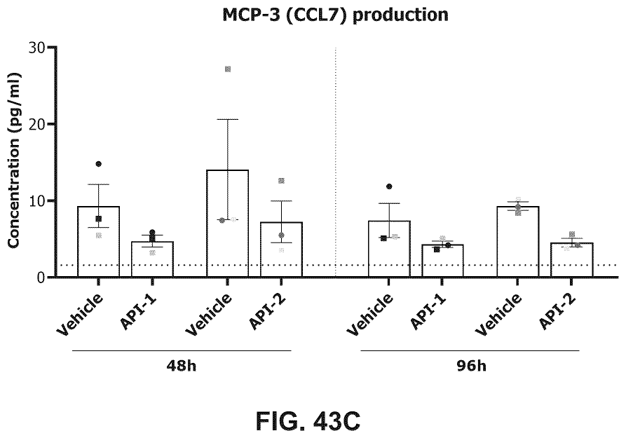

In FIGS. 43A-43C, IL-10, MCP-1 (CCL2), and MCP-3 (CC7) productions are examined in API-1 and API-2. From FIGS. 43A-C, API 1 and API 2 decreased chemotactic cytokines MCP-1, MCP-3, CCL3, and CCL4 which stimulate the migration of different inflammatory cells.

### Example 13N: Acute Alopecia Summary of Results

The following summary is provided

| Read out | API-1 | API-2 |
|---|---|---|
| Hair cycle (anagen maintenance) | = | = |
| Melanin clumping (hair follicle dystrophy) | = | = |
| Hair matrix keratinocytes | ↑* | ↑ |
| MHC-I ORS/gHM/DC/DPst | ↓*/↓*/↓*/= | =/=/=/= |
| MHC-II ORS | ↑ | ↑ |
| MHC-II+ cells ORS/CTS/PFT | ↑/↑/↓ | ↑/↑/↓ |
| CD8+ cells epithelium/CTS+PFT | =/= | =/= |
| CD8+CD69+ cells CTS+PFT | ↓ | = |
| CD8−CD69+ cells CTS+PFT | = | = |
| Percentage CD8+CD69+ cells of total | ↓ | = |
| CD8+ CTS+PFT | | |
| CD8+NKG2D+ cells CTS+PFT | ↓ | ↓ |
| CD8−NKG2D+ cells CTS+PFT | = | = |
| Percentage CD8+NKG2D+ cells of total | ↓ | = |
| CD8+ CTS+PFT | | |

Legend:
↓ decreased;
↑ increased;
*significant;
= unaffected
ORS = outer root sheath,
gHM = germinative hair matrix,
Dc = dermal cup,
DPst = dermal papilla stalk,
CTS = connective tissue sheath,
PFT = perifollicular tissue,
n.a. = not applicable;
*indicates statistical significance

### Example 130: Chronic Alopecia Summary of Results

| Read out | API-1 | API-2 |
|---|---|---|
| CD3+ cells epithelium/CTS+PFT | =/= | ↓/= |
| CD8+ cells epithelium/CTS+PFT | ↓/= | ↓*/↓ |
| Proliferating CD3+ cells epithelium/CTS+PFT | ↓/= | ↓/↓ |
| Proliferating CD8+ cells epithelium/CTS+PFT | n.a./↑ | ↓/↓ |
| CD3+CD69+ epithelium/CTS+PFT | ↓/↓ | ↓/↓ |
| CD3−CD69+ epithelium/CTS+PFT | ↓/= | n.a./= |
| Percentage CD3+CD69+ cells of total CD3+ | =/↓* | =/↑ |
| epithelium/CTS+PFT | | |
| CD8+NKG2D+ cells CTS+PFT | ↓ | ↓* |
| CD8−NKG2D+ cells CTS+PFT | = | = |
| Percentage CD8+NKG2D+ cells of total CD8+ | = | = |
| CTS+PFT | | |
| CD3+FOXP3+ cells papillary dermis | ↓* | = |

Legend:
↓ decreased;
↑ increased;
*significant;
= unaffected
CTS = connective tissue sheath,
PFT = perifollicular tissue,
n.a. = not applicable,
*indicates statistical significance

### Example 13P: Summary of Chronic Donor Pool

| | API-1 | | API-2 | |
|---|---|---|---|---|
| Read out | Epi | CTS/PFT | Epi | CTS/PFT |
| CD3+ cells | = | = | ↓ | = |
| CD8+ cells | ↓ | = | ↓* | ↓ |
| Proliferating CD3+ cells | ↓ | = | ↓ | ↓ |
| Proliferating CD8+ cells | n.a. | ↑ | ↓ | ↓ |
| CD3+CD69+ | ↓ | ↓ | ↓ | ↓ |
| CD3−CD69+ | ↓ | = | n.a. | = |
| Percentage CD3+CD69+ | = | ↓* | = | ↑ |
| cells of total CD3+ | | | | |
| CD8+NKG2D+ cells | n.a. | ↓ | n.a. | ↓* |
| CD8−NKG2D+ cells | n.a. | = | n.a. | = |
| Percentage CD8+NKG2D+ | n.a. | = | n.a. | = |
| cells of total CD8+ | | | | |

| | | |
|---|---|---|
| | Dermis | Dermis |
| CD3+FOXP3+ cells | ↓* | = |

Legend:
↓ decreased;
↑ increased;
*significant;
= unaffected
Epi: epithelium,
CTS: connective tissue sheath,
PFT: perifollicular tissue,
n.a.: not applicable,
*indicates statistical significance,
n.a. = not assessed

Summary of Acute Alopecia Samples from Example 13

(1) With the acute treatment of the skin samples there was a maintenance of the anagen phase in the non-lesional samples. This was not observed in lesional samples treated with API 1 and API 2.

(2) Melanin clumping is an indication of hair follicle dystrophy. The application of API 1 and API 2 did not lead to hair follicle dystrophy which can be induced by chemical, biological and physical damage. This may indicate both applications do not induce hair follicle dystrophy and may not be toxic to the hair follicles.

(3) Matrix keratinocyte proliferation was observed for API 1 in lesional and non-lesional samples (result was statistically significant. This effect was less pronounced for API 2 and a slight increase in keratinocyte proliferation was observed.

(4) A restorative effect of the immune privilege was observed with the decrease in MHC 1 levels. This was robustly observed for API 1 and tendentially for API 2.

(5) The effect for (4) was not observed with MHC 2 (increase was observed) which is in line with prior research which indicates pre-administration (e.g., in a murine vibrissae organ culture model) of ruxolitinib before the stimulation with IFNg to inhibit immune privilege collapse. However, API 2 treatment tendentially decreased the number of MHC 2 cells in perifollicular tissue. This suggests API 2 may result in the modulation of the peri-follicular antigen presenting cell activation/survival.

(6) Both API-1 and API-2 tendentially decreased CD8 positive/NKG2D positive cells. This is a pathogenic CD8 positive T-cell population. This is can indicate a reduction in the T-cell immune response.

(7) API-1 tendentially decrease the number and percentage of CD8 positive/CD69 positive cells in lesional follicles. This can represent the decrease in the either activated or resident memory T-cells.

(8) API-1 may be more efficacious in subjects with acute alopecia.

Summary of Chronic Alopecia Samples from Example 13

(1) Literature indicates that there is variability of patients with chronic alopecia areata which may be due to age, sex, onset of the lesions, and type.

(2) API-1 tendentially reduced CD3/CD8 dual positive when compared to the vehicle treated lesional samples. API-2 tendentially reduced CD3 positive cells and significantly reduced CD3/CD8 dual positive cells compared to the vehicle. This signifies the reduced activation of an immune response in the epithelium.

(3) API-2 had a significant reduction in CD8 positive cells in the perifollicular tissue.

(4) Ki-67 expression coupled with CD3 and CD8 positive indicators suggest the proliferation of T cells. API-1 slightly reduces CD3/Ki-67 dual positive cells in the epithelium. API-2 tendentially reduced CD3/Ki-67 and CD3/CD8/Ki-67 positive cells in the epithelium.

(5) Ki-67 expression coupled with CD3 and CD8 positive T cells indicate

proliferation. API-2 treated samples compared to vehicle had a reduction in CD3/Ki-67 dual positive and CD3/CD8/Ki-67 positive cells.

(6) CD69 indicates the activation of T cells. API-1 treated chronic AA lesions had a reduction of CD3/CD69 double positive and CD69 positive cells. This indicates the reduction of activated T cells in the epithelium in chronic AA.

(7) CD69 indicates the activation of T cells. API-1 showed a significant decrease in the percentage of CD3/CD69 dual positive cells over the total number of CD3 positive cells in the perifollicular tissue. There was a tendentially reduction for API-2 for CD3/CD69 dual positive and CD69 positive cells in the perifollicular tissue. The reduction of CD69 indicates a reduction of activity of T cells in the samples treated with API compared to the vehicle.

(8) NKG2D is an activating receptor which results in the reduction of the threshold for T-cell activation. API-1 tendentially reduced and API-2 was significantly reduced in CD8/NKG2D dual positive cells in the perifollicular tissue in chronic AA.

(9) FoxP3 indicates the normal regulation of T cells and alopecia areata has shown a deficiency of FoxP3+CD39+ and T regulatory cells. API-1 showed a reduction in CD3/FoxP3 double positive cells and API-2 indicates an increase in CD3/FoxP3 dual positive cells. This may indicate API-2 may be beneficial in the immune regulation in the hair bulb of chronic alopecia patients.

### Example 14: Mini-Pig GLP Toxicology Study

Selection of formulations for the GLP toxicology study

From Tables 55A-D, the following formulations were included in the GLP toxicology study. The noted noted formulations below were prepared by the above manufacturing method B.

TABLE 55A
Ruxolitinib and deuruxolitinib foam formulations for GLP toxicology

| | | Product Description | | | |
|---|---|---|---|---|---|
| | | 2.5% | 2.5% | 2.5% | 2.5% |
| | | Ruxolitinib | Deuruxolitinib | Ruxolitinib | Deuruxolitinib |
| | | Foam (2.5% | Foam (2.5% | Foam (1% | Foam (1% |
| | | Emollient) | Emollient) | Emollient) | Emollient) |
| | | Lot number | | | |
| | | 268-23-04 | 268-24-01 | 268-23-05 | 268-24-02 |
| | | Emollients and permeation enhancers | | | |
| | | PEG300/Glycerin | | ML/Transcutol | |
| Item # | Ingredients | % w/w | % w/w | % w/w | % w/w |
| 1 | Purified water | 31.95 | 31.96 | 33.45 | 33.46 |
| 2 | Tween 60 | 1.35 | 1.35 | 1.35 | 1.35 |
| | (Polysorbate 60) | | | | |
| 3 | Propylene Glycol | 5.00 | 5.00 | 5.00 | 5.00 |
| 4 | Glycerin | 0.50 | 0.5 | — | — |
| 5 | Polyethylene | 2.00 | 2.00 | — | — |
| | Glycol 300 | | | | |
| 6 | Myristyl Lactate | — | — | 1.00 | 1.00 |
| 7 | Diethylene Glycol | — | — | 0.50 | 0.50 |
| | Monoethyl Ether | | | | |
| | (Transcutol-P) | | | | |
| 8 | Cetyl Alcohol | 2.20 | 2.20 | 2.20 | 2.20 |
| 9 | Stearyl Alcohol | 0.35 | 0.35 | 0.75 | 0.75 |
| 10 | Ethanol 100HG | 50.15 | 50.16 | 51.25 | 51.26 |
| 11 | Ruxolitinib | 3.30 | — | 3.30 | — |
| | Phosphate | | | | |
| | (salt form) | | | | |
| 12 | Deuruxolitinib | — | 3.281 | — | 3.281 |
| | Phosphate | | | | |
| | (salt form) | | | | |
| 13 | Triethanolamine | 1.20 | 1.20 | 1.20 | 1.20 |
| | 99% USP | | | | |
| | Total | 100.00 | 100.00 | 100.00 | 100.0 |
| | Total | 2% PEG 300 | 2% PEG 300 | 1% Myristyl | 1% Myristyl |
| | emollients | 0.5% Glycerin | 0.5% Glycerin | lactate | lactate |
| | Total Permeation | 0% | 0% | 0.5% | 0.5% |
| | enhancer | | | Transcutol-P | Transcutol-P |
| | pH range | 5-6 | 5-6 | 5-6 | 5-6 |

TABLE 55B
Additional Placebo and Ruxolitinib Foam Formulations for Stability Studies

| | | PRODUCT DESCRIPTION | | |
|---|---|---|---|---|
| | | PLACEBO | PLACEBO | PLACEBO FOAM, |
| | | FOAM, NE | FOAM, LE | 1E−0.5T |
| | | Formulation ID | | |
| | | 268-23-03 | 268-23-01 | 268-23-02 |
| | | Lot ID | | |
| | | 268-23-03 | 268-23-01 | 268-23-02 |
| | | % w/w | % w/w | % w/w |
| ITEM# | Ingredients IID Name | Required | Required | Required |
| 1 | Water | 34.34 | 31.95 | 33.45 |
| 2 | Polysorbate 60 | 1.35 | 1.35 | 1.35 |
| 3 | Propylene glycol | 5.00 | 5.00 | 5.00 |
| 4 | Glycerin | | 0.50 | |
| 5 | Polyethylene Glycol 300 | | 2.00 | |
| 6 | Myristyl Lactate | | | 1.00 |
| 7 | Diethylene Glycol | | | 0.50 |
| | Monoethyl Ether | | | |
| 8 | Cetyl Alcohol | 2.20 | 2.20 | 2.20 |
| 9 | Stearyl Alcohol | 0.75 | 0.35 | 0.75 |
| 10 | Ethanol | 51.86 | 52.15 | 51.25 |
| 11 | Active substance | 0.00 | 0.00 | 0.00 |
| 12 | Trolamine | 0.00 | 0.25 | 0.25 |
| | | 95.50 | 95.75 | 95.75 |

| | | | |
|---|---|---|---|
| Equivalent Ruxolitinib, Freebase | 0.00 | 0.00 | 0.00 |
| Emollients | 0% | 2% PEG 300 | 1% Myristyl |
| | | 0.5% Glycerin | lactate |
| Permeation enhancer/solubilising | 0% | 0% | 0.5% Transcutol-P |
| agent | | | |

TABLE 55C
Additional Ruxolitinib Foam Formulations for Stability Studies

| | | Formulation ID | | | |
|---|---|---|---|---|---|
| | | 268-23-06 | 268-23-07 | 268.23-08 | 268--23-09 |
| | | Lot ID | | | |
| | | 268-23-06 | 268-23-07 | 268-23-08 | 268--23-09 |
| | | % w/w | % w/w | % w/w | % w/w |
| ITEM # | Ingredients IID Name | Required | Required | Required | Required |
| 1 | Water | 34.34 | 36.07 | 32.63 | 34.15 |
| 2 | Polysorbate 60 | 1.35 | 1.35 | 1.35 | 1.35 |
| 3 | Propylene glycol | 5.00 | 5.00 | 5.00 | 5.00 |
| 4 | Glycerin | | | 0.50 | |
| 5 | Polyethylene | | | 2.00 | |
| | Glycol 300 | | | | |
| 6 | Myristyl Lactate | | | | 1.00 |
| 7 | Diethylene Glycol | | | | 0.50 |
| | Monoethyl Ether | | | | |
| 8 | Cetyl Alcohol | 2.20 | 2.20 | 2.20 | 2.20 |
| 9 | Stearyl Alcohol | 0.75 | 0.75 | 0.35 | 0.75 |
| 10 | Ethanol | 51.86 | 51.90 | 53.24 | 52.32 |
| 11 | Ruxolitinib | 3.298 | 1.979 | 1.979 | 1.979 |
| | phosphate | | | | |
| 12 | Trolamine | 1.20 | 0.75 | 0.75 | 0.75 |
| | | 100.00 | 100.00 | 100.00 | 100.00 |

| | | | | |
|---|---|---|---|---|
| Equiv. Ruxolitinib, freebase | 2.50 | 1.50 | 1.50 | 1.50 |
| Emollients | 0% | 0% | 2% PEG 300 | 1% Myristyl |
| | | | 0.5% Glycerin | lactate |
| Permeation enhancer | 0% | 0% | 0% | 0.5% Transcutol-P |

TABLE 55D
Additional Deuruxolitinib Foam Formulations for Stability Studies

| | | Product Name | | | |
|---|---|---|---|---|---|
| | | | | | 1.5% |
| | | 1.5% | 2.5% | 1.5% | Deuruxolitinib |
| | | Deuruxolitinib | Deuruxolitinib | Deuruxolitinib | Foam |
| | | Foam (LE) | Foam (NE) | Foam (NE) | (E−0.5T) |
| | | Formulation ID | | | |
| | | 268-24-05 | 268-24-03 | 268-24-04 | 268-24-06 |
| IT | | Lot ID | | | |
| EM | Ingredients | 268-24-05 | 268-24-03 | 268-24-04 | 268-24-06 |
| # | IID Name | % w/w Required | % w/w Required | % w/w Required | % w/w Required |
| 1 | Water | 32.63 | 34.35 | 36.08 | 34.15 |
| 2 | Polysorbate 60 | 1.35 | 1.35 | 1.35 | 1.35 |
| 3 | Propylene | 5.00 | 5.00 | 5.00 | 5.00 |
| | glycol | | | | |
| 4 | Glycerin | 0.50 | | | |
| 5 | Polyethylene | 2.00 | | | |
| | Glycol 300 | | | | |
| 6 | Myristyl | | | | 1.00 |
| | Lactate | | | | |
| 7 | Diethylene | | | | 0.50 |
| | Glycol | | | | |
| | Monoethyl | | | | |
| | Ether | | | | |
| 8 | Cetyl Alcohol | 2.20 | 2.20 | 2.20 | 2.20 |
| 9 | Stearyl | 0.35 | 0.75 | 0.75 | 0.75 |
| | Alcohol | | | | |
| 10 | Ethanol | 53.25 | 51.87 | 51.90 | 52.33 |
| 11 | Deuruxolitinib | 1.9685 | 3.281 | 1.9685 | 1.9685 |
| | Phosphate, API | | | | |
| 12 | Trolamine | 0.75 | 1.20 | 0.75 | 0.75 |
| | | 100.00 | 100.00 | 100.00 | 100.00 |

| Equiv. Ruxolitinib, | 1.50 | 2.50 | 1.50 | 1.50 |
|---|---|---|---|---|
| freebase | | | | |
| Emollients | 2% PEG 300 | 0% | 0% | 1% Myristyl |
| | 0.5% Glycerin | | | lactate |
| Permeation | 0% | 0% | 0% | 0.5% |
| enhancer/solubiliser | | | | Transcutol-P |

The ruxolitinib formulations for the GLP toxicology study demonstrated 3 months stability and were placed on stability for long term measurements. As provided in Tables 56 and 57 below, the three-month pH stability data is provided.

TABLE 56
Ruxolitinib foam formulations pH study to 3-month timepoint

| | Product | Foam | | Sample | | | |
|---|---|---|---|---|---|---|---|
| Samples | name and | base pH, | Sample | Storage, | | Foam | pH drift |
| Formula/Lot | Description | 35-40° C. | Can # | ° C. | Timepoint | pH | (Tx − T0) |
| 268-23-01/ | Placebo, | 6.65 | 36 | 40 | 0 | 7.95 | 0.0 |
| 268-23-01 | LE | | 25 | 40 | 1 | 7.05 | −0.90 |
| | | | 30 | 40 | 3 | 6.95 (n = 2) | −1.00 |
| 268-23-02/ | Placebo, | 5.61 | 35 | 40 | 0 | 8.14 | 0.0 |
| 268-23-02 | E-0.5T | | 25 | 40 | 1 | 7.03 | −1.11 |
| | | | 30 | 40 | 3 | 6.13 | −2.01 |
| 268-23-03/ | Placebo, | 6.50 | 1 | 40 | 0 | 6.12 | 0.00 |
| 268-23-03 | NE | | 4 | 40 | 1 | 7.16 | +1.04 |
| | | | 6 | 40 | 3 | 6.65 | +0.53 |
| 268-23-04/ | 2.5% | 5.22 | 1, 60 | ambient | 0 | 5.32 (n = 2) | 0.00 |
| 268-23-04 | Ruxolitinib, | | 33 | 5 | 1 | 5.41 | +0.09 |
| | LE | | 56 | 40 | | 5.38 | +0.06 |
| | | | 39 | 5 | 3 | 5.47 (n = 2) | +0.15 |
| | | | 48 | 25 | | 5.38 (n = 2) | +0.06 |
| | | | 58 | 40 | | 5.60 (n = 2) | +0.32 |
| 268-23-05/ | 2.5% | 5.25 | 1, 60 | ambient | 0 | 5.49 (n = 2) | 0.00 |
| 268-23-05 | Ruxolitinib, | | 50 | 40 | 1 | 5.28 | −0.21 |
| | E-0.5T | | 37 | 25 | 3 | 5.33 (n = 2) | −0.16 |
| | | | 47 | 40 | | 5.33 (n = 3) | −0.16 |
| 268-23-06/ | 2.5% | 5.25 | 1, 35 | ambient | 0 | 5.29 (n = 2) | 0.00 |
| 268-23-06 | Ruxolitinib, | | 23 | 40 | 1 | 5.46 | +0.17 |
| | NE | | 3 | 25 | 3 | 5.68 (n = 2) | +0.39 |
| | | | 16 | 40 | | 5.54 (n = 3) | +0.25 |

TABLE 57
Additional ruxolitinib foam formulations pH study to 3-month timepoint

| | Product | Foam | | Sample | | | | |
|---|---|---|---|---|---|---|---|---|
| Samples | name and | base pH, | Sample | Storage, | | Foam | | pH drift, |
| Formula/Lot | Description | 35° C.-40° C. | Can # | ° C. | Timepoint | pH | | (Tx − T0) |
| 268-23-07/ | 1.5% | 5.42 | 1, 26 | ambient | 0 | 5.39 | (n = 2) | 0.00 |
| 268-23-07 | Ruxolitinib, | | 21 | 40 | 1 | 5.68 | | +0.29 |
| | NE | | 6 | 25 | 3 | 5.59 | (n = 2 | +0.20 |
| | | | 16 | 40 | | 5.81 | (n = 2 | +0.42 |
| 268-23-08/ | 1.5% | 5.59 | 1, 35 | ambient | 0 | 5.84 | (n = 2) | 0.00 |
| 268-23-08 | Ruxolitinib, | | 21 | 40 | 1 | 5.83 | | −0.01 |
| | LE | | 17 | 25 | 3 | 5.89 | (n = 2) | +0.05 |
| | | | 7 | 40 | | 5.80 | (n = 2) | −0.04 |
| 268-23-09/ | 1.5% | 5.47 | 1, 34 | ambient | 0 | 5.70 | (n = 2) | 0.00 |
| 268-23-09 | Ruxolitinib, | | 23 | 40 | 1 | 5.60 | | −0.10 |
| | E-0.5T | | 3 | 25 | 3 | 5.78 | (n = 2) | +0.08 |
| | | | 13 | 40 | | 5.49 | (n = 2) | −0.21 |

In addition to the pH stability, HPLC analysis was also performed on the ruxolitinib formulations. Tables 58 and 59 summarize the HPLC results.

TABLE 58
HPLC data detailing 3-month stability for ruxolitinib foam formulations

| | | | | | P75 | Storage | | % LC | Total |
|---|---|---|---|---|---|---|---|---|---|
| SAMPLE | | Formulation | % Label | Timepoint | content, | condition, | % w/w | average, | Impurities, |
| NAME | DESCRIPTION | and Lot # | claim | month | % w/w | ° C. | Average | (% RSD) | % area |
| Placebo | Nil Emollient | 268-23-01/ | 0 | 0 | 4.15, 4.20 | ambient | ND | NA | ND |
| Foam, NE | | 268-23-01 | | 1 | 4.15 | 40 | ND | NA | ND |
| | | | | 3 | 4.08 | 40 | ND | NA | ND |
| Placebo | 2.5% (PEG 300 | 268-23-02/ | 0 | 0 | 4.11, 4.05 | ambient | ND | NA | ND |
| Foam, LE | and, Glycerin) | 268-23-02 | | 1 | 4.17 | 40 | ND | NA | ND |
| | | | | 3 | 4.03 | 40 | ND | NA | ND |
| Placebo | 1.5% | 268-23-03/ | 0 | 0 | 4.15 | ambient | ND | NA | ND |
| Foam, | (ML and | 268-23-03 | | 1 | 4.16 | 40 | ND | NA | ND |
| (E-0.5T) | Transcutol-P | | | 3 | 4.03 | 40 | ND | NA | ND |
| 2.5% | 2.5% (PEG 300 | 268-23-04/ | 2.5 | 0 | 4.18, 4.27 | ambient | 2.546 | 101.8 (0.2) | ND |
| Ruxolitinib | and, Glycerin) | 268-23-04 | | 1 | 4.16 | 5 | 2.534 | 101.4 (0.0) | ND |
| Foam, LE | | | | | 4.07 | 40 | 2.540 | 101.6 (0.7) | ND |
| | | | | 3 | 4.12 | 5 | 2.542 | 101.7 (0.1) | ND |
| | | | | | 4.22 | 25 | 2.536 | 101.4 (0.9) | ND |
| | | | | | 4.05 | 40 | 2.539 | 101.6 (0.0) | ND |
| 2.5% | 1.5% | 268-23-05/ | 2.5 | 0 | 4.11, 4.02 | ambient | 2.540 | 101.6 (0.3) | ND |
| Ruxolitinib, | (ML and | 268-23-05 | | 1 | 4.20 | 40 | 2.546 | 101.8 (0.1) | ND |
| Foam, | Transcutol-P | | | 3 | 4.01 | 25 | 2.548 | 101.9 (0.2) | ND |
| (E-0.5T) | | | | | 4.07 | 40 | 2.542 | 101.7 (0.1) | ND |
| 2.5% | Nil Emollient | 268-23-06/ | 2.5 | 0 | 4.12, 4.23 | ambient | 2.574 | 103.0 (0.5) | ND |
| Ruxolitinib, | | 268-23-06 | | 1 | 4.19 | 40 | 2.543 | 101.7 (0.3) | ND |
| Foam NE | | | | 3 | 4.01 | 25 | 2.565 | 102.6 (0.3) | ND |
| | | | | | 3.98 | 40 | 2.544 | 101.8 (1.1) | ND |

TABLE 59
HPLC data detailing 3-month stability for additional ruxolitinib foam formulations

| | | | | | P75 | Storage | | % LC | Total |
|---|---|---|---|---|---|---|---|---|---|
| SAMPLE | | Formulation | % Label | Timepoint | content, | condition, | % w/w | average, | Impurities, |
| NAME | DESCRIPTION | and Lot # | claim | month | % w/w | ° C. | Average | (% RSD) | % area |
| 1.5% | Nil | 268-23-07/ | 1.5 | 0 | 4.16, 4.18 | ambient | 1.527 | 101.8 (0.3) | ND |
| Ruxolitinib | Emollient | 268-23-07 | | 1 | 4.04 | 40 | 1.518 | 101.2 (0.6) | ND |
| Foam, NE | | | | 3 | 4.09 | 25 | 1.513 | 100.9 (0.1) | ND |
| | | | | | 4.08 | 40 | 1.516 | 101.1 (0.0) | ND |
| 1.5% | | 268-23-08/ | 1.5 | 0 | 4.03, 4.16 | ambient | 1.542 | 102.8 (0.8) | ND |
| Ruxolitinib, | | 268-23-08 | | 1 | 4.11 | 40 | 1.519 | 101.3 (0.4) | ND |
| Foam, LE | | | | 3 | 4.24 | 25 | 1.514 | 100.9 (0.2) | ND |
| | | | | | 4.13 | 40 | 1.515 | 101.0 (0.1) | ND |
| 1.5% | | 268-23-09/ | 1.5 | 0 | 4.17, 4.10 | ambient | 1.533 | 102.2 (0.9) | ND |
| Ruxolitinib, | | 268-23-09 | | 1 | 4.13 | 40 | 1.516 | 101.1 (0.2) | ND |
| Foam | | | | 3 | 4.00 | 25 | 1.509 | 100.6 (0.5) | ND |
| (E-0.5T) | | | | | 4.14 | 40 | 1.522 | 101.5 (0.2) | ND |

The Deuruxolitinib formulations for the GLP toxicology study demonstrated 3 months stability and were placed on stability for long term measurements. As provided in Tables 60, 61, and 62, the three-month pH stability data and HPLC data are provided.

TABLE 60
Ruxolitinib foam formulations pH study to 3-month timepoint

| | | | | Foam pH at T = 0 | | Foam pH at T = 3-months | |
|---|---|---|---|---|---|---|---|
| | | | Foam Base | | Foam pH | | Foam pH |
| | | | Given pH | Sample | at T = 0, | Sample | at T = 3 |
| pH data, SP2024.06 stability study | | | of foam | form and | ambient | Form and | months, |
| Samples, | Product | | Base | Storage | 9 Aug. | Storage | 30 Oct. |
| F and L | name | DOM | 35-40° C. | ° C. | 2024 | ° C. | 2024 |
| 268-24-01/ | 2.5% | 18 Jul. 2024 | 5.34 | Foam, | 5.41, 5.44 | Foam, | 5.59 (n = 2) |
| 268-24-01 | Ruxolitinib, | | | ambient, | | 25° C. | |
| | LE | | | cans 1, 68 | | Foam, | 5.56 (n = 2) |
| | | | | | | 40° C. | |
| 268-24-02/ | 2.5% | 19 Jul. 2024 | 5.62 | Foam, | 5.56, 5.56 | Foam, | 5.73 (n = 2) |
| 268-24-02 | Ruxolitinib, | | | ambient, | | 5° C. | |
| | 1E-0.5T | | | cans 1, 68 | | Foam, | 5.64 (n = 2) |
| | | | | | | 25° C. | |
| | | | | | | Foam, | 5.44 (n = 2) |
| | | | | | | 40° C. | |
| 268-24-03/ | 2.5% | 22 Jul. 2024 | 5.36 | Foam, | 5.58, 5.67 | Foam, | 5.70 (n = 2) |
| 268-24-03 | Ruxolitinib, | | | ambient | | 25° C. | |
| | NE | | | #1, 28 | | Foam, | 5.62 (n = 2) |
| | | | | | | 40° C. | |
| 268-24-04/ | 1.5% | 22 Jul. 2024 | 5.81 | Foam, | 5.89, 5.94 | Foam, | 6.04 (n = 2) |
| 268-24-04 | Ruxolitinib, | | | ambient | | 25° C. | |
| | NE | | | #1, 28 | | Foam, | 6.06 (n = 2) |
| | | | | | | 40° C. | |
| 268-24-05/ | 1.5% | 23 Jul. 2024 | 5.83 | Foam, | 5.92, 5.81 | Foam, | 6.24 (n = 2) |
| 268-24-05 | Ruxolitinib, | | | ambient | | 25° C. | |
| | LE | | | #1, 28 | | Foam, | 6.15 (n = 2) |
| | | | | | | 40° C. | |
| 268-24-06/ | 1.5% | 23 Jul. 2024 | 5.45 | Foam, | 5.46, 5.45 | Foam, | 5.47 (n = 3) |
| 268-24-06 | Ruxolitinib, | | | ambient | | 25° C. | |
| | 1E-0.5T | | | #1, 28 | | Foam, | 5.47 (n = 2) |
| | | | | | | 40° C. | |

TABLE 61
HPLC data detailing 3-month stability for deuruxolitinib foam formulations

| | | | | | sample | P75 | Storage | | % LC | | Total |
|---|---|---|---|---|---|---|---|---|---|---|---|
| SAMPLE | | Formulation | % Label | Timepoint | can/s, | content, | condition, | % w/w | average, | | Impurities, |
| NAME | DESCRIPTION | and Lot # | claim | month | # | % w/w | ° C. | Average | (% RSD) | | % area |
| Placebo | 2.5% (PEG | 268-23-01/ | 0 | 0 | 1, 36 | 4.15, 4.20 | ambient | ND | NA | | ND |
| Foam, LE | 300 and, | 268-23-01 | | 1 | 25 | 4.15 | 40 | ND | NA | | ND |
| | Glycerin) | | | 3 | 30 | 4.08 | 40 | ND | NA | | ND |
| Placebo | 1.5% | 268-23-02/ | 0 | 0 | 1, 35 | 4.11, 4.05 | ambient | ND | NA | | ND |
| Foam, | (ML and | 268-23-02 | | 1 | 25 | 4.17 | 40 | ND | NA | | ND |
| E-0.5T | Transcutol-P | | | 3 | 30 | 4.03 | 40 | ND | NA | | ND |
| Placebo | Nil Emollient | 268-23-03/ | 0 | 0 | 1 | 4.15 | ambient | ND | NA | | ND |
| Foam, NE | | 268-23-03 | | 1 | 4 | 4.16 | 40 | ND | NA | | ND |
| | | | | 3 | 6 | 4.03 | 40 | ND | NA | | ND |
| 2.5% | 2.5% (PEG | 268-24-01/ | 2.5 | 0 | 1, 68 | 4.07, 4.22 | ambient | 2.526 | 101.0 | (0.2) | ND |
| Deuruxolitinib | 300 and, | 268-24-01 | | 1 | 52 | 4.03 | 40 | 2.512 | 100.5 | (0.0) | ND |
| Foam, LE | Glycerin) | | | 3 | 42 | 4.17 | 25 | 2.487 | 99.5 | (1.1) | ND |
| | | | | | 55 | 4.05 | 40 | 2.523 | 100.9 | (0.1) | ND |
| 2.5% | 1.5% | 268-24-02/ | 2.5 | 0 | 1, 68 | 4.02, 4.02 | ambient | 2.526 | 101.0 | (0.2) | ND |
| Deuruxolitinib, | (ML and | 268-24-02 | | 1 | 36 | 4.09 | 5 | 2.519 | 100.8 | (0.1) | ND |
| Foam, | Transcutol-P | | | | 55 | 4.04 | 40 | 2.525 | 101.0 | (0.4) | ND |
| (E-0.5T) | | | | 3 | 41 | 4.11 | 5 | 2.527 | 101.1 | (0.1) | ND |
| | | | | | 48 | 4.12 | 25 | 2.517 | 100.7 | (0.1) | ND |
| | | | | | 67 | 4.08 | 40 | 2.536 | 101.4 | (0.1) | ND |

TABLE 62
HPLC data detailing 3-month stability for additional deuruxolitinib foam formulations

| | | | | | sample | P75 | Storage | | % LC | | Total |
|---|---|---|---|---|---|---|---|---|---|---|---|
| SAMPLE | | Formulation | % Label | Timepoint | can/s, | content, | condition, | % w/w | average, | | Impurities, |
| NAME | DESCRIPTION | and Lot # | claim | month | # | % w/w | ° C. | Average | (% RSD) | | % area |
| 2.5% | Nil | 268-24-03/ | 2.5 | 0 | 1, 28 | 4.05, 4.12 | ambient | 2.510 | 100.4 | (0.2) | ND |
| Deuruxolitinib, | Emollient | 268-24-03 | | 1 | 19 | 4.07 | 40 | 2.516 | 100.6 | (0.0) | ND |
| Foam NE | | | | 3 | 6 | 4.09 | 25 | 2.527 | 101.1 | (0.9) | ND |
| | | | | | 24 | 4.11 | 40 | 2.521 | 100.8 | (0.4) | ND |
| 1.5% | Nil | 268-24-04/ | 2.5 | 0 | 1, 28 | 4.09, 4.08 | ambient | 1.512 | 100.8 | (0.2) | ND |
| Deuruxolitinib | Emollient | 268-24-04 | | 1 | 22 | 4.12 | 40 | 1.501 | 100.1 | (0.4) | ND |
| Foam, NE | | | | 3 | 6 | 4.01 | 25 | 1.508 | 100.5 | (0.2) | ND |
| | | | | | 25 | 4.20 | 40 | 1.507 | 100.5 | (0.1) | ND |
| 1.5% | 2.5% | 268-24-05/ | 1.5 | 0 | 1, 28 | 4.18, 4.15 | ambient | 1.511 | 100.7 | (0.2) | ND |
| Deuruxolitinib, | (PEG 300 | 268-24-05 | | 1 | 22 | 4.20 | 40 | 1.507 | 100.5 | (0.0) | ND |
| LE | and, Glycerin) | | | 3 | 6 | 4.11 | 25 | 1.506 | 100.4 | (0.2) | ND |
| | | | | | 25 | 4.10 | 40 | 1.509 | 100.6 | (0.2) | ND |
| 1.5% | 1.5% | 268-24-06/ | 1.5 | 0 | 1, 28 | 4.14, 4.16 | ambient | 1.490 | 99.4 | (0.4) | ND |
| Deuruxolitinib, | (ML and | 268-24-06 | | 1 | 22 | 4.20 | 40 | 1.492 | 99.4 | (0.3) | ND |
| Foam | Transcutol-P | | | 3 | 6 | 4.08 | 25 | 1.481 | 98.7 | (0.2) | ND |
| (E-0.5T) | | | | | 25 | 4.04 | 40 | 1.486 | 99.1 | (0.1) | ND |

### Physical Stability-Propellant-Foam Base Miscibility

### Ruxolitinib

For this investigation, the foam formulation samples were filled, crimped and gassed with propellant (4.0% P75) in glass aerosol bottles for regular monitoring to show clear uniform homogeneous foam products to 3-months evaluation timepoint. In Table A, only 1-formulation showed physical instability by showing cloudiness upon agitation in the bottle, i.e., 1.5% Ruxolitinib Foam, NE (Nil emollient).

TABLE A
Base-propellant miscibility visual inspection.

| | | | | T = 1-month | Visual |
|---|---|---|---|---|---|
| | Product | | | visual | examination |
| | name, | T = 1-month visual inspection, | | observation | T = 3-month/ |
| Sample | Formulation/ | images from | | after 4 days at | 25° C./60% RH |
| # | Lot | 25° C./60% RH | | 40° C./75% RH, | storage |
| 1 | 268-23-04/ | Clear | Retains | Clear, | Clear, |
| | 268-23-04, | uniform, | clarity and | homogeneous | homogeneous |
| | 2.5% | homogeneous. | homogeneity | with | no layering, |
| | Ruxolitinib | No sediment | after shaking | negligible | remain clear |
| | Foam, LE | observed. | | residue only | upon |
| | | | | seen against | agitation |
| | | | | light. | |
| 2 | 268-23-05/ | Clear | Retains | Clear, | Clear, |
| | 268-23-05, | uniform, | clarity and | homogeneous | homogeneous |
| | 2.5% | homogeneous. | homogeneity | with | no layering, |
| | Ruxolitinib | No sediment | after shaking | negligible | remain clear |
| | Foam, 1E−0.5T | observed. | | residue ir | upon |
| | | | | only seen | agitation |
| | | | | against light. | |
| 3 | 268-23-06/ | Clear | Retains | Clear, | Clear, |
| | 268-23-06, | uniform, | clarity and | homogeneous | homogeneous |
| | 2.5% | homogeneous. | homogeneity | with | no layering, |
| | Ruxolitinib | No sediment | after shaking | negligible | remain clear |
| | Foam, NE | observed. | | residue only | upon |
| | | | | seen against | agitation |
| | | | | light. | |
| 4 | 268-23-07/ | Clear | Turns hazy | Clear, | Clear, |
| | 268-23-07, | uniform, | upon | homogeneous | homogeneous |
| | 1.5% | homogeneous. | agitation | with | no layering |
| | Ruxolitinib | No sediment | | negligible | observed, but |
| | Foam, NE | observed. | | residue only | turns slightly |
| | | | | seen against | cloudy upon |
| | | | | light. | agitation |
| | | | | Remains | |
| | | | | clear on | |
| | | | | shaking | |
| 5 | 268-23-08/ | Clear | Retains | Clear, | Clear, |
| | 268-23-08, | uniform, | clarity and | homogeneous | homogeneous |
| | 1.5% | homogeneous. | homogeneity | with | no layering, |
| | Ruxolitinib | No sediment | after shaking | negligible | remain clear |
| | Foam, LE | observed. | | residue only | upon |
| | | | | seen against | agitation |
| | | | | light. | |
| 6 | 268-23-09/ | Clear | Retains | Clear, | Clear, |
| | 268-23-09, | uniform, | clarity and | homogeneous | homogeneous |
| | 1.5% | homogeneous. | homogeneity | with | no layering, |
| | Ruxolitinib | No sediment | after shaking | negligible | remain clear |
| | Foam, 1E−0.5T | observed. | | residue only | upon |
| | | | | seen against | agitation |
| | | | | light. | |

A foam collapse test at 32° C. was also conducted. The results are presented in Table B.

TABLE B
Foam collapse test 32° C., T = 0 vs 1-month

| | | T = 0 Foam Collapse results | | | T = 1 month Foam Collapse results | | |
|---|---|---|---|---|---|---|---|
| | | | Min. time | Calculated | | Min. time | Calculated Rate |
| | Product | Sample test | foam | foam Collapse, | Sample test | test foam | of foam collapse, |
| | formulation/ | weight, g | collapse, | mins/g [(B)/ | weight, g | collapse, | mins./g foam |
| Test sample | Lot | foam (A) | mins. (B) | (A)] = (C) | foam (A) | minutes (B) | [(B)/(A)] = (C) |
| Placebo | F 268-23-01/ | 0.94 g | 5 mins. with | PASS - 5.32 | 1.27 g foam | 7 mins + | PASS - 5.5 |
| Foam LE | L 268-23-01 | foam | residue | | | residue | |
| Placebo | F 268-23-02/ | 1.05 g | 3.5 to 3.75 | PASS- 3.45 | 1.24 g foam | 3.5-3.75 | BELOW spec - 2.8 |
| Foam 1E-0.5T | L268-23-02 | foam | mins. | | | | to 3.0 |
| Placebo | F 268-23-03/ | 0.96 g | >>11 mins. | FAIL- Off- | 0.84 g foam | >>11 | ABOVE spec, >>13 |
| Foam NE | L 268-23-03 | foam | | spec, >>11 | | | |
| 2.5% | F 268-23-04/ | 1.11 g | 4 mins. with | PASS - 3.6 | 1.25 g foam | 3.75 mins | PASS - 3.0 |
| Ruxolitinib | L 268-23-04 | foam | residue | | (5° C. foam) | with residue | |
| Foam, LE | | | | | 1.22 g foam | 3.0 mins with | BELOW spec - 2.46 |
| | | | | | (40° C. foam) | residue | |
| 2.5% | F 268-23-05/ | 1.09 g | 2.5 to 3 | FAIL- Off- | 1.23 g foam | 3.0 mins. No | BELOW spec - 2.44 |
| Ruxolitinib | L 268-23-05 | foam | mins. No | spec - 2.52 | (40° C. foam) | residue | |
| Foam, | | | residue | | | | |
| 1E-0.5T | | | | | | | |
| 2.5% | F 268-23-06/ | 1.19 g | >>11 mins. | FAIL OFF- | 1.10 g foam | >>11 mins. | ABOVE spec, >>10 |
| Ruxolitinib | L 268-23-06 | foam | | spec, >>9 | (40° C. foam) | | |
| Foam, NE | | | | | | | |
| 1.5% | F 268-23-07/ | 1.17 g | >>11 mins. | FAIL OFF- | 1.45 g foam | >>11 mins. | ABOVE spec, >>10 |
| Ruxolitinib | L 268-23-07 | foam | | spec, >>9 | (40° C. foam) | | |
| Foam, NE | | | | | | | |
| 1.5% | F 268-23-08/ | 0.98 g | 4 mins. with | PASS - 4.08 | 1.12 g foam | 2.25 mins | BELOW spec - 2.0 |
| Ruxolitinib | L 268-23-08 | foam | residue | | (40° C. foam) | with residue | |
| Foam, LE | | | | | | | |
| 1.5% | F 268-23-09/ | 1.11 g | 3.0 to 3.5 | Slightly | 1.37 g foam | 2.0 mins. No | BELOW spec - 1.46 |
| Ruxolitinib | L 268-23-09 | foam | mins. No | BELOW spec - | (40° C. foam) | residue | |
| Foam, | | | residue | 2.93 | | | |
| 1E-0.5T | | | | | | | |

Additional foam collapse tests at the 3-month time point are also presented below in Table C.

TABLE C
T = 3-months Foam Collapse Test results, 32° C.
T = 3-months Foam Collapse Test results, 32° C.

| | | | | | Calculated |
|---|---|---|---|---|---|
| | | | Test foam | Minimum time foam | Collapse time, |
| | Product formulation/ | Sample | sample wt, (A), | samples collapsed (B), | [(B)/(A)] = (C) |
| Test sample | Lot | storage, ° C. | gram | minutes. | mins./g-foam |
| Placebo Foam LE | F 268-23-01/ L 268- | 40 | 1.20 | 6.0 mins. with residue | PASS- 5.0 mins/g |
| | 23-01 | | | | |
| Placebo Foam | F 268-23-02/L | 40 | 1.01 | 3.5 mins. with slight | PASS- 3.46 |
| 1E−0.5T | 26823-02 | | | residue | |
| 2.5% Ruxolitinib | F 268-23-04/ | 5 | 1.20 | 3.5 mins. with slight | LOW- 2.91 |
| Foam, LE | L26823-04 | | | residue | |
| | | 25 | 1.07 | 3.5 mins. with slight | PASS- 3.27 |
| | | | | residue | |
| | | 40 | 1.30 | 4.5 mins. with residue | PASS- 3.46 |
| 2.5% Ruxolitinib | F 268-23-05/L | 25 | 1.32 | 3.5 mins. | LOW- 2.65 |
| Foam, 1E−0.5T | 26823-05 | 40 | 1.24 | 2.5 mins. | LOW- 2.0 |
| | | 25 | 1.33 | 2.75 mins. | LOW- 2.06 |
| 1.5% Ruxolitinib | F 268-23-08/L | 40 | 1.26 | 3.25 mins. | LOW- 2.57 |
| Foam, LE | 26823-08 | | | | |
| 1.5% Ruxolitinib | F 268-23-09/L | 25 | 1.35 | 2.5 mins. | LOW- 1.85 |
| Foam, 1E−0.5T | 26823-09 | 40 | 1.15 | 3.5 mins. | PASS- 3.04 |

### Summary of Findings for Ruxolitinib Physical Stability

The drug Ruxolitinib, remains chemically stable to 3-months timepoint at 25° C./60% RH and 40° C./75% RH for all formulations. The content Ruxolitinib is within 2% of initial content at time zero and also well within specification limit of 90-110% label claim. The impurities are all below the method detection limit of 0.05% area.

pH readings T-0 to 3-months all are maintained within pH5-6 range in the active foam products. The pH values of Placebos are expected to drift slightly outside the range pH 6-7, there is no pH adjusting excipient is in any of the SP202.05 placebo formulations.

### Physical Evaluation Results:

Miscibility between propellant P75 and the foam base show similar result as in T=0 with 1 formulation i.e., 1.5% Ruxolitinib Foam with no emollient (268-23-07/268-23-07), showing layer on the surface turning hazy on shaking the bottle. Other than that, all other formulations in appear clear, homogeneous without phase separation.

The 6-active formulations show full, formed foams with well-defined stable peaks. There was no collapse in the foams from time 0 to 2 minutes observation, ambient.

Rate of Foam collapse 32° C.—considering (3-6) mins./gram foam as the provisional specification: (1) Formulations containing PEG 300-Glycerin as emollient combination including Placebo got the PASS i.e., meeting the minimum Rate of Foam collapse i.e., 3-mins. per gram foam. (2) Formulations containing ML and Transcutol-P as emollients got the FAIL with Rate of Foam Collapse below the spec. <3 minutes/gram foam. (3) All NE formulations both active and placebo got the FAIL showing no collapse even after 11 mins time (see result at T=1-month timepoint).

Test for Packaging stability is to be done at next timepoint, T=6 months. (Note 6-month packaging stability (not shown) indicates the primary packaging is compatible with the product.).

Samples of the formulation on PK and TOX studies are sent for AET tests at T-0 of the stability study. These formulations i.e., 2.5% Ruxolitinib Foam, LE and 2.5% Ruxolitinib Foam, E-0.5T are reported compliant with the current USP-NF<51> Antimicrobial Effectiveness Testing criteria for Category 2 products.

### Deuruxolitinib

In Table D, pH stability is summarized at three months for deuruxolitinib.

TABLE D
pH study, stability at T0 and 3-months

| | | | Foam Base | Foam pH at T = 0 | | Foam pH at T = 3-months | |
|---|---|---|---|---|---|---|---|
| | | | Given pH | Sample | | Sample | |
| pH data - stability study | | | of foam | form and | Foam pH | Form and | Foam pH |
| Samples, | Product | | Base | Storage | at T = 0, | Storage | at T = 3 |
| F and L | name | DOM | 35-40° C. | ° C. | ambient | ° C. | months |
| 268-24-01/ | 2.5% | 18 Jul. 2024 | 5.34 | Foam, | 5.41, 5.44 | Foam, | 5.59 (n = 2) |
| 268-24-01 | Ruxolitinib, | | | ambient, | | 25° C. | |
| | LE | | | cans 1, 68 | | Foam, | 5.56 (n = 2) |
| | | | | | | 40° C. | |
| 268-24-02/ | 2.5% | 19 Jul. 2024 | 5.62 | Foam, | 5.56, 5.56 | Foam, | 5.73 (n = 2) |
| 268-24-02 | Ruxolitinib, | | | ambient, | | 5° C. | |
| | 1E-0.5T | | | cans 1, 68 | | Foam, | 5.64 (n = 2) |
| | | | | | | 25° C. | |
| | | | | | | Foam, | 5.44 (n = 2) |
| | | | | | | 40° C. | |
| 268-24-03/ | 2.5% | 22 Jul. 2024 | 5.36 | Foam, | 5.58, 5.67 | Foam, | 5.70 (n = 2) |
| 268-24-03 | Ruxolitinib, | | | ambient | | 25° C. | |
| | NE | | | #1, 28 | | Foam, | 5.62 (n = 2) |
| | | | | | | 40° C. | |
| 268-24-04/ | 1.5% | 22 Jul. 2024 | 5.81 | Foam, | 5.89, 5.94 | Foam, | 6.04 (n = 2) |
| 268-24-04 | Ruxolitinib, | | | ambient | | 25° C. | |
| | NE | | | #1, 28 | | Foam, | 6.06 (n = 2) |
| | | | | | | 40° C. | |
| 268-24-05/ | 1.5% | 23 Jul. 2024 | 5.83 | Foam, | 5.92, 5.81 | Foam, | 6.24 (n = 2) |
| 268-24-05 | Ruxolitinib, | | | ambient | | 25° C. | |
| | LE | | | #1, 28 | | Foam, | 6.15 (n = 2) |
| | | | | | | 40° C. | |
| 268-24-06/ | 1.5% | 23 Jul. 2024 | 5.45 | Foam, | 5.46, 5.45 | Foam, | 5.47 (n = 3) |
| 268-24-06 | Ruxolitinib, | | | ambient | | 25° C. | |
| | 1E-0.5T | | | #1, 28 | | Foam, | 5.47 (n = 2) |
| | | | | | | 40° C. | |

In Tables E and F, HPLC data is presented for deuruxolitinib foam formulations.

TABLE E
HPLC data- Deuruxolitinib Foam

| | | | | | sample | P75 | Storage | | % LC | | Total |
|---|---|---|---|---|---|---|---|---|---|---|---|
| SAMPLE | | Formulation | % Label | Timepoint | can/s, | content, | condition, | % w/w | average, | | Impurities, |
| NAME | DESCRIPTION | and Lot # | claim | month | # | % w/w | ° C. | Average | (% RSD) | | % area |
| Placebo | 2.5% (PEG | 268-23-01/ | 0 | 0 | 1, 36 | 4.15, 4.20 | ambient | ND | NA | | ND |
| Foam, LE | 300 and, | 268-23-01 | | 1 | 25 | 4.15 | 40 | ND | NA | | ND |
| | Glycerin) | | | 3 | 30 | 4.08 | 40 | ND | NA | | ND |
| Placebo | 1.5% | 268-23-02/ | 0 | 0 | 1, 35 | 4.11, 4.05 | ambient | ND | NA | | ND |
| Foam, | (ML and | 268-23-02 | | 1 | 25 | 4.17 | 40 | ND | NA | | ND |
| E-0.5T | Transcutol-P | | | 3 | 30 | 4.03 | 40 | ND | NA | | ND |
| Placebo | Nil Emollient | 268-23-03/ | 0 | 0 | 1 | 4.15 | ambient | ND | NA | | ND |
| Foam, NE | | 268-23-03 | | 1 | 4 | 4.16 | 40 | ND | NA | | ND |
| | | | | 3 | 6 | 4.03 | 40 | ND | NA | | ND |
| 2.5% | 2.5% (PEG | 268-24-01/ | 2.5 | 0 | 1, 68 | 4.07, 4.22 | ambient | 2.526 | 101.0 | (0.2) | ND |
| Deuruxolitinib | 300 and, | 268-24-01 | | 1 | 52 | 4.03 | 40 | 2.512 | 100.5 | (0.0) | ND |
| Foam, LE | Glycerin) | | | 3 | 42 | 4.17 | 25 | 2.487 | 99.5 | (1.1) | ND |
| | | | | | 55 | 4.05 | 40 | 2.523 | 100.9 | (0.1) | ND |
| 2.5% | 1.5% | 268-24-02/ | 2.5 | 0 | 1, 68 | 4.02, 4.02 | ambient | 2.526 | 101.0 | (0.2) | ND |
| Deuruxolitinib, | (ML and | 268-24-02 | | 1 | 36 | 4.09 | 5 | 2.519 | 100.8 | (0.1) | ND |
| Foam, | Transcutol-P | | | | 55 | 4.04 | 40 | 2.525 | 101.0 | (0.4) | ND |
| (E-0.5T) | | | | 3 | 41 | 4.11 | 5 | 2.527 | 101.1 | (0.1) | ND |
| | | | | | 48 | 4.12 | 25 | 2.517 | 100.7 | (0.1) | ND |
| | | | | | 67 | 4.08 | 40 | 2.536 | 101.4 | (0.1) | ND |

TABLE F
HPLC data - Deuruxolitinib Foam

| | | | | | sample | P75 | Storage | | % LC | | Total |
|---|---|---|---|---|---|---|---|---|---|---|---|
| SAMPLE | | Formulation | % Label | Timepoint | can/s, | content, | condition, | % w/w | average, | | Impurities, |
| NAME | DESCRIPTION | and Lot # | claim | month | # | % w/w | ° C. | Average | (% RSD) | | % area |
| 2.5% | Nil | 268-24-03/ | 2.5 | 0 | 1, 28 | 4.05, 4.12 | ambient | 2.510 | 100.4 | (0.2) | ND |
| Deuruxolitinib, | Emollient | 268-24-03 | | 1 | 19 | 4.07 | 40 | 2.516 | 100.6 | (0.0) | ND |
| Foam NE | | | | 3 | 6 | 4.09 | 25 | 2.527 | 101.1 | (0.9) | ND |
| | | | | | 24 | 4.11 | 40 | 2.521 | 100.8 | (0.4) | ND |
| 1.5% | Nil | 268-24-04/ | 2.5 | 0 | 1, 28 | 4.09, 4.08 | ambient | 1.512 | 100.8 | (0.2) | ND |
| Deuruxolitinib | Emollient | 268-24-04 | | 1 | 22 | 4.12 | 40 | 1.501 | 100.1 | (0.4) | ND |
| Foam, NE | | | | 3 | 6 | 4.01 | 25 | 1.508 | 100.5 | (0.2) | ND |
| | | | | | 25 | 4.20 | 40 | 1.507 | 100.5 | (0.1) | ND |
| 1.5% | 2.5% | 268-24-05/ | 1.5 | 0 | 1, 28 | 4.18, 4.15 | ambient | 1.511 | 100.7 | (0.2) | ND |
| Deuruxolitinib, | (PEG 300 and, | 268-24-05 | | 1 | 22 | 4.20 | 40 | 1.507 | 100.5 | (0.0) | ND |
| LE | Glycerin) | | | 3 | 6 | 4.11 | 25 | 1.506 | 100.4 | (0.2) | ND |
| | | | | | 25 | 4.10 | 40 | 1.509 | 100.6 | (0.2) | ND |
| 1.5% | 1.5% | 268-24-06/ | 1.5 | 0 | 1, 28 | 4.14, 4.16 | ambient | 1.490 | 99.4 | (0.4) | ND |
| Deuruxolitinib, | (ML and | 268-24-06 | | 1 | 22 | 4.20 | 40 | 1.492 | 99.4 | (0.3) | ND |
| Foam (E-0.5T) | Transcutol-P | | | 3 | 6 | 4.08 | 25 | 1.481 | 98.7 | (0.2) | ND |
| | | | | | 25 | 4.04 | 40 | 1.486 | 99.1 | (0.1) | ND |

### Physical Stability-Propellant-Foam Base Miscibility

Foam formulations on stability study were prepared in gassed with propellant (4.0% P75) in glass aerosol bottles and were regularly checked and monitored for miscibility and homogeneity between the propellant and foam base. Those formulations and their results are summarized in Table G.

All 6 active formulations in glass aerosol bottles were kept at 25° C./60% RH or 40° C./75% RH and checked visually at every timepoint.

Of 6-formulation, samples 5 appear clear, uniform and homogeneous with no indication of layer forming/phase separation.

Of the 6-formulations samples, 1-formulation showed a clear layer on the surface and turning cloudy when the bottle is agitated. This observation was a sign physical instability which over time may lead to phase separation. This sample with the sign of physical instability in the formulation was the 1.5% Ruxolitinib Foam, NE (Nil emollient), F268-24-04/L268-24-04.

TABLE G
Foam Base-Propellant Miscibility Visual inspection, 3-months

| | | | | | Visual |
|---|---|---|---|---|---|
| | | | | T = 1-month | examination |
| | | | | visual check | T = 3-month/ |
| | Product name, | T = 1-month visual | | after 4 days at | 25° C./ |
| Sample | Formulation/ | inspection, | | 40° C./75% RH, | 60% RH |
| # | Lot | 25° C./ 60% RH, date | | date examined | storage |
| 1 | 268-24-01/ | Clear-uniform | Retains | Clear-uniform | Clear- |
| | 268-24-01 | liquid | clarity and | liquid with | uniform |
| | 2.5% | homogeneous. | uniformity | minimal | liquid, no |
| | Deuruxolitinib | No sediment | after | whitish | layering, |
| | Foam, LE | observed. | shaking | residue only | remain |
| | | | | seen against | clear upon |
| | | | | light. | shaking |
| 2 | 268-24-02/ | Clear-uniform | Retains | Clear-uniform | Clear- |
| | 268-24-02 | liquid | clarity and | liquid with | uniform |
| | 2.5% | homogeneous. | uniformity | minimal | liquid, no |
| | Deuruxolitinib | No sediment | after | whitish | layering, |
| | Foam, | observed. | shaking | residue only | remain |
| | E−0.5T | | | seen against | clear upon |
| | | | | light. | shaking |
| 3 | 268-24-03/ | Clear-uniform | Retains | Clear-uniform | Clear- |
| | 268-24-03 | liquid | clarity and | liquid with | uniform |
| | 2.5% | homogeneous. | uniformity | minimal | liquid, no |
| | Deuruxolitinib | No sediment | after | whitish | layering, |
| | Foam, NE | observed. | shaking | residue only | remain |
| | | | | seen against | clear upon |
| | | | | light. | shaking |
| 4 | 268-24-04/ | Clear-uniform | Retains | Clear-uniform | Clear- |
| | 268-24-04 | liquid | clarity and | liquid with | uniform |
| | 1.5% | homogeneous. | uniformity | minimal | liquid no |
| | Deuruxolitinib | No sediment | after | whitish | layering |
| | Foam, NE | observed. | shaking | residue only | observed, |
| | | | | seen against | but turns |
| | | | | light. | slightly |
| | | | | Remains clear | cloudy |
| | | | | on shaking | upon |
| | | | | | shaking |
| 5 | 268-24-05/ | Clear-uniform | Retains | Clear-uniform | Clear- |
| | 268-24-05 | liquid | clarity and | liquid with | uniform, no |
| | 1.5% | homogeneous. | uniformity | minimal | layering, |
| | Deuruxolitinib | No sediment | after | whitish | remain |
| | Foam, LE | observed. | shaking | residue only | clear upon |
| | | | | seen against | shaking |
| | | | | light. | |
| 6 | 268-24-06/ | Clear-uniform | Retains | Clear-uniform | Clear- |
| | 268-24-06 | liquid | clarity and | liquid with | uniform, no |
| | 1.5% | homogeneous. | uniformity | minimal | layering, |
| | Deuruxolitinib | No sediment | after | whitish | remain |
| | Foam, | observed. | shaking | residue only | clear upon |
| | E−0.5T | | | seen against | shaking |
| | | | | light. | |

Foam collapse rates of the deuruxolitinib foams are presented in Table H at timepoint=0.

TABLE H
Rate of Foam Collapse- Deuruxolitinib Foam, T = 0

| | | Weight of | | Calculated |
|---|---|---|---|---|
| | Product | foam sample | Minimum time foam | Collapse time, |
| | formulation/ | applied, gram | collapsed, minutes | minutes/gram |
| Test sample | Lot # | (A) | (B) | foam, (C) = [(B)/(A)] |
| Placebo Foam | F268-23-01/ | 0.94 g foam | 5 mins with residue | PASS - 5.32 |
| LE | L26823-01 | | | minutes/g foam |
| Placebo Foam | F268-23-02/ | 1.05 g foam | 3.5 to 3.75 mins | PASS - 3.45 |
| 1E−0.5T | L26823-02 | | | minutes/g foam |
| Placebo Foam | F268-23-03/ | 0.96 g foam | >>11 mins (ie still | FAIL, Off spec |
| NE | L26823-03 | | full foam after 11 | |
| | | | mins). | |
| 2.5% | F268-24-01/ | 0.90 g foam | 3.5-3.75 mins. with | PASS - 3.9 |
| Deuruxolitinib | L268-24-01 | | residue (minute | minutes/g foam |
| Foam, LE | | | bubbles) | |
| 2.5% | F268-24-02/ | 1.08 g foam | 3 mins., no residue | 2.9 mins./g foam - |
| Deuruxolitinib | L268-24-02 | | | BELOW spec |
| Foam, 1E−0.5T | | | | |
| 2.5% | F268-24-03/ | 1.10 g foam | No sign of collapse | FAIL, Off spec |
| Deuruxolitinib | L268-24-03 | | after 5 mins, very | |
| Foam, NE | | | slow collapse from | |
| | | | base still full foam | |
| | | | after 11 mins. | |
| 1.5% | F268-24-04/ | 0.96 g foam | Same observation as | FAIL, Off spec |
| Deuruxolitinib | L268-24-04 | | F/L 268-24-03, both | |
| Foam, NE | | | NE | |
| 1.5% | F 268-24- | 1.10 g foam | 4.5 mins. + residue | PASS- 4.1 |
| Deuruxolitinib | 05/ | | | minutes/g foam |
| Foam, LE | L268-24-05 | | | |
| 1.5% | F 268-24- | 1.46 g foam | 3.0 mins., no residue | 2.06 mins./g |
| Deuruxolitinib | 06/ | | | foam- BELOW |
| Foam, 1E−0.5T | L268-24-06 | | | spec |

Foam collapse rates of the deuruxolitinib foams are presented in Table I at timepoint=3 months.

TABLE I
Rate of Foam Collapse- Deuruxolitinib Foam, T = 3-months
Rate of Foam Collapse, 32° C. T = 3-months

| | | | | Minimum time | Calculated |
|---|---|---|---|---|---|
| | Product | Sample | Test foam | foam samples | Collapse time, |
| | formulation/ | storage, | sample wt, | collapsed (B), | [(B)/(A)] = (C) |
| Test sample | Lot | ° C. | (A), gram | minutes. | mins./g-foam |
| Placebo Foam | F 268-23- | 40 | 1.20 | 6.0 (with | PASS- 5.0 |
| LE | 01/ L 268- | | | residue) | |
| | 23-01 | | | | |
| Placebo Foam | F 268-23- | 40 | 1.01 | 3.5 (with slight | PASS- 3.46 |
| 1E−0.5T | 02/ L | | | residue) | |
| | 26823-02 | | | | |
| 2.5% | F268-24-01/ | 25 | 1.17 | 7.0 (with slight | PASS- 6.0 |
| Deuruxolitinib | L268-24-01 | | | residue) | |
| Foam, LE | | 40 | 1.03 | 3.5 (with | PASS- 3.4 |
| | | | | residue) | |
| 2.5% | F268-24-02/ | 5 | 1.23 | 3.5 | BELOW- 2.84 |
| Deuruxolitinib | L268-24-02 | 25 | 1.18 | 2.75 | BELOW-2.33 |
| Foam, 1E−0.5T | | 40 | 1.13 | 2.75 | BELOW- 2.43 |
| 1.5% | F 268-24- | 25 | 1.40 | 3.5 (with slight | BELOW- 2.5 |
| Deuruxolitinib | 05/ | | | residue) | |
| Foam, LE | L268-24-05 | 40 | 1.33 | 4.0 (with slight | PASS- 3.0 |
| | | | | residue) | |
| 1.5% | F 268-24- | 25 | 1.22 | 2.42 | BELOW- 2.0 |
| Deuruxolitinib | 06/ | 40 | 1.69 | 3.0 | BELOW- 1.8 |
| Foam, 1E−0.5T | L268-24-06 | | | | |

### Summary of Findings for Deuruxolitinib Physical Stability

The drug-deuruxolitinib-remained chemically stable to 3-months timepoint at 25° C. and 40° C. for all formulations tested.

The content deuruxolitinib is within 2% of initial content at time zero and well within the specification limit of 90-110% of the label claim. The impurities were all below the detection limit of 0.05% area.

pH readings to 3-months of the drug active foam products were all maintained within pH 5-6 range with the exception of 1-formulation, i.e., F268-24-05/L268-24-05.

### Physical Evaluation Results:

Miscibility between propellant P75 and the foam base showed similar result as in T-O with 1 formulation, i.e., 1.5% Ruxolitinib Foam with no emollient (268-23-07/268-23-07), showing layer on the surface turning hazy on shaking the bottle. Other than that, all other formulations tested appeared clear, homogeneous without phase separation.

The 6-active formulations showed full, formed foams with well-defined stable peaks at ambient. There was no collapse in the foams from time 0 to 2 minutes observation when exposed at ambient temperature.

Microscopic examination of the foam samples suggested the propellant was well-dispersed within foam base. No evidence of drug recrystallisation was observed, indicating deuruxolitinib remained soluble in the foam formulation.

Rate of Foam collapse 32° C.—considering (3-6) mins./gram-foam as target rate of foam collapse:

(1) Formulations containing PEG 300-Glycerin as emollient combination including Placebo achieved the minimum and PASS the Rate of Foam collapse, i.e., 3-mins. per gram-foam. Note the undissolved residue these formulations leave after each test, 32° C. (2) Formulations containing Myristyl Lactate consistently showed faster but neat collapse resulting in rate of foam collapse falling below the minimum, i.e., <3 minutes/gram-foam. These formulations fell out of the target provisional range 3-6 mins/gram foam. (3) All NE formulations both active and placebo received a FAIL, i.e., showed no collapse even after >11 mins time (see result at T=1-month timepoint).

Test for packaging stability is done at 6-months timepoint.

Formulations on PK and TOX studies were sent for antimicrobial effectiveness test (AET). These 2-formulations, i.e., 2.5% Deuruxolitinib Foam, LE and 2.5% Deuruxolitinib Foam, E-0.5T were both reported to comply with the current USP-NF<51> Antimicrobial Effectiveness testing criteria for Category 2 products.

### Protocol for Ruxolitinib Foam Min-Pig Good Laboratory Procedures (GLP) Toxicology Study

The study was a 7-day local tolerance and toxicokinetic study of ruxolitinib foam formulations by dermal administration in minipigs. The objectives of this study were to determine the potential local toxicity and toxicokinetic profile of two ruxolitinib foam formulations, when given by dermal administration for 7 days (each material administered over a separate 7-day interval) to minipigs and to evaluate the potential reversibility of any findings. Additionally, skin biopsy and plasma ruxolitinib concentrations were to be evaluated as non-GLP exploratory endpoints.

The Göttingen minipig was chosen as the animal model for this study as it is an accepted nonrodent species for preclinical toxicity testing by regulatory agencies.

The dermal route of exposure was selected because this is the intended route of human exposure.

Ruxolitinib topical cream at concentration up to 1.5% was fully tested in pig model and currently approved for use in patients. Oral ruxolitinib is also approved for clinical use. 2.5% dermal foam formulation is expected to provide lower systemic exposure compared to oral administration. As noted above, this study compared two different formulations of ruxolitinib.

### Surgical Procedure and Schedule for Skin Sample Collection

Samples were collected as indicated in the Sample Collection table below:

Sample Collection

| | | Target Time post first | | |
|---|---|---|---|---|
| Group | Study | daily dose | Collection | |
| No. | Day | 4 hr | Site | Purpose |
| 1 | 7 | X | 1^{a} | Bioanalysis |
| | | | 2^{a} | Histopathology |
| | | | 3^{b} | Histopathology |
| | 21 | X | 4^{a} | Bioanalysis |
| | | | 5^{a} | Histopathology |

X = Sample to be collected; hr = hour
Note:
Sample collection sites as indicated below are general locations. The locations of collections may be modified as per the Study Director, in consultation with the Sponsor.
^{a}Sample within designated test site.
^{b}Sample outside designated test site. Will be as far away as feasible from test site (actual distance will be measured).

### Samples for Bioanalysis:

Prior to collection of samples, the area was rinsed thoroughly by wiping 10 times with gauze soaked in reverse osmosis (RO) water and then wiped with dry gauze. Prior to skin collection, the designated area was clipped and tape-stripped 10 times (1 pull per piece of Blenderm™ tape) to remove the stratum corneum. All tape strips were discarded following completion of tape-stripping procedures.

On days of collection, with a new scalpel the initial skin incision was made in a double crescent (football) shape (approximately 2 cm×1.5 cm) at the collection site. A different scalpel or clean scissors was used to isolate the skin sample from the subcutaneous layer. An intact layer of fat with thickness of approximately 0.5 cm on the skin sample was collected, if possible. Each skin sample was divided into two samples, with one being designated as the primary sample and the second as a backup. The samples were placed in appropriately labeled foil, frozen on dry ice and placed in a freezer set to maintain −70° C., until shipped. Samples were maintained on dry ice prior to being placed in the freezer.

The wounds remained open (not sutured) following creation. The wounds were packed with gauze and covered with Tegaderm™ adhesive dressing, with dressing removed for wound inspection or cleaning. The sites were wrapped with Co-Flex™ bandage and/or with Elastikon® as necessary to keep the Tegaderm™ in place. Tegaderm™/Co-Flex™ bandage and/or with Elastikon® were removed as necessary for observation of the sites and reapplied as above.

### Samples for Histopathology

On days of collection, 8 mm full thickness excisional wounds were created via punch on the dorsal surface. All skin samples for microscopic evaluation were at least 5 cm away from any previous skin incisions. Skin samples were collected, stored and preserved in 10% neutral buffered formalin until processed for evaluation.

The wounds remained open (not sutured) following creation. The wounds were be packed with gauze and covered with Tegaderm™ adhesive dressing, with dressing removed for wound inspection or cleaning. The sites were wrapped with Co-Flex™ bandage and/or with Elastikon® as necessary to keep the Tegaderm™ in place. Tegaderm™/Co-Flex™ bandage and/or with Elastikon® were removed as necessary for observation of the sites and reapplied as above.

### Analgesic/Antibiotic Therapy

Each animal was given buprenorphine (0.05 mg/kg IM) approximately every 8 to 12 hours after surgery at the discretion of the veterinarian. Meloxicam (0.2 mg/kg, SID PO) was given once daily after surgery at the discretion of the veterinarian. Should the animal show any classic signs of pain, including but not limited to anorexia, hyperpnea, pyrexia, or depression, additional analgesic agents were administered, if deemed appropriate by the Testing Facility veterinarian in conjunction with the Study Director. All analgesic changes, including drug, dose, route and site of administration were documented in the surgical records.

An appropriate antibiotic was administered. Drug, dose, route and site of administration were documented in the study records. Additional antibiotics were administered throughout the study, if deemed appropriate by the Testing Facility veterinarian in conjunction with the Study Director. All antibiotic changes were documented.

### Postoperative Monitoring

The Testing Facility Veterinary staff monitored the recovery of all animals and

evaluated the general health for at least 3 days following the surgical procedures. Individual observations (if any) were recorded.

### Anesthesia Reversal

Atipamezole (0.2 mg/kg, intramuscular injection) was administered as needed to reverse the effects of the sedatives used. Any resulting treatments were documented.

### Experimental Design for Toxicology Study on Ruxolitinib and Deuruxolitinib Foam Formulations

| | | | | | Dose | No. of Animals |
|---|---|---|---|---|---|---|
| Group | | Dose Level^{a} | Test | Dosing | Concentration | Main Study |
| No. | Test Material | (mg/cm²/dose) | Site | Days | (%) | Females^{b} |
| 1 | Test Article 1 | 4 | 1 | 1 to 7 | 2.5 | 4 |
| | (2.5% Ruxolitinib | | (left | | | |
| | Foam (LE, | | side) | | | |
| | 268-23-04)) | | | | | |
| | Test Article 2 | 4 | 2 | 15 to 21 | 2.5 | |
| | (2.5% Ruxolitinib | | (right | | | |
| | Foam (1E-05T, | | side) | | | |
| | 268-23-05)) | | | | | |

^{a}Based on the most recent body weight measurement.
^{b}The same animals will be dosed in 2 phases with at least a 7-day washout period between phases.

| | | | | | Dose | No. of Animals |
|---|---|---|---|---|---|---|
| Group | | Dose Level^{a} | Test | Dosing | Concentration | Main Study |
| No. | Test Material | (mg/cm²/dose) | Site | Days | (%) | Females^{b} |
| 2 | Test Article 1 | 4 | 1 | 1 to 7 | 2.5 | 4 |
| | (2.5% Deuruxolitinib | | (left | | | |
| | Foam (LE, | | side) | | | |
| | 268-24-01)) | | | | | |
| | Test Article 2 | 4 | 2 | 15 to 21 | 2.5 | |
| | (2.5% Deuruxolitinib | | (right | | | |
| | Foam (1E-05T, | | side) | | | |
| | 268-24-02)) | | | | | |

^{a}Based on the most recent body weight measurement.
^{b}The same animals will be dosed in 2 phases with at least a 7-day washout period between phases.

### Route of Administration/Application: Dermal

Treatment Frequency: Daily

Treatment Frequency per Day: BID (2×)-Days 1 to 6 and Days 15 to 20.

Target 6 hours (+15 minutes). SID (1×)-Days 7 and 21. On the final day of each phase, a single dose will be administered (following collection of the 24-hour TK sample collection)

Treatment Duration: Days 1 to 7 and Days 15 to 21

### Method:

The intended dose sites were prepared by close clipping of the hair, care was taken during the clipping procedure to avoid abrasion of the skin.

The dosing materials were applied directly to the skin over each designated area by gentle inunction with a disposable plastic applicator. Canisters were shaken a minimum of 5 times prior to each day's use. Material was applied to the treatment area within 3 minutes of dispensation from the test article canister. The target area (5% of the total body surface area per test site/test article) was covered with the appropriate dosing material. The area of application was estimated. All animals had intact skin. Animals were placed in a sling for dosing procedures and maintained in the sling for approximately 15 minutes following completion of dosing and rinsing procedures.

The target surface area for each animal was re-calculated once per week and marked accordingly (during the applicable dosing phase for that site) using the formula below for a 6 to 30 kg pig.

$$Total BSA ( {m}^{2} ) = ( 70 \times {BW ( kg )}^{0.75} ) / 1000$$

If the actual area covered by the dosing materials differs from the calculated test area, the actual application site was measured and documented in the raw data. In addition, the corners of the application site were marked to allow proper identification of the treated and untreated skin as often as needed.

Residual dosing materials were removed approximately 15 minutes after each administration by gently wiping the site with gauze soaked in reverse osmosis (RO) water, followed by dry gauze. The RO water was warmed in a water bath to 37° C. to 43° C. to aid in the removal of the dosing materials.

The first day of dosing for each animal was designated as Day 1 (alternate animals used for replacement after the initial Day 1 had their own unique Day 1 so all animals receive a full 7 days of dosing for each phase).

Dosing holidays were given as warranted by clinical signs, body weight losses, or other indications of severe toxicity. The duration of any dosing holiday and the animal(s) affected was documented in the study records.

### Clinical Pathology

### Sample Collection

### Clinical Pathology Sample Collection

| Group Nos. | Time Point | Hematology | Coagulation | Clinical Chemistry |
|---|---|---|---|---|
| All animals | Pretreatment | X | X | X |

| Unscheduled euthanasia | X | X | X |
|---|---|---|---|
| (when possible) | | | |
| Method/Comments: | Venipuncture of the | Venipuncture of the | Venipuncture of the |
| | Vena Cava^{a} | Vena Cava^{a} | Vena Cava^{a} |
| Target Volume (mL)^{b}: | 2 | 1.8 | 2 |
| Anticoagulant: | (K₂) EDTA | Sodium citrate | None |
| Special Requirements: | — | — | — |
| Processing: | None | Plasma | Serum |

X = Sample to be collected; — = Not applicable.
^{a}A peripheral vein may be used if necessary.
^{b}Additional samples may be obtained (e.g., due to clotting of non-serum samples) if permissible sampling frequency and volume are not exceeded.

### Hematology

### Hematology Parameters

| | |
|---|---|
| Red blood cell count | White blood cell count |
| Hemoglobin concentration | Neutrophil count (absolute) |
| Hematocrit | Lymphocyte count (absolute) |
| Mean corpuscular volume | Monocyte count (absolute) |
| Red blood cell distribution width | Eosinophil count (absolute) |
| Mean corpuscular hemoglobin concentration | Basophil count (absolute) |
| Mean corpuscular hemoglobin | Large unstained cells (absolute) |
| Reticulocyte count (absolute) | Other cells (as appropriate) |
| Platelet count | |

One blood smear was prepared from each hematology sample. The slide was labeled, stained, and archived. Slide review was only be performed on samples that meet flagging criteria in order to confirm accurate hematology analyzer results. If additional examination of blood smears was deemed necessary, the smears were subsequently evaluated at additional cost by protocol amendment.

### Coagulation

### Coagulation Parameters

| | |
|---|---|
| Activated partial thromboplastin time | Prothrombin time |
| Fibrinogen | Sample quality |

### Clinical Chemistry

### Clinical Chemistry Parameters

| | |
|---|---|
| Alanine aminotransferase | Albumin |
| Aspartate aminotransferase | Globulin (calculated) |
| Alkaline phosphatase | Albumin/globulin ratio |
| Gamma-glutamyltransferase | Glucose |
| Creatine kinase | Cholesterol |
| Total bilirubin^{a} | Triglycerides |
| Urea nitrogen | Sodium |
| Creatinine | Potassium |
| Calcium | Chloride |
| Phosphorus | Sorbitol Dehydrogenase^{b} |
| Total protein | Lactate Dehydrogenase |
| | Sample quality |

^{a}When total bilirubin is >1.0 mg/dL, direct bilirubin was measured and indirect bilirubin was calculated.
^{b}When an unscheduled collection occurs, Sorbitol Dehydrogenase was not analyzed and reported.

### Bioanalysis and Toxicokinetic Evaluation

### Bioanalytical Sample Collection

### Bioanalytical Sample Collection

| | Target Time Post first daily dose on | | | | | | | |
|---|---|---|---|---|---|---|---|---|
| | Days 1, 6, 15 and 20 | | | | | | | |
| | 0 | 1 | 3 | 6 | 7 | 9 | 12 | 24 |
| Group Nos. | hr ^{a} | hr | hr | hr ^{b} | hr | hr | hr | hr ^{a} |
| 1 | X | X | X | X | X | X | X | X |

| | |
|---|---|
| Unscheduled euthanasia | Before euthanasia |
| (when possible) | |
| Method/Comments: | Venipuncture of a suitable peripheral vein |
| | or the vena cava |
| Target Volume (mL): | 1 |
| Anticoagulant: | K₃EDTA |
| Special Requirements: | Tubes will be chilled after blood collection. |
| Processing: | Plasma |

X = Sample to be collected; hr = hour.
^{a} Sample will be collected before the first daily dose.
^{b} Sample will be collected before the second daily dose.

| Group Nos. | Target Time Post Dose on Day 21 |
|---|---|
| 1 | 4 hr^{a} |
| Unscheduled euthanasia (when | Before euthanasia |
| possible) | |
| Method/Comments: | Venipuncture of a suitable peripheral vein or the vena |
| | cava |
| Target Volume (mL): | 1 |
| Anticoagulant: | K₃EDTA |
| Special Requirements: | Tubes will be chilled after blood collection. |
| Processing: | Plasma |

X = Sample to be collected; hr = hour.
^{a}Sample will be collected prior to biopsy collection.

### Bioanalytical Sample Processing

The samples were centrifuged within 1 hour of collection and the resultant plasma was separated, split into two approximately equal aliquots, transferred to uniquely labeled polypropylene tubes, and frozen in a freezer set to maintain −70° C. If necessary, the samples were frozen on dry ice prior to being placed in the freezer.

### Bioanalytical Sample Analysis

Plasma samples were analyzed for concentration of ruxolitinib using a qualified analytical procedure.

Analysis was performed according to the SOPs of the performing laboratory. Analyst software will be used for data acquisitions and for chromatographic peak integrations and Watson LIMS was used for the calculations of the concentration data. Specific software versions were noted in the bioanalytical report.

Incurred sample reanalysis was not be performed as part of this study.

### Toxicokinetic Evaluation

A non-compartmental approach consistent with the dermal route of administration was used for parameter estimation. All TK parameters were generated from individual plasma concentrations of Ruxolitinib on Days 1, 6, 15, and 20 for female minipigs in Groups 1. The toxicokinetic parameters that were calculated (as data permit) include, but are not limited to, Cmax, Css, and area under the plasma concentration versus time curve (AUC). Toxicokinetic parameters were calculated using Phoenix® WinNonlin® Version 8.3.4. Concentration values reported as below the quantitation limit were assigned a value of zero.

### Parameters to be Estimated

| Parameter | Description of Parameter |
|---|---|
| Cₛₛ | Mean concentration at steady-state was calculated by dividing the AUC value by |
| | the dosing interval (24 hour). |
| Cₘₐₓ | The maximum observed concentration measured. |
| AUC₀₋ₜ | The area under the concentration time curve from time zero to t, where t denotes |
| | a specific sampling time following dosing calculated using the linear trapezoidal |
| | linear interpolation method. |
| AUC₀₋ₜ/Dose | The AUC₀₋ₜ normalized for dose. |
| tₗₐₛₜ | The time after dosing at which the last quantifiable concentration was observed. |

### Results

Dermal Observations for Ruxolitinib Foam Formulations. The ruxolitinib foam formulations did not show dermal signs of edema or erythema resulting from application of the test article (i.e., showed Grade 0). While there was eschar grade 1 observed, this was not test article related, but rather due to the wound healing from biopsy. Further, there were no microscopic findings related to application of test article.

TABLE 62
2.5% Topical Ruxolitinib Foam (2.5% Emollient, 268-23-04) PEG300/Glycerin (Days
1-4) (PRD1 = PreDose 1; PRD2 = PreDose 2; X = Present)

| 4 mg/cm2/ | | | | | | | | |
|---|---|---|---|---|---|---|---|---|
| dose | | | | | | | | |
| Group 1 | | Day(s) Relative to Start Date | | | | | | |
| Sex: | Observation Type: | 1 | 1 | 2 | 2 | 3 | 3 | 4 |
| Female | Local Irritation Ext 1 | PRD1 | PRD2 | PRD1 | PRD2 | PRD1 | PRD2 | PRD1 |
| 1501 | Edema, Site No. 01, Grade 0 | X | X | X | X | X | X | X |
| | Eschar, Site No. 01, Grade 1 | — | — | — | — | X | X | X |
| | Erythema, Site No. 01, Grade 0 | X | X | X | X | X | X | X |
| 1502 | Edema, Site No. 01, Grade 0 | X | X | X | X | X | X | X |
| | Erythema, Site No. 01, Grade 0 | X | X | X | X | X | X | X |
| 1503 | Edema, Site No. 01, Grade 0 | X | X | X | X | X | X | X |
| | Erythema, Site No. 01, Grade 0 | X | X | X | X | X | X | X |
| 1504 | Edema, Site No. 01, Grade 0 | X | X | X | X | X | X | X |
| | Erythema, Site No. 01, Grade 0 | X | X | X | X | X | X | X |

TABLE 63
2.5% Topical Ruxolitinib Foam (2.5% Emollient, 268-23-04) PEG300/Glycerin (Days
4-7) (PRD1 = PreDose 1; PRD2 = PreDose 2; X = Present)

| 4 mg/cm2/ | | | | | | | |
|---|---|---|---|---|---|---|---|
| dose | | | | | | | |
| Group 1 | | Day(s) Relative to Start Date | | | | | |
| Sex: | Observation Type: | 4 | 5 | 5 | 6 | 6 | 7 |
| Female | Local Irritation Ext 1 | PRD2 | PRD1 | PRD2 | PRD1 | PRD2 | PRD1 |
| 1501 | Edema, Site No. 01, Grade 0 | X | X | X | X | X | X |
| | Eschar, Site No. 01, Grade 1 | X | X | X | X | X | — |
| | Erythema, Site No. 01, Grade 0 | X | X | X | X | X | X |
| 1502 | Edema, Site No. 01, Grade 0 | X | X | X | X | X | X |
| | Erythema, Site No. 01, Grade 0 | X | X | X | X | X | X |
| 1503 | Edema, Site No. 01, Grade 0 | X | X | X | X | X | X |
| | Erythema, Site No. 01, Grade 0 | X | X | X | X | X | X |
| 1504 | Edema, Site No. 01, Grade 0 | X | X | X | X | X | X |
| | Erythema, Site No. 01, Grade 0 | X | X | X | X | X | X |

TABLE 64
2.5% Topical Ruxolitinib (1% Emollient, 268-23-05) Myristyl Lactate/Transcutol-P
(Days 15-18) (PRD1 = PreDose 1; PRD2 = PreDose 2; X = Present)

| 4 mg/cm2/ | | | | | | | | |
|---|---|---|---|---|---|---|---|---|
| dose | | | | | | | | |
| Group 1 | | Day(s) Relative to Start Date | | | | | | |
| Sex: | Observation Type: | 15 | 15 | 16 | 16 | 17 | 17 | 18 |
| Female | Local Irritation Ext 1 | PRD1 | PRD2 | PRD1 | PRD2 | PRD1 | PRD2 | PRD1 |
| 1501 | Edema, Site No. 02, Grade 0 | X | X | X | X | X | X | X |
| | Erythema, Site No. 02, Grade 0 | X | X | X | X | X | X | X |
| 1502 | Edema, Site No. 02, Grade 0 | X | X | X | X | X | X | X |
| | Erythema, Site No. 02, Grade 0 | X | X | X | X | X | X | X |
| 1503 | Edema, Site No. 02, Grade 0 | X | X | X | X | X | X | X |
| | Erythema, Site No. 02, Grade 0 | X | X | X | X | X | X | X |
| 1504 | Edema, Site No. 02, Grade 0 | X | X | X | X | X | X | X |
| | Erythema, Site No. 02, Grade 0 | X | X | X | X | X | X | X |

TABLE 65
2.5% Topical Ruxolitinib (1% Emollient, 268-23-05) Myristyl Lactate/Transcutol-P
(Days 18-21) (PRD1 = PreDose 1; PRD2 = PreDose 2; X = Present)

| 4 mg/cm2/ | | | | | | | |
|---|---|---|---|---|---|---|---|
| dose | | | | | | | |
| Group 1 | | Day(s) Relative to Start Date | | | | | |
| Sex: | Observation Type: | 18 | 19 | 19 | 20 | 20 | 21 |
| Female | Local Irritation Ext 1 | PRD2 | PRD1 | PRD2 | PRD1 | PRD2 | PRD1 |
| 1501 | Edema, Site No. 02, Grade 0 | X | X | X | X | X | X |
| | Erythema, Site No. 02, Grade 0 | X | X | X | X | X | X |
| 1502 | Edema, Site No. 02, Grade 0 | X | X | X | X | X | X |
| | Erythema, Site No. 02, Grade 0 | X | X | X | X | X | X |
| 1503 | Edema, Site No. 02, Grade 0 | X | X | X | X | X | X |
| | Erythema, Site No. 02, Grade 0 | X | X | X | X | X | X |
| 1504 | Edema, Site No. 02, Grade 0 | X | X | X | X | X | X |
| | Erythema, Site No. 02, Grade 0 | X | X | X | X | X | X |

Dermal observations for Deuruxolitinib formulations. The deuruxolitinib foam formulations did not show dermal signs of edema or erythema resulting from application of the test article (i.e., showed Grade 0). While there was eschar grade 1 observed, this was not test article related, but rather due to the wound healing from biopsy. Further, there were no microscopic findings related to application of test article.

TABLE 66
2.5% Topical Deuruxolitinib Foam (2.5% Emollient, 268-24-01) PEG300/Glycerin
(Days 1-4) (PRD1 = PreDose 1; PRD2 = PreDose 2; X = Present)

| 4 mg/cm2/ | | | | | | | | |
|---|---|---|---|---|---|---|---|---|
| dose | | | | | | | | |
| Group 1 | | Day(s) Relative to Start Date | | | | | | |
| Sex: | Observation Type: | 1 | 1 | 2 | 2 | 3 | 3 | 4 |
| Female | Local Irritation Ext 1 | PRD1 | PRD2 | PRD1 | PRD2 | PRD1 | PRD2 | PRD1 |
| 1501 | Edema, Site No. 01, Grade 0 | X | X | X | X | X | X | X |
| | Eschar, Site No. 01, Grade 1 | X | X | X | X | X | X | X |
| | Erythema, Site No. 01, Grade 0 | X | X | X | X | X | X | X |
| 1502 | Edema, Site No. 01, Grade 0 | X | X | X | X | X | X | X |
| | Erythema, Site No. 01, Grade 0 | X | X | X | X | X | X | X |
| 1503 | Edema, Site No. 01, Grade 0 | X | X | X | X | X | X | X |
| | Erythema, Site No. 01, Grade 0 | X | X | X | X | X | X | X |
| 1504 | Edema, Site No. 01, Grade 0 | X | X | X | X | X | X | X |
| | Erythema, Site No. 01, Grade 0 | X | X | X | X | X | X | X |

TABLE 67
2.5% Topical Deuruxolitinib Foam (2.5% Emollient, 268-24-01) PEG300/Glycerin
(Days 4-7) (PRD1 = PreDose 1; PRD2 = PreDose 2; X = Present)

| 4 mg/cm2/ | | | | | | | |
|---|---|---|---|---|---|---|---|
| dose | | | | | | | |
| Group 1 | | Day(s) Relative to Start Date | | | | | |
| Sex: | Observation Type: | 4 | 5 | 5 | 6 | 6 | 7 |
| Female | Local Irritation Ext 1 | PRD2 | PRD1 | PRD2 | PRD1 | PRD2 | PRD1 |
| 1501 | Edema, Site No. 01, Grade 0 | X | X | X | X | X | X |
| | Eschar, Site No. 01, Grade 1 | X | X | X | X | X | X |
| | Erythema, Site No. 01, Grade 0 | X | X | X | X | X | X |
| 1502 | Edema, Site No. 01, Grade 0 | X | X | X | X | X | X |
| | Erythema, Site No. 01, Grade 0 | X | X | X | X | X | X |
| 1503 | Edema, Site No. 01, Grade 0 | X | X | X | X | X | X |
| | Erythema, Site No. 01, Grade 0 | X | X | X | X | X | X |
| | Edema, Site No. 01, Grade 0 | X | X | X | X | X | X |
| 1504 | Edema, Site No. 01, Grade 0 | X | X | X | X | X | X |
| | Erythema, Site No. 01, Grade 0 | X | X | X | X | X | X |

TABLE 68
2.5% Topical Deuruxolitinib (1% Emollient, 268-24-02) Myristyl Lactate/Transcutol-P
(Days 14-18) (PRD1 = PreDose 1; PRD2 = PreDose 2; X = Present)

| 4 mg/cm2/ | | | | | | | | |
|---|---|---|---|---|---|---|---|---|
| dose | | | | | | | | |
| Group 1 | | Day(s) Relative to Start Date | | | | | | |
| Sex: | Observation Type: | 15 | 15 | 16 | 16 | 17 | 17 | 18 |
| Female | Local Irritation Ext 1 | PRD1 | PRD2 | PRD1 | PRD2 | PRD1 | PRD2 | PRD1 |
| 1501 | Edema, Site No. 02, Grade 0 | X | X | X | X | X | X | X |
| | Erythema, Site No. 02, Grade 0 | X | X | X | X | X | X | X |
| 1502 | Edema, Site No. 02, Grade 0 | X | X | X | X | X | X | X |
| | Erythema, Site No. 02, Grade 0 | X | X | X | X | X | X | X |
| 1503 | Edema, Site No. 02, Grade 0 | X | X | X | X | X | X | X |
| | Erythema, Site No. 02, Grade 0 | X | X | X | X | X | X | X |
| | | — | — | — | — | — | — | — |
| 1504 | Edema, Site No. 02, Grade 0 | X | X | X | X | X | X | X |
| | Erythema, Site No. 02, Grade 0 | X | X | X | X | X | X | X |

TABLE 69
2.5% Topical Deuruxolitinib (1% Emollient, 268-24-02) Myristyl Lactate/Transcutol-P
(Days 18-21) (PRD1 = PreDose 1; PRD2 = PreDose 2; X = Present)

| 4 mg/cm2/ | | | | | | | |
|---|---|---|---|---|---|---|---|
| dose | | | | | | | |
| Group 1 | | Day(s) Relative to Start Date | | | | | |
| Sex: | Observation Type: | 18 | 19 | 19 | 20 | 20 | 21 |
| Female | Local Irritation Ext 1 | PRD2 | PRD1 | PRD2 | PRD1 | PRD2 | PRD1 |
| 1501 | Edema, Site No. 02, Grade 0 | X | X | X | X | X | X |
| | Erythema, Site No. 02, Grade 0 | X | X | X | X | X | X |
| 1502 | Edema, Site No. 02, Grade 0 | X | X | X | X | X | X |
| | Erythema, Site No. 02, Grade 0 | X | X | X | X | X | X |
| 1503 | Edema, Site No. 02, Grade 0 | X | X | X | X | X | X |
| | Erythema, Site No. 02, Grade 0 | — | X | X | X | X | X |
| | Erythema, Site No 02, Grade 0 | X | — | — | — | — | — |
| 1504 | Edema, Site No. 01, Grade 0 | X | X | X | X | X | X |
| | Erythema, Site No. 01, Grade 0 | X | X | X | X | X | X |

### Systemic Exposure of Topical Foams (Ruxolitinib)

The measurement of the systemic exposure of ruxolitinib and deuruxolitinib did not indicate therapeutic relevant concentrations.

TABLE 70
Ruxolitinib plasma concentrations after the topical application of foam formulations
(BLOQ = Below the limit of quantitation (0.300 ng/ml or 0.979 nM)

| | | | Time | Ruxolitinib | Ruxolitinib |
|---|---|---|---|---|---|
| | Animal | Study | points | concentration | concentration |
| Formulation | ID | Day | (Hr) | (ng/ml) | (nM) |
| 2.5% Ruxolitinib | 1501 | 6 | 0 | BLOQ | BLOQ |
| PEG300/Glycerin | 1501 | 6 | 1 | BLOQ | BLOQ |
| 268-23-04 | 1501 | 6 | 3 | 0.538 | 1.76 |
| | 1501 | 6 | 6 | BLOQ | BLOQ |
| | 1501 | 6 | 7 | BLOQ | BLOQ |
| | 1501 | 6 | 9 | 0.567 | 1.85 |
| | 1501 | 6 | 12 | BLOQ | BLOQ |
| | 1501 | 6 | 24 | 0.475 | 1.55 |
| | 1502 | 6 | 0 | 0.572 | 1.87 |
| | 1502 | 6 | 1 | BLOQ | BLOQ |
| | 1502 | 6 | 3 | BLOQ | BLOQ |
| | 1502 | 6 | 6 | 0.72 | 2.35 |
| | 1502 | 6 | 7 | BLOQ | BLOQ |
| | 1502 | 6 | 9 | 1.61 | 5.25 |
| | 1502 | 6 | 12 | BLOQ | BLOQ |
| | 1502 | 6 | 24 | BLOQ | BLOQ |
| | 1503 | 6 | 0 | BLOQ | BLOQ |
| | 1503 | 6 | 1 | BLOQ | BLOQ |
| | 1503 | 6 | 3 | BLOQ | BLOQ |
| | 1503 | 6 | 6 | 0.575 | 1.88 |
| | 1503 | 6 | 7 | 1.83 | 5.97 |
| | 1503 | 6 | 9 | 0.497 | 1.62 |
| | 1503 | 6 | 12 | BLOQ | BLOQ |
| | 1503 | 6 | 24 | BLOQ | BLOQ |
| | 1504 | 6 | 0 | 0.42 | 1.37 |
| | 1504 | 6 | 1 | BLOQ | BLOQ |
| | 1504 | 6 | 3 | BLOQ | BLOQ |
| | 1504 | 6 | 6 | 0.551 | 1.8 |
| | 1504 | 6 | 7 | BLOQ | BLOQ |
| | 1504 | 6 | 9 | 0.735 | 2.4 |
| | 1504 | 6 | 12 | BLOQ | BLOQ |
| | 1504 | 6 | 24 | 1.34 | 4.36 |
| 2.5% Ruxolitinib | 1501 | 20 | 0 | BLOQ | BLOQ |
| ML/Transcutol-P | 1501 | 20 | 1 | BLOQ | BLOQ |
| 268-23-05 | 1501 | 20 | 3 | BLOQ | BLOQ |
| | 1501 | 20 | 6 | 0.38 | 1.24 |
| | 1501 | 20 | 7 | BLOQ | BLOQ |
| | 1501 | 20 | 9 | BLOQ | BLOQ |
| | 1501 | 20 | 12 | 1.5 | 4.91 |
| | 1501 | 20 | 24 | 0.791 | 2.58 |
| | 1502 | 20 | 0 | BLOQ | BLOQ |
| | 1502 | 20 | 1 | 0.374 | 1.22 |
| | 1502 | 20 | 3 | 0.54 | 1.76 |
| | 1502 | 20 | 6 | 0.349 | 1.14 |
| | 1502 | 20 | 7 | 0.482 | 1.57 |
| | 1502 | 20 | 9 | BLOQ | BLOQ |
| | 1502 | 20 | 12 | 1.07 | 3.49 |
| | 1502 | 20 | 24 | 0.303 | 0.988 |
| | 1503 | 20 | 0 | BLOQ | BLOQ |
| | 1503 | 20 | 1 | 1.36 | 4.45 |
| | 1503 | 20 | 3 | 0.316 | 1.03 |
| | 1503 | 20 | 6 | BLOQ | BLOQ |
| | 1503 | 20 | 7 | BLOQ | BLOQ |
| | 1503 | 20 | 9 | 0.75 | 2.45 |
| | 1503 | 20 | 12 | 0.697 | 2.27 |
| | 1503 | 20 | 24 | 0.452 | 1.48 |
| | 1504 | 20 | 0 | 0.412 | 1.35 |
| | 1504 | 20 | 1 | 0.38 | 1.24 |
| | 1504 | 20 | 3 | BLOQ | BLOQ |
| | 1504 | 20 | 6 | 1.87 | 6.1 |
| | 1504 | 20 | 7 | 0.746 | 2.44 |
| | 1504 | 20 | 9 | 1.51 | 4.92 |
| | 1504 | 20 | 12 | 1.18 | 3.86 |
| | 1504 | 20 | 24 | 0.349 | 1.14 |

From Table 70, the systemic concentration measured were below therapeutic levels.

### Determining Skin Concentration of Ruxolitinib or Deuruxolitinib

The procedure for sampling of skin from the minipig after topical applications of the ruxolitinib foams is outlined above. The procedure for the separation of the skin layers is listed below.
(1) Pig skin samples are allowed to thaw at room temperature. Once thawed, keep on wet ice until needed.
(2) Handling
(a) Cutting board preparation
(i) Use a totally flat, solid cutting board with no indentations or depressions
(ii) Apply a strip of ½″ lab tape along the edges
(ii) Apply a second strip of lab tape on top of the first
(iii) Press down lab tape firmly
(b) Place a piece of foil on the benchtop
(c) Remove pig skin sample from plastic bag and place on foil, epidermis side up
(d) Blot the epidermal surface dry with kimwipes
(e) Place a piece of surgical adhesive on the epidermis and press down very firmly to ensure it sticks well
(f) Take the skin sample and place it epidermis side down (which is now covered with surgical adhesive) on a cutting board
(g) With a clean scalpel, cut a ˜1″×1″ piece of skin from a corner.
(i) Remove the adhesive from this piece of skin
(ii) Wrap in a single layer of aluminum foil
(iii) Place in a labeled zip lock bag
(iv) Return to −80 C for later use
(h) With a clean 6 mm biopsy punch, and with skin still epidermis side down, make a sample punch near the middle of the skin sample
(i) Apply a single drop of super glue to the prepared cutting board, right next to the lab tape
(j) Press the skin punch, epidermis side down, into the super glue and allow to dry (˜10 min)
(k) Place the used biopsy punch in a beaker filled with ethanol for cleaning and re-use
(l) Repeat steps h-j twice more for the skin sample to generate a total of three punches. Be sure to use a clean, dry 6 mm punch each time.
(m) Separation of skin layers
(i) Using clean forceps, press down firmly on the top of one of the skin punches
(ii) Using a clean #22 scalpel, and the double strips of lab tape as a guide, cut the punch into dermis and epidermis sections
(iii) Transfer the dermis to a pre-weighed, labelled homog tube
(iv) Peel the epidermis off the surgical adhesive with forceps, and transfer to a pre-weighed, labelled homog tube
(v) Rinse forceps with ethanol and dry
(vi) Place the scalpel in a beaker with ethanol

The samples were homogenized with an extraction solution and the amount of ruxolitinib was quantified in the epidermis/upper dermis and dermis.

TABLE 71
Ruxolitinib skin Concentrations

| | | Animal # | | | | |
|---|---|---|---|---|---|---|
| | Skin | 1501 | 1502 | 1503 | 1504 | Mean |
| Formulation | layer | Ruxolitinib amount (ng) | | | | |
| PEG300/ | Dermis | 57.03 ± 34.98 | 26.43 ± 7.33 | 20.57 ± 3.77 | 48.00 ± 10.48 | 38.01 ± 17.32 |
| Glycerin | Epidermis/ | 548.00 ± 134.55 | 237.33 ± 27.50 | 124.67 ± 21.08 | 163.20 ± 78.70 | 268.30 ± 192.24 |
| 268-23-04 | Upper | | | | | |
| | dermis | | | | | |
| ML/ | Dermis | 39.60 ± 19.86 | 80.87 ± 28.20 | 135.87 ± 175.66 | 33.67 ± 24.87 | 72.5 ± 47.17 |
| Transcutol | Epidermis/ | 666.67 ± 306.89 | 463.33 ± 189.41 | 214.87 ± 151.40 | 84.63 ± 34.70 | 357.38 ± 259.22 |
| 268-23-05 | Upper | | | | | |
| | dermis | | | | | |

### Systemic Exposure of Topical Foams (Deuruxolitinib)

TABLE 72
Deuruxolitinib plasma concentrations after the topical application of foam formulations
(BLOQ = Below the limit of quantification (0.300 ng/ml or 0.954 nM)

| | | | Time | Ruxolitinib | Ruxolitinib |
|---|---|---|---|---|---|
| | Animal | Study | points | concentration | concentration |
| Formulation | ID | Day | (Hr) | (ng/ml) | (nM) |
| 2.5% Deurruxolitinib | 1501 | 6 | 0 | BLOQ | BLOQ |
| PEG300/Glycerin | 1501 | 6 | 1 | BLOQ | BLOQ |
| 268-24-01 | 1501 | 6 | 3 | 0.367 | 1.17 |
| | 1501 | 6 | 6 | BLOQ | BLOQ |
| | 1501 | 6 | 7 | BLOQ | BLOQ |
| | 1501 | 6 | 9 | 0.453 | 1.44 |
| | 1501 | 6 | 12 | BLOQ | BLOQ |
| | 1501 | 6 | 24 | 1.74 | 5.54 |
| | 1502 | 6 | 0 | BLOQ | BLOQ |
| | 1502 | 6 | 1 | BLOQ | BLOQ |
| | 1502 | 6 | 3 | BLOQ | BLOQ |
| | 1502 | 6 | 6 | 1.47 | 4.67 |
| | 1502 | 6 | 7 | BLOQ | BLOQ |
| | 1502 | 6 | 9 | 0.386 | 1.23 |
| | 1502 | 6 | 12 | 1.09 | 3.46 |
| | 1502 | 6 | 24 | 2.03 | 6.47 |
| | 1503 | 6 | 0 | BLOQ | BLOQ |
| | 1503 | 6 | 1 | BLOQ | BLOQ |
| | 1503 | 6 | 3 | 0.730 | 2.32 |
| | 1503 | 6 | 6 | 0.594 | 1.89 |
| | 1503 | 6 | 7 | 0.346 | 1.10 |
| | 1503 | 6 | 9 | 1.17 | 3.74 |
| | 1503 | 6 | 12 | 0.308 | 0.979 |
| | 1503 | 6 | 24 | 2.40 | 7.62 |
| | 1504 | 6 | 0 | BLOQ | BLOQ |
| | 1504 | 6 | 1 | 1.84 | 5.86 |
| | 1504 | 6 | 3 | BLOQ | BLOQ |
| | 1504 | 6 | 6 | 0.704 | 2.24 |
| | 1504 | 6 | 7 | BLOQ | BLOQ |
| | 1504 | 6 | 9 | 1.97 | 6.28 |
| | 1504 | 6 | 12 | BLOQ | BLOQ |
| | 1504 | 6 | 24 | 7.70 | 24.5 |
| 2.5% Deurruxolitinib | 1501 | 20 | 0 | BLOQ | BLOQ |
| ML/Transcutol-P | 1501 | 20 | 1 | BLOQ | BLOQ |
| 268-24-02 | 1501 | 20 | 3 | 0.310 | 0.987 |
| | 1501 | 20 | 6 | 0.452 | 1.44 |
| | 1501 | 20 | 7 | 1.06 | 3.38 |
| | 1501 | 20 | 9 | BLOQ | BLOQ |
| | 1501 | 20 | 12 | BLOQ | BLOQ |
| | 1501 | 20 | 24 | 0.566 | 1.80 |
| | 1502 | 20 | 0 | BLOQ | BLOQ |
| | 1502 | 20 | 1 | 0.395 | 1.26 |
| | 1502 | 20 | 3 | BLOQ | BLOQ |
| | 1502 | 20 | 6 | 0.348 | 1.11 |
| | 1502 | 20 | 7 | BLOQ | BLOQ |
| | 1502 | 20 | 9 | BLOQ | BLOQ |
| | 1502 | 20 | 12 | BLOQ | BLOQ |
| | 1502 | 20 | 24 | 0.364 | 1.16 |
| | 1503 | 20 | 0 | 1.43 | 4.55 |
| | 1503 | 20 | 1 | BLOQ | BLOQ |
| | 1503 | 20 | 3 | 0.368 | 1.17 |
| | 1503 | 20 | 6 | BLOQ | BLOQ |
| | 1503 | 20 | 7 | BLOQ | BLOQ |
| | 1503 | 20 | 9 | 0.316 | 1.01 |
| | 1503 | 20 | 12 | BLOQ | BLOQ |
| | 1503 | 20 | 24 | 0.385 | 1.22 |
| | 1504 | 20 | 0 | 0.448 | 1.42 |
| | 1504 | 20 | 1 | 1.40 | 4.44 |
| | 1504 | 20 | 3 | 0.699 | 2.22 |
| | 1504 | 20 | 6 | 1.14 | 3.63 |
| | 1504 | 20 | 7 | 4.08 | 13.0 |
| | 1504 | 20 | 9 | BLOQ | BLOQ |
| | 1504 | 20 | 12 | BLOQ | BLOQ |
| | 1504 | 20 | 24 | 0.738 | 2.35 |

Note:
Sample 1502 Day 6 1 hr had aberrant IS response at 175% of the mean.

### From the GLP Minipig Toxicology Study:

GLP measurement: No dermal findings (e.g., irritation, edema, redness, etc.) were observed for the 4 formulations listed.

Histology did not indicate any dermal findings.

Minimal systemic exposure and measurement indicated concentrations below therapeutic levels.

Skin concentration indicated the delivery to the epidermis and dermis in a minipig model.

TABLE 13
Deuruxolitinib skin concentrations

| | | Animal # | | | | |
|---|---|---|---|---|---|---|
| | Skin | 1501 | 1502 | 1503 | 1504 | Mean |
| Formulation | layer | deuruxolitinib amount (ng) | | | | |
| PEG300/ | Dermis | 128.54 ± 198.78 | 98.27 ± 97.46 | 15.31 ± 6.59 | 1.87 ± 3.24 | 61.00 ± 62.00 |
| Glycerin | Epidermis/ | 241.33 ± 85.29 | 368.00 ± 264.02 | 200.33 ± 131.15 | 149.47 ± 62.96 | 239.78 ± 93.37 |
| 268-24-01 | Upper | | | | | |
| | dermis | | | | | |
| 2.5% | Dermis | 128.33 ± 75.17 | 38.63 ± 29.24 | 18.10 ± 8.68 | 10.76 ± 3.66 | 48.96 ± 54.22 |
| Deuterated | Epidermis/ | 153.93 ± 94.49 | 257.67 ± 136.24 | 108.97 ± 20.28 | 271.33 ± 121.14 | 197.98 ± 78.18 |
| ruxolitinib | Upper | | | | | |
| ML/Transcutol-P | dermis | | | | | |
| 268-24-02 | | | | | | |

1-73. (canceled)

74. A foamable composition suitable for application as a foam to a body surface area affected by seborrheic dermatitis in a human patient, comprising a foamable carrier component and a propellant component; wherein the foamable carrier component comprises a compound, which is ruxolitinib or deuterated ruxolitinib, or a pharmaceutically acceptable salt of any of the aforementioned.

75. The foamable composition according to claim 74, wherein the foamable carrier component is a homogeneous emulsion.

76. The foamable composition according to claim 74, wherein the foamable composition is a homogeneous emulsion.

77. The foamable composition according to claim 74, wherein the foamable carrier component further comprises water, a solvent component, and an oil phase.

78. The foamable composition according to claim 77, wherein the oil phase comprises about 0.5% to about 20% by weight of the foamable carrier composition.

79. The foamable composition according to claim 77, wherein the oil phase comprises about 0.5% to about 10% by weight of the foamable carrier composition.

80. The foamable composition according to claim 77, wherein the oil phase comprises about 1% to about 10% by weight of the foamable carrier composition.

81. The foamable composition according to claim 74, wherein the compound is ruxolitinib, or a pharmaceutically acceptable salt thereof.

82. The foamable composition according to claim 74, wherein the compound is ruxolitinib phosphate.

83. The foamable composition according to claim 74, wherein the compound is deuruxolitinib or a pharmaceutically acceptable salt thereof.

84. A method for treating seborrheic dermatitis in a human patient in need thereof comprising administering to a body surface area affected by the seborrheic dermatitis of the patient a foam produced according to claim 74.

85. A foamable composition suitable for application as a foam to a body surface area affected by an inflammatory or autoimmune skin or hair disease in a human patient, comprising a foamable carrier component and a propellant component, wherein the foamable carrier component comprises:
from about 0.5% to about 3%, of a compound, which is ruxolitinib or deuruxolitinib, or a pharmaceutically acceptable salt thereof, by weight of the foamable carrier composition,
from about 80% to about 90% of a hydroethanolic mixture, by weight of the foamable carrier composition,
from about 1% to about 3% of an emollient component, by weight of the foamable carrier composition, wherein the emollient component comprises at least one emollient and at least one co-solvent,
from about 1% to about 5% of one or more C₁₆₋₁₈ fatty alcohols, by weight of the foamable carrier composition,
from about 0.5% to about 3% of an emulsifier component, by weight of the foamable carrier composition, and
from about 4% to about 6% of a solvent, by weight of the foamable carrier composition;
wherein the hydroethanolic mixture is a mixture of ethanol and water, the ethanol is present in amount ranging from about 50% to about 70% of the hydroethanolic mixture, and the water is present in an amount ranging from about 30% to about 50% of the hydroethanolic mixture.

86. The foamable composition according to claim 85, wherein:
the emulsifier component is polysorbate 60 (Tween 60);
the emollient is glycerin and the co-solvent is polyethylene glycol 300;
the one or more C₁₆₋₁₈ fatty alcohols are cetyl alcohol and stearyl alcohol;
the solvent is propylene glycol; and
the compound is ruxolitinib phosphate in an amount of about 2.5% on a free base basis, of the foamable carrier component.

87. The foamable composition according to claim 85, wherein:
the emulsifier component is polysorbate 60 (Tween 60);
the emollient is glycerin and the co-solvent is polyethylene glycol 300;
the one or more C₁₆₋₁₈ fatty alcohols are cetyl alcohol and stearyl alcohol;
the solvent is propylene glycol; and
the compound is ruxolitinib phosphate in an amount of about 1.5% on a free base basis, of the foamable carrier component.

88. The foamable composition according to claim 85, wherein:
the emulsifier component is polysorbate 60 (Tween 60);
the emollient is glycerin and the co-solvent is polyethylene glycol 300;
the one or more C₁₆₋₁₈ fatty alcohols are cetyl alcohol and stearyl alcohol;
the solvent is propylene glycol; and
the compound is deuruxolitinib phosphate in an amount of about 2.5% on a free base basis, of the foamable carrier component.

89. The foamable composition according to claim 85, wherein:
the emulsifier component is polysorbate 60 (Tween 60);
the emollient is glycerin and the co-solvent is polyethylene glycol 300;
the one or more C₁₆₋₁₈ fatty alcohols are cetyl alcohol and stearyl alcohol;
the solvent is propylene glycol; and
the compound is deuruxolitinib phosphate in an amount of about 1.5% on a free base basis, of the foamable carrier component.

90. The foamable composition according to claim 85, wherein:
the emulsifier component is polysorbate 60 (Tween 60);
the emollient is myristyl lactate and the co-solvent is transcutol-P;
the one or more C₁₆₋₁₈ fatty alcohols are cetyl alcohol and stearyl alcohol;
the solvent is propylene glycol; and
the compound is ruxolitinib phosphate in an amount of about 2.5% on a free base basis, of the foamable carrier component.

91. The foamable composition according to claim 85, wherein:
the emulsifier component is polysorbate 60 (Tween 60);
the emollient is myristyl lactate and the co-solvent is transcutol-P;
the one or more C₁₆₋₁₈ fatty alcohols are cetyl alcohol and stearyl alcohol;
the solvent is propylene glycol; and
the compound is ruxolitinib phosphate in an amount of about 1.5% on a free base basis, of the foamable carrier component.

92. The foamable composition according to claim 85, wherein:
the emulsifier component is polysorbate 60 (Tween 60);
the emollient is myristyl lactate and the co-solvent is transcutol-P;
the one or more C₁₆₋₁₈ fatty alcohols are cetyl alcohol and stearyl alcohol;
the solvent is propylene glycol; and
the compound is deuruxolitinib phosphate in an amount of about 2.5% on a free base basis, of the foamable carrier component.

93. The foamable composition according to claim 85, wherein:
the emulsifier component is polysorbate 60 (Tween 60);
the emollient is myristyl lactate and the co-solvent is transcutol-P;
the one or more C₁₆₋₁₈ fatty alcohols are cetyl alcohol and stearyl alcohol;
the solvent is propylene glycol; and
the compound is deuruxolitinib phosphate in an amount of about 1.5% on a free base basis, of the foamable carrier component.

94. The foamable composition according to claim 85, wherein:
the compound or the salt is present in an amount of 1.5% or 2.5%, by weight of the foamable carrier composition,
the hydroethanolic mixture is present in an amount from about 80% to about 90%, by weight of the foamable carrier composition,
the emollient component is present in an amount from about 2% to about 3%, by weight of the foamable carrier composition, wherein the emollient component comprises about 0.3% to about 0.6% of an emollient and about 1.8% to about 2.2% of a co-solvent, by weight of the foamable carrier composition,
the one or more C₁₆₋₁₈ fatty alcohols are cetyl alcohol and stearyl alcohol,
the cetyl alcohol is present in an amount from about 2% to about 2.5%, by weight of the foamable carrier composition,
the stearyl alcohol is present in an amount from about 0.25% to about 0.5%, by weight of the foamable carrier composition, and
the emulsifier component is present in an amount from about 1% to about 2%, by weight of the foamable carrier composition,
the solvent is present in an amount from about 4% to about 6%, by weight of the foamable carrier composition,
wherein the hydroethanolic mixture is a mixture of ethanol and water, the ethanol is present in amount ranging from about 55% to about 65% of the hydroethanolic mixture, and the water is present in an amount ranging from about 35% to about 45% of the hydroethanolic mixture.

95. The foamable composition according to claim 94, wherein:
the emulsifier component is polysorbate 60 (Tween 60);
the emollient is glycerin and the co-solvent is polyethylene glycol 300,
the solvent is propylene glycol; and
the compound is ruxolitinib phosphate in an amount of about 2.5% on a free base basis, of the foamable carrier component.

96. The foamable composition according to claim 94, wherein:
the emulsifier component is polysorbate 60 (Tween 60);
the emollient is glycerin and the co-solvent is polyethylene glycol 300;
the solvent is propylene glycol; and
the compound is ruxolitinib phosphate in an amount of about 1.5% on a free base basis, of the foamable carrier component.

97. The foamable composition according to claim 94, wherein:
the emulsifier component is polysorbate 60 (Tween 60);
the emollient is glycerin and the co-solvent is polyethylene glycol 300;
the solvent is propylene glycol; and
the compound is deuruxolitinib phosphate in an amount of about 2.5% on a free base basis, of the foamable carrier component.

98. The foamable composition according to claim 94, wherein:
the emulsifier component is polysorbate 60 (Tween 60);
the emollient is glycerin and the co-solvent is polyethylene glycol 300;
the solvent is propylene glycol; and
the compound is deuruxolitinib phosphate in an amount of about 1.5% on a free base basis, of the foamable carrier component.

99. The foamable composition according to claim 85, wherein:
the compound or the salt is present in an amount of 1.5% or 2.5%, by weight of the foamable carrier composition,
the hydroethanolic mixture is present in an amount from about 80% to about 90%, by weight of the foamable carrier composition,
the emollient component is present in an amount from about 1% to about 2%, by weight of the foamable carrier composition, wherein the emollient component comprises about 0.8% to about 1.2% of an emollient and about 0.3% to about 0.6% of a co-solvent, by weight of the foamable carrier composition,
the one or more C₁₆₋₁₈ fatty alcohols are cetyl alcohol and stearyl alcohol;
the cetyl alcohol is present in an amount from about 2% to about 2.5%, by weight of the foamable carrier composition,
the stearyl alcohol is present in an amount of from about 0.6% to about 0.9%, by weight of the foamable carrier composition,
the emulsifier component is present in an amount from about 1% to about 2%, by weight of the foamable carrier composition, and
the solvent is present in an amount from about 4% to about 6%, by weight of the foamable carrier composition,
wherein the hydroethanolic mixture is a mixture of ethanol and water, the ethanol is present in amount ranging from about 55% to about 65% of the hydroethanolic mixture, and the water is present in an amount ranging from about 35% to about 45% of the hydroethanolic mixture.

100. The foamable composition according to claim 99, wherein:
the emulsifier component is polysorbate 60 (Tween 60);
the emollient is myristyl lactate and the co-solvent is transcutol-P,
the solvent is propylene glycol; and
the compound is ruxolitinib phosphate in an amount of about 2.5% on a free base basis, of the foamable carrier component.

101. The foamable composition according to claim 99, wherein:
the emulsifier component is polysorbate 60 (Tween 60);
the emollient is myristyl lactate and the co-solvent is transcutol-P;
the solvent is propylene glycol; and
the compound is ruxolitinib phosphate in an amount of about 1.5% on a free base basis, of the foamable carrier component.

102. The foamable composition according to claim 99, wherein:
the emulsifier component is polysorbate 60 (Tween 60);
the emollient is myristyl lactate and the co-solvent is transcutol-P;
the solvent is propylene glycol; and
the compound is deuruxolitinib phosphate in an amount of about 2.5% on a free base basis, of the foamable carrier component.

103. The foamable composition according to claim 99, wherein:
the emulsifier component is polysorbate 60 (Tween 60);
the emollient is myristyl lactate and the co-solvent is transcutol-P the solvent is propylene glycol; and
the compound is deuruxolitinib phosphate in an amount of about 1.5% on a free base basis, of the foamable carrier component.

104. The foamable composition according to claim 94, wherein:
the emollient component comprises PEG 300 in an amount of about 2% and glycerin in an amount of about 0.5% by weight of the foamable carrier component.

105. The foamable composition according to claim 99, wherein:
the emollient component comprises myristyl lactate in an amount of about 1%, by weight of the foamable carrier component and transcutol-P in an amount of about 0.5% by weight of the foamable carrier component.

106. The foamable composition according to claim 85, wherein the pH of the foamable composition ranges from about 5.0 to about 8.0.

107. The foamable composition according to claim 106, wherein the pH of the foamable composition ranges from about 5.0 to about 6.0.

108. The foamable composition according to claim 106, wherein the pH is adjusted by addition of trolamine to the foamable carrier composition.

109. The foamable composition according to claim 107, wherein the pH is adjusted by addition of trolamine to the foamable carrier composition.

110. A foam produced by expelling the foamable composition according to claim 85 from a pressurized container.

111. The foam according to claim 110, wherein the foamable composition is aerosolized.

112. A method for treating an inflammatory or autoimmune skin or hair disease in a human patient in need thereof comprising administering to a body surface area affected by the disease of the patient a foam produced according to claim 110.

113. The method of claim 112, wherein the inflammatory or autoimmune skin or hair disease is alopecia.

114. The method according to claim 112, wherein the alopecia is alopecia areata.

115. The method according to claim 114, wherein the alopecia areata is mild to moderate.

116. The method according to claim 114, wherein the alopecia areata is severe.

117. The method according to claim 114, wherein the alopecia areata is acute.

118. The method according to claim 114, wherein the alopecia areata is chronic.

119. The method according to claim 112, wherein the inflammatory or autoimmune skin or hair disease is seborrheic dermatitis.
